(19) United States
(12) Patent Application Publication
(10) Pub. No.: US 2017/0133601 A1
(43) Pub. Date: May 11, 2017

(54) COMPOUND AND ORGANIC LIGHT EMITTING DEVICE INCLUDING SAME

(71) Applicant: SAMSUNG DISPLAY CO., LTD., Yongin-si (KR)

(72) Inventors: Munki Sim, Yongin-si (KR); Youngkook Kim, Yongin-si (KR); Junha Park, Yongin-si (KR); Eunyoung Lee, Yongin-si (KR); Hyoyoung Lee, Yongin-si (KR); Eunjae Jeong, Yongin-si (KR); Seokhwan Hwang, Yongin-si (KR)

(21) Appl. No.: 15/179,788

(22) Filed: Jun. 10, 2016

(30) Foreign Application Priority Data
Nov. 6, 2015 (KR) 10-2015-0155519

(51) Int. Cl. H01L 51/00; C07D 495/04; C07F 9/6561; H01L 27/32; C07D 491/048

(52) U.S. Cl. CPC H01L 51/0071; H01L 27/3244; C07D 491/048; H01L 51/0067; H01L 51/0072; H01L 51/0058; H01L 51/0052; C07F 9/6561; C07D 495/04; H01L 51/0073; H01L 51/5072

(57) ABSTRACT

An organic light-emitting device includes: a first electrode; a second electrode facing the first electrode; and an organic layer between the first electrode and the second electrode, the organic layer including an emission layer, wherein the organic layer includes a compound represented by Formula 1. The organic light-emitting device including the compound may have high efficiency, low voltage, high luminance, and a long lifespan.

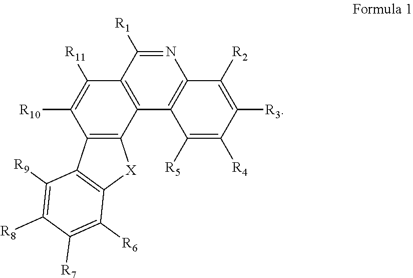

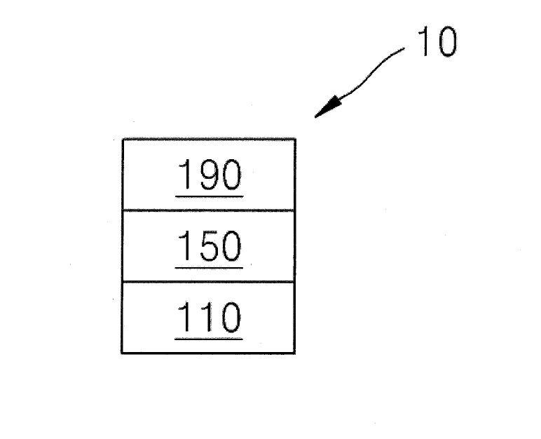

### CROSS-REFERENCE TO RELATED APPLICATION

This application claims priority to and the benefit of Korean Patent Application No. 10-2015-0155519, filed on Nov. 6, 2015, in the Korean Intellectual Property Office, the entire content of which is incorporated herein by reference.

### BACKGROUND

1. Field

One or more aspects of embodiments of the present disclosure relate to a compound and an organic light-emitting device including the same.

2. Description of the Related Art

Organic light-emitting devices are self-emission devices that have wide viewing angles, high contrast ratios, short response times, and excellent brightness, driving voltage, and response speed characteristics, and can produce full-color images.

An organic light-emitting device may include a first electrode disposed (e.g., positioned) on a substrate, and a hole transport region, an emission layer, an electron transport region, and a second electrode, which are sequentially disposed on the first electrode. Holes provided from the first electrode, for example, may move toward the emission layer through the hole transport region, and electrons provided from the second electrode, for example, may move toward the emission layer through the electron transport region. Carriers, such as holes and electrons, may then recombine in the emission layer to produce excitons. These excitons transition from an excited state to a ground state, thereby generating light.

### SUMMARY

One or more aspects of embodiments of the present disclosure are directed toward a compound that is suitable for use as an electron injection material or electron transport material, and that has high electron transport capability and high material stability, and an organic light-emitting device including the compound and having high efficiency, low voltage, high luminance, and a long lifespan.

Additional aspects will be set forth in part in the description which follows and, in part, will be apparent from the description, or may be learned by practice of the presented embodiments.

According to one or more embodiments, a compound according to embodiments is represented by Formula 1:

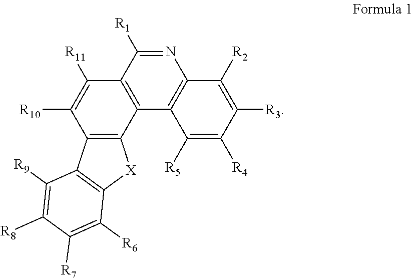

In Formula 1,

X may be O or S;

R₁ to R₁₁ may each independently be selected from hydrogen, deuterium, a halogen, a nitro group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

two adjacent substituents selected from R₁ to R₁₁ may be optionally linked to each other to form a ring; and

at least one substituent of the substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₂-C₁₀ heterocycloalkyl group, substituted C₃-C₁₀ cycloalkenyl group, substituted C₂-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group may be selected from the group consisting of:

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—P(═O)(Q₂₈)(Q₂₉),

wherein Q₁₁ to Q₁₇ and Q₂₁ to Q₂₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

According to one or more embodiments, an organic light-emitting device includes a first electrode; a second electrode facing the first electrode; and an organic layer between the first electrode and the second electrode, the organic layer including an emission layer, wherein the organic layer includes the compound represented by Formula 1.

According to one or more embodiments, a display apparatus includes the organic light-emitting device, wherein the first electrode of the organic light-emitting device is electrically coupled to a source electrode or drain electrode of a thin film transistor.

### BRIEF DESCRIPTION OF THE DRAWING

These and/or other aspects will become apparent and more readily appreciated from the following description of the embodiments, taken in conjunction with the drawing, which is a schematic view of an organic light-emitting device according to an embodiment.

### DETAILED DESCRIPTION

Reference will now be made in more detail to embodiments, examples of which are illustrated in the accompanying drawing, wherein like reference numerals refer to like elements throughout. In this regard, the present embodiments may have different forms and should not be construed as being limited to the descriptions set forth herein. Accordingly, the embodiments are merely described below, by referring to the drawing, to explain aspects of the present description. As used herein, the term “and/or” includes any and all combinations of one or more of the associated listed items. Expressions such as “at least one of,” “one of,” and “selected from,” when preceding a list of elements, modify the entire list of elements and do not modify the individual elements of the list. Further, the use of “may” when describing embodiments of the present invention refers to one or more embodiments of the present invention.”

A compound according to one or more embodiments of the present disclosure is represented by Formula 1:

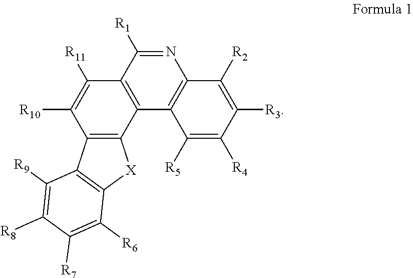

In Formula 1,

X may be O or S;

R₁ to R₁₁ may each independently be selected from hydrogen, deuterium, a halogen, a nitro group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

two adjacent substituents selected from R₁ to R₁₁ may be optionally linked to each other to form a ring; and

at least one substituent of the substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₂-C₁₀ heterocycloalkyl group, substituted C₃-C₁₀ cycloalkenyl group, substituted C₂-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, and substituted monovalent non-aromatic condensed polycyclic group, and substituted monovalent non-aromatic condensed heteropolycyclic group may be selected from the group consisting of:

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—P(═O)(Q₂₈)(Q₂₉),

wherein Q₁₁ to Q₁₇ and Q₂₁ to Q₂₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

For example, organic mono-molecular materials selected from organometallic complexes may be suitable for use as electron transport materials, due to their stability with respect to electrons and electron movement speed characteristics. For example, Alq3 has been described as having high stability and high electron affinity. However, when Alq3 is used in a blue light-emitting device, due to exciton diffusion-derived emission, color purity may deteriorate. Also, flavone derivatives and derivatives of germanium and silicon chloropentadiene may be used as electron transport materials.

Non-limiting examples of the organic mono-molecular material include a 2-biphenyl-4-yl-5-(4-t-butylphenyl)-1,3,4-oxadiazole (PBD) derivative linked to a spiro compound, and 2,2′,2″-(benzene-1,3,5-triyl)-tris(1 -phenyl-1 H-benzimidazole) (TPBI) having a hole blocking capability and an excellent electron transport capability. For example, a benzoimidazole derivative has been described as having excellent durability.

However, an organic light-emitting device including an electron transport layer having the organic mono-molecular material as described above has a short emission lifespan, and low preservative durability and reliability. This is at least in part due to physical and/or chemical change of an organic material, photochemical and/or electrochemical change of an organic material, oxidation of a cathode, exfoliation, and/or durability.

One or more embodiments of the present disclosure provide a novel heterocyclic compound and an organic light-emitting device including an organic film having the compound. The compound including a heteroring (e.g., a ring containing at least one heteroatom) according to an embodiment of the present disclosure may have excellent (or suitable) electric characteristics, a high charge transport capability, a high luminescent capability, a high glass transition temperature, crystallization-prevention (or crystallization-reduction) characteristics, and may be used as an electron transport material that is suitable for red, green, blue, and/or white fluorescent and/or phosphorescent device. When the compound is used in manufacturing an organic light-emitting device, the manufactured organic light-emitting device may have high efficiency, low voltage, high brightness, and a long lifespan.

In various embodiments, R₂ to R₆ in Formula 1 may each independently be hydrogen or deuterium.

In various embodiments, in Formula 1, R₁ may be -(L₁)ₐ-(R₂₁)_{b}; and R₃ may be -(L₃)_{c}-(R₂₃)_{d};

descriptions of R₂₁ and R₂₃ may each independently be the same as the description provided in connection with R₁ to R₁₁;

L₁ and L₃ may each independently be selected from an unsubstituted C₆-C₆₀ arylene group, an unsubstituted C₁-C₆₀ heteroarylene group, an unsubstituted divalent non-aromatic condensed polycyclic group, and an unsubstituted divalent non-aromatic condensed heteropolycyclic group;

a, b, c, and d may each independently be an integer selected from 0 to 3.

In various embodiments, L₁ and L₃ may each independently be represented by one selected from Formulae 2a to 2e:

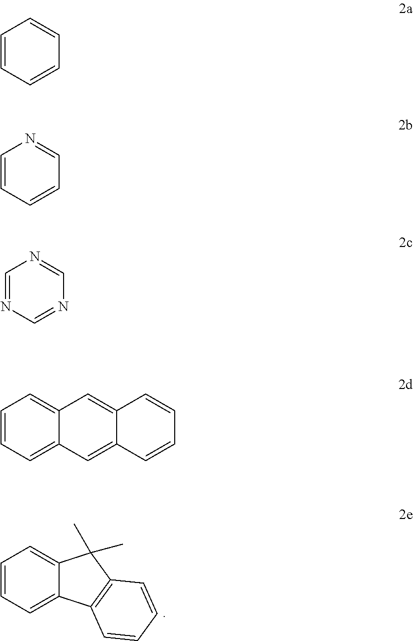

L₁ and L₃ may each independently be a divalent or trivalent linking group, and in Formulae 2a to 2e, linking may occur where hydrogen is present (e.g., any hydrogen in Formulae 2a to 2e may be substituted for a binding site).

In various embodiments, R₂₁ and R₂₃ may each independently be selected from hydrogen, deuterium, and a group represented by one selected from Formulae 3a to 3k:

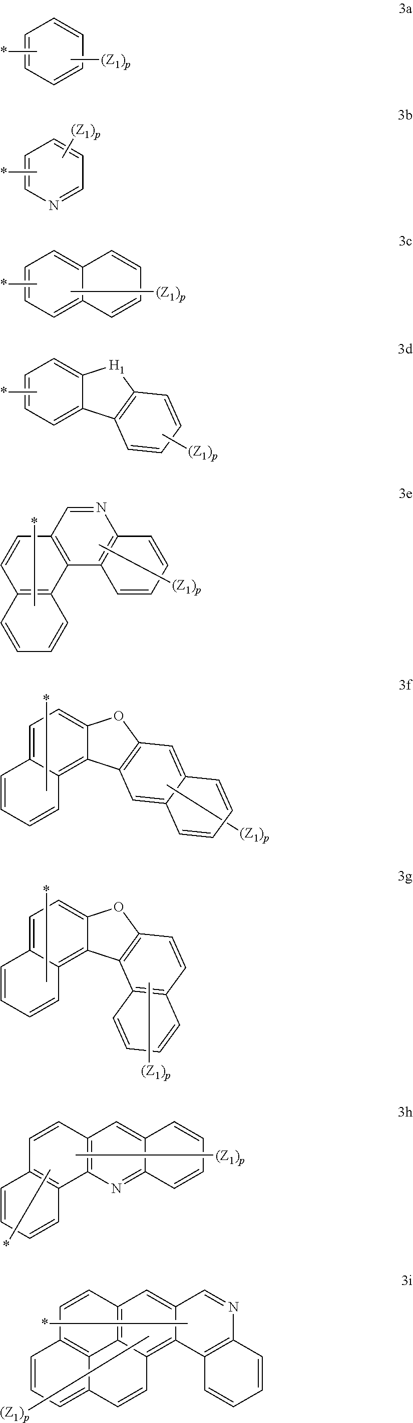

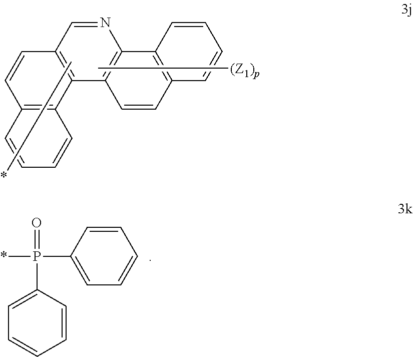

In Formulae 3a to 3k, H₁ may be CR₃₁R₃₂, N R₃₃, O, or S,

R₃₁ to R₃₃, and Z₁ may each independently be selected from hydrogen, deuterium, a halogen, a cyano group, a nitro group, a hydroxyl group, a carboxy group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, and a substituted or unsubstituted monovalent non-aromatic condensed heteropolycyclic group;

p in Formulae 3a and 3e may be an integer selected from 1 to 5, p in Formulae 3b and 3d may be an integer selected from 1 to 4, p in Formulae 3c, 3h, and 3j may be an integer selected from 1 to 7, p in Formulae 3f, 3g, and 3i may be an integer selected from 1 to 6, and when p is two or more, a plurality of Z₁(s) may be identical to or different from each other; and

*indicates a bonding site.

In various embodiments, when H₁ in Formula 3d is CR₃₁R₃₂, R₃₁ and R₃₂ may be linked to each other to form a ring. For example, Formula 3d may represent a spiro compound.

In various embodiments, the compound represented by Formula 1 may be represented by Formula 2:

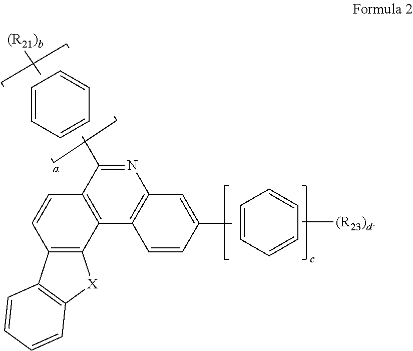

In Formula 2, descriptions of R₂₁ and R₂₃ may each independently be the same as the description provided in connection with R₁ to R₁₁; and

a, b, c, and d may each independently be an integer selected from 0 to 3.

In Formula 2, X is the same as described above (hereinafter, X is regarded as the same as described above).

In various embodiments, the compound represented by Formula 1 may be represented by Formula 3:

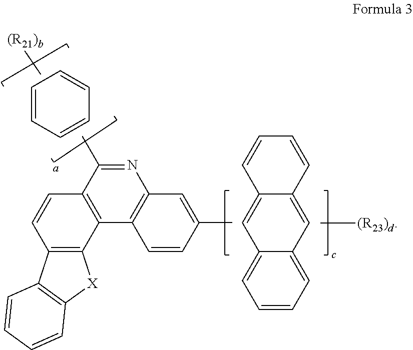

In Formula 3, descriptions of R₂₁ and R₂₃ may each independently be the same as the description provided in connection with R₁ to R₁₁; and

a, b, c, and d may each independently an integer selected from 0 to 3.

In various embodiments, the compound represented by Formula 1 may be represented by Formula 4:

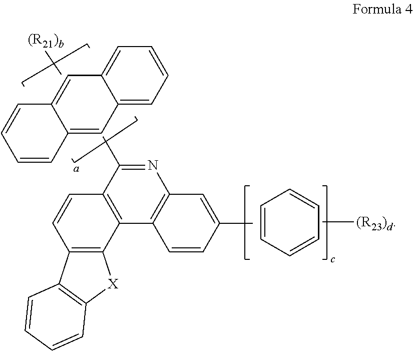

In Formula 4, descriptions of R₂₁ and R₂₃ may each independently be the same as the description provided in connection with R₁ to R₁₁; and

a, b, c, and d may each independently an integer selected from 0 to 3.

In various embodiments, the compound represented by Formula 1 may be represented by Formula 5:

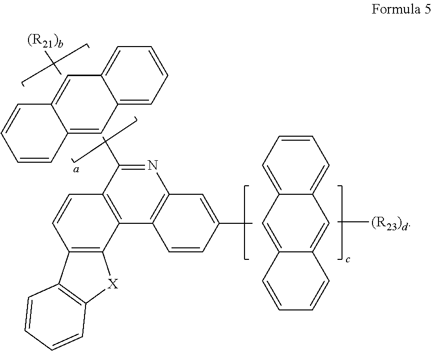

In Formula 5, descriptions of R₂₁ and R₂₃ may each independently be the same as the description provided in connection with R₁ to R₁₁; and

a, b, c, and d are each independently an integer selected from 0 to 3.

In various embodiments, the compound represented by Formula 1 may be represented by Formula 6:

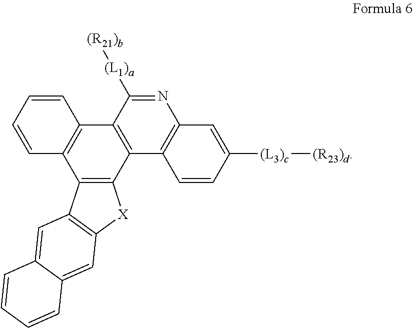

In Formula 6, descriptions of R₂₁ and R₂₃ may each independently be the same as the description provided in connection with R₁ to R₁₁;

L₁ and L₃ may each independently be selected from an unsubstituted C₆-C₆₀ arylene group, an unsubstituted C₁-C₆₀ heteroarylene group, an unsubstituted divalent non-aromatic condensed polycyclic group, and an unsubstituted divalent non-aromatic condensed heteropolycyclic group; and

a, b, c, and d may each independently be an integer selected from 0 to 3.

In various embodiments, the compound represented by Formula 1 may be represented by Formula 7:

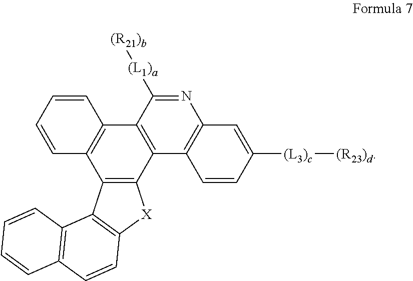

In Formula 7, descriptions of R₂₁ and R₂₃ may each independently be the same as the description provided in connection with R₁ to R₁₁;

L₁ and L₃ may each independently be selected from an unsubstituted C₆-C₆₀ arylene group, an unsubstituted C₁-C₆₀ heteroarylene group, an unsubstituted divalent non-aromatic condensed polycyclic group, and an unsubstituted divalent non-aromatic condensed heteropolycyclic group;

a, b, c, and d may each independently be an integer selected from 0 to 3.

In various embodiments, the compound of Formula 1 may be a compound selected from the following Compounds1 to 172, but is not limited thereto:

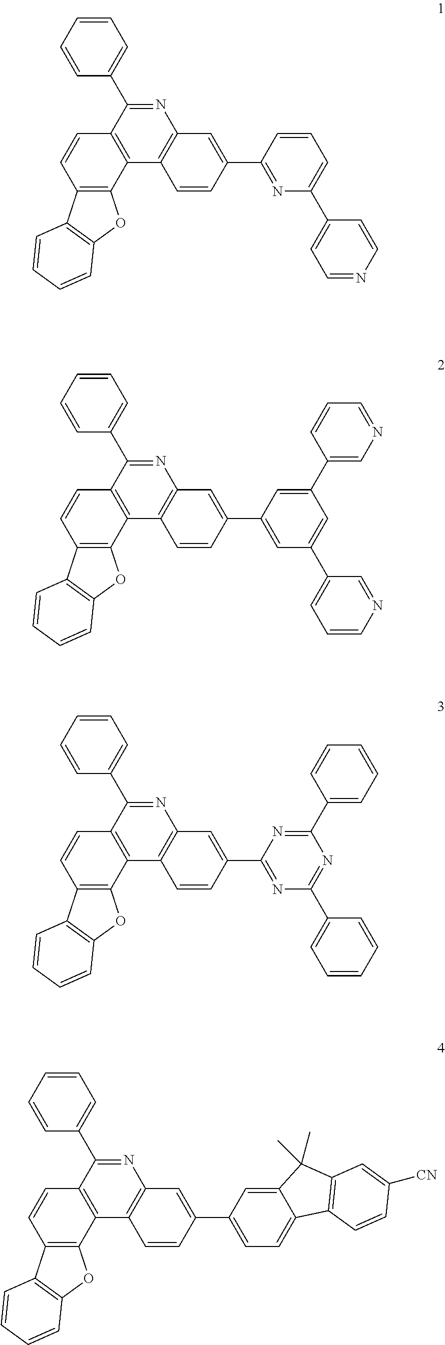 [SMILES: c1ccc(-c2nc3cc(-c4cccc(-c5ccncc5)n4)ccc3c3c2ccc2c4ccccc4oc23)cc1 ; c1ccc(-c2nc3cc(-c4cc(-c5cccnc5)cc(-c5cccnc5)c4)ccc3c3c2ccc2c4ccccc4oc23)cc1 ; c1ccc(-c2nc(-c3ccccc3)nc(-c3ccc4c(c3)nc(-c3ccccc3)c3ccc5c6ccccc6oc5c34)n2)cc1 ; CC1(C)c2cc(C#N)ccc2-c2ccc(-c3ccc4c(c3)nc(-c3ccccc3)c3ccc5c6ccccc6oc5c34)cc21]

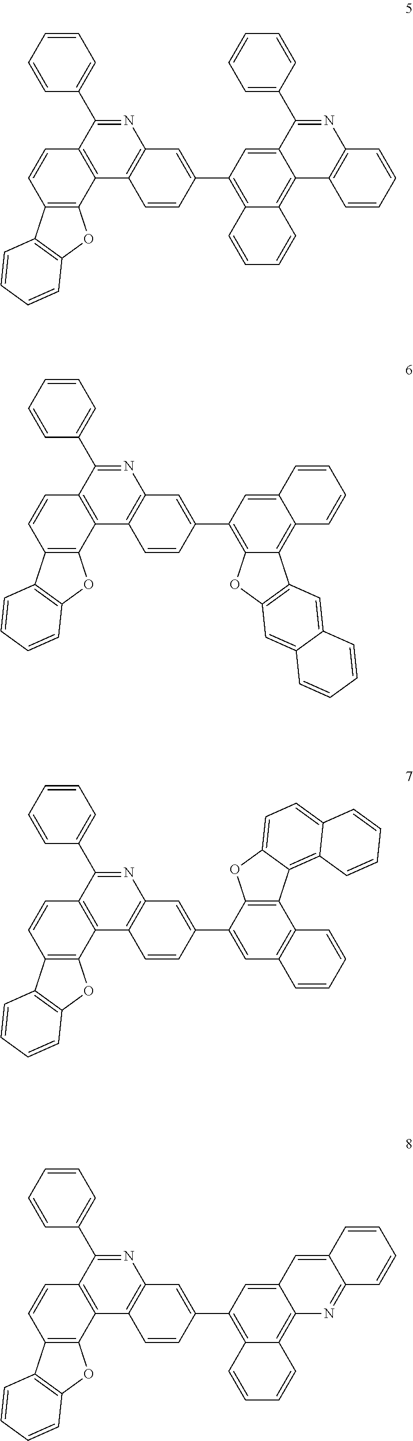 [SMILES: c1ccc(-c2nc3ccccc3c3c2cc(-c2ccc4c(c2)nc(-c2ccccc2)c2ccc5c6ccccc6oc5c24)c2ccccc23)cc1 ; c1ccc(-c2nc3cc(-c4cc5ccccc5c5c4oc4cc6ccccc6cc45)ccc3c3c2ccc2c4ccccc4oc23)cc1 ; c1ccc(-c2nc3cc(-c4cc5ccccc5c5c4oc4ccc6ccccc6c45)ccc3c3c2ccc2c4ccccc4oc23)cc1 ; c1ccc(-c2nc3cc(-c4cc5cc6ccccc6nc5c5ccccc45)ccc3c3c2ccc2c4ccccc4oc23)cc1]

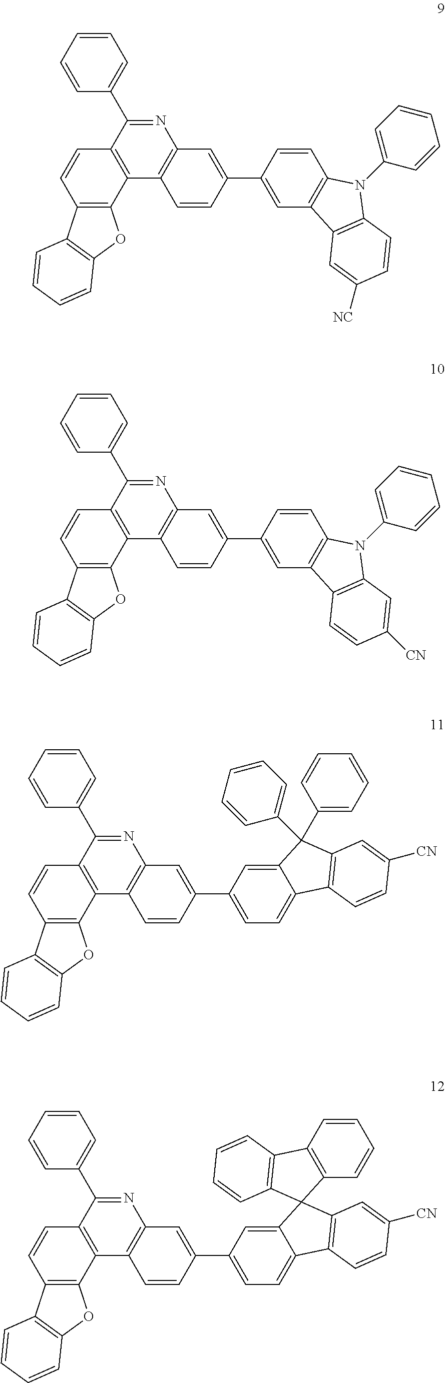 [SMILES: N#Cc1ccc2c(c1)c1cc(-c3ccc4c(c3)nc(-c3ccccc3)c3ccc5c6ccccc6oc5c34)ccc1n2-c1ccccc1 ; N#Cc1ccc2c3cc(-c4ccc5c(c4)nc(-c4ccccc4)c4ccc6c7ccccc7oc6c45)ccc3n(-c3ccccc3)c2c1 ; N#Cc1ccc2c(c1)C(c1ccccc1)(c1ccccc1)c1cc(-c3ccc4c(c3)nc(-c3ccccc3)c3ccc5c6ccccc6oc5c34)ccc1-2 ; N#Cc1ccc2c(c1)C1(c3ccccc3-c3ccccc31)c1cc(-c3ccc4c(c3)nc(-c3ccccc3)c3ccc5c6ccccc6oc5c34)ccc1-2]

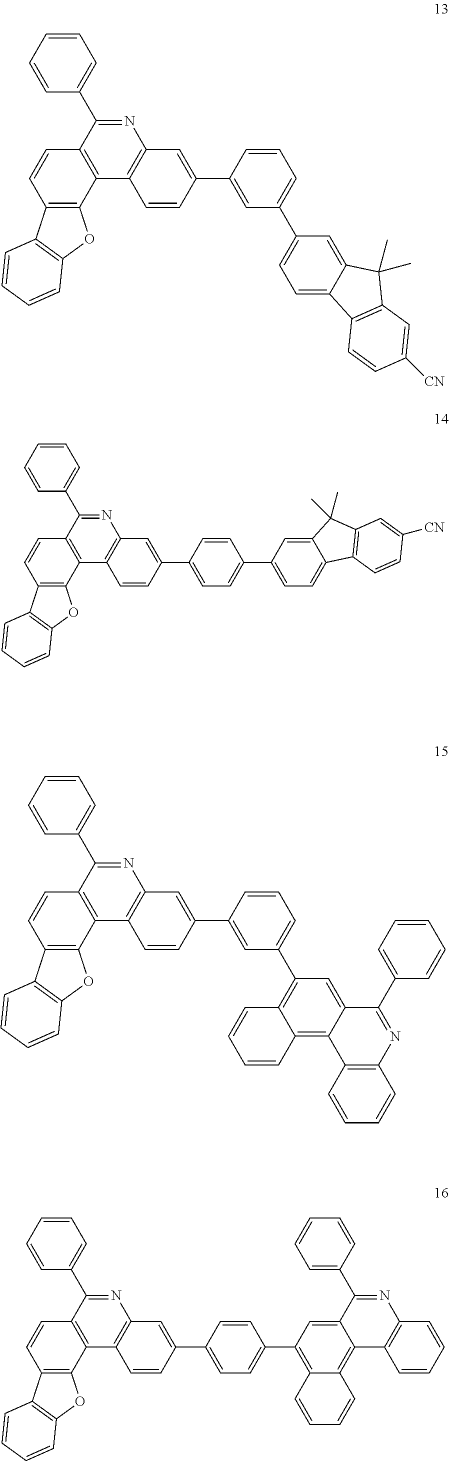 [SMILES: CC1(C)c2cc(C#N)ccc2-c2ccc(-c3cccc(-c4ccc5c(c4)nc(-c4ccccc4)c4ccc6c7ccccc7oc6c45)c3)cc21 ; CC1(C)c2cc(C#N)ccc2-c2ccc(-c3ccc(-c4ccc5c(c4)nc(-c4ccccc4)c4ccc6c7ccccc7oc6c45)cc3)cc21 ; c1ccc(-c2nc3ccccc3c3c2cc(-c2cccc(-c4ccc5c(c4)nc(-c4ccccc4)c4ccc6c7ccccc7oc6c45)c2)c2ccccc23)cc1 ; c1ccc(-c2nc3ccccc3c3c2cc(-c2ccc(-c4ccc5c(c4)nc(-c4ccccc4)c4ccc6c7ccccc7oc6c45)cc2)c2ccccc23)cc1]

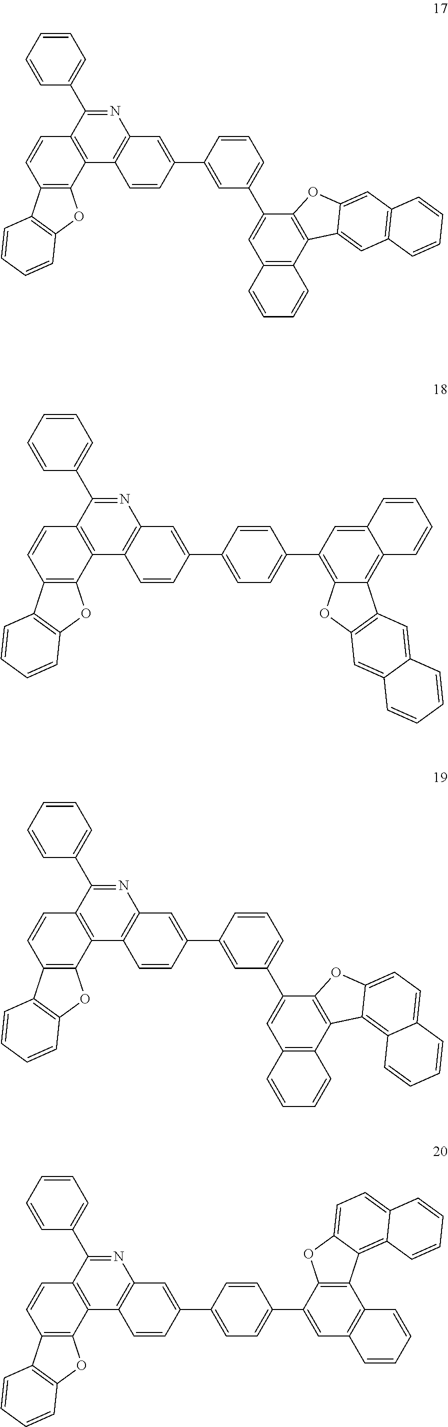 [SMILES: c1ccc(-c2nc3cc(-c4cccc(-c5cc6ccccc6c6c5oc5cc7ccccc7cc56)c4)ccc3c3c2ccc2c4ccccc4oc23)cc1 ; c1ccc(-c2nc3cc(-c4ccc(-c5cc6ccccc6c6c5oc5cc7ccccc7cc56)cc4)ccc3c3c2ccc2c4ccccc4oc23)cc1 ; c1ccc(-c2nc3cc(-c4cccc(-c5cc6ccccc6c6c5oc5ccc7ccccc7c56)c4)ccc3c3c2ccc2c4ccccc4oc23)cc1 ; c1ccc(-c2nc3cc(-c4ccc(-c5cc6ccccc6c6c5oc5ccc7ccccc7c56)cc4)ccc3c3c2ccc2c4ccccc4oc23)cc1]

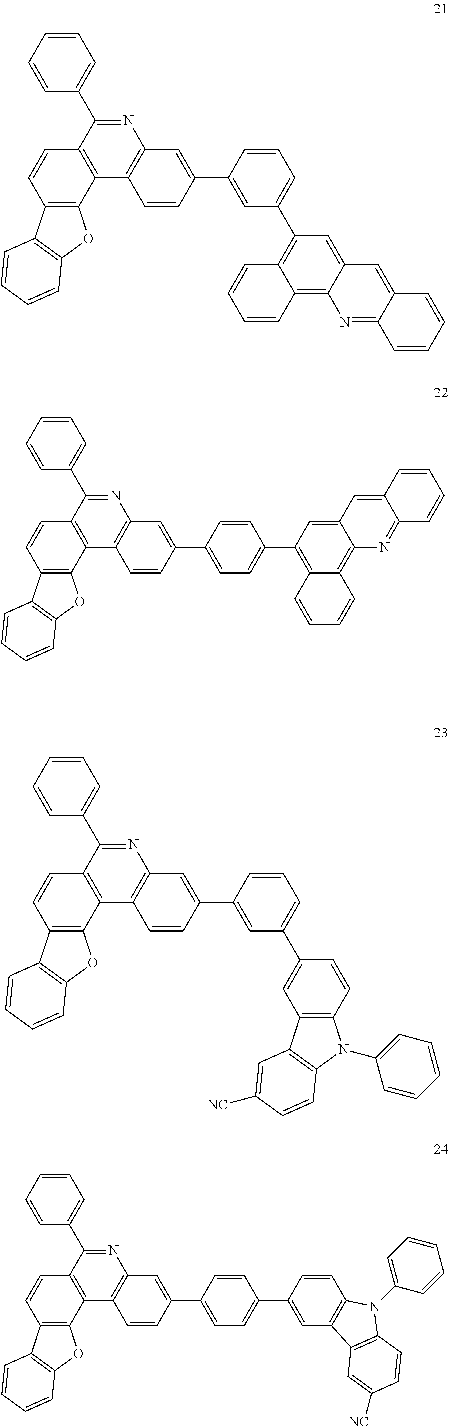 [SMILES: c1ccc(-c2nc3cc(-c4cccc(-c5cc6cc7ccccc7nc6c6ccccc56)c4)ccc3c3c2ccc2c4ccccc4oc23)cc1 ; c1ccc(-c2nc3cc(-c4ccc(-c5cc6cc7ccccc7nc6c6ccccc56)cc4)ccc3c3c2ccc2c4ccccc4oc23)cc1 ; N#Cc1ccc2c(c1)c1cc(-c3cccc(-c4ccc5c(c4)nc(-c4ccccc4)c4ccc6c7ccccc7oc6c45)c3)ccc1n2-c1ccccc1 ; N#Cc1ccc2c(c1)c1cc(-c3ccc(-c4ccc5c(c4)nc(-c4ccccc4)c4ccc6c7ccccc7oc6c45)cc3)ccc1n2-c1ccccc1]

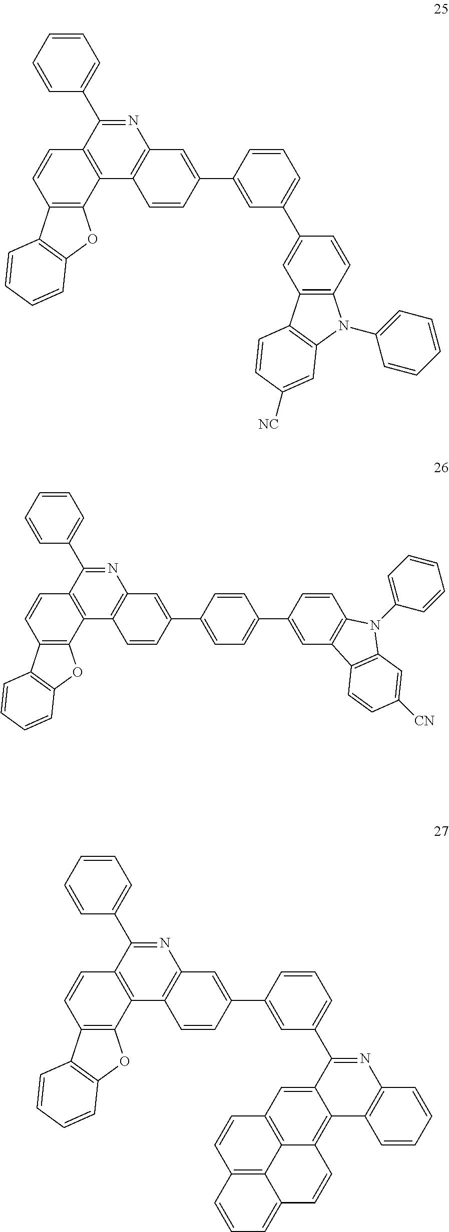 [SMILES: N#Cc1ccc2c3cc(-c4cccc(-c5ccc6c(c5)nc(-c5ccccc5)c5ccc7c8ccccc8oc7c56)c4)ccc3n(-c3ccccc3)c2c1 ; N#Cc1ccc2c3cc(-c4ccc(-c5ccc6c(c5)nc(-c5ccccc5)c5ccc7c8ccccc8oc7c56)cc4)ccc3n(-c3ccccc3)c2c1 ; c1ccc(-c2nc3cc(-c4cccc(-c5nc6ccccc6c6c5cc5ccc7cccc8ccc6c5c78)c4)ccc3c3c2ccc2c4ccccc4oc23)cc1]

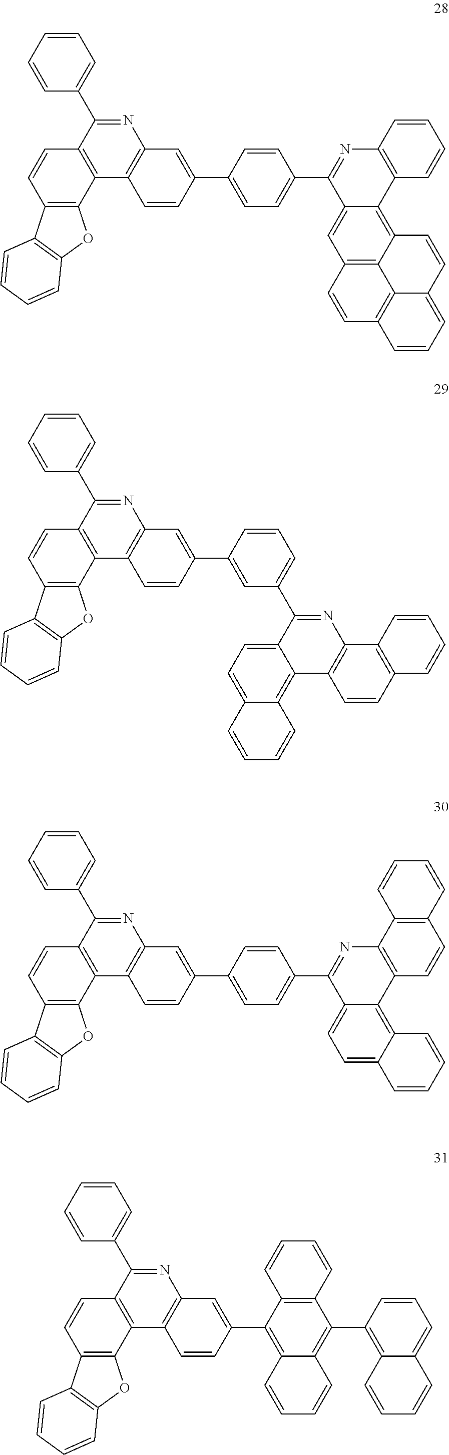 [SMILES: c1ccc(-c2nc3cc(-c4ccc(-c5nc6ccccc6c6c5cc5ccc7cccc8ccc6c5c78)cc4)ccc3c3c2ccc2c4ccccc4oc23)cc1 ; c1ccc(-c2nc3cc(-c4cccc(-c5nc6c7ccccc7ccc6c6c5ccc5ccccc56)c4)ccc3c3c2ccc2c4ccccc4oc23)cc1 ; c1ccc(-c2nc3cc(-c4ccc(-c5nc6c7ccccc7ccc6c6c5ccc5ccccc56)cc4)ccc3c3c2ccc2c4ccccc4oc23)cc1 ; c1ccc(-c2nc3cc(-c4c5ccccc5c(-c5cccc6ccccc56)c5ccccc45)ccc3c3c2ccc2c4ccccc4oc23)cc1]

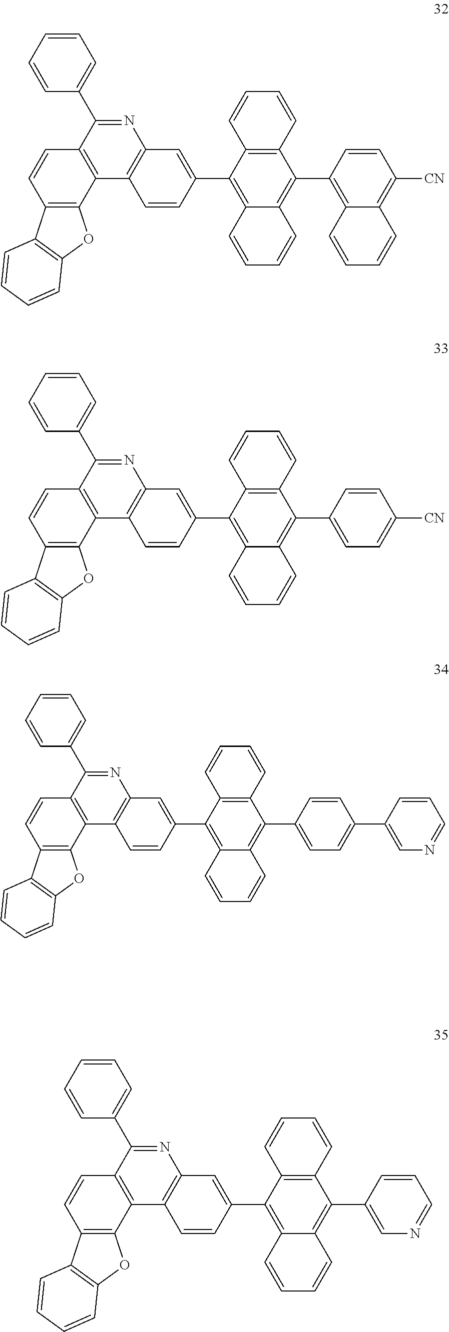 [SMILES: N#Cc1ccc(-c2c3ccccc3c(-c3ccc4c(c3)nc(-c3ccccc3)c3ccc5c6ccccc6oc5c34)c3ccccc23)c2ccccc12 ; N#Cc1ccc(-c2c3ccccc3c(-c3ccc4c(c3)nc(-c3ccccc3)c3ccc5c6ccccc6oc5c34)c3ccccc23)cc1 ; c1ccc(-c2nc3cc(-c4c5ccccc5c(-c5ccc(-c6cccnc6)cc5)c5ccccc45)ccc3c3c2ccc2c4ccccc4oc23)cc1 ; c1ccc(-c2nc3cc(-c4c5ccccc5c(-c5cccnc5)c5ccccc45)ccc3c3c2ccc2c4ccccc4oc23)cc1]

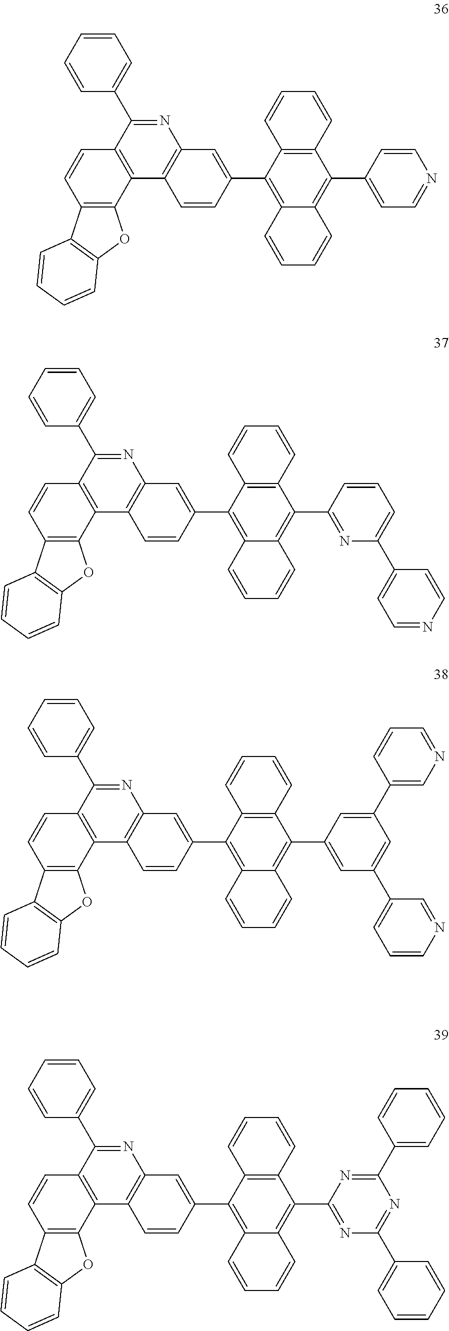 [SMILES: c1ccc(-c2nc3cc(-c4c5ccccc5c(-c5ccncc5)c5ccccc45)ccc3c3c2ccc2c4ccccc4oc23)cc1 ; c1ccc(-c2nc3cc(-c4c5ccccc5c(-c5cccc(-c6ccncc6)n5)c5ccccc45)ccc3c3c2ccc2c4ccccc4oc23)cc1 ; c1ccc(-c2nc3cc(-c4c5ccccc5c(-c5cc(-c6cccnc6)cc(-c6cccnc6)c5)c5ccccc45)ccc3c3c2ccc2c4ccccc4oc23)cc1 ; c1ccc(-c2nc(-c3ccccc3)nc(-c3c4ccccc4c(-c4ccc5c(c4)nc(-c4ccccc4)c4ccc6c7ccccc7oc6c45)c4ccccc34)n2)cc1]

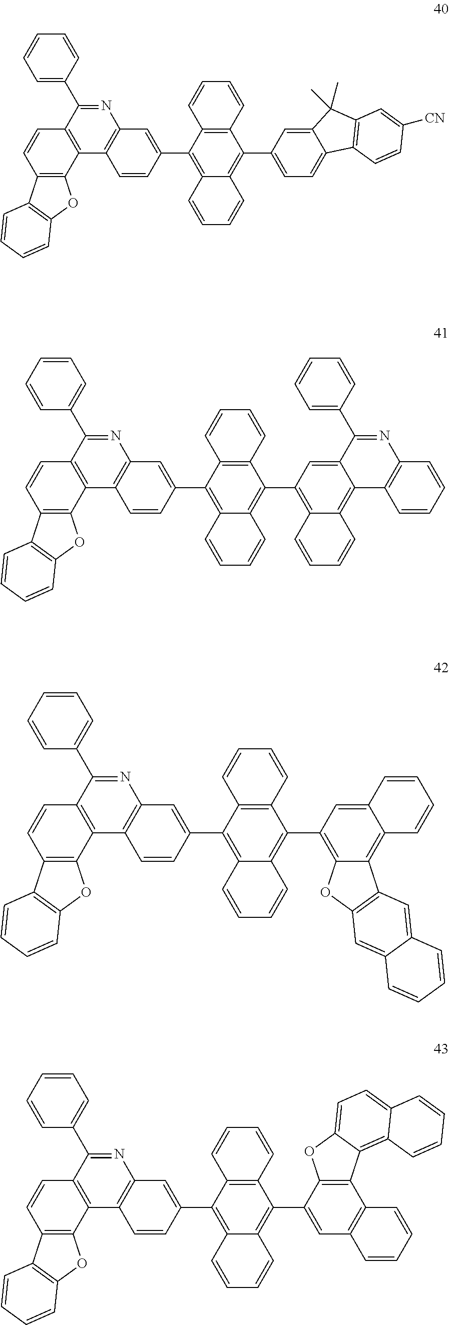 [SMILES: CC1(C)c2cc(C#N)ccc2-c2ccc(-c3c4ccccc4c(-c4ccc5c(c4)nc(-c4ccccc4)c4ccc6c7ccccc7oc6c45)c4ccccc34)cc21 ; c1ccc(-c2nc3ccccc3c3c2cc(-c2c4ccccc4c(-c4ccc5c(c4)nc(-c4ccccc4)c4ccc6c7ccccc7oc6c45)c4ccccc24)c2ccccc23)cc1 ; c1ccc(-c2nc3cc(-c4c5ccccc5c(-c5cc6ccccc6c6c5oc5cc7ccccc7cc56)c5ccccc45)ccc3c3c2ccc2c4ccccc4oc23)cc1 ; c1ccc(-c2nc3cc(-c4c5ccccc5c(-c5cc6ccccc6c6c5oc5ccc7ccccc7c56)c5ccccc45)ccc3c3c2ccc2c4ccccc4oc23)cc1]

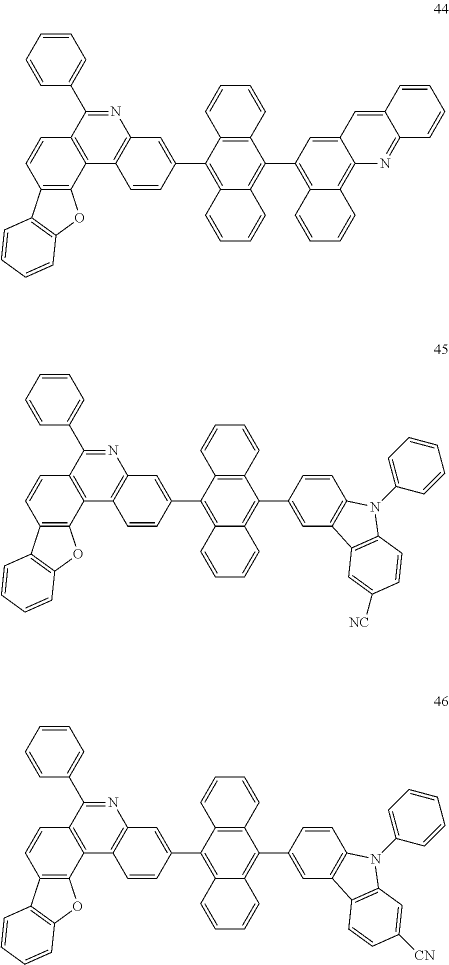 [SMILES: c1ccc(-c2nc3cc(-c4c5ccccc5c(-c5cc6cc7ccccc7nc6c6ccccc56)c5ccccc45)ccc3c3c2ccc2c4ccccc4oc23)cc1 ; N#Cc1ccc2c(c1)c1cc(-c3c4ccccc4c(-c4ccc5c(c4)nc(-c4ccccc4)c4ccc6c7ccccc7oc6c45)c4ccccc34)ccc1n2-c1ccccc1 ; N#Cc1ccc2c3cc(-c4c5ccccc5c(-c5ccc6c(c5)nc(-c5ccccc5)c5ccc7c8ccccc8oc7c56)c5ccccc45)ccc3n(-c3ccccc3)c2c1]

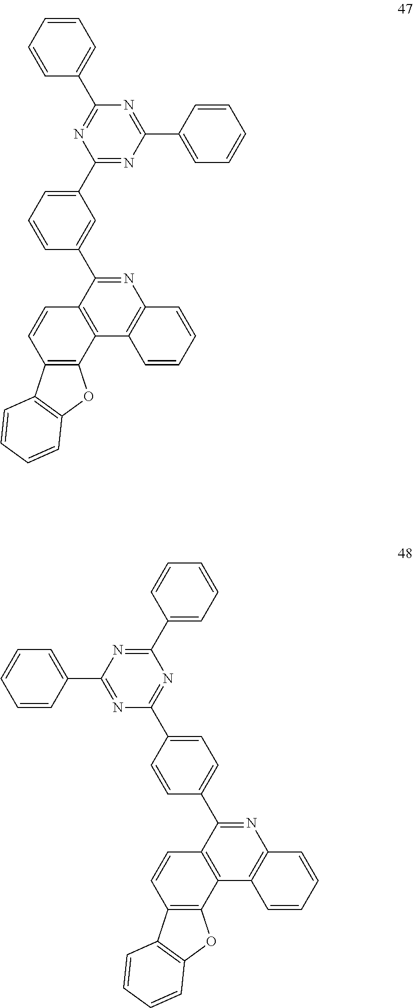 [SMILES: c1ccc(-c2nc(-c3ccccc3)nc(-c3cccc(-c4nc5ccccc5c5c4ccc4c6ccccc6oc45)c3)n2)cc1 ; c1ccc(-c2nc(-c3ccccc3)nc(-c3ccc(-c4nc5ccccc5c5c4ccc4c6ccccc6oc45)cc3)n2)cc1]

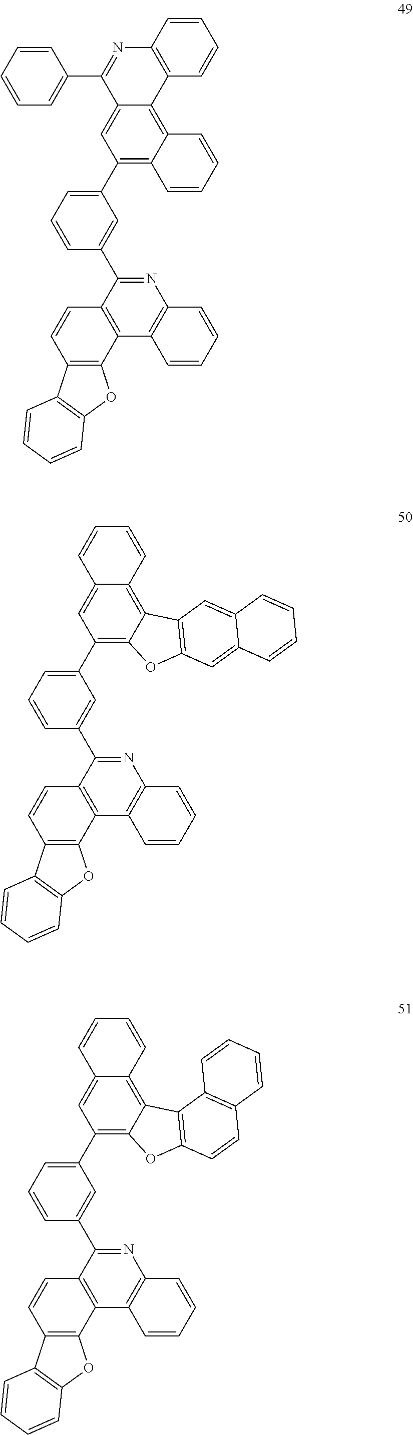 [SMILES: c1ccc(-c2nc3ccccc3c3c2cc(-c2cccc(-c4nc5ccccc5c5c4ccc4c6ccccc6oc45)c2)c2ccccc23)cc1 ; c1cc(-c2nc3ccccc3c3c2ccc2c4ccccc4oc23)cc(-c2cc3ccccc3c3c2oc2cc4ccccc4cc23)c1 ; c1cc(-c2nc3ccccc3c3c2ccc2c4ccccc4oc23)cc(-c2cc3ccccc3c3c2oc2ccc4ccccc4c23)c1]

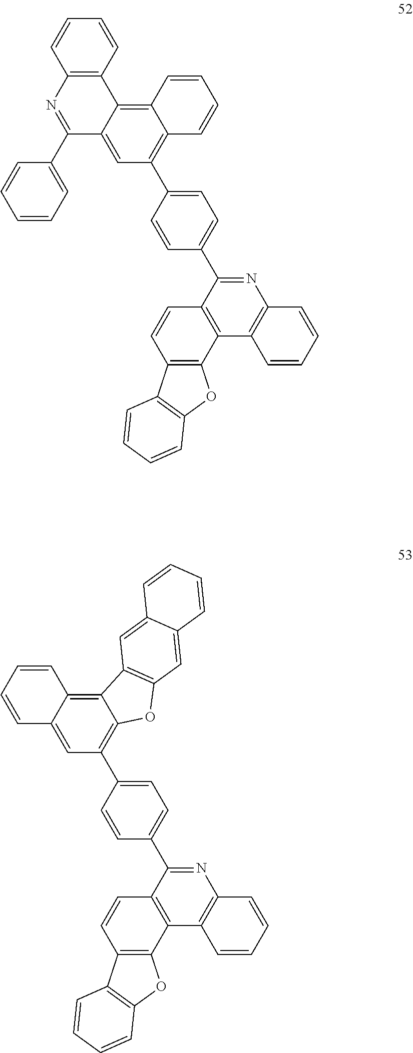 [SMILES: c1ccc(-c2nc3ccccc3c3c2cc(-c2ccc(-c4nc5ccccc5c5c4ccc4c6ccccc6oc45)cc2)c2ccccc23)cc1 ; c1ccc2cc3c(cc2c1)oc1c(-c2ccc(-c4nc5ccccc5c5c4ccc4c6ccccc6oc45)cc2)cc2ccccc2c13]

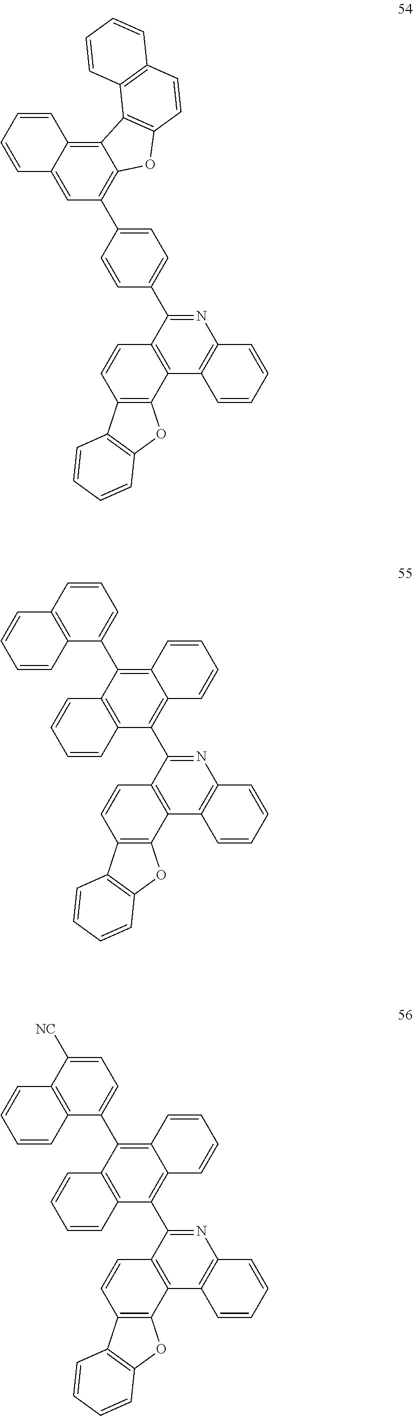 [SMILES: c1ccc2c(c1)ccc1oc3c(-c4ccc(-c5nc6ccccc6c6c5ccc5c7ccccc7oc56)cc4)cc4ccccc4c3c12 ; c1ccc2c(-c3c4ccccc4c(-c4nc5ccccc5c5c4ccc4c6ccccc6oc45)c4ccccc34)cccc2c1 ; N#Cc1ccc(-c2c3ccccc3c(-c3nc4ccccc4c4c3ccc3c5ccccc5oc34)c3ccccc23)c2ccccc12]

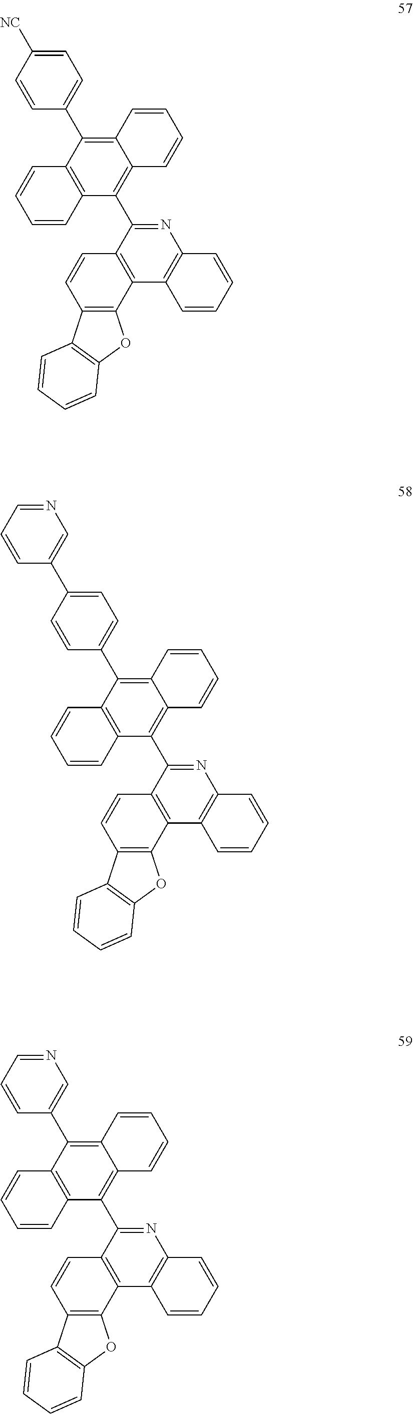 [SMILES: N#Cc1ccc(-c2c3ccccc3c(-c3nc4ccccc4c4c3ccc3c5ccccc5oc34)c3ccccc23)cc1 ; c1cncc(-c2ccc(-c3c4ccccc4c(-c4nc5ccccc5c5c4ccc4c6ccccc6oc45)c4ccccc34)cc2)c1 ; c1cncc(-c2c3ccccc3c(-c3nc4ccccc4c4c3ccc3c5ccccc5oc34)c3ccccc23)c1]

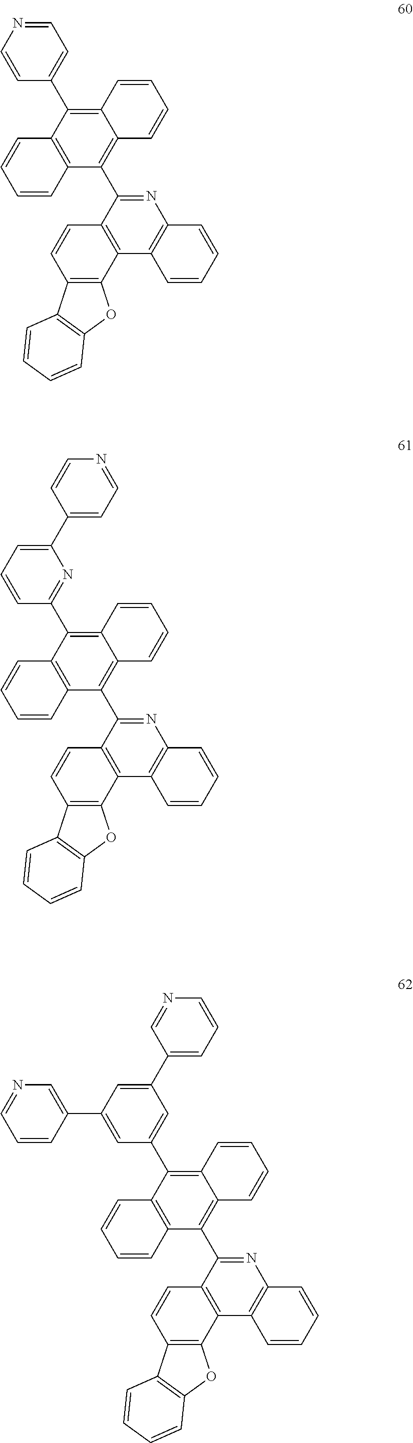 [SMILES: c1ccc2c(c1)nc(-c1c3ccccc3c(-c3ccncc3)c3ccccc13)c1ccc3c4ccccc4oc3c12 ; c1cc(-c2ccncc2)nc(-c2c3ccccc3c(-c3nc4ccccc4c4c3ccc3c5ccccc5oc34)c3ccccc23)c1 ; c1cncc(-c2cc(-c3cccnc3)cc(-c3c4ccccc4c(-c4nc5ccccc5c5c4ccc4c6ccccc6oc45)c4ccccc34)c2)c1]

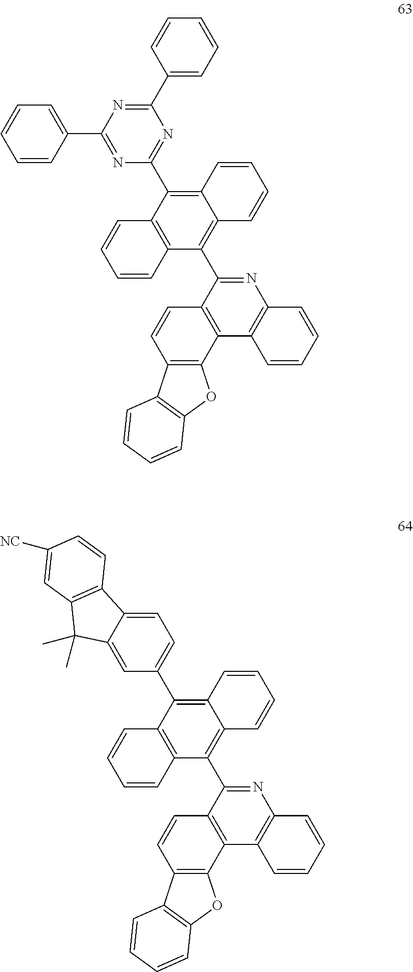 [SMILES: c1ccc(-c2nc(-c3ccccc3)nc(-c3c4ccccc4c(-c4nc5ccccc5c5c4ccc4c6ccccc6oc45)c4ccccc34)n2)cc1 ; CC1(C)c2cc(C#N)ccc2-c2ccc(-c3c4ccccc4c(-c4nc5ccccc5c5c4ccc4c6ccccc6oc45)c4ccccc34)cc21]

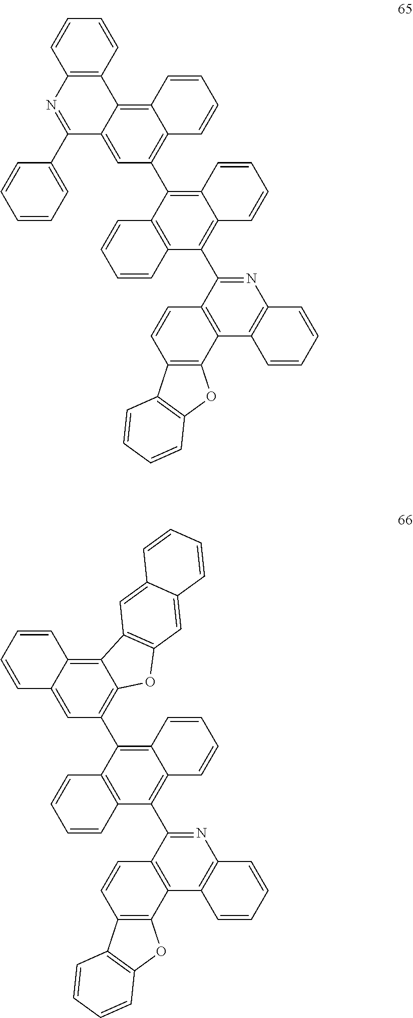 [SMILES: c1ccc(-c2nc3ccccc3c3c2cc(-c2c4ccccc4c(-c4nc5ccccc5c5c4ccc4c6ccccc6oc45)c4ccccc24)c2ccccc23)cc1 ; c1ccc2cc3c(cc2c1)oc1c(-c2c4ccccc4c(-c4nc5ccccc5c5c4ccc4c6ccccc6oc45)c4ccccc24)cc2ccccc2c13]

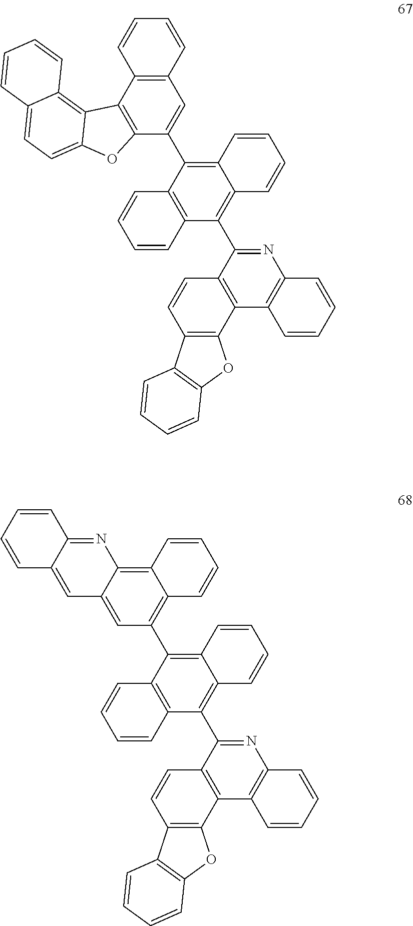 [SMILES: c1ccc2c(c1)ccc1oc3c(-c4c5ccccc5c(-c5nc6ccccc6c6c5ccc5c7ccccc7oc56)c5ccccc45)cc4ccccc4c3c12 ; c1ccc2nc3c(cc(-c4c5ccccc5c(-c5nc6ccccc6c6c5ccc5c7ccccc7oc56)c5ccccc45)c4ccccc43)cc2c1]

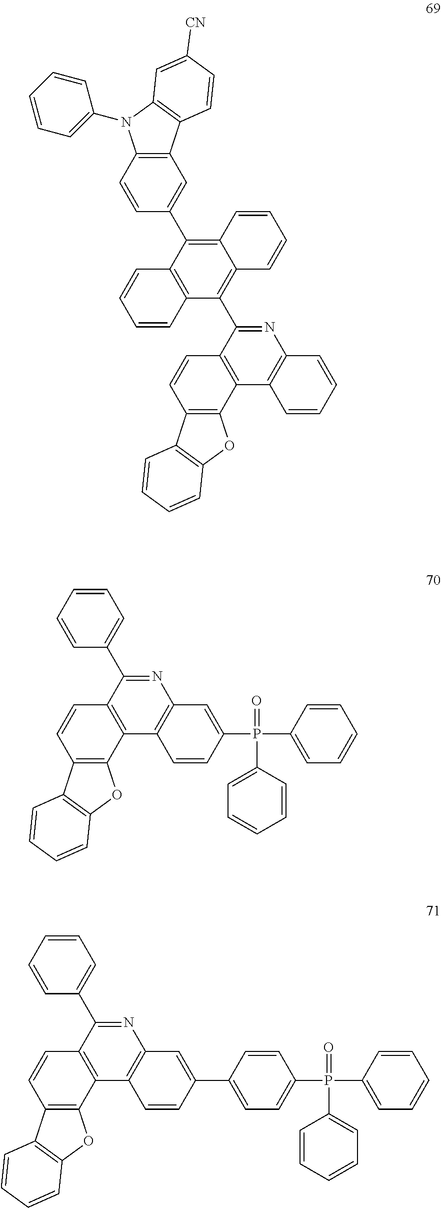 [SMILES: N#Cc1ccc2c3cc(-c4c5ccccc5c(-c5nc6ccccc6c6c5ccc5c7ccccc7oc56)c5ccccc45)ccc3n(-c3ccccc3)c2c1 ; O=P(c1ccccc1)(c1ccccc1)c1ccc2c(c1)nc(-c1ccccc1)c1ccc3c4ccccc4oc3c12 ; O=P(c1ccccc1)(c1ccccc1)c1ccc(-c2ccc3c(c2)nc(-c2ccccc2)c2ccc4c5ccccc5oc4c23)cc1]

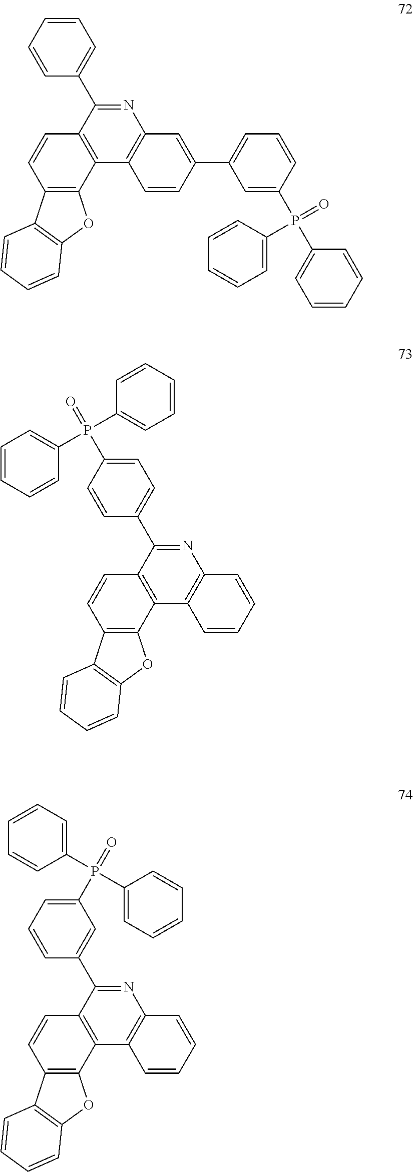 [SMILES: O=P(c1ccccc1)(c1ccccc1)c1cccc(-c2ccc3c(c2)nc(-c2ccccc2)c2ccc4c5ccccc5oc4c23)c1 ; O=P(c1ccccc1)(c1ccccc1)c1ccc(-c2nc3ccccc3c3c2ccc2c4ccccc4oc23)cc1 ; O=P(c1ccccc1)(c1ccccc1)c1cccc(-c2nc3ccccc3c3c2ccc2c4ccccc4oc23)c1]

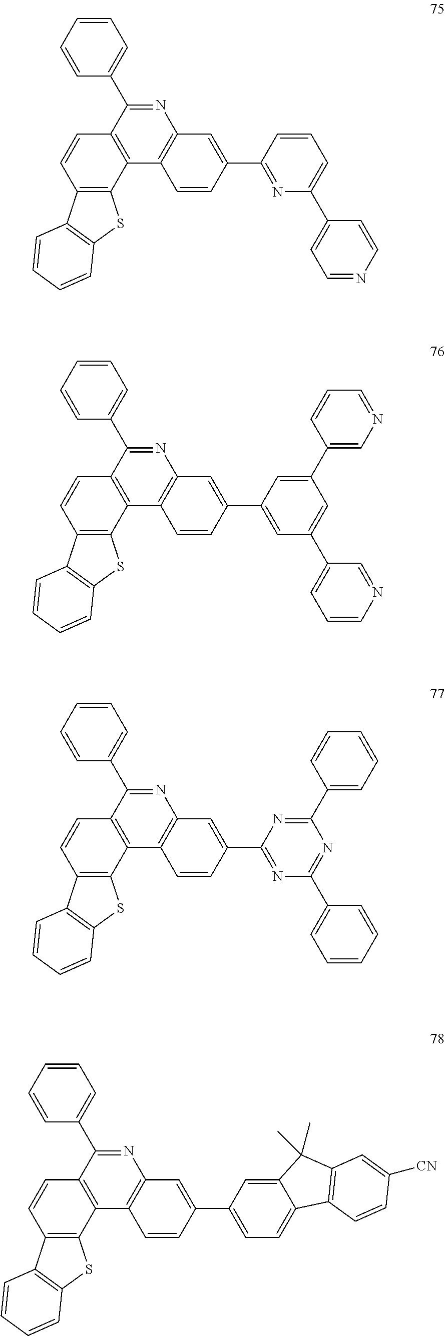 [SMILES: c1ccc(-c2nc3cc(-c4cccc(-c5ccncc5)n4)ccc3c3c2ccc2c4ccccc4sc23)cc1 ; c1ccc(-c2nc3cc(-c4cc(-c5cccnc5)cc(-c5cccnc5)c4)ccc3c3c2ccc2c4ccccc4sc23)cc1 ; c1ccc(-c2nc(-c3ccccc3)nc(-c3ccc4c(c3)nc(-c3ccccc3)c3ccc5c6ccccc6sc5c34)n2)cc1 ; CC1(C)c2cc(C#N)ccc2-c2ccc(-c3ccc4c(c3)nc(-c3ccccc3)c3ccc5c6ccccc6sc5c34)cc21]

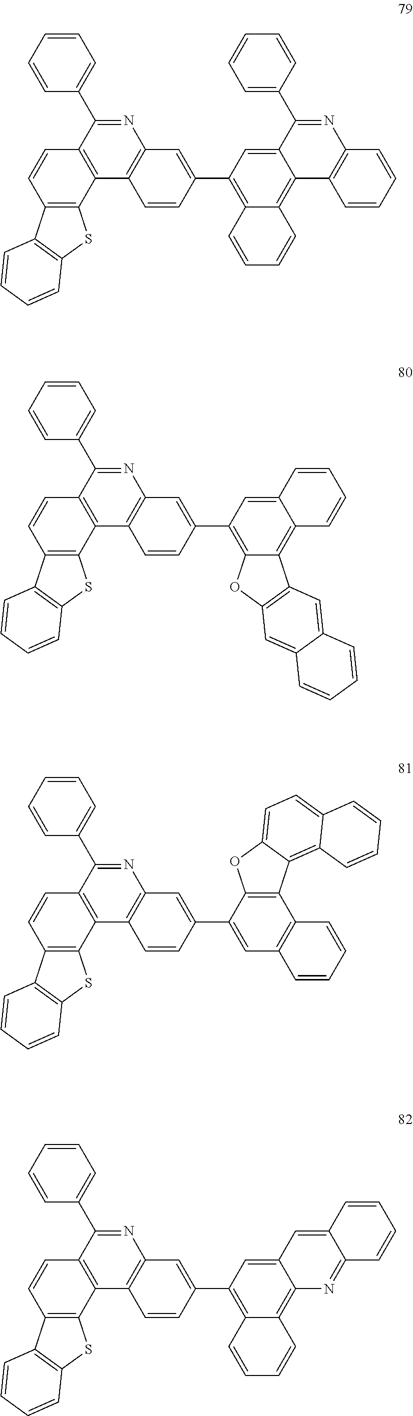 [SMILES: c1ccc(-c2nc3ccccc3c3c2cc(-c2ccc4c(c2)nc(-c2ccccc2)c2ccc5c6ccccc6sc5c24)c2ccccc23)cc1 ; c1ccc(-c2nc3cc(-c4cc5ccccc5c5c4oc4cc6ccccc6cc45)ccc3c3c2ccc2c4ccccc4sc23)cc1 ; c1ccc(-c2nc3cc(-c4cc5ccccc5c5c4oc4ccc6ccccc6c45)ccc3c3c2ccc2c4ccccc4sc23)cc1 ; c1ccc(-c2nc3cc(-c4cc5cc6ccccc6nc5c5ccccc45)ccc3c3c2ccc2c4ccccc4sc23)cc1]

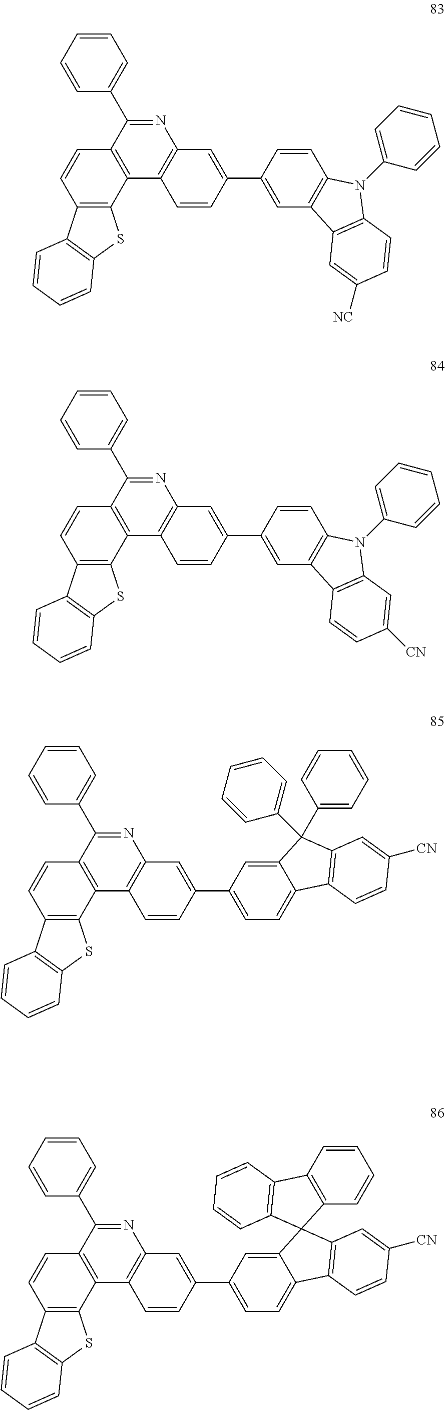 [SMILES: N#Cc1ccc2c(c1)c1cc(-c3ccc4c(c3)nc(-c3ccccc3)c3ccc5c6ccccc6sc5c34)ccc1n2-c1ccccc1 ; N#Cc1ccc2c3cc(-c4ccc5c(c4)nc(-c4ccccc4)c4ccc6c7ccccc7sc6c45)ccc3n(-c3ccccc3)c2c1 ; N#Cc1ccc2c(c1)C(c1ccccc1)(c1ccccc1)c1cc(-c3ccc4c(c3)nc(-c3ccccc3)c3ccc5c6ccccc6sc5c34)ccc1-2 ; N#Cc1ccc2c(c1)C1(c3ccccc3-c3ccccc31)c1cc(-c3ccc4c(c3)nc(-c3ccccc3)c3ccc5c6ccccc6sc5c34)ccc1-2]

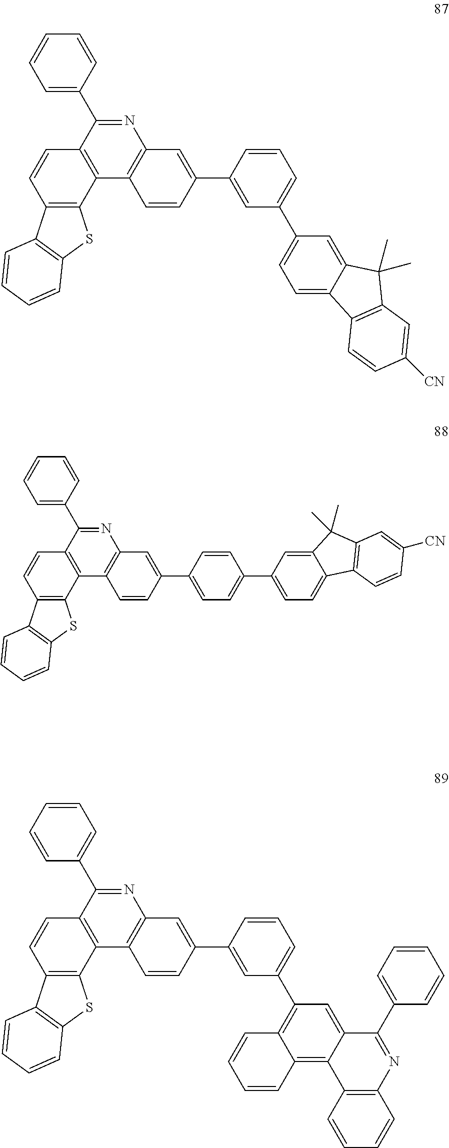 [SMILES: CC1(C)c2cc(C#N)ccc2-c2ccc(-c3cccc(-c4ccc5c(c4)nc(-c4ccccc4)c4ccc6c7ccccc7sc6c45)c3)cc21 ; CC1(C)c2cc(C#N)ccc2-c2ccc(-c3ccc(-c4ccc5c(c4)nc(-c4ccccc4)c4ccc6c7ccccc7sc6c45)cc3)cc21 ; c1ccc(-c2nc3ccccc3c3c2cc(-c2cccc(-c4ccc5c(c4)nc(-c4ccccc4)c4ccc6c7ccccc7sc6c45)c2)c2ccccc23)cc1]

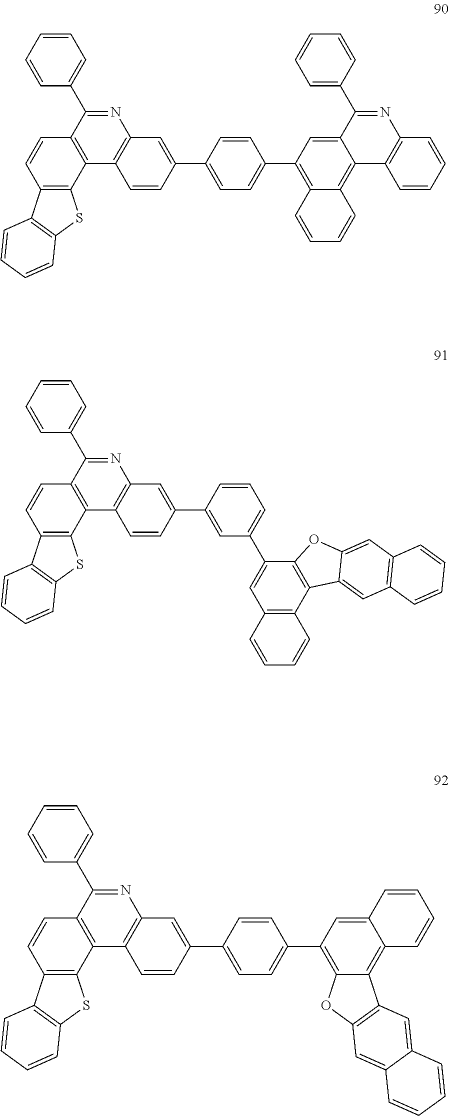 [SMILES: c1ccc(-c2nc3ccccc3c3c2cc(-c2ccc(-c4ccc5c(c4)nc(-c4ccccc4)c4ccc6c7ccccc7sc6c45)cc2)c2ccccc23)cc1 ; c1ccc(-c2nc3cc(-c4cccc(-c5cc6ccccc6c6c5oc5cc7ccccc7cc56)c4)ccc3c3c2ccc2c4ccccc4sc23)cc1 ; c1ccc(-c2nc3cc(-c4ccc(-c5cc6ccccc6c6c5oc5cc7ccccc7cc56)cc4)ccc3c3c2ccc2c4ccccc4sc23)cc1]

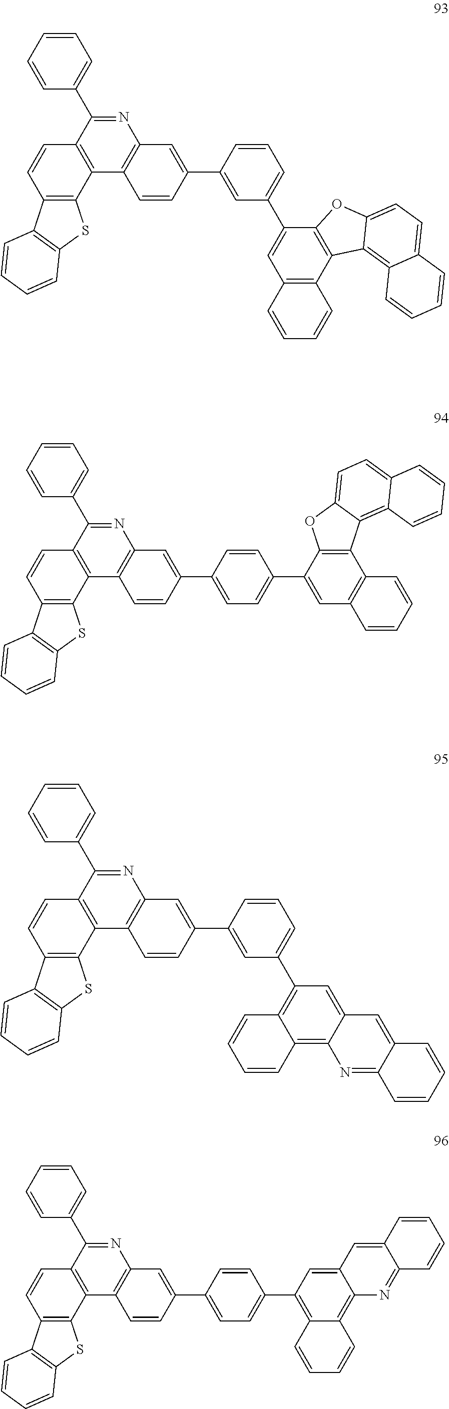 [SMILES: c1ccc(-c2nc3cc(-c4cccc(-c5cc6ccccc6c6c5oc5ccc7ccccc7c56)c4)ccc3c3c2ccc2c4ccccc4sc23)cc1 ; c1ccc(-c2nc3cc(-c4ccc(-c5cc6ccccc6c6c5oc5ccc7ccccc7c56)cc4)ccc3c3c2ccc2c4ccccc4sc23)cc1 ; c1ccc(-c2nc3cc(-c4cccc(-c5cc6cc7ccccc7nc6c6ccccc56)c4)ccc3c3c2ccc2c4ccccc4sc23)cc1 ; c1ccc(-c2nc3cc(-c4ccc(-c5cc6cc7ccccc7nc6c6ccccc56)cc4)ccc3c3c2ccc2c4ccccc4sc23)cc1]

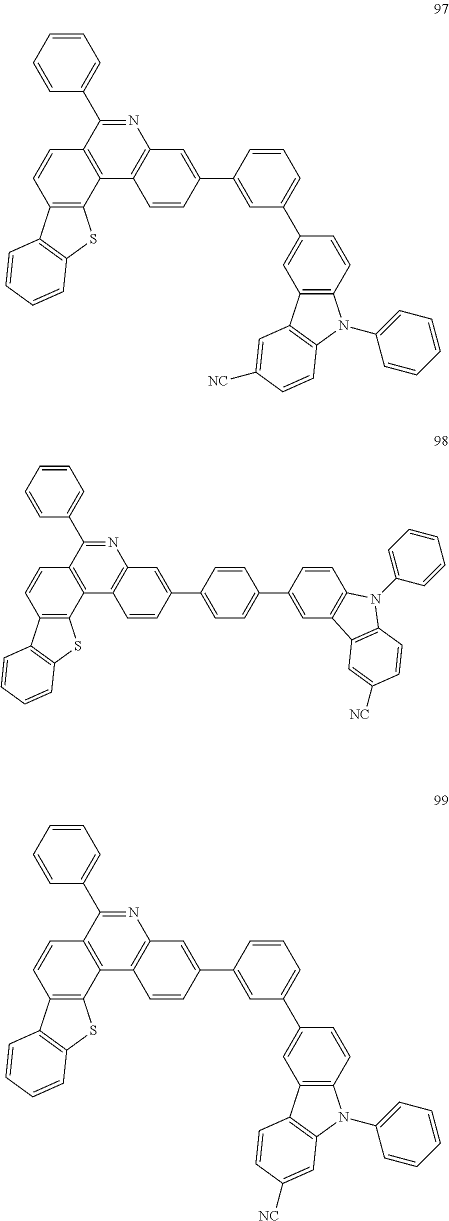 [SMILES: N#Cc1ccc2c(c1)c1cc(-c3cccc(-c4ccc5c(c4)nc(-c4ccccc4)c4ccc6c7ccccc7sc6c45)c3)ccc1n2-c1ccccc1 ; N#Cc1ccc2c(c1)c1cc(-c3ccc(-c4ccc5c(c4)nc(-c4ccccc4)c4ccc6c7ccccc7sc6c45)cc3)ccc1n2-c1ccccc1 ; N#Cc1ccc2c3cc(-c4cccc(-c5ccc6c(c5)nc(-c5ccccc5)c5ccc7c8ccccc8sc7c56)c4)ccc3n(-c3ccccc3)c2c1]

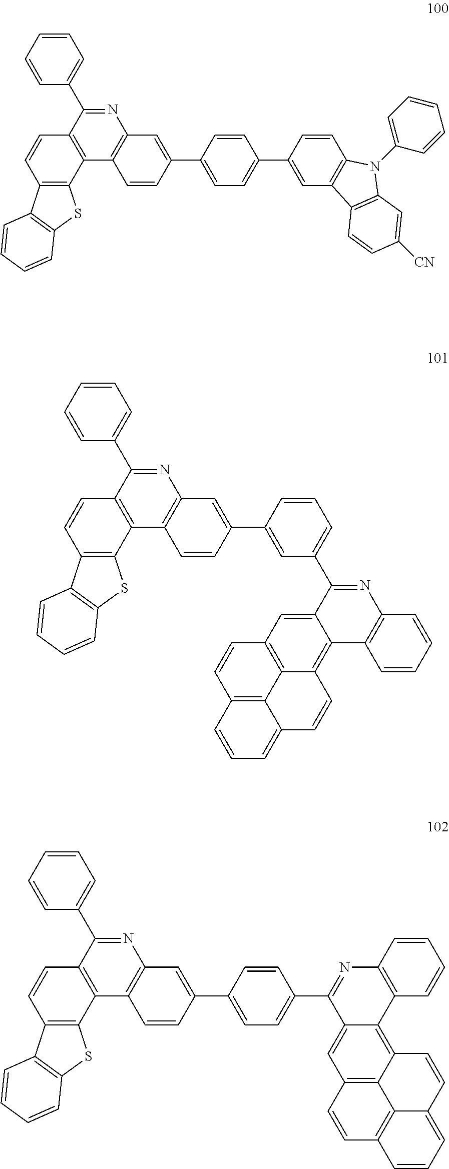 [SMILES: N#Cc1ccc2c3cc(-c4ccc(-c5ccc6c(c5)nc(-c5ccccc5)c5ccc7c8ccccc8sc7c56)cc4)ccc3n(-c3ccccc3)c2c1 ; c1ccc(-c2nc3cc(-c4cccc(-c5nc6ccccc6c6c5cc5ccc7cccc8ccc6c5c78)c4)ccc3c3c2ccc2c4ccccc4sc23)cc1 ; c1ccc(-c2nc3cc(-c4ccc(-c5nc6ccccc6c6c5cc5ccc7cccc8ccc6c5c78)cc4)ccc3c3c2ccc2c4ccccc4sc23)cc1]

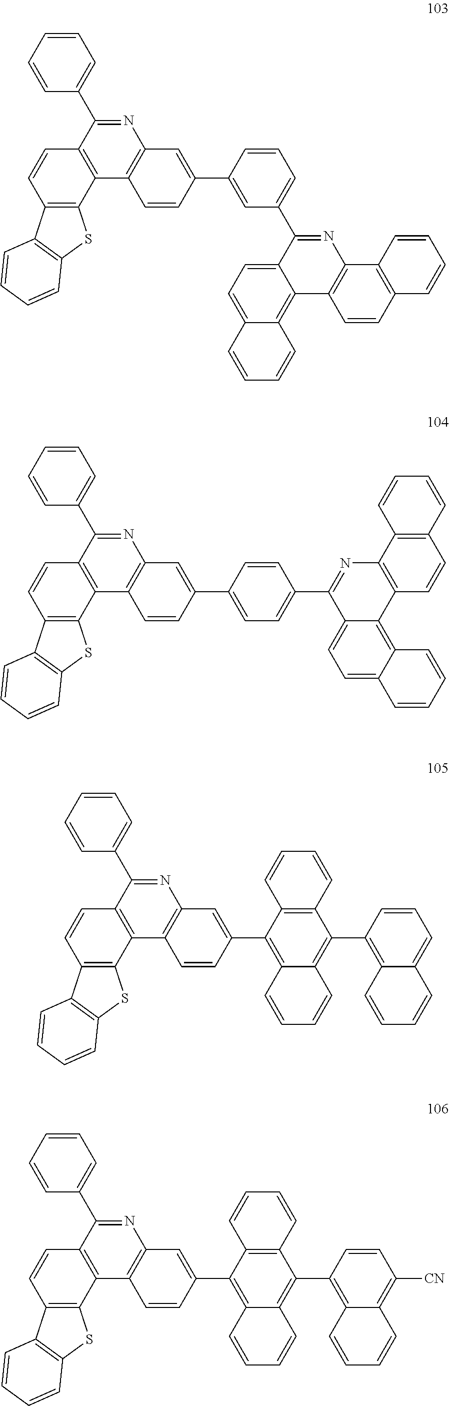 [SMILES: c1ccc(-c2nc3cc(-c4cccc(-c5nc6c7ccccc7ccc6c6c5ccc5ccccc56)c4)ccc3c3c2ccc2c4ccccc4sc23)cc1 ; c1ccc(-c2nc3cc(-c4ccc(-c5nc6c7ccccc7ccc6c6c5ccc5ccccc56)cc4)ccc3c3c2ccc2c4ccccc4sc23)cc1 ; c1ccc(-c2nc3cc(-c4c5ccccc5c(-c5cccc6ccccc56)c5ccccc45)ccc3c3c2ccc2c4ccccc4sc23)cc1 ; N#Cc1ccc(-c2c3ccccc3c(-c3ccc4c(c3)nc(-c3ccccc3)c3ccc5c6ccccc6sc5c34)c3ccccc23)c2ccccc12]

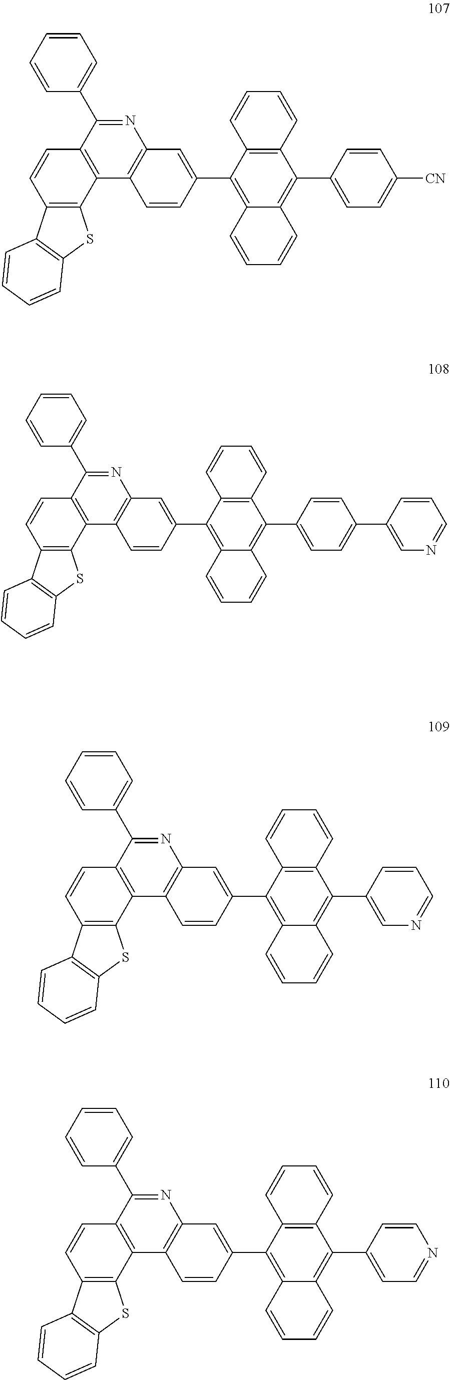 [SMILES: N#Cc1ccc(-c2c3ccccc3c(-c3ccc4c(c3)nc(-c3ccccc3)c3ccc5c6ccccc6sc5c34)c3ccccc23)cc1 ; c1ccc(-c2nc3cc(-c4c5ccccc5c(-c5ccc(-c6cccnc6)cc5)c5ccccc45)ccc3c3c2ccc2c4ccccc4sc23)cc1 ; c1ccc(-c2nc3cc(-c4c5ccccc5c(-c5cccnc5)c5ccccc45)ccc3c3c2ccc2c4ccccc4sc23)cc1 ; c1ccc(-c2nc3cc(-c4c5ccccc5c(-c5ccncc5)c5ccccc45)ccc3c3c2ccc2c4ccccc4sc23)cc1]

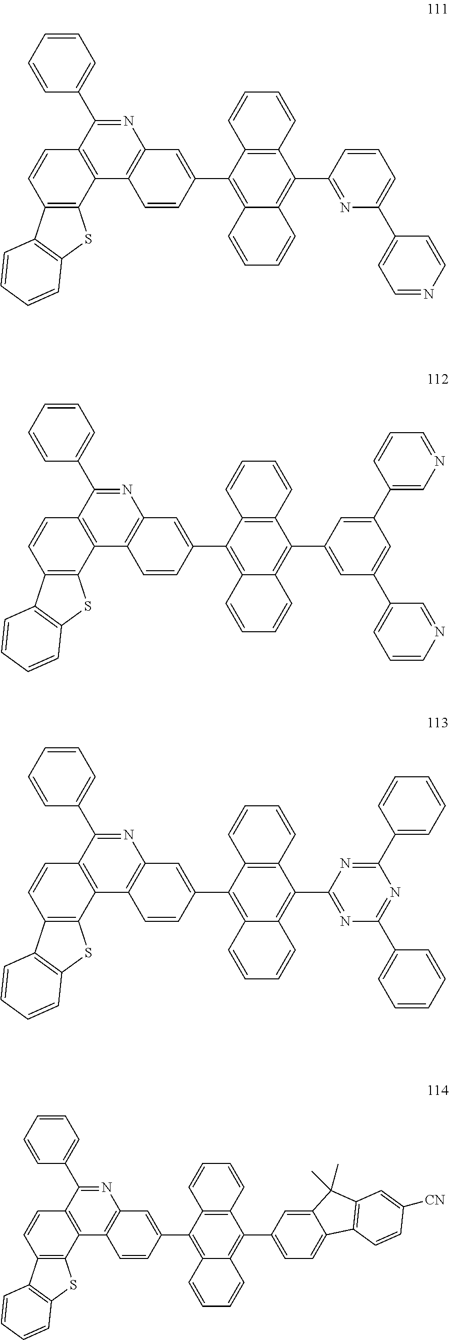 [SMILES: c1ccc(-c2nc3cc(-c4c5ccccc5c(-c5cccc(-c6ccncc6)n5)c5ccccc45)ccc3c3c2ccc2c4ccccc4sc23)cc1 ; c1ccc(-c2nc3cc(-c4c5ccccc5c(-c5cc(-c6cccnc6)cc(-c6cccnc6)c5)c5ccccc45)ccc3c3c2ccc2c4ccccc4sc23)cc1 ; c1ccc(-c2nc(-c3ccccc3)nc(-c3c4ccccc4c(-c4ccc5c(c4)nc(-c4ccccc4)c4ccc6c7ccccc7sc6c45)c4ccccc34)n2)cc1 ; CC1(C)c2cc(C#N)ccc2-c2ccc(-c3c4ccccc4c(-c4ccc5c(c4)nc(-c4ccccc4)c4ccc6c7ccccc7sc6c45)c4ccccc34)cc21]

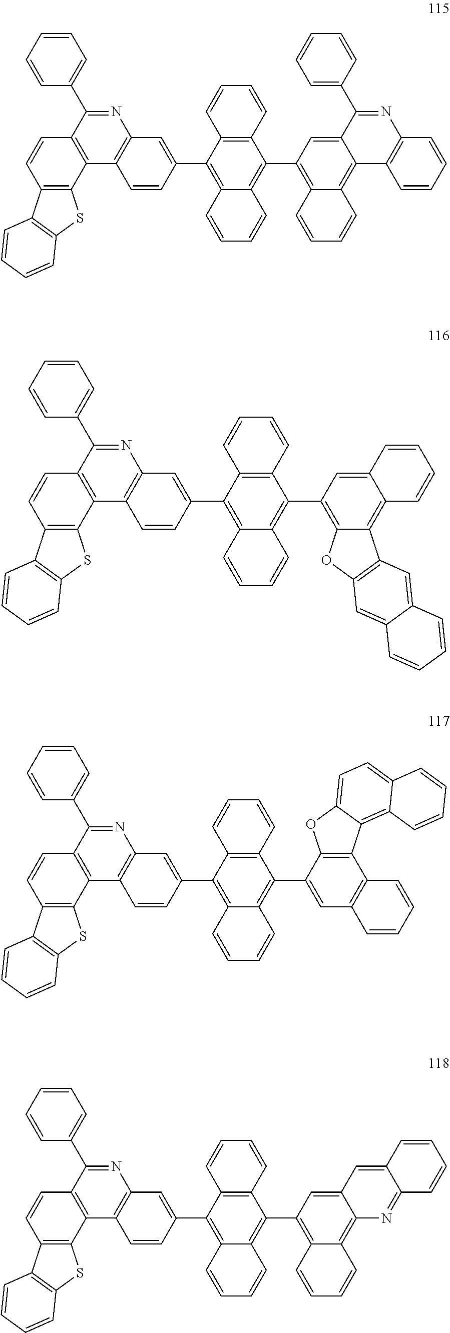 [SMILES: c1ccc(-c2nc3ccccc3c3c2cc(-c2c4ccccc4c(-c4ccc5c(c4)nc(-c4ccccc4)c4ccc6c7ccccc7sc6c45)c4ccccc24)c2ccccc23)cc1 ; c1ccc(-c2nc3cc(-c4c5ccccc5c(-c5cc6ccccc6c6c5oc5cc7ccccc7cc56)c5ccccc45)ccc3c3c2ccc2c4ccccc4sc23)cc1 ; c1ccc(-c2nc3cc(-c4c5ccccc5c(-c5cc6ccccc6c6c5oc5ccc7ccccc7c56)c5ccccc45)ccc3c3c2ccc2c4ccccc4sc23)cc1 ; c1ccc(-c2nc3cc(-c4c5ccccc5c(-c5cc6cc7ccccc7nc6c6ccccc56)c5ccccc45)ccc3c3c2ccc2c4ccccc4sc23)cc1]

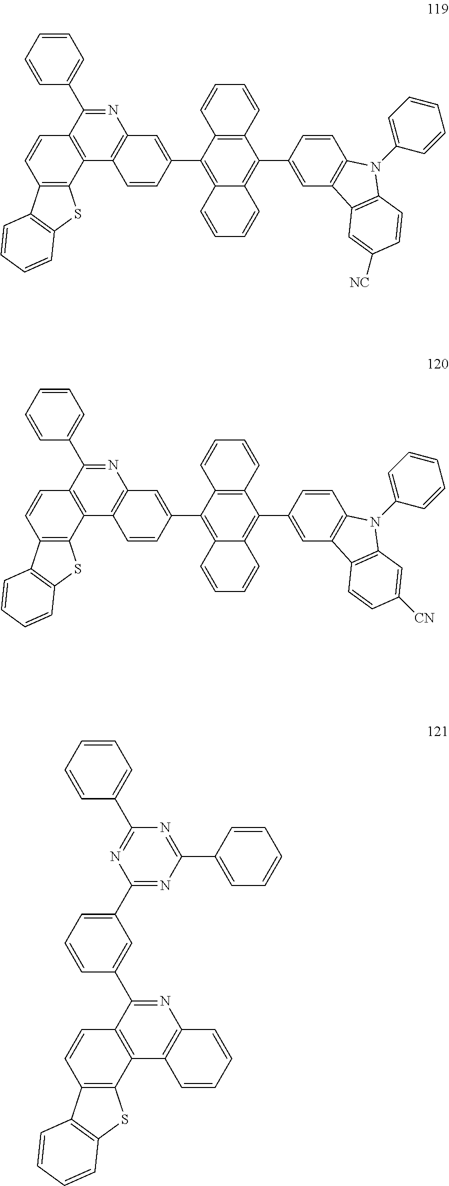 [SMILES: N#Cc1ccc2c(c1)c1cc(-c3c4ccccc4c(-c4ccc5c(c4)nc(-c4ccccc4)c4ccc6c7ccccc7sc6c45)c4ccccc34)ccc1n2-c1ccccc1 ; N#Cc1ccc2c3cc(-c4c5ccccc5c(-c5ccc6c(c5)nc(-c5ccccc5)c5ccc7c8ccccc8sc7c56)c5ccccc45)ccc3n(-c3ccccc3)c2c1 ; c1ccc(-c2nc(-c3ccccc3)nc(-c3cccc(-c4nc5ccccc5c5c4ccc4c6ccccc6sc45)c3)n2)cc1]

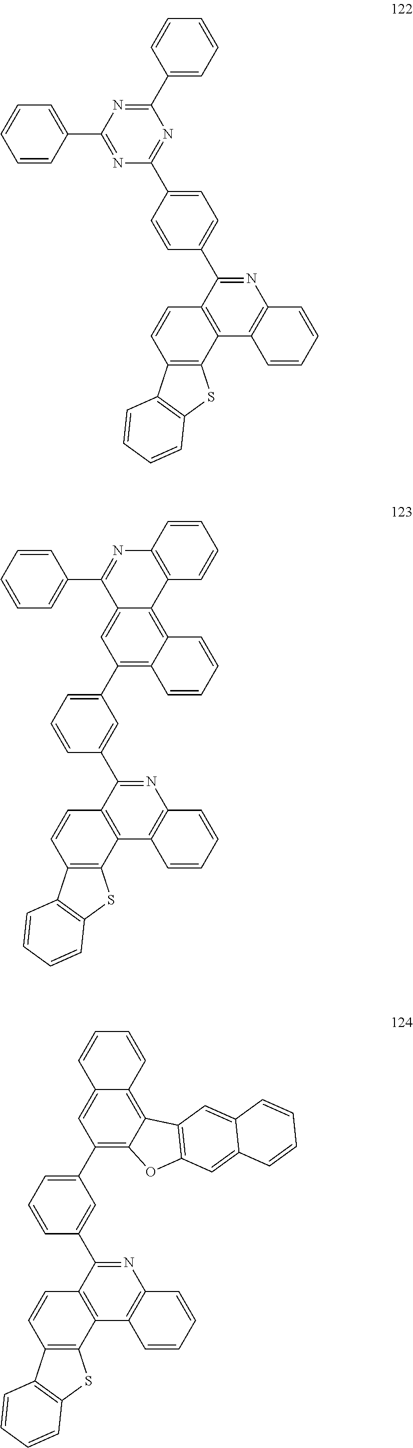 [SMILES: c1ccc(-c2nc(-c3ccccc3)nc(-c3ccc(-c4nc5ccccc5c5c4ccc4c6ccccc6sc45)cc3)n2)cc1 ; c1ccc(-c2nc3ccccc3c3c2cc(-c2cccc(-c4nc5ccccc5c5c4ccc4c6ccccc6sc45)c2)c2ccccc23)cc1 ; c1cc(-c2nc3ccccc3c3c2ccc2c4ccccc4sc23)cc(-c2cc3ccccc3c3c2oc2cc4ccccc4cc23)c1]

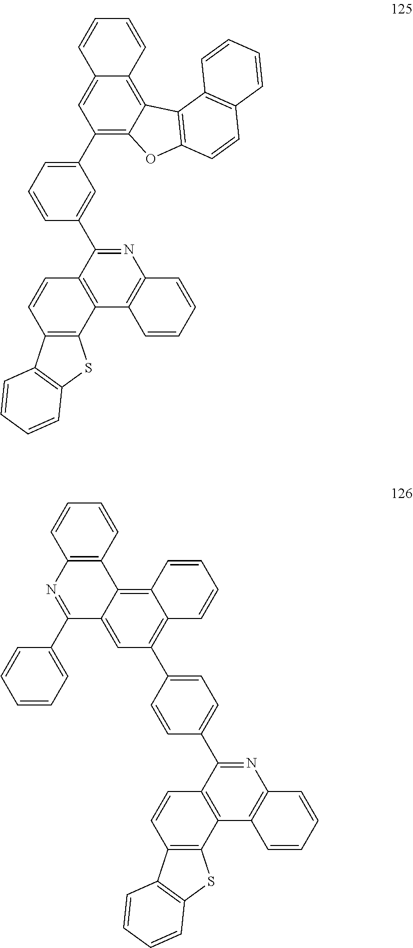 [SMILES: c1cc(-c2nc3ccccc3c3c2ccc2c4ccccc4sc23)cc(-c2cc3ccccc3c3c2oc2ccc4ccccc4c23)c1 ; c1ccc(-c2nc3ccccc3c3c2cc(-c2ccc(-c4nc5ccccc5c5c4ccc4c6ccccc6sc45)cc2)c2ccccc23)cc1]

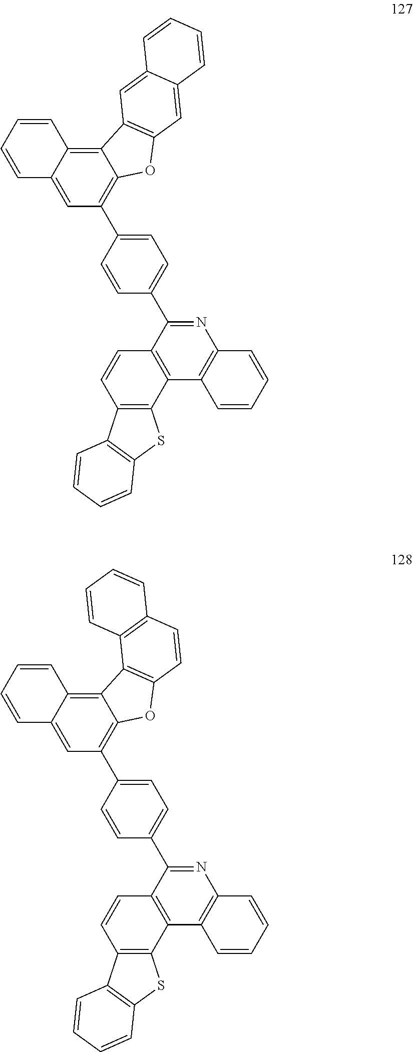 [SMILES: c1ccc2cc3c(cc2c1)oc1c(-c2ccc(-c4nc5ccccc5c5c4ccc4c6ccccc6sc45)cc2)cc2ccccc2c13 ; c1ccc2c(c1)ccc1oc3c(-c4ccc(-c5nc6ccccc6c6c5ccc5c7ccccc7sc56)cc4)cc4ccccc4c3c12]

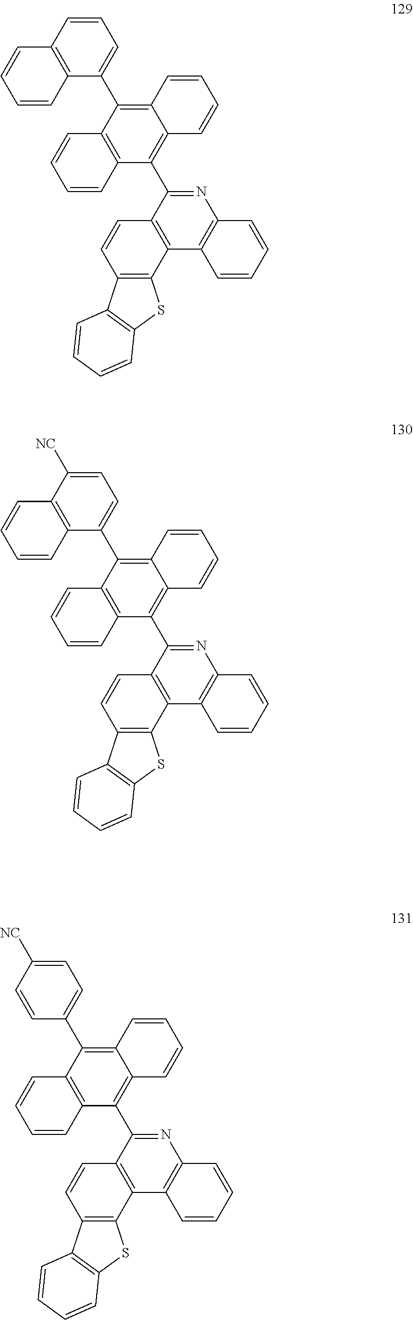 [SMILES: c1ccc2c(-c3c4ccccc4c(-c4nc5ccccc5c5c4ccc4c6ccccc6sc45)c4ccccc34)cccc2c1 ; N#Cc1ccc(-c2c3ccccc3c(-c3nc4ccccc4c4c3ccc3c5ccccc5sc34)c3ccccc23)c2ccccc12 ; N#Cc1ccc(-c2c3ccccc3c(-c3nc4ccccc4c4c3ccc3c5ccccc5sc34)c3ccccc23)cc1]

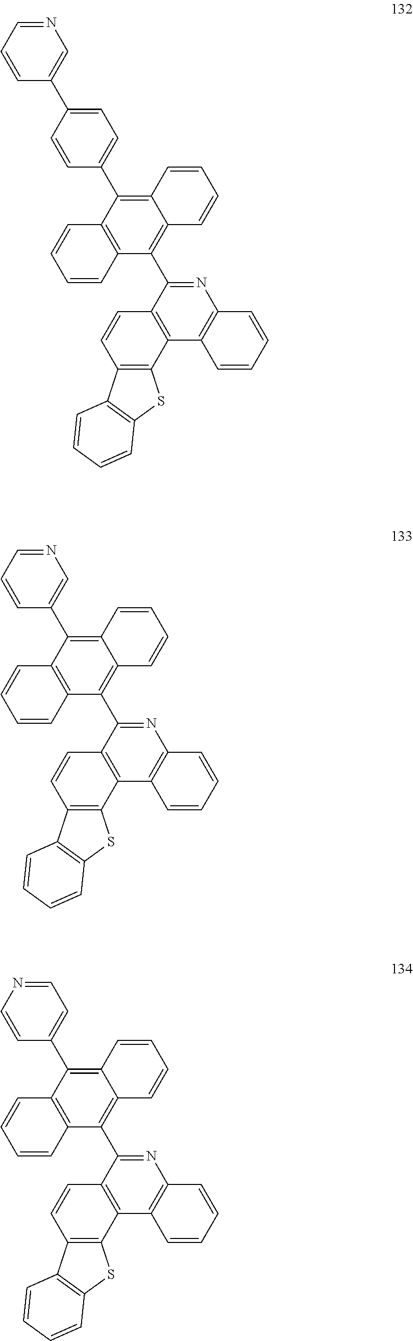 [SMILES: c1cncc(-c2ccc(-c3c4ccccc4c(-c4nc5ccccc5c5c4ccc4c6ccccc6sc45)c4ccccc34)cc2)c1 ; c1cncc(-c2c3ccccc3c(-c3nc4ccccc4c4c3ccc3c5ccccc5sc34)c3ccccc23)c1 ; c1ccc2c(c1)nc(-c1c3ccccc3c(-c3ccncc3)c3ccccc13)c1ccc3c4ccccc4sc3c12]

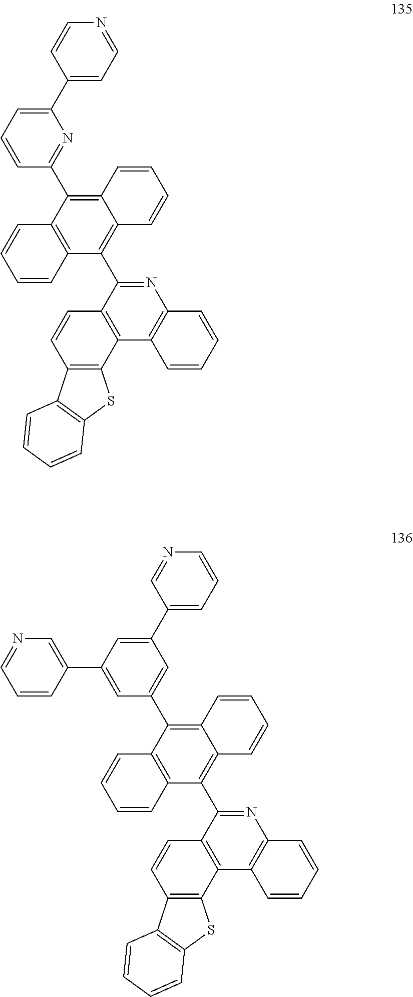 [SMILES: c1cc(-c2ccncc2)nc(-c2c3ccccc3c(-c3nc4ccccc4c4c3ccc3c5ccccc5sc34)c3ccccc23)c1 ; c1cncc(-c2cc(-c3cccnc3)cc(-c3c4ccccc4c(-c4nc5ccccc5c5c4ccc4c6ccccc6sc45)c4ccccc34)c2)c1]

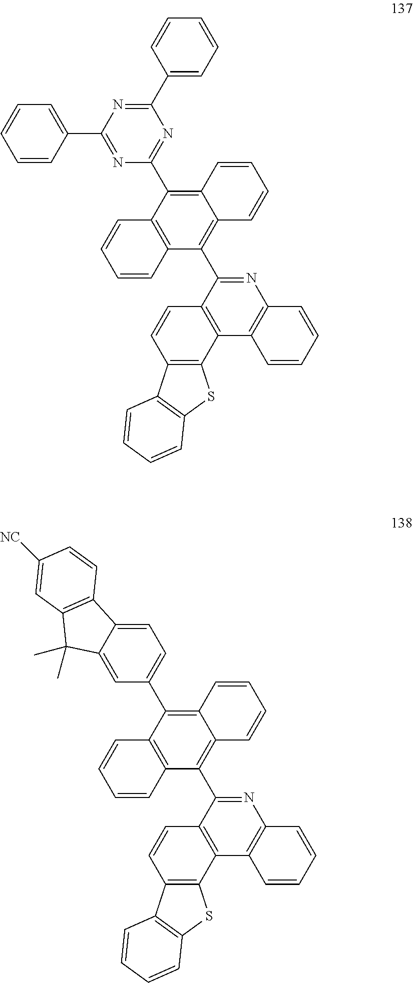 [SMILES: c1ccc(-c2nc(-c3ccccc3)nc(-c3c4ccccc4c(-c4nc5ccccc5c5c4ccc4c6ccccc6sc45)c4ccccc34)n2)cc1 ; CC1(C)c2cc(C#N)ccc2-c2ccc(-c3c4ccccc4c(-c4nc5ccccc5c5c4ccc4c6ccccc6sc45)c4ccccc34)cc21]

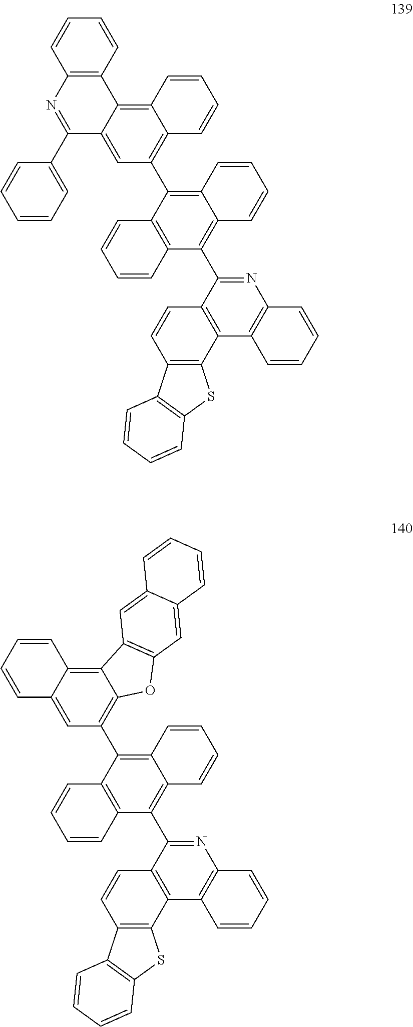 [SMILES: c1ccc(-c2nc3ccccc3c3c2cc(-c2c4ccccc4c(-c4nc5ccccc5c5c4ccc4c6ccccc6sc45)c4ccccc24)c2ccccc23)cc1 ; c1ccc2cc3c(cc2c1)oc1c(-c2c4ccccc4c(-c4nc5ccccc5c5c4ccc4c6ccccc6sc45)c4ccccc24)cc2ccccc2c13]

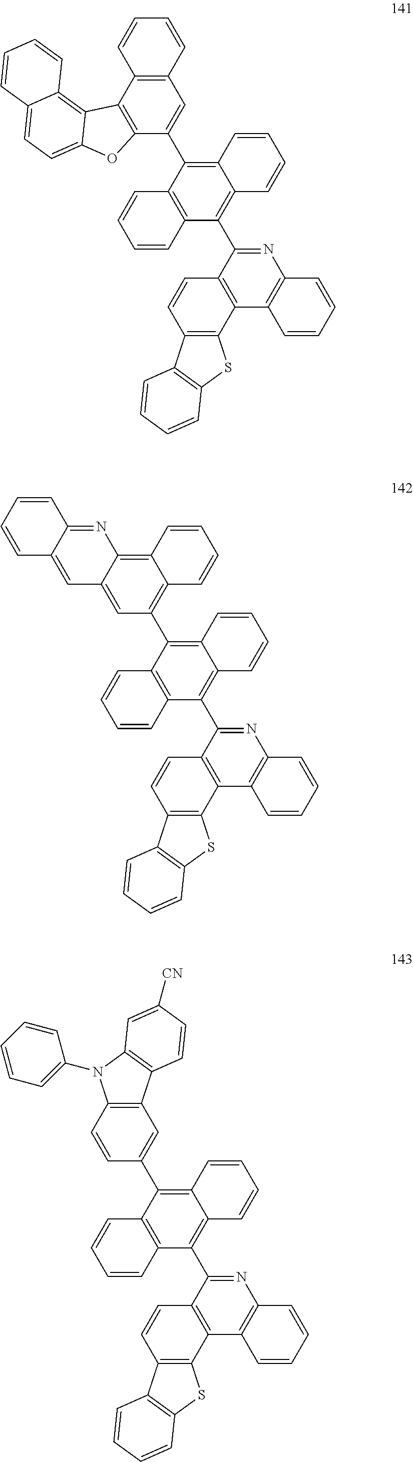 [SMILES: c1ccc2c(c1)ccc1oc3c(-c4c5ccccc5c(-c5nc6ccccc6c6c5ccc5c7ccccc7sc56)c5ccccc45)cc4ccccc4c3c12 ; c1ccc2nc3c(cc(-c4c5ccccc5c(-c5nc6ccccc6c6c5ccc5c7ccccc7sc56)c5ccccc45)c4ccccc43)cc2c1 ; N#Cc1ccc2c3cc(-c4c5ccccc5c(-c5nc6ccccc6c6c5ccc5c7ccccc7sc56)c5ccccc45)ccc3n(-c3ccccc3)c2c1]

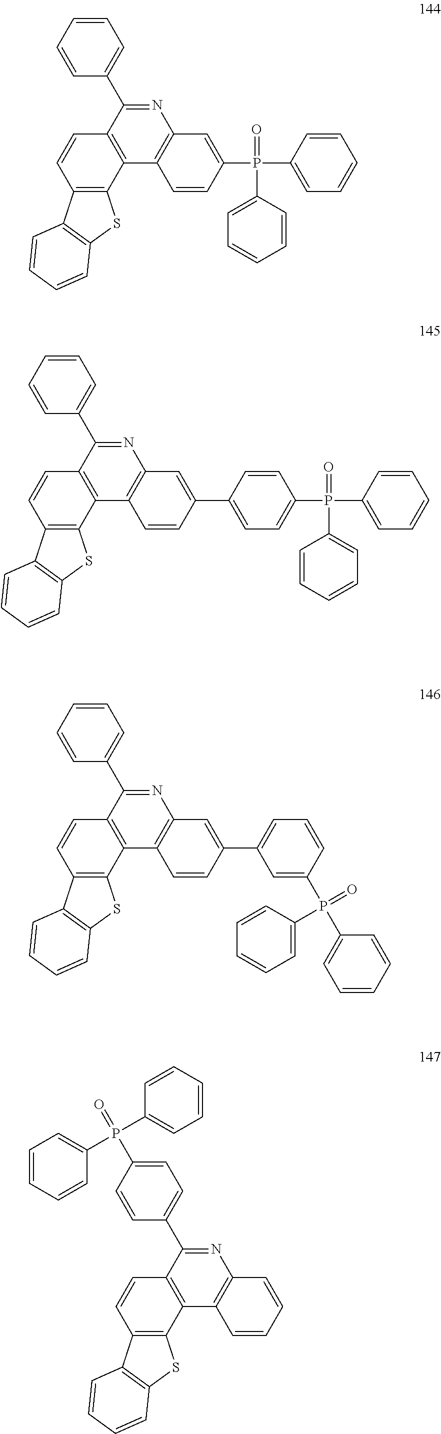 [SMILES: O=P(c1ccccc1)(c1ccccc1)c1ccc2c(c1)nc(-c1ccccc1)c1ccc3c4ccccc4sc3c12 ; O=P(c1ccccc1)(c1ccccc1)c1ccc(-c2ccc3c(c2)nc(-c2ccccc2)c2ccc4c5ccccc5sc4c23)cc1 ; O=P(c1ccccc1)(c1ccccc1)c1cccc(-c2ccc3c(c2)nc(-c2ccccc2)c2ccc4c5ccccc5sc4c23)c1 ; O=P(c1ccccc1)(c1ccccc1)c1ccc(-c2nc3ccccc3c3c2ccc2c4ccccc4sc23)cc1]

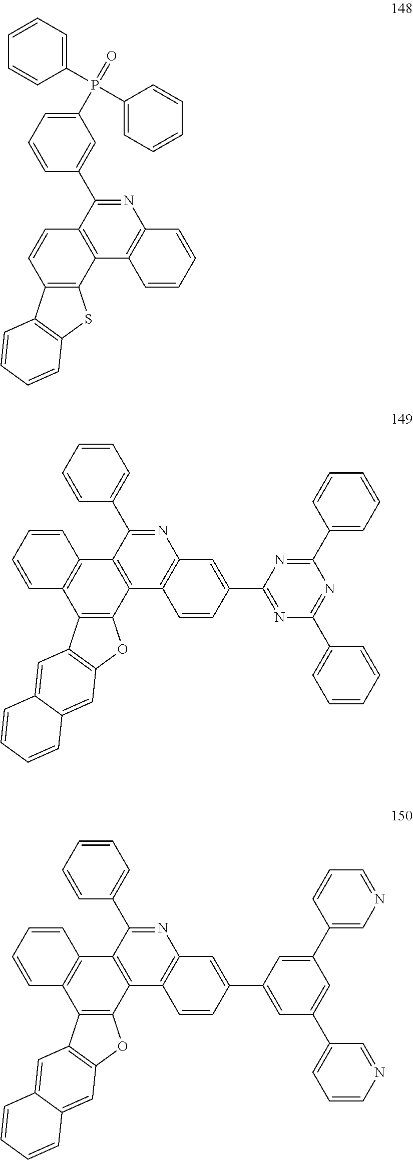 [SMILES: O=P(c1ccccc1)(c1ccccc1)c1cccc(-c2nc3ccccc3c3c2ccc2c4ccccc4sc23)c1 ; c1ccc(-c2nc(-c3ccccc3)nc(-c3ccc4c(c3)nc(-c3ccccc3)c3c5ccccc5c5c6cc7ccccc7cc6oc5c43)n2)cc1 ; c1ccc(-c2nc3cc(-c4cc(-c5cccnc5)cc(-c5cccnc5)c4)ccc3c3c4oc5cc6ccccc6cc5c4c4ccccc4c23)cc1]

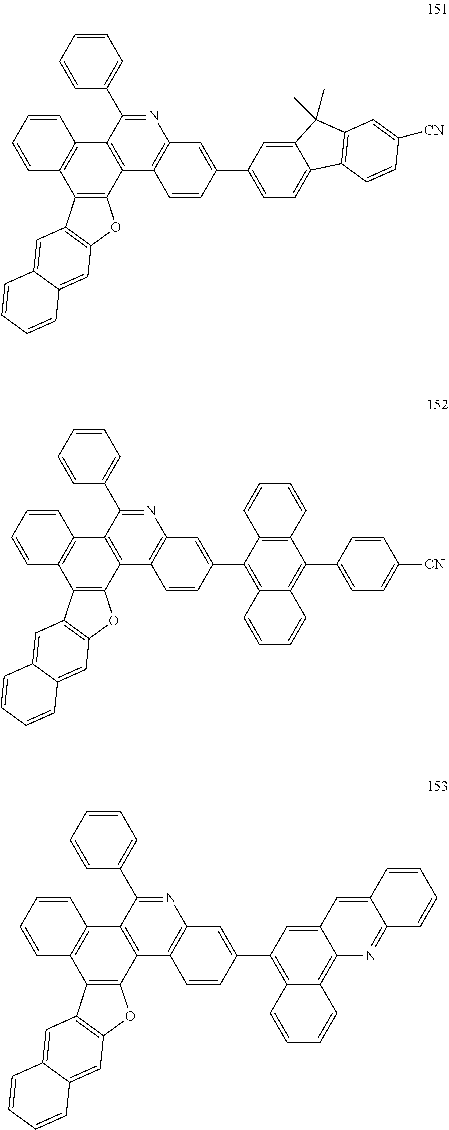 [SMILES: CC1(C)c2cc(C#N)ccc2-c2ccc(-c3ccc4c(c3)nc(-c3ccccc3)c3c5ccccc5c5c6cc7ccccc7cc6oc5c43)cc21 ; N#Cc1ccc(-c2c3ccccc3c(-c3ccc4c(c3)nc(-c3ccccc3)c3c5ccccc5c5c6cc7ccccc7cc6oc5c43)c3ccccc23)cc1 ; c1ccc(-c2nc3cc(-c4cc5cc6ccccc6nc5c5ccccc45)ccc3c3c4oc5cc6ccccc6cc5c4c4ccccc4c23)cc1]

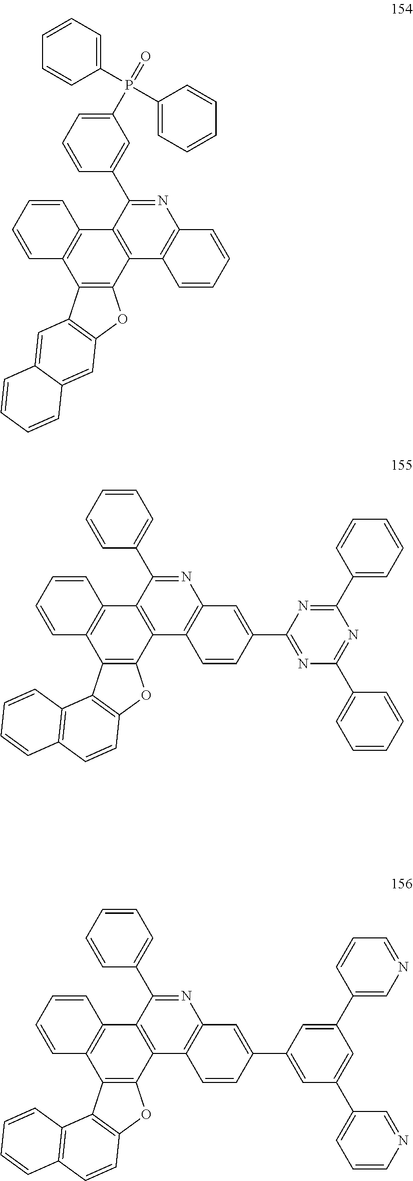 [SMILES: O=P(c1ccccc1)(c1ccccc1)c1cccc(-c2nc3ccccc3c3c4oc5cc6ccccc6cc5c4c4ccccc4c23)c1 ; c1ccc(-c2nc(-c3ccccc3)nc(-c3ccc4c(c3)nc(-c3ccccc3)c3c5ccccc5c5c(oc6ccc7ccccc7c65)c43)n2)cc1 ; c1ccc(-c2nc3cc(-c4cc(-c5cccnc5)cc(-c5cccnc5)c4)ccc3c3c4oc5ccc6ccccc6c5c4c4ccccc4c23)cc1]

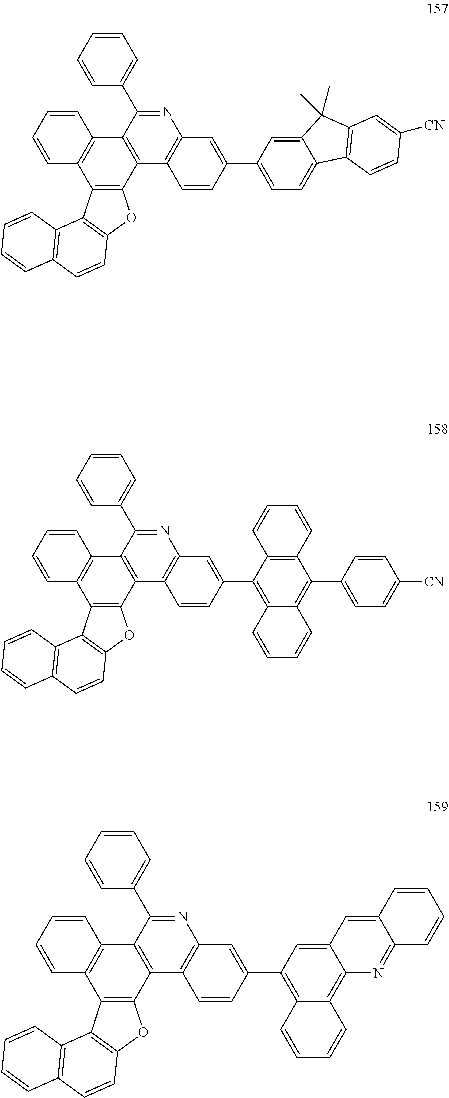 [SMILES: CC1(C)c2cc(C#N)ccc2-c2ccc(-c3ccc4c(c3)nc(-c3ccccc3)c3c5ccccc5c5c(oc6ccc7ccccc7c65)c43)cc21 ; N#Cc1ccc(-c2c3ccccc3c(-c3ccc4c(c3)nc(-c3ccccc3)c3c5ccccc5c5c(oc6ccc7ccccc7c65)c43)c3ccccc23)cc1 ; c1ccc(-c2nc3cc(-c4cc5cc6ccccc6nc5c5ccccc45)ccc3c3c4oc5ccc6ccccc6c5c4c4ccccc4c23)cc1]

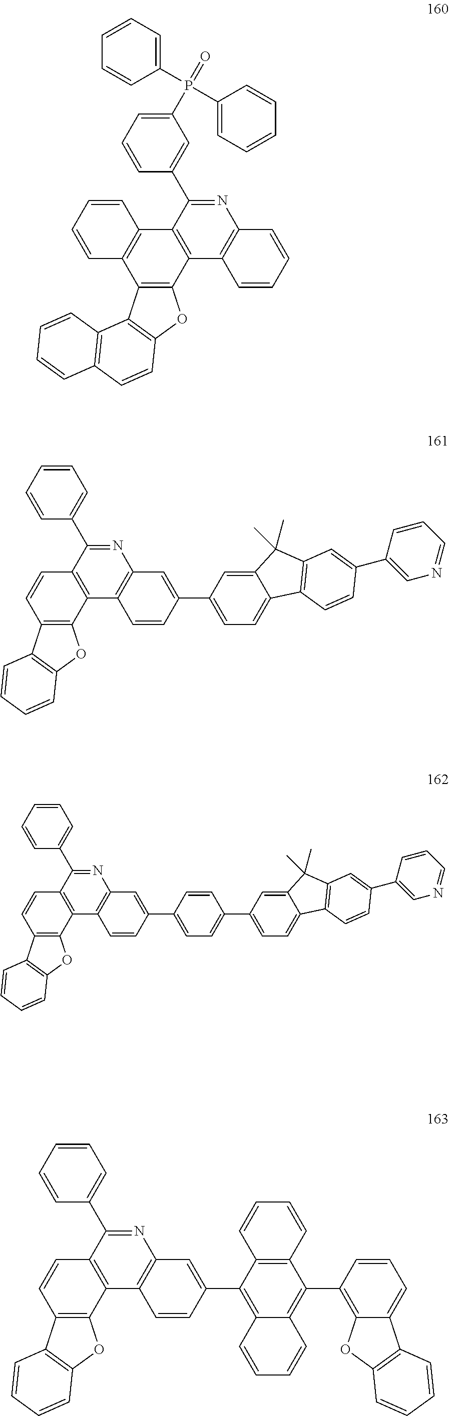 [SMILES: O=P(c1ccccc1)(c1ccccc1)c1cccc(-c2nc3ccccc3c3c4oc5ccc6ccccc6c5c4c4ccccc4c23)c1 ; CC1(C)c2cc(-c3cccnc3)ccc2-c2ccc(-c3ccc4c(c3)nc(-c3ccccc3)c3ccc5c6ccccc6oc5c34)cc21 ; CC1(C)c2cc(-c3ccc(-c4ccc5c(c4)nc(-c4ccccc4)c4ccc6c7ccccc7oc6c45)cc3)ccc2-c2ccc(-c3cccnc3)cc21 ; c1ccc(-c2nc3cc(-c4c5ccccc5c(-c5cccc6c5oc5ccccc56)c5ccccc45)ccc3c3c2ccc2c4ccccc4oc23)cc1]

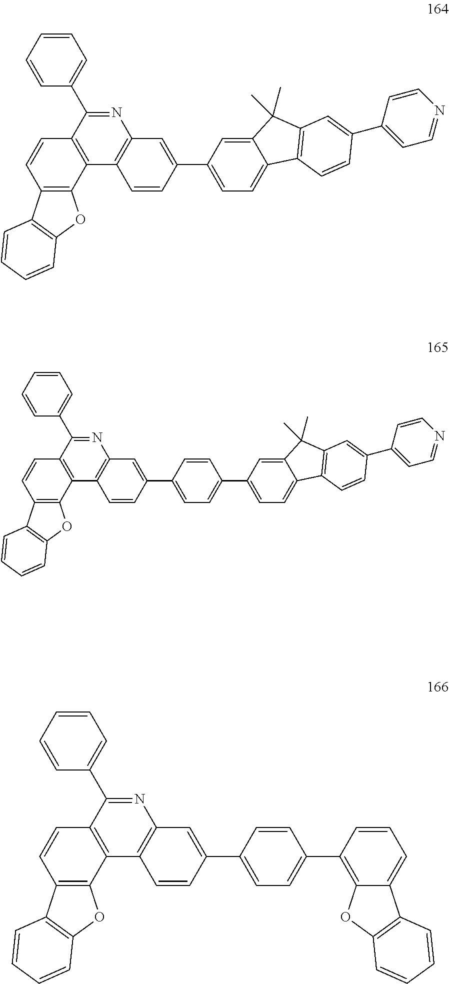 [SMILES: CC1(C)c2cc(-c3ccncc3)ccc2-c2ccc(-c3ccc4c(c3)nc(-c3ccccc3)c3ccc5c6ccccc6oc5c34)cc21 ; CC1(C)c2cc(-c3ccncc3)ccc2-c2ccc(-c3ccc(-c4ccc5c(c4)nc(-c4ccccc4)c4ccc6c7ccccc7oc6c45)cc3)cc21 ; c1ccc(-c2nc3cc(-c4ccc(-c5cccc6c5oc5ccccc56)cc4)ccc3c3c2ccc2c4ccccc4oc23)cc1]

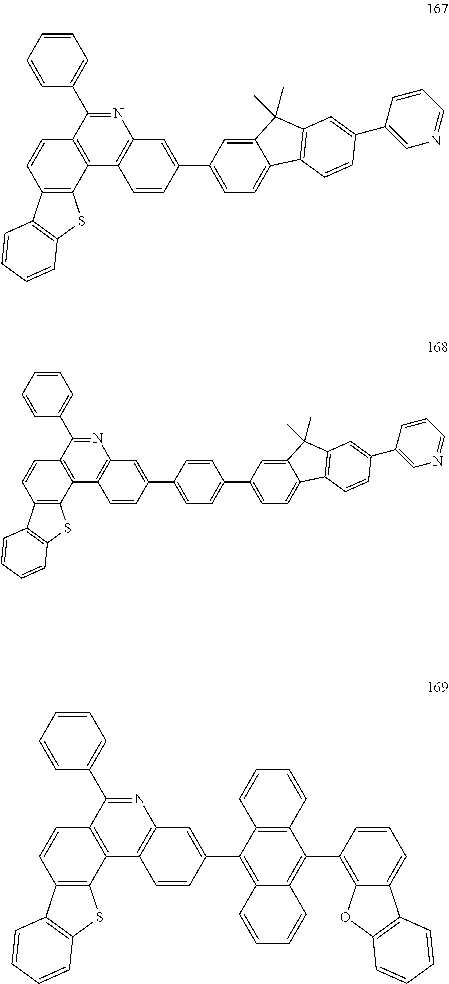 [SMILES: CC1(C)c2cc(-c3cccnc3)ccc2-c2ccc(-c3ccc4c(c3)nc(-c3ccccc3)c3ccc5c6ccccc6sc5c34)cc21 ; CC1(C)c2cc(-c3ccc(-c4ccc5c(c4)nc(-c4ccccc4)c4ccc6c7ccccc7sc6c45)cc3)ccc2-c2ccc(-c3cccnc3)cc21 ; c1ccc(-c2nc3cc(-c4c5ccccc5c(-c5cccc6c5oc5ccccc56)c5ccccc45)ccc3c3c2ccc2c4ccccc4sc23)cc1]

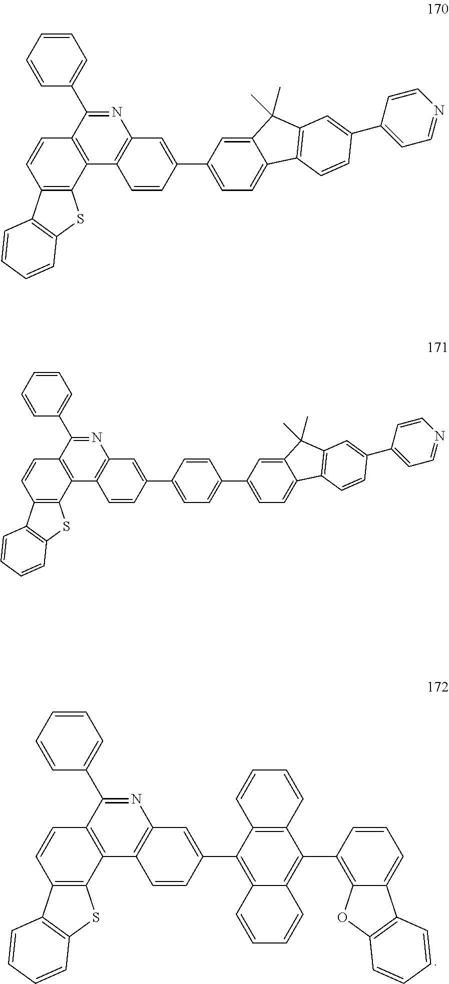 [SMILES: CC1(C)c2cc(-c3ccncc3)ccc2-c2ccc(-c3ccc4c(c3)nc(-c3ccccc3)c3ccc5c6ccccc6sc5c34)cc21 ; CC1(C)c2cc(-c3ccncc3)ccc2-c2ccc(-c3ccc(-c4ccc5c(c4)nc(-c4ccccc4)c4ccc6c7ccccc7sc6c45)cc3)cc21 ; c1ccc(-c2nc3cc(-c4c5ccccc5c(-c5cccc6c5oc5ccccc56)c5ccccc45)ccc3c3c2ccc2c4ccccc4sc23)cc1]

The term “organic layer” used herein may refer to a single layer and/or a plurality of layers disposed (e.g., positioned) between the first electrode and the second electrode of an organic light-emitting device. A material included in the “organic layer” is not limited to an organic material.

The drawing is a schematic view of an organic light-emitting device 10 according to an embodiment. The organic light-emitting device 10 includes a first electrode 110, an organic layer 150, and a second electrode 190.

Hereinafter, the structure of an organic light-emitting device according to an embodiment and a method of manufacturing an organic light-emitting device according to an embodiment will be described in connection with the drawing.

In the drawing, a substrate may be additionally disposed under the first electrode 110 or above the second electrode 190. The substrate may be a glass substrate or a transparent plastic substrate, each having excellent mechanical strength, thermal stability, transparency, surface smoothness, ease of handling, and/or water-resistance.

The first electrode 110 may be formed by depositing or sputtering a material for forming the first electrode 110 on the substrate. When the first electrode 110 is an anode, the material for forming the first electrode 110 may be selected from materials with a high work function to facilitate hole injection. The first electrode 110 may be a reflective electrode, a semi-transmissive electrode, or a transmissive electrode. The material for forming the first electrode may be a transparent and highly conductive material, and non-limiting examples of such material include indium tin oxide (ITO), indium zinc oxide (IZO), tin oxide (SnO₂), and zinc oxide (ZnO). When the first electrode 110 is a semi-transmissive electrode or a reflective electrode, at least one selected from magnesium (Mg), aluminum(Al), aluminum-lithium (Al—Li), calcium (Ca), magnesium-indium(Mg—In), and magnesium-silver (Mg—Ag) may be used as a material for forming the first electrode 110.

The first electrode 110 may have a single-layer structure, or a multi-layer structure including two or more layers. For example, the first electrode 110 may have a three-layered structure of ITO/Ag/ITO, but the structure of the first electrode 110 is not limited thereto.

The organic layer 150 may be disposed on the first electrode 110. The organic layer 150 may include an emission layer.

The organic layer 150 may further include a hole transport region between the first electrode and the emission layer, and an electron transport region between the emission layer and the second electrode.

In some embodiments, the hole transport region may include at least one selected from a hole transport layer (HTL), a hole injection layer (HIL), a buffer layer, and an electron blocking layer; and an electron transport region may include at least one selected from a hole blocking layer (HBL), an electron transport layer (ETL), and an electron injection layer (EIL). However, it may be understood that embodiments are not limited thereto.

The hole transport region may have a single-layered structure formed of a single material, a single-layered structure formed of a plurality of different materials, or a multi-layered structure having a plurality of layers formed of a plurality of different materials.

For example, the hole transport region may have a single-layered structure formed of a plurality of different materials, or a structure of hole injection layer/hole transport layer, a structure of hole injection layer/hole transport layer/buffer layer, a structure of hole injection layer/buffer layer, a structure of hole transport layer/buffer layer, or a structure of hole injection layer/hole transport layer/electron blocking layer, wherein the layers of each structure are sequentially stacked from the first electrode 110 in this stated order, but the structure of the hole transport region is not limited thereto.

When the hole transport region includes a hole injection layer, the hole injection layer may be formed on the first electrode 110 by using one or more suitable methods such as vacuum deposition, spin coating, casting, a Langmuir-Blodgett (LB) method, ink-jet printing, laser-printing, and/or laser-induced thermal imaging.

When a hole injection layer is formed by vacuum deposition, for example, the vacuum deposition may be performed at a deposition temperature of about 100 to about 500° C., at a vacuum degree of about 10⁻⁸ to about 10⁻³ torr, and at a deposition rate of about 0.01 to about 100 Å/sec, by taking into account a compound for forming the hole injection layer to be deposited, and the structure of the hole injection layer to be formed.

When a hole injection layer is formed by spin coating, for example, the spin coating may be performed at a coating rate of about 2,000 rpm to about 5,000 rpm, and at a temperature of about 80° C. to 200° C., by taking into account a compound for forming the hole injection layer to be deposited, and the structure of the hole injection layer to be formed.

When the hole transport region includes a hole transport layer, the hole transport layer may be formed on the first electrode 110 or the hole injection layer by using one or more suitable methods such as vacuum deposition, spin coating, casting, a LB method, ink-jet printing, laser-printing, and/or laser-induced thermal imaging. When the hole transport layer is formed by vacuum deposition and/or spin coating, deposition and coating conditions for the hole transport layer may be the same as (or similar to) the deposition and coating conditions for the hole injection layer.

The hole transport region may include m-MTDATA, TDATA, 2-TNATA, NPB, β-NPB, TPD, Spiro-TPD, Spiro-NPB, methylated NPB, TAPC, HMTPD, 4,4′, 4″-tris(N-carbazolyl)triphenylamine (TCTA), polyaniline/dodecylbenzenesulfonic acid (Pani/DBSA), poly(3,4-ethylenedioxythiophene)/poly(4-styrenesulfonate) (PEDOT/PSS), polyaniline/camphor sulfonicacid (Pani/CSA), (polyaniline)/poly(4-styrenesulfonate) (PANI/PSS), a compound represented by Formula 201 below, and/or a compound represented by Formula 202 below:

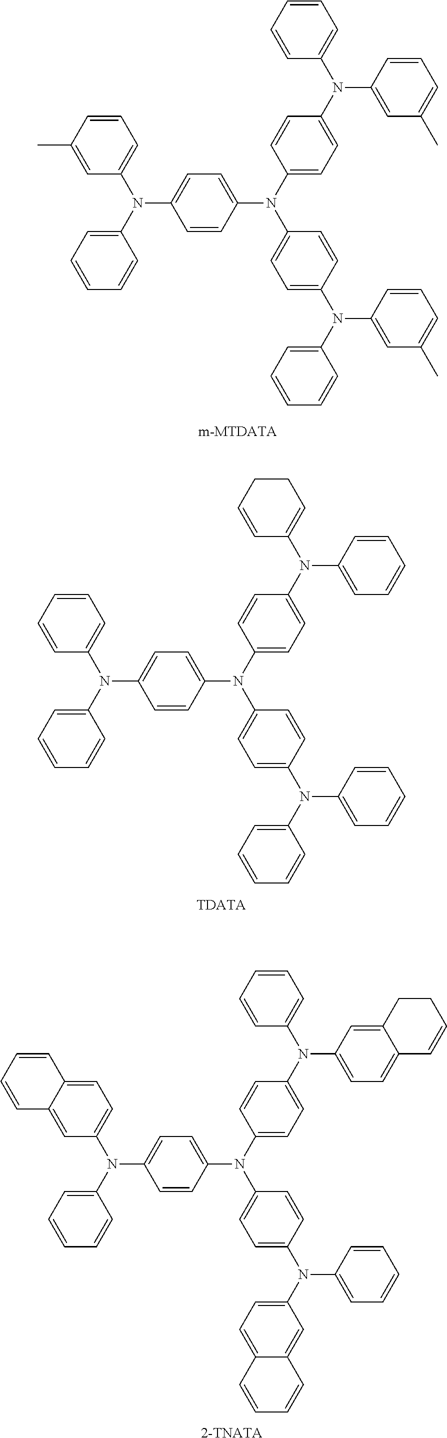

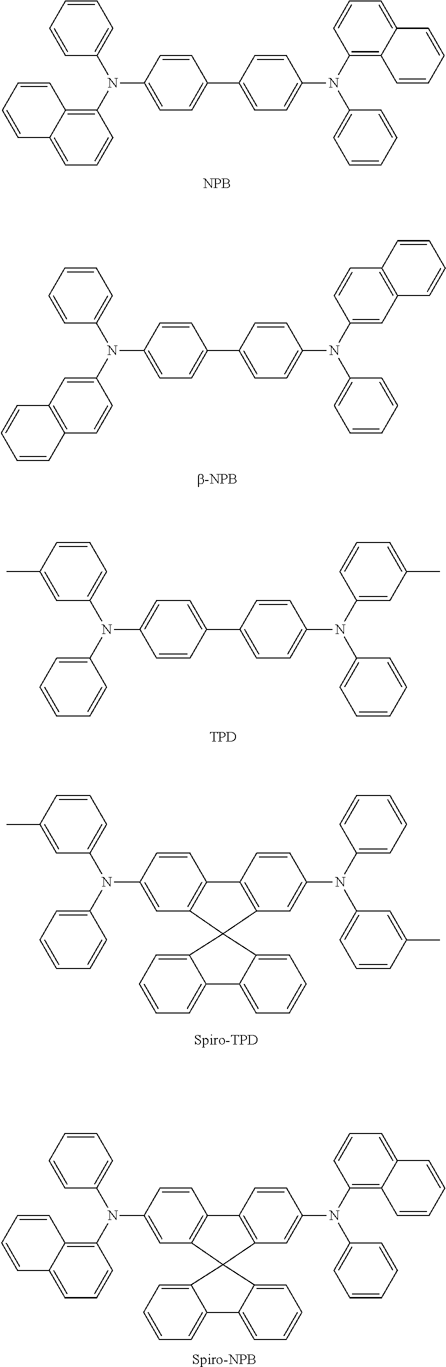

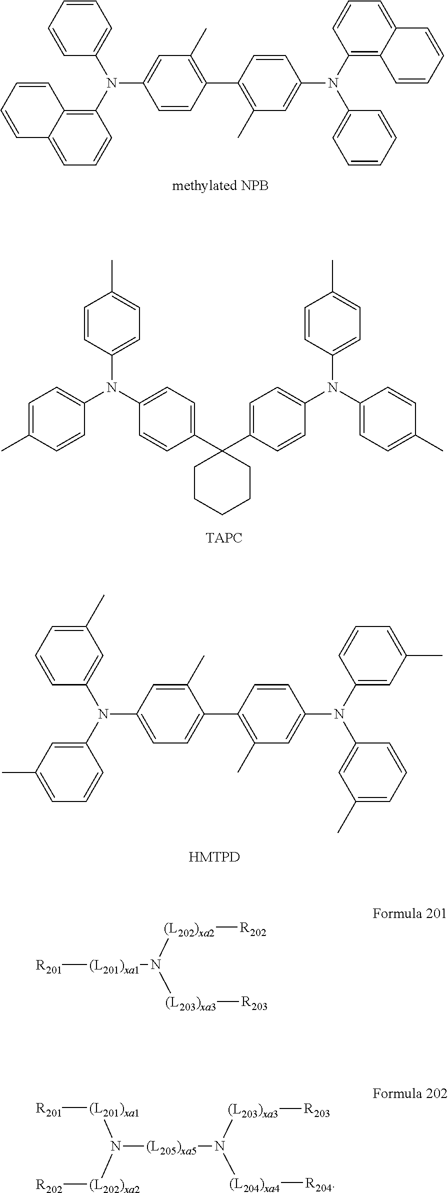

In Formulae 201 and 202,

L₂₀₁ to L₂₀₅ may each independently be selected from a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group;

xa1 to xa4 may each independently be selected from 0, 1, 2, and 3;

xa5 may be selected from 1, 2, 3, 4, and 5; and

R₂₀₁ to R₂₀₄ may each independently be selected from a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In some embodiments, in Formulae 201 and 202,

L₂₀₁ to L₂₀₅ may each independently be selected from the group consisting of:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from deuterium, —F, —Cl, —Br, —I, a hydroxyl group, a cyano group, a nitro group, an amino group, an am idino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, an isoindolyl group, a quinolinyl group, an isoquinolinyl group, a quinoxalinyl group, a quinazolinyl group, a carbazolyl group, and a triazinyl group;

xa1 to xa4 may each independently be 0, 1, or 2;

xa5 may be 1, 2, or 3;

R₂₀₁ to R₂₀₄ may each independently be selected from the group consisting of:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, an azulenyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, but embodiments of the present disclosure are not limited thereto.

The compound represented by Formula 201 may be represented by Formula 201A:

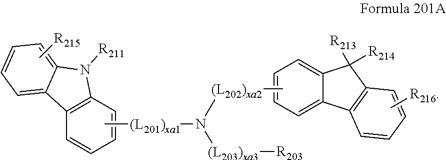

For example, the compound represented by Formula 201 may be represented by Formula 201A-1 below, but is not limited thereto:

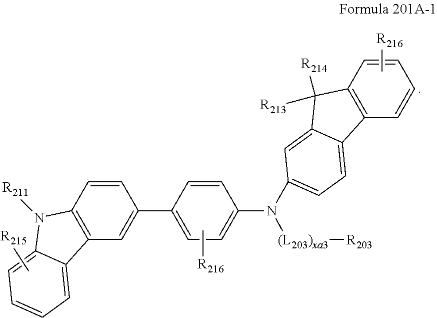

For example, the compound represented by Formula 202 may be represented by Formula 202A below, but is not limited thereto:

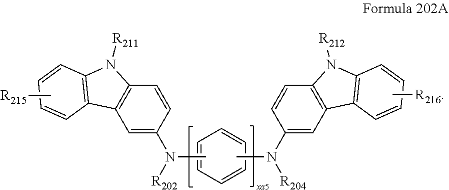

L₂₀₁ to L₂₀₃, xa1 to xa3, xa5, and R₂₀₂ to R₂₀₄ in Formulae 201A, 201A-1, and 202A are already described above; description of R₂₁₁ may be the same as the description provided in connection with R₂₀₃; and R₂₁₃ to R₂₁₆ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

For example, in Formulae 201A, 201A-1, and 202A,

L₂₀₁ to L₂₀₃ may each independently be selected from the group consisting of:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

xa1 to xa3 may each independently be 0 or 1;

R₂₀₃, R₂₁₁, and R₂₁₂ may each independently be selected from the group consisting of:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

R₂₁₃ and R₂₁₄ may each independently be selected from the group consisting of:

a C₁-C₂₀ alkyl group and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

R₂₁₅ and R₂₁₆ may each independently be selected from the group consisting of:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, and a triazinyl group; and

a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

xa5 may be 1 or 2.

In some embodiments, R₂₁₃ and R₂₁₄ in Formulae 201A and/or 201A-1 may be linked to each other to form a saturated or unsaturated ring.

The compound represented by Formula 201, and the compound represented by Formula 202 may each independently include any of compounds HT1 to HT20 illustrated below, but are not limited thereto.

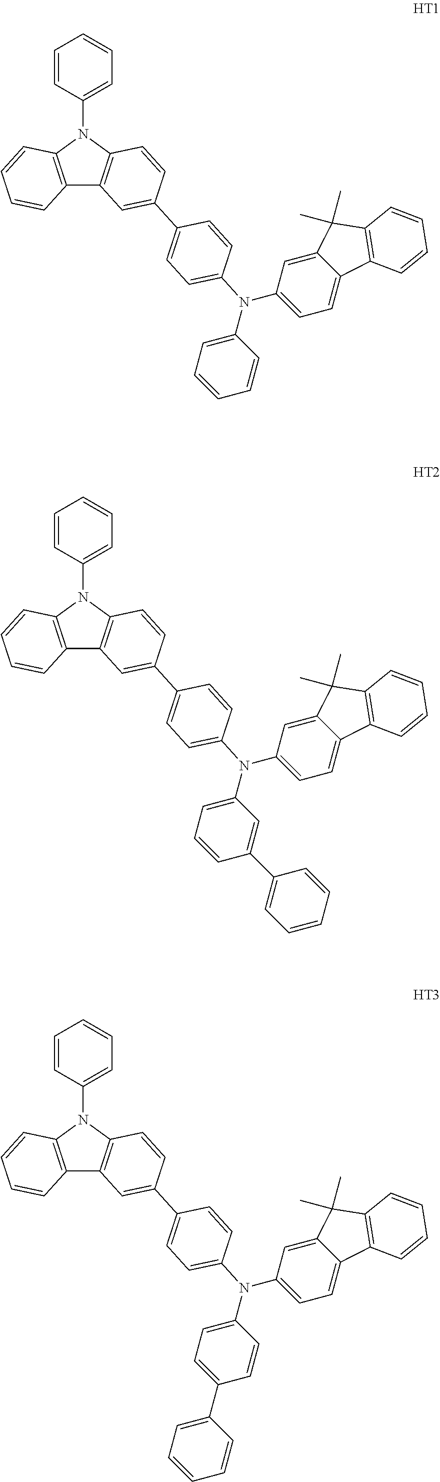 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

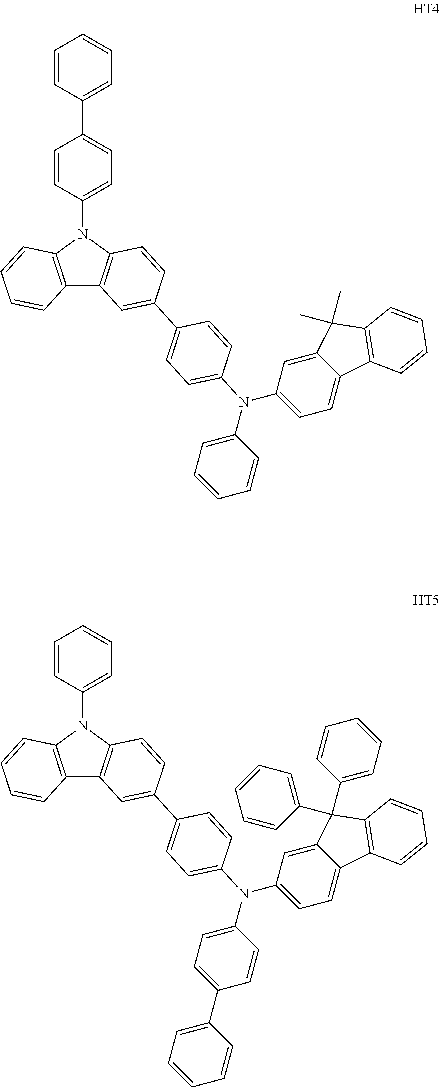 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

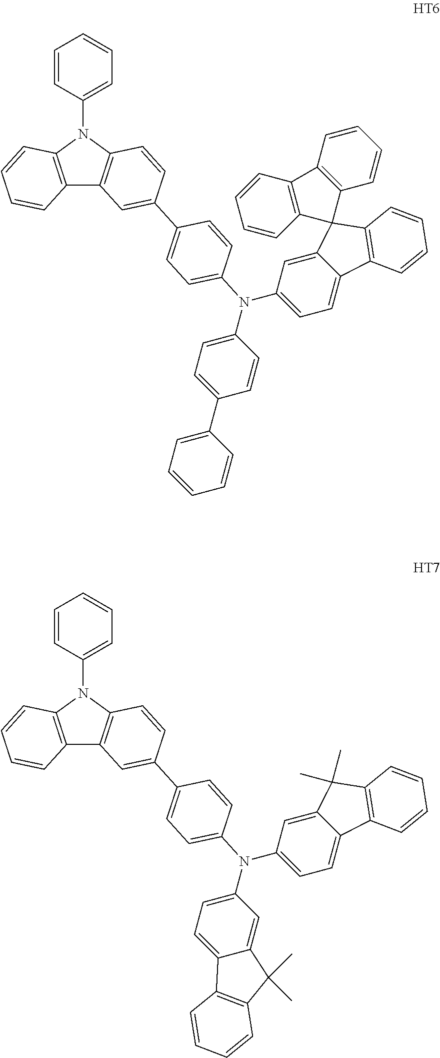 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

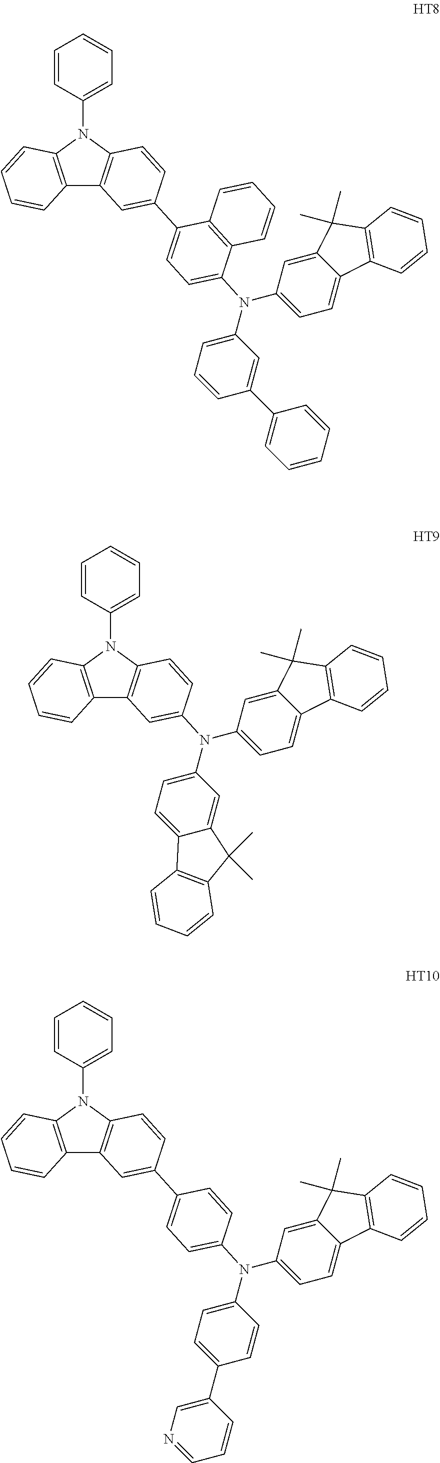 [SMILES: CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

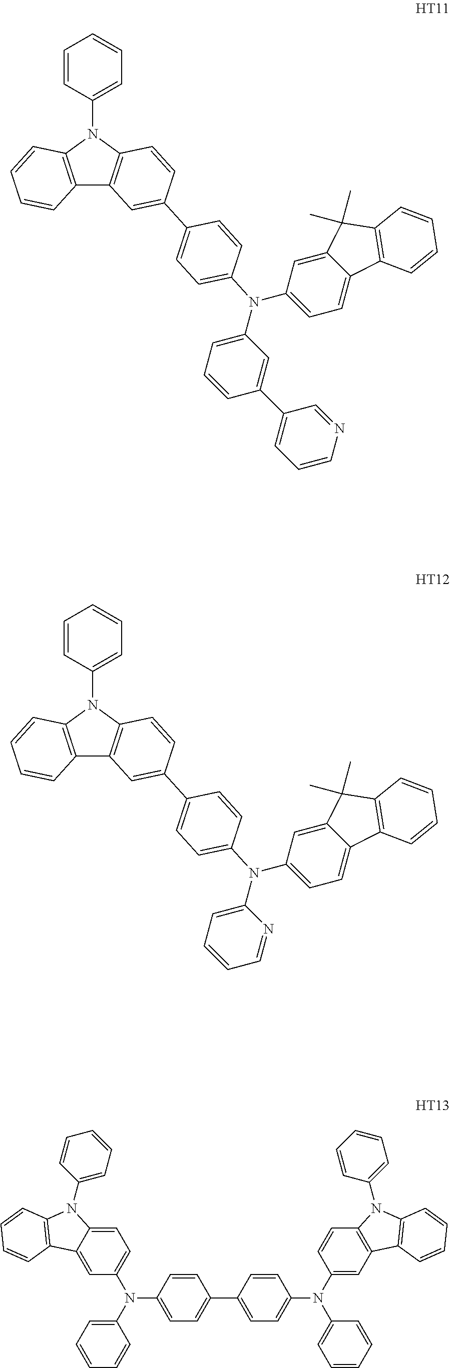 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

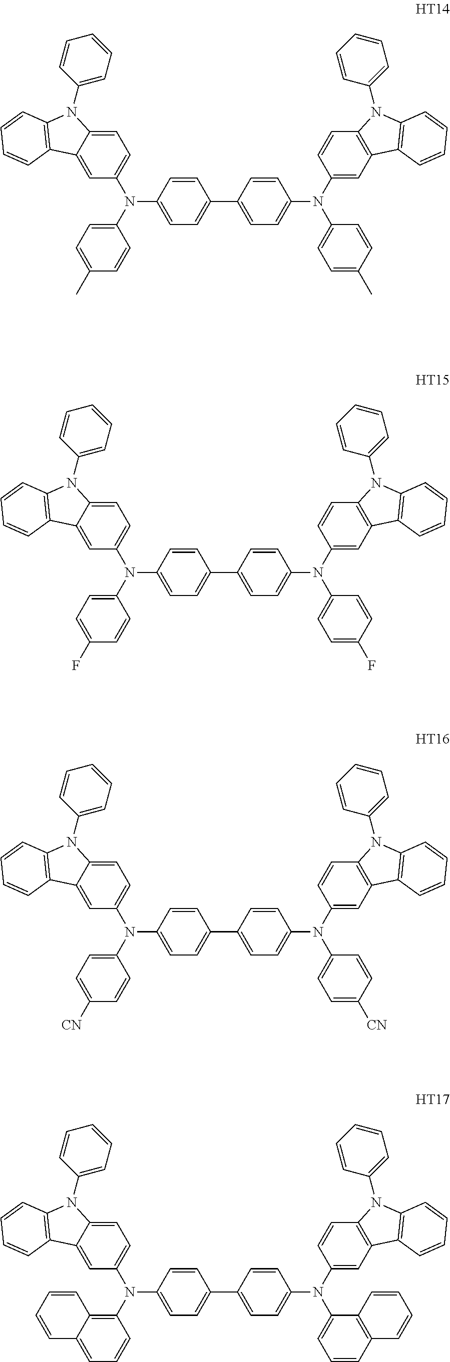

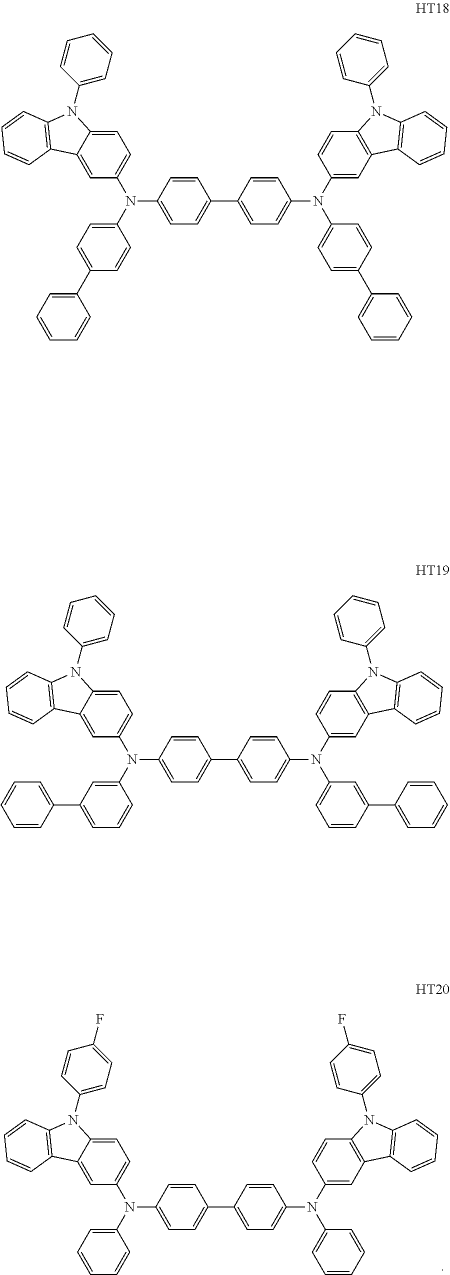 [SMILES: c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes a hole injection layer and a hole transport layer, the thickness of the hole injection layer may be in a range of about 100 Å to about 10,000 Å, for example, about 100 Å to about 1,000 Å; and the thickness of the hole transport layer may be in a range of about 50 Å to about 2,000 Å, for example, about 100 Å to about 1,500 Å. When the thicknesses of the hole transport region, the hole injection layer, and the hole transport layer are within any of these ranges, satisfactory (or suitable) hole transporting characteristics may be obtained without a substantial increase in driving voltage.

The hole transport region may further include, in addition to the materials described above, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments are not limited thereto. Non-limiting examples of the p-dopant include quinone derivatives (such as tetracyanoquinonedimethane (TCNQ) and/or 2,3,5,6-tetrafluoro-tetracyano-1,4-benzoquinonedimethane (F4-TCNQ)), metal oxides (such as tungsten oxide and/or molybdenum oxide), and Compound HT-D1 illustrated below.

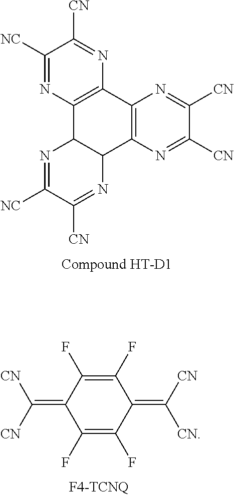

The hole transport region may further include a buffer layer and/or an electron blocking layer, in addition to a hole injection layer and/or a hole transport layer. Since the buffer layer may compensate for an optical resonance distance according to a wavelength of light emitted from the emission layer, light-emission efficiency of a formed organic light-emitting device may be improved. For use as a material included in the buffer layer, any of the materials that are to be included in the hole transport region may be used. The electron blocking layer may function to prevent or reduce the injection of electrons from the electron transport region.

An emission layer may be formed on the first electrode 110 or the hole transport region by using one or more suitable methods such as vacuum deposition, spin coating, casting, a LB method, ink-jet printing, laser-printing, and/or laser-induced thermal imaging. When an emission layer is formed by vacuum deposition and/or spin coating, deposition and coating conditions for the emission layer may be the same as (or similar to) those for the hole injection layer.

When the organic light-emitting device 10 is a full color organic light-emitting device, the emission layer may be patterned into a red emission layer, a green emission layer, and/or a blue emission layer, according to a sub pixel. In some embodiments, the emission layer may have a stacked structure including a red emission layer, a green emission layer, and a blue emission layer, or may include a red-light emission material, a green-light emission material, and a blue-light emission material, which are mixed with each other in a single layer, to emit white light.

The emission layer may include a host and a dopant.

For example, the host may include at least one selected from TPBi, TBADN, ADN (herein also referred to as “DNA”), CBP, CDBP, and TCP:

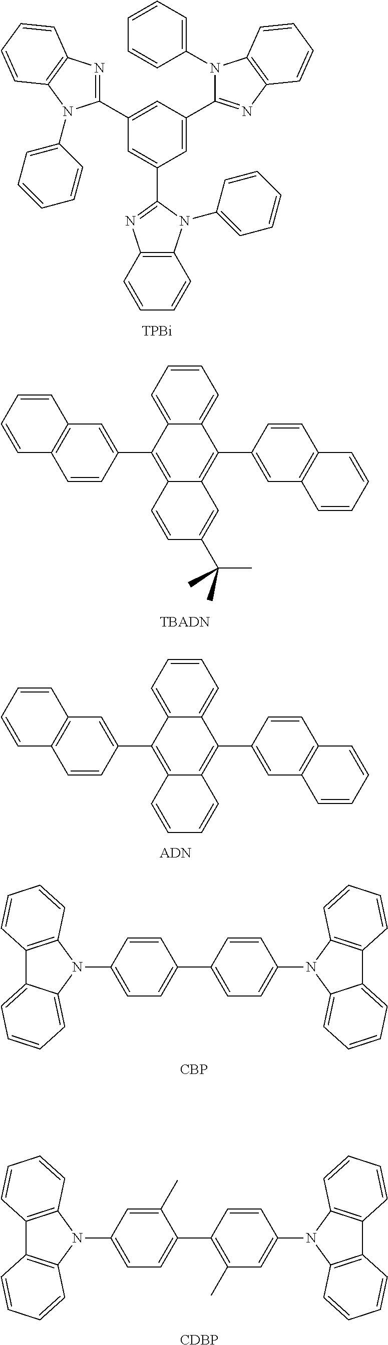

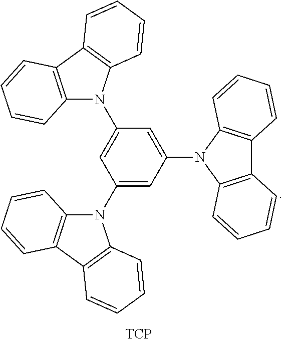

In some embodiments, the host may include a compound represented by Formula 301 below.

Ar₃₀₁-[(L₃₀₁)_{xb1}-R₃₀₁]_{xb2}. Formula 301

In Formula 301,

Ar₃₀₁ may be selected from the group consisting of:

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene; and

a naphthalene group, a heptalene group, a fluorene group, a spiro-fluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, and an indenoanthracene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₀₁)(Q₃₀₂)(Q₃₀₃) (wherein Q₃₀₁ to Q₃₀₃ may each independently be selected from hydrogen, a C₁-C₆₀ alkyl group, a C₂-C₆₀ alkenyl group, a C₁-C₆₀ aryl group, and a C₁-C₆₀ heteroaryl group);

L₃₀₁ may be selected from the group consisting of:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, an isoindolyl group, a quinolinyl group, an isoquinolinyl group, a quinoxalinyl group, a quinazolinyl group, a carbazolyl group, and a triazinyl group;

R₃₀₁ may be selected from the group consisting of:

a C₁-C₂₀ alkyl group and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

xb1 may be selected from 0, 1, 2, and 3; and

xb2 may be selected from 1, 2, 3, and 4.

For example, in Formula 301,

L₃₀₁ may be selected from the group consisting of:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, and a chrysenylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, and a chrysenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group,

R₃₀₁ may be selected from the group consisting of:

a C₁-C₂₀ alkyl group and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group, but is not limited thereto.

For example, the host may include a compound represented by Formula 301A below:

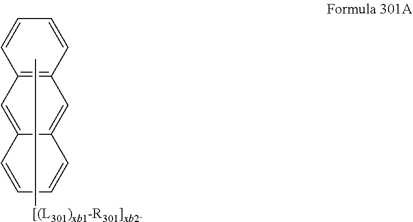

Descriptions of substituents of Formula 301A may be understood by referring to the descriptions thereof provided herein.

The compound represented by Formula 301 may include at least one of Compounds H1 to H42, but is not limited thereto:

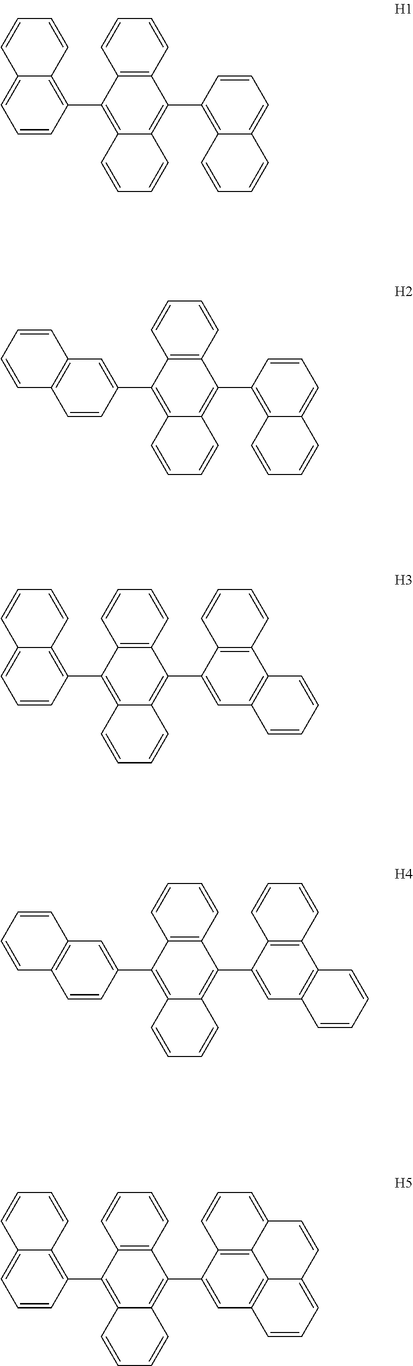

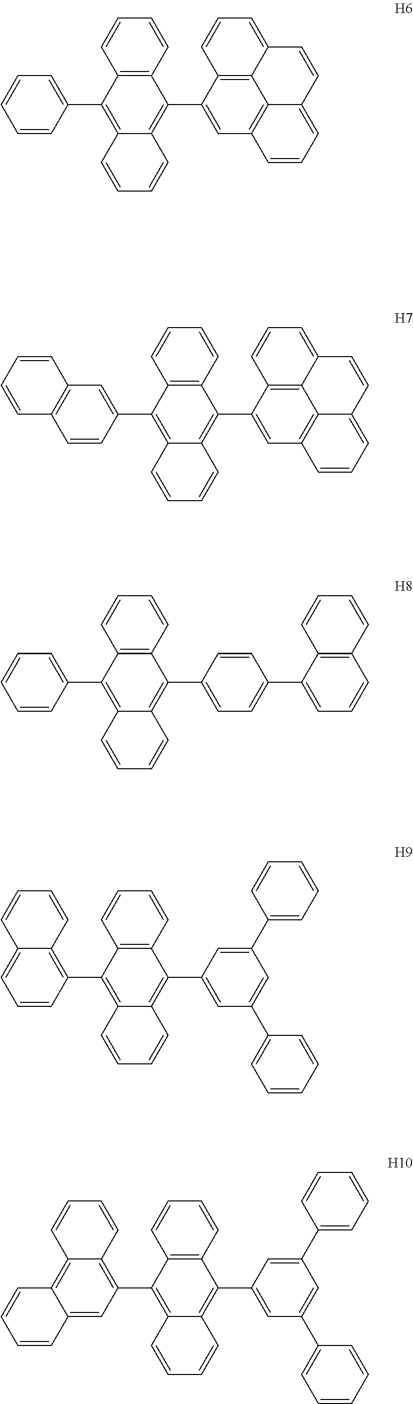

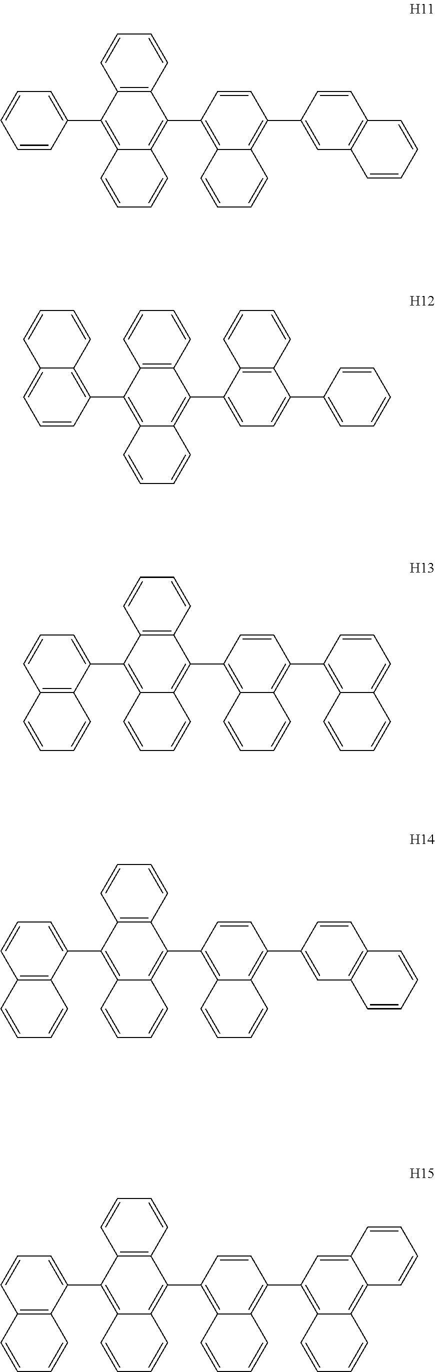

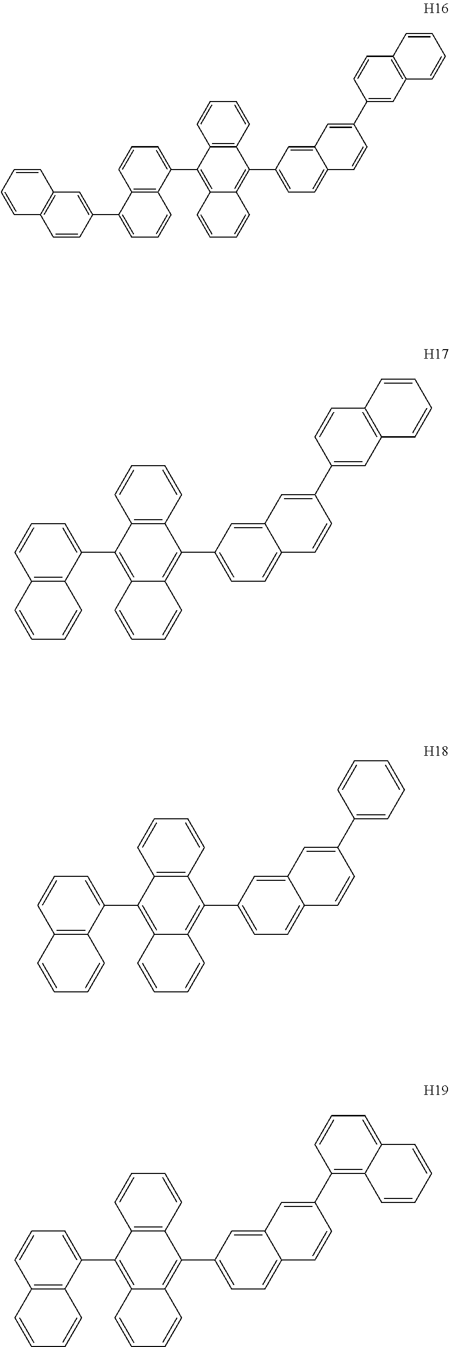

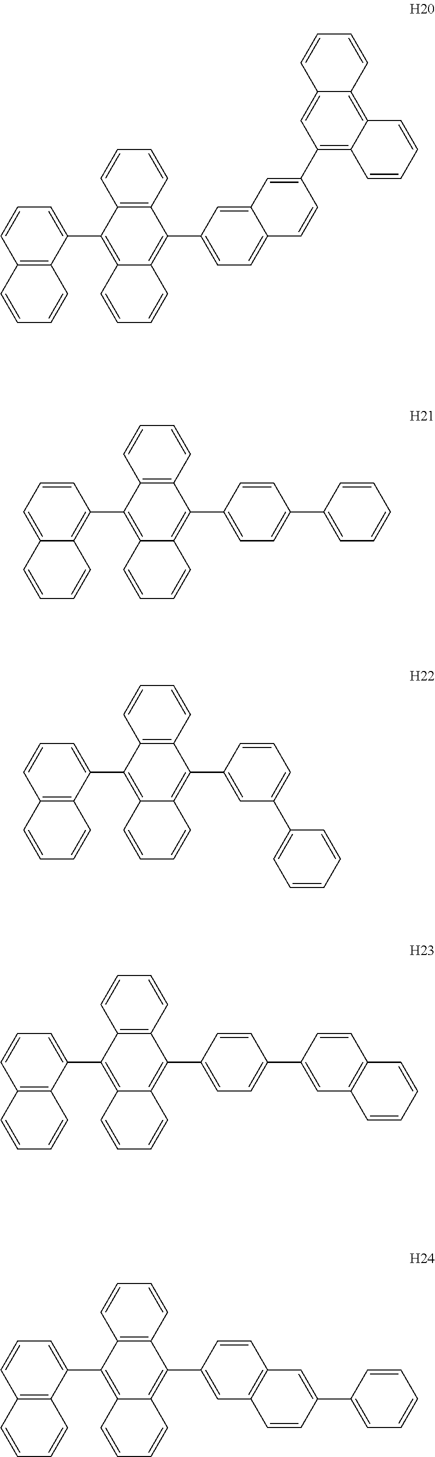

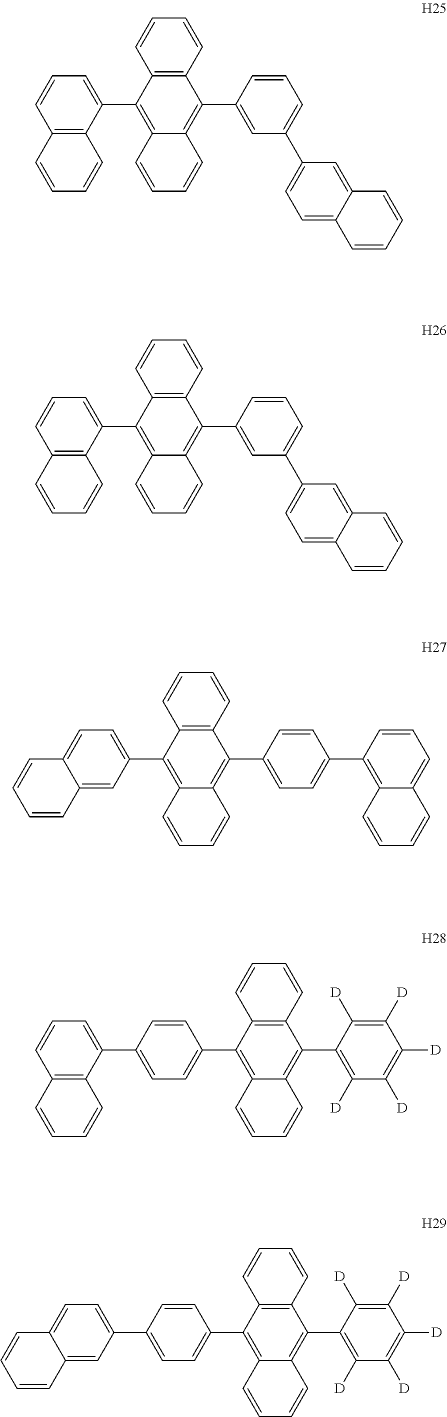

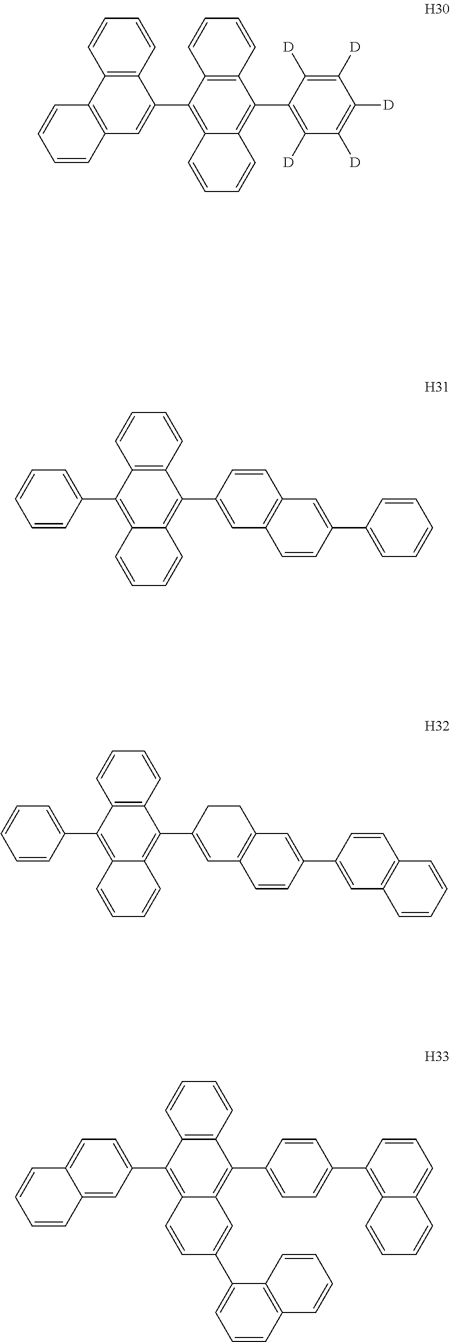

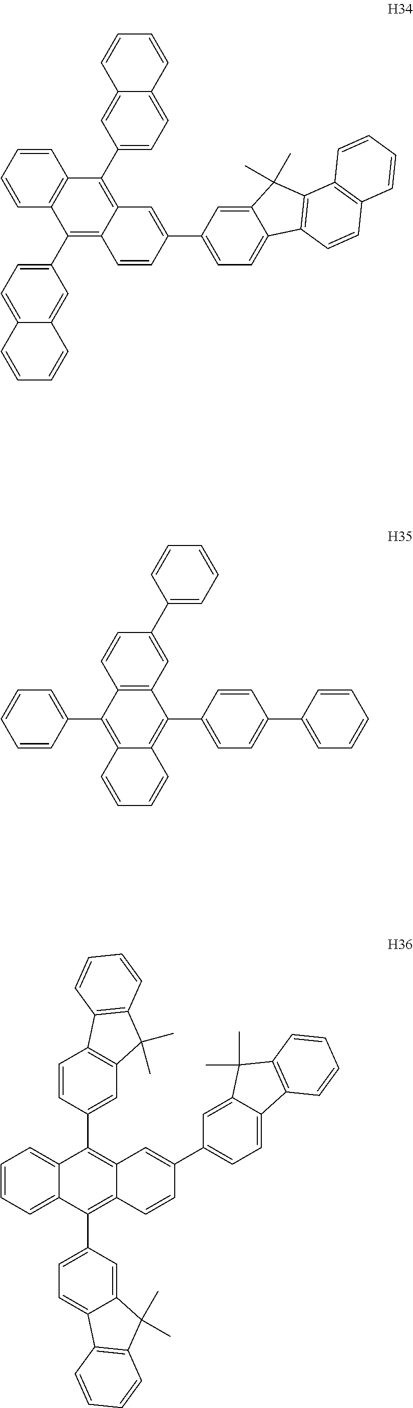

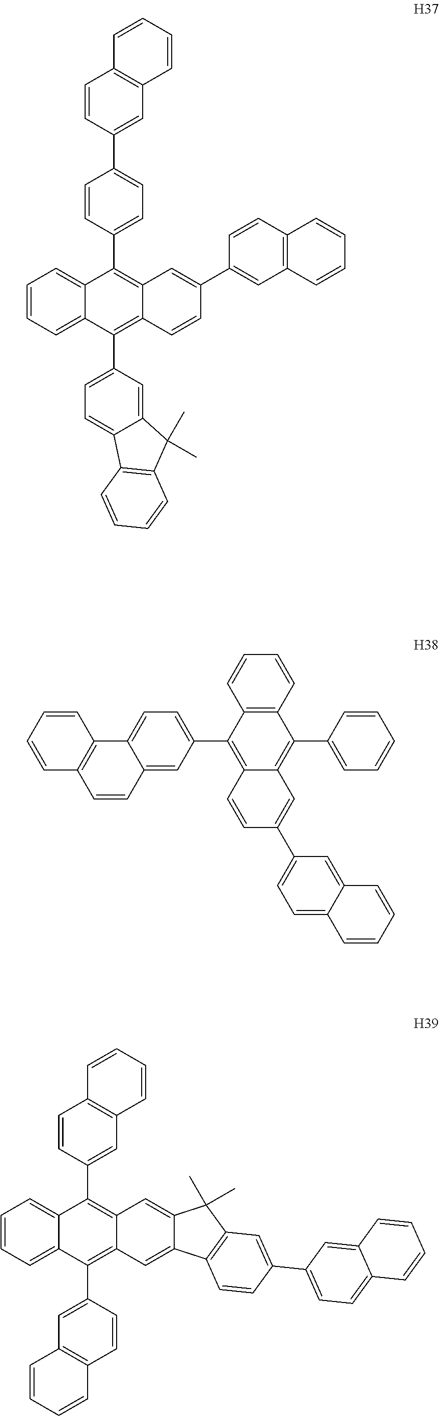

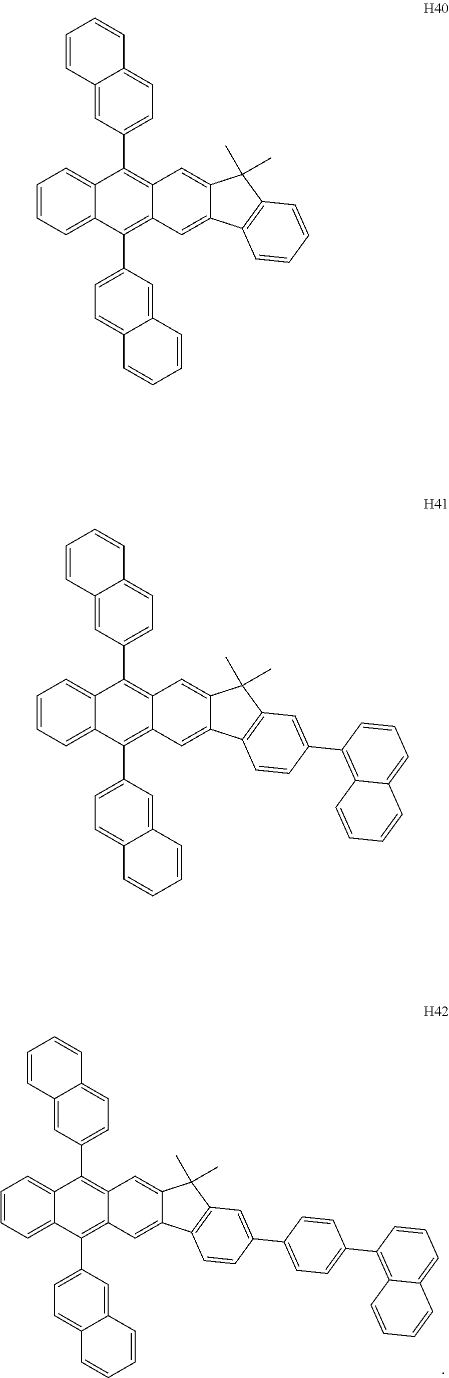

In some embodiments, the host may include at least one of Compounds H43 to H49 below, but is not limited thereto:

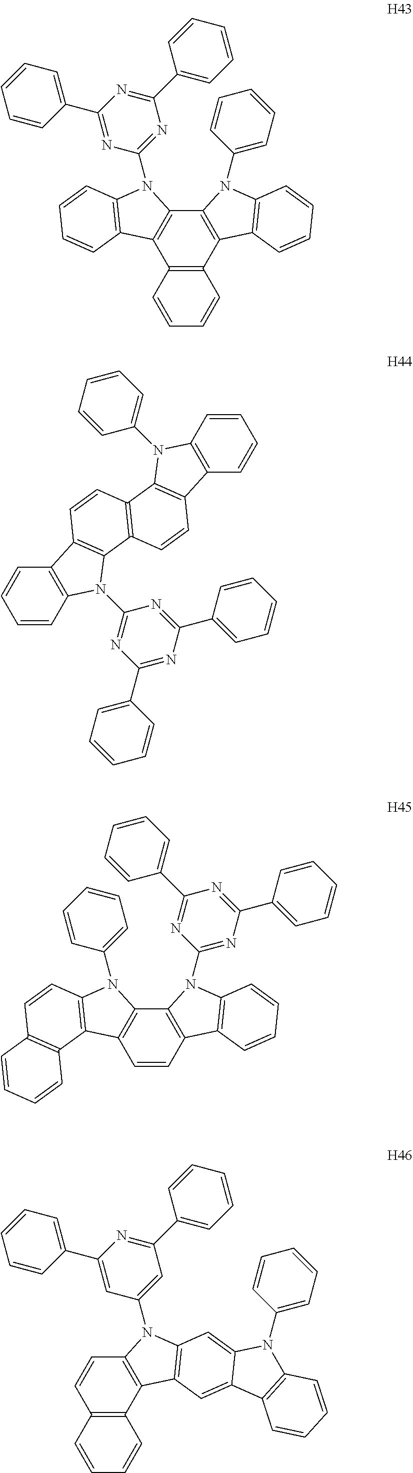 [SMILES: c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-c3ccccc3)nc(-n3c4ccccc4c4ccc5c(ccc6c7ccccc7n(-c7ccccc7)c65)c43)n2)cc1 ; c1ccc(-c2nc(-c3ccccc3)nc(-n3c4ccccc4c4ccc5c6c7ccccc7ccc6n(-c6ccccc6)c5c43)n2)cc1 ; c1ccc(-c2cc(-n3c4cc5c(cc4c4c6ccccc6ccc43)c3ccccc3n5-c3ccccc3)cc(-c3ccccc3)n2)cc1]

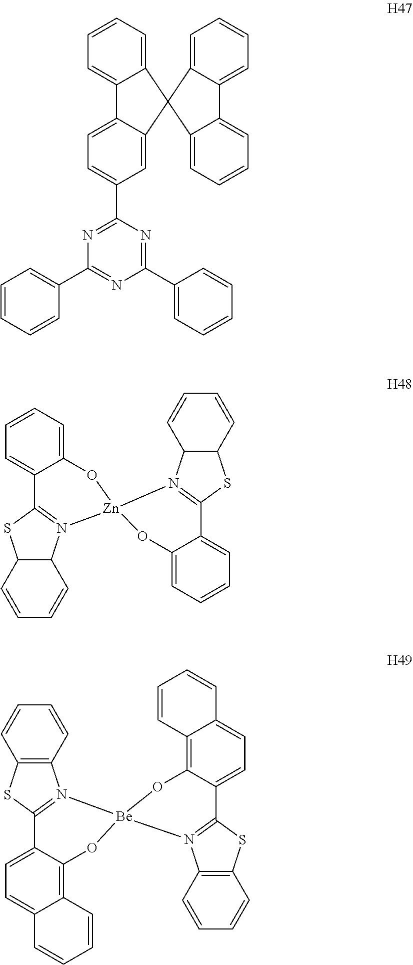

The dopant may include any suitable fluorescent dopant and/or any suitable phosphorescent dopant.

The phosphorescent dopant may include an organometallic complex represented by Formula 401 below:

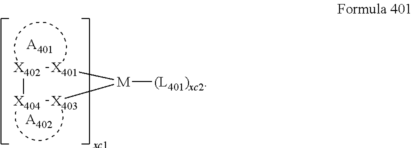

In Formula 401,

M may be selected from iridium (Ir), platinum (Pt), osmium (Os), titanium (Ti), zirconium (Zr), hafnium (Hf), europium (Eu), terbium (Tb), and thulium (Tm);

X₄₀₁ to X₄₀₄ may each independently be nitrogen or carbon;

A₄₀₁ and A₄₀₂ rings may each independently be selected from a substituted or unsubstituted benzene, a substituted or unsubstituted naphthalene, a substituted or unsubstituted fluorene, a substituted or unsubstituted spiro-fluorene, a substituted or unsubstituted indene, a substituted or unsubstituted pyrrole, a substituted or unsubstituted thiophene, a substituted or unsubstituted furan, a substituted or unsubstituted imidazole, a substituted or unsubstituted pyrazole, a substituted or unsubstituted thiazole, a substituted or unsubstituted isothiazole, a substituted or unsubstituted oxazole, a substituted or unsubstituted isoxazole, a substituted or unsubstituted pyridine, a substituted or unsubstituted pyrazine, a substituted or unsubstituted pyrimidine, a substituted or unsubstituted pyridazine, a substituted or unsubstituted quinoline, a substituted or unsubstituted isoquinoline, a substituted or unsubstituted benzoquinoline, a substituted or unsubstituted quinoxaline, a substituted or unsubstituted quinazoline, a substituted or unsubstituted carbazol, a substituted or unsubstituted benzoimidazole, a substituted or unsubstituted benzofuran, a substituted or unsubstituted benzothiophene, a substituted or unsubstituted isobenzothiophene, a substituted or unsubstituted benzoxazole, a substituted or unsubstituted isobenzoxazole, a substituted or unsubstituted triazole, a substituted or unsubstituted oxadiazole, a substituted or unsubstituted triazine, a substituted or unsubstituted dibenzofuran, and a substituted or unsubstituted dibenzothiophene; and

at least one substituent of the substituted benzene, substituted naphthalene, substituted fluorene, substituted spiro-fluorene, substituted indene, substituted pyrrole, substituted thiophene, substituted furan, substituted imidazole, substituted pyrazole, substituted thiazole, substituted isothiazole, substituted oxazole, substituted isoxazole, substituted pyridine, substituted pyrazine, substituted pyrimidine, substituted pyridazine, substituted quinoline, substituted isoquinoline, substituted benzoquinoline, substituted quinoxaline, substituted quinazoline, substituted carbazol, substituted benzoimidazole, substituted benzofuran, substituted benzothiophene, substituted isobenzothiophene, substituted benzoxazole, substituted isobenzoxazole, substituted triazole, substituted oxadiazole, substituted triazine, substituted dibenzofuran, and substituted dibenzothiophene may be selected from the group consisting of:

deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₀₁)(Q₄₀₂), —Si(Q₄₀₃)(Q₄₀₄)(Q₄₀₅), or —B(Q₄₀₆)(Q₄₀₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₁₁)(Q₄₁₂), —Si(Q₄₁₃)(Q₄₁₄)(Q₄₁₅), and —B(Q₄₁₆)(Q₄₁₇); and

—N(Q₄₂₁)(Q₄₂₂)(Q₄₂₃)(Q₄₂₄)(Q₄₂₅), and —B(Q₄₂₆)(Q₄₂₇),

wherein Q₄₀₁ to Q₄₀₇, Q₄₁₁ to Q₄₁₇, and Q₄₂₁ to Q₄₂₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group,

L₄₀₁ may be an organic ligand;

xc1 may be 1, 2, or 3; and

xc2 may be 0, 1, 2, or 3.

In Formula 401, L₄₀₁ may be a monovalent, divalent, or trivalent organic ligand. For example, L₄₀₁ may be selected from a halogen ligand (e.g., CI and/or F), a diketone ligand (e.g., acetylacetonate, 1,3-diphenyl-1,3-propanedionate, 2,2,6,6-tetramethyl-3,5-heptanedionate, and/or hexafluoroacetonate), a carboxylic acid ligand (e.g., picolinate, dimethyl-3-pyrazolecarboxylate, and/or benzoate), a carbon monooxide ligand, an isonitrile ligand, a cyano ligand, and a phosphorous ligand (e.g., phosphine and/or phosphite), but is not limited thereto.

When A₄₀₁ in Formula 401 has two or more substituents, the plurality of substituents of A₄₀₁ may be linked to form a saturated or unsaturated ring.

When A₄₀₂ in Formula 401 has two or more substituents, the plurality of substituents of A₄₀₂ may be linked to form a saturated or unsaturated ring.

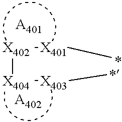

When xc1 in Formula 401 is two or more, a plurality of ligands in Formula 401 may be identical to or different from each other. When xc1 in Formula 401 is two or more, A₄₀₁ and A₄₀₂ of one ligand may each independently be connected (e.g., coupled) to A₄₀₁ and A₄₀₂ of other neighboring ligands, respectively, either directly (e.g., via a bond such as a single bond) or with a linker (e.g., a C₁-C₅ alkylene group, or —N(R′)—(wherein R′ may be a C₁-C₁₀ alkyl group or a C₆-C₂₀ aryl group) and/or —C(═O)—) therebetween.

The phosphorescent dopant may include at least one of Compounds PD1 to PD74 below, but is not limited thereto:

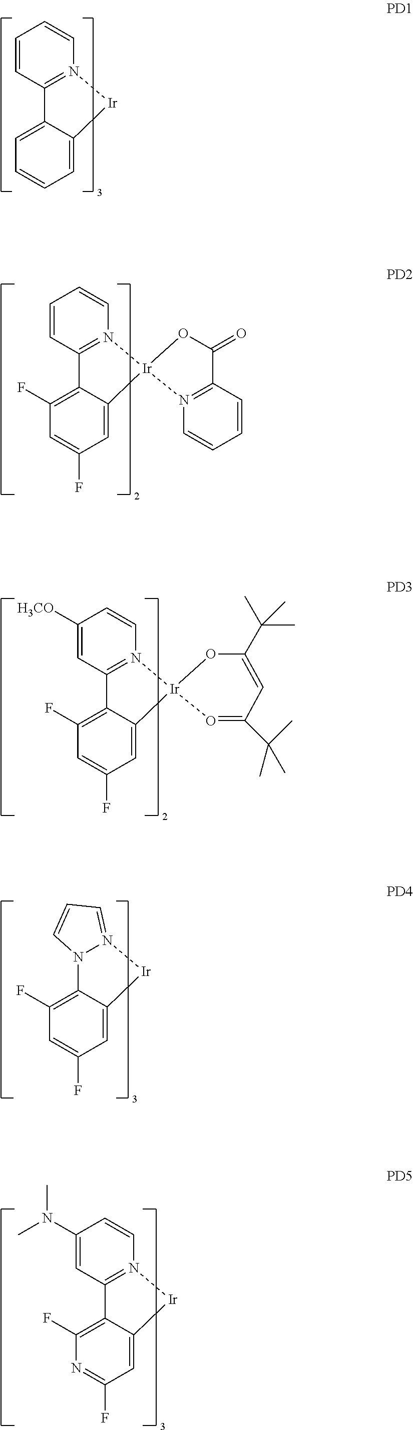

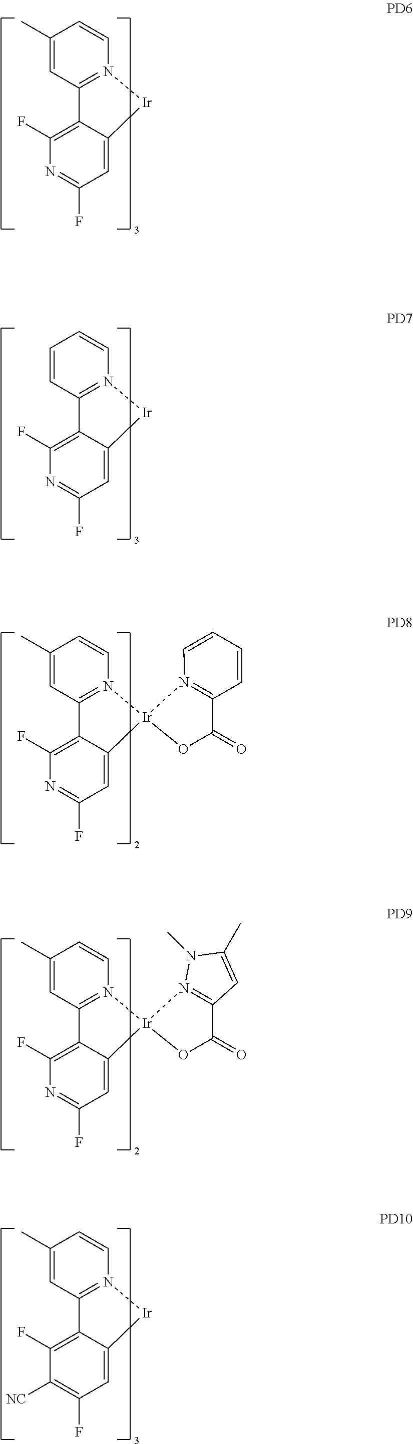

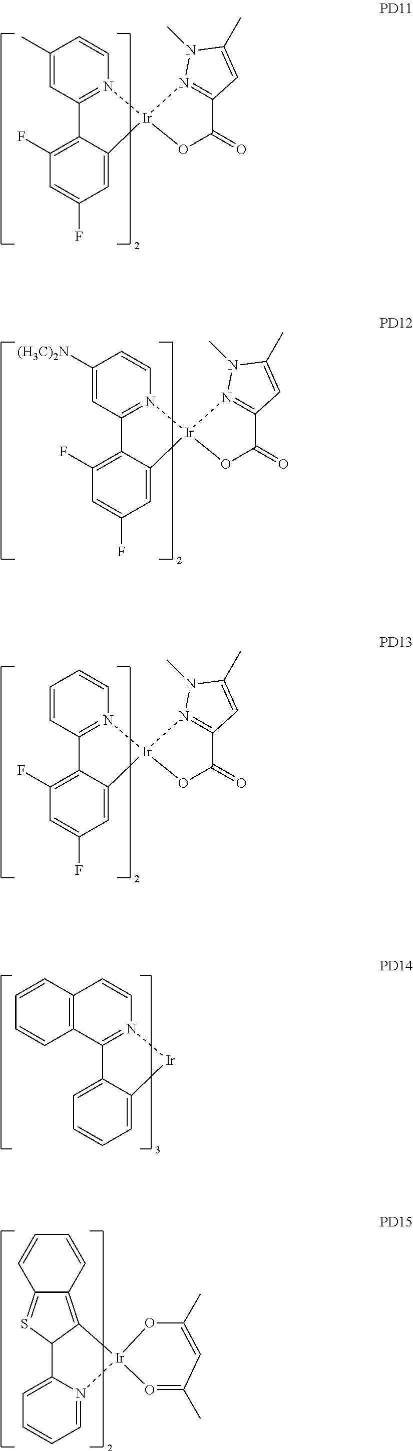

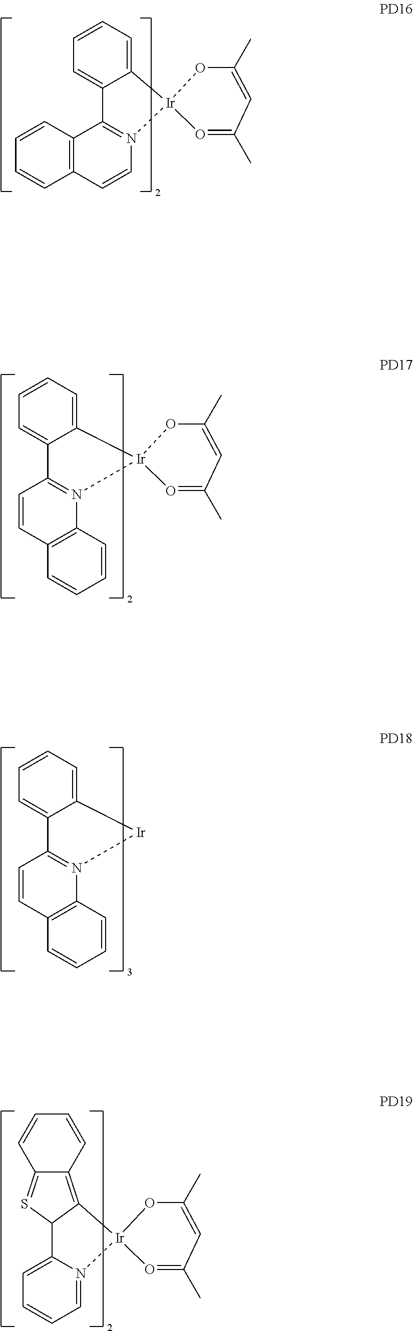

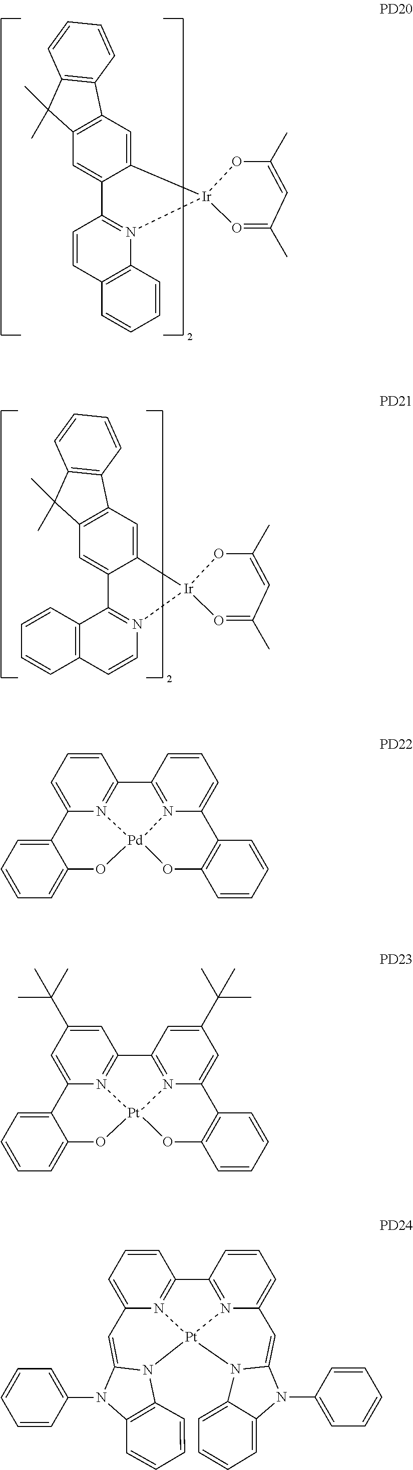

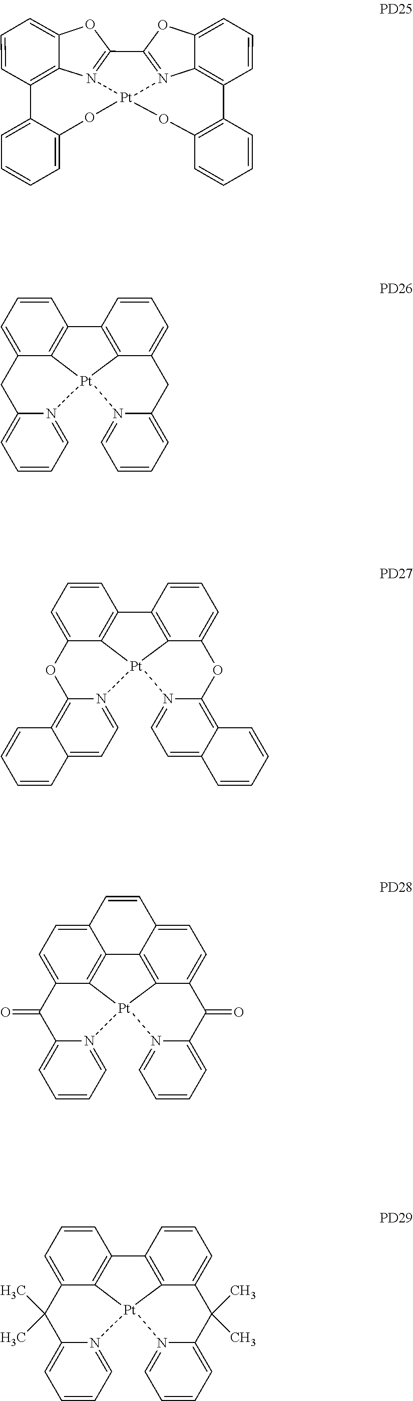

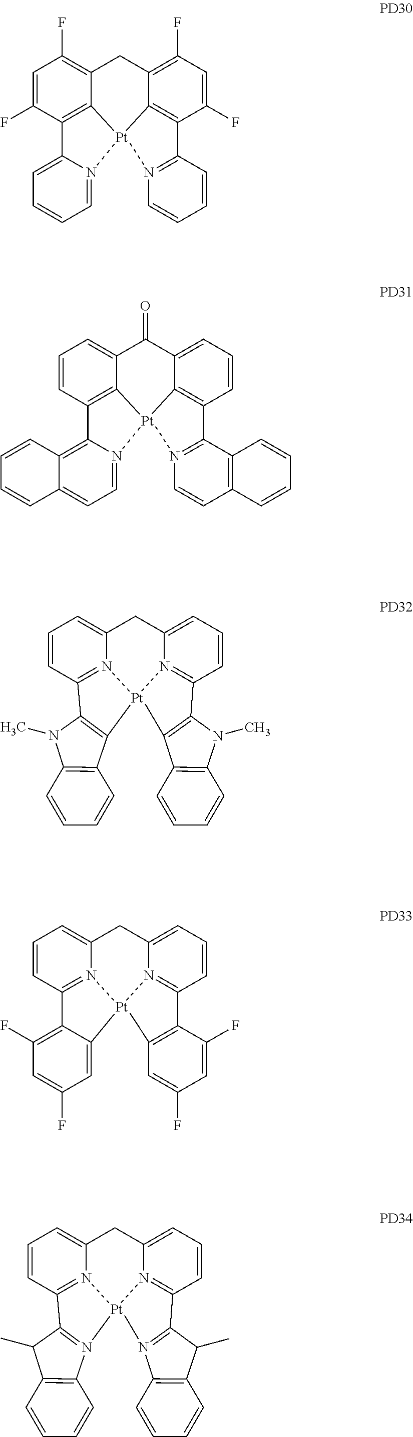

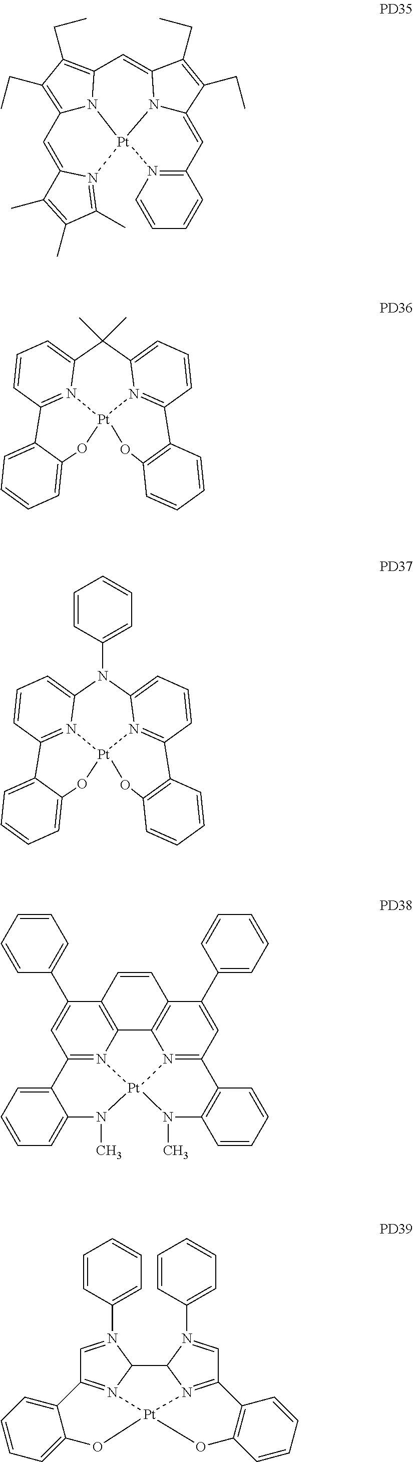

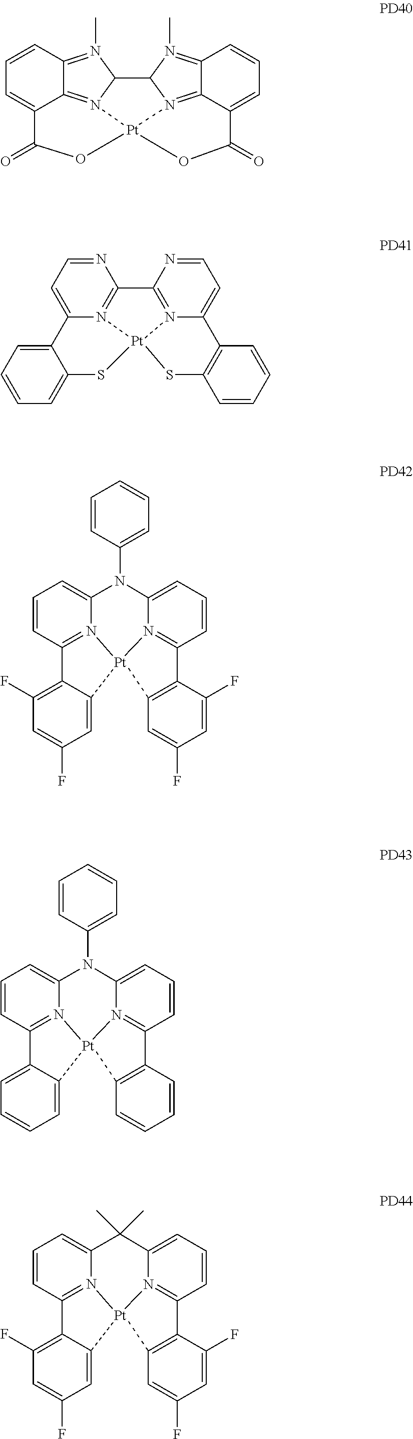

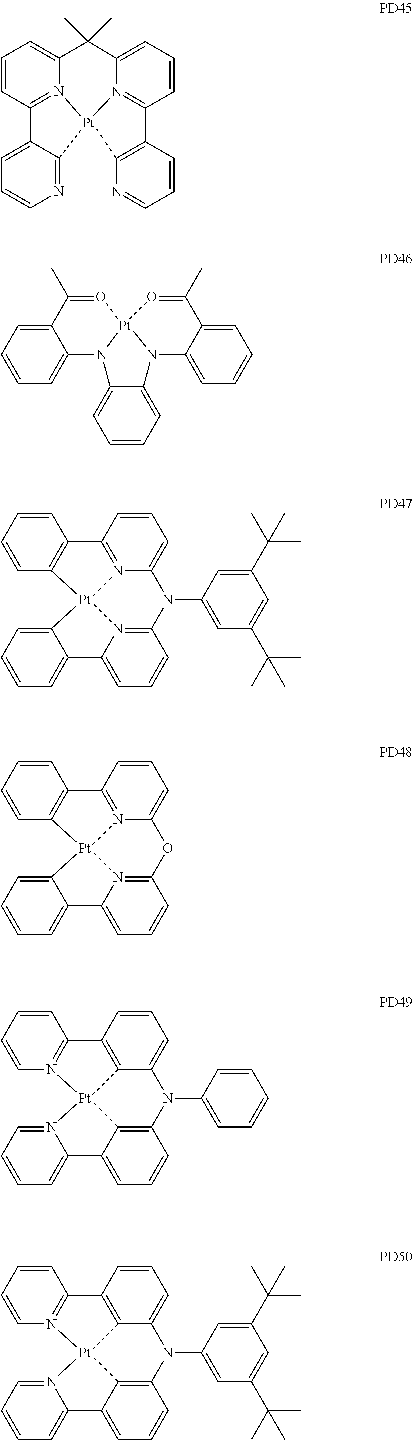

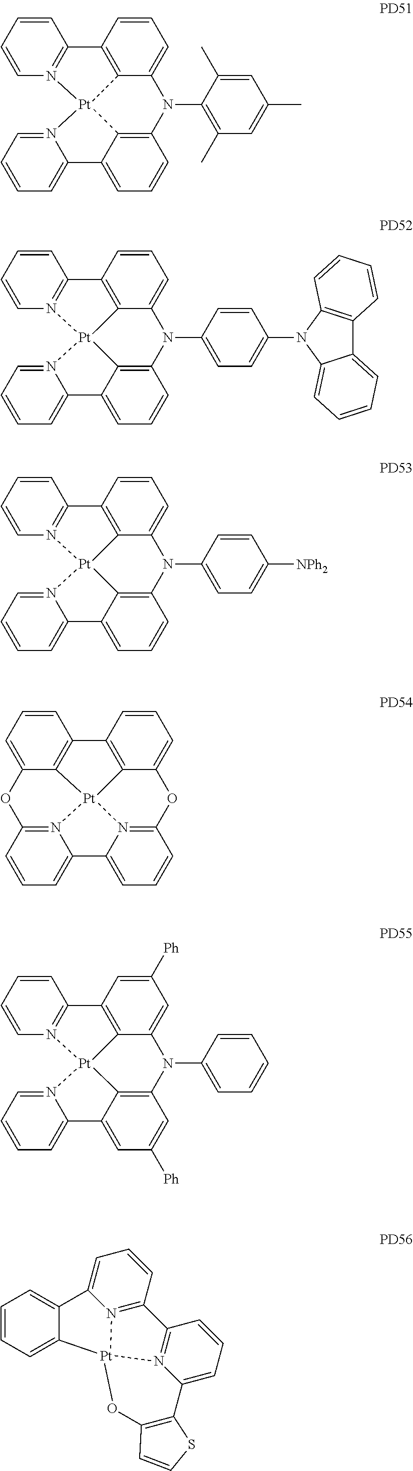

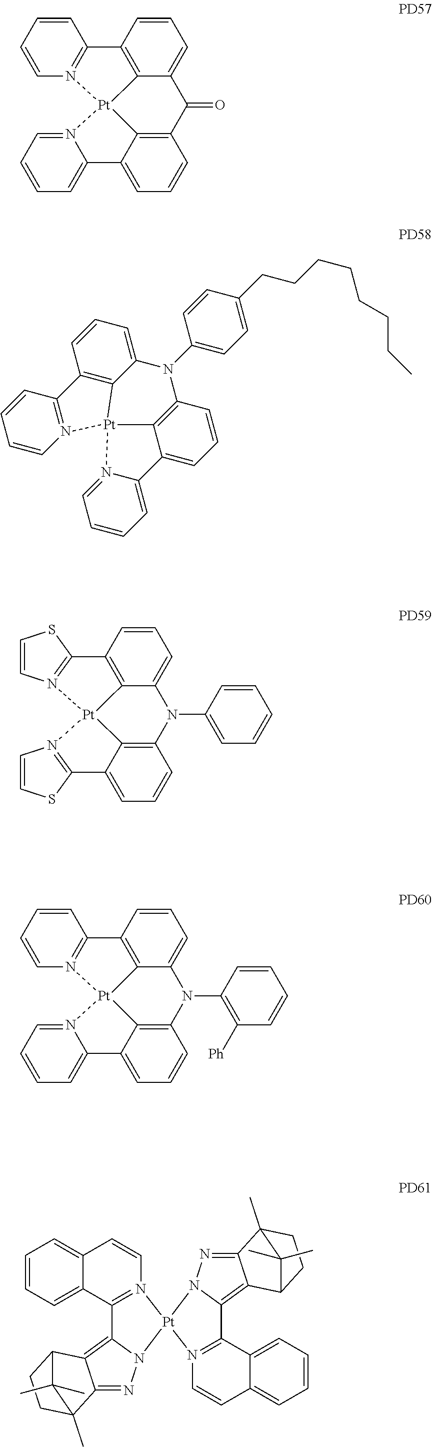

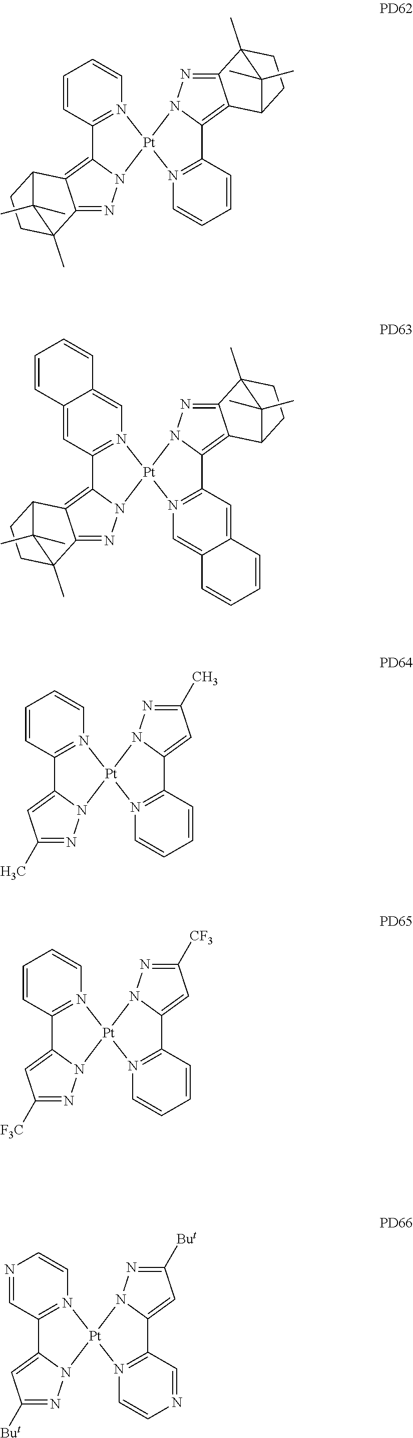

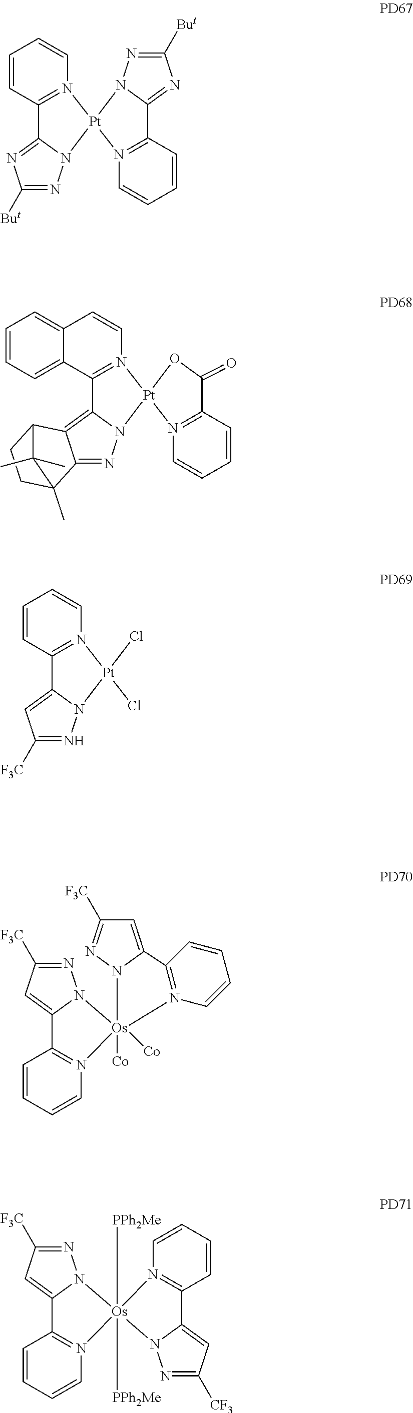

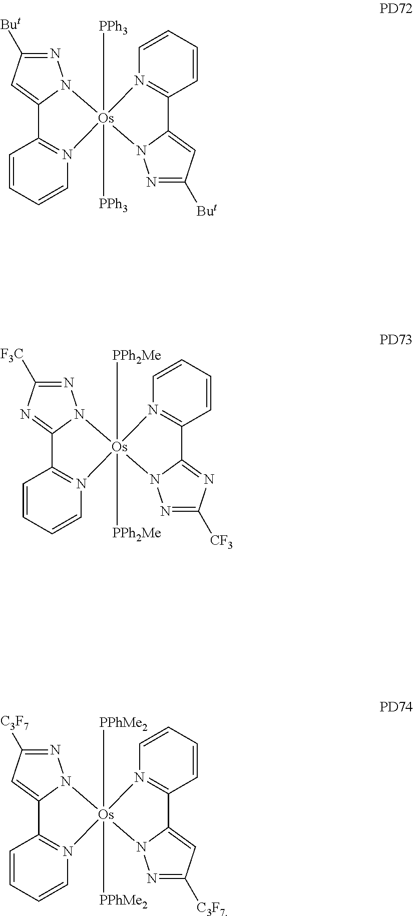

In some embodiments, the phosphorescent dopant may include PtOEP:

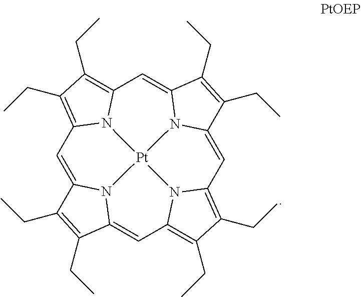

The fluorescent dopant may include at least one selected from DPVBi, DPAVBi, TBPe, DCM, DCJTB, Coumarin 6, and C545T.

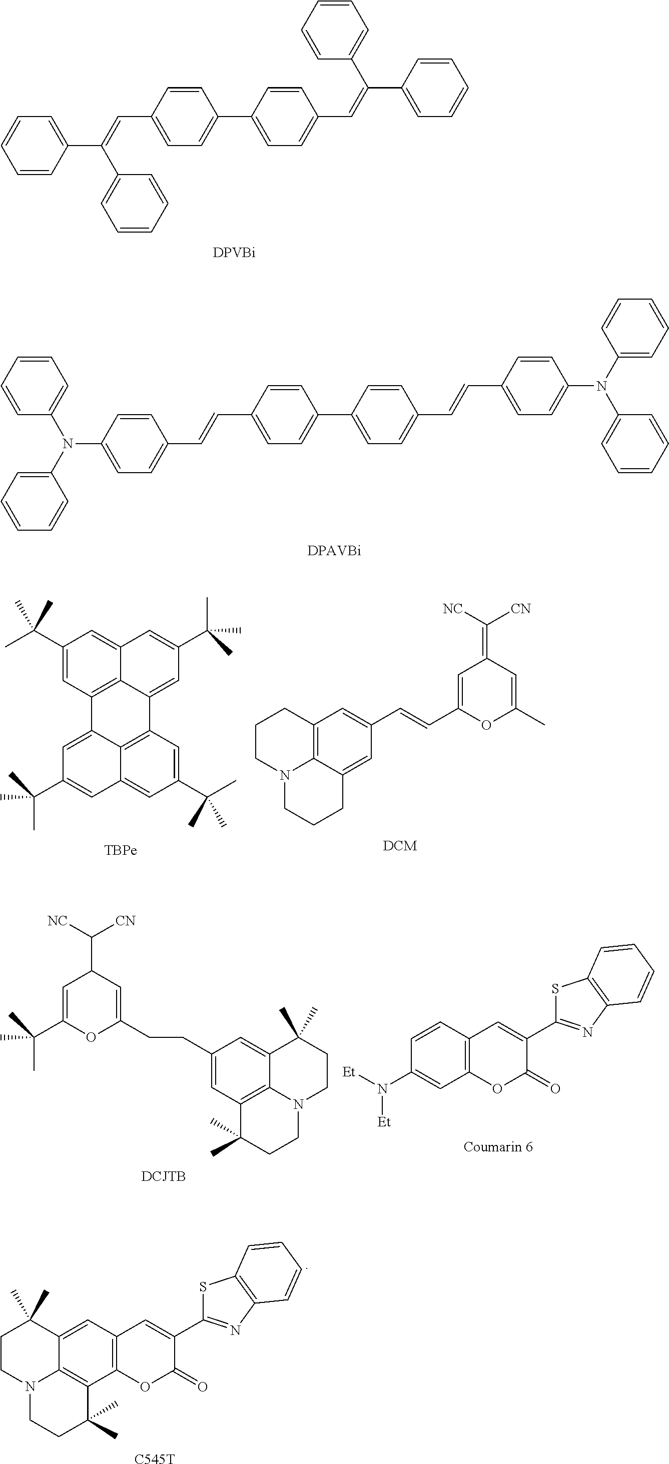

In some embodiments, the fluorescent dopant may include a compound represented by Formula 501 below.

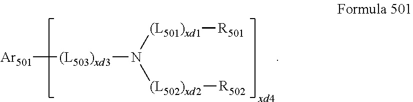

In Formula 501,

Ar₅₀₁ may be selected from the group consisting of:

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene;

a naphthalene group, a heptalene group, a fluorene group, a spiro-fluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, and an indenoanthracene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₅₀₁)(Q₅₀₂)(Q₅₀₃) (wherein Q₅₀₁ to Q₅₀₃ may each independently be selected from hydrogen, a C₁-C₆₀ alkyl group, a C₂-C₆₀ alkenyl group, a C₆-C₆₀ aryl group, and a C₁-C₆₀ heteroaryl group);

descriptions of L₅₀₁ to L₅₀₃ may each independently be understood by referring to the description of L₂₀₃;

R₅₀₁ and R₅₀₂ may each independently be selected from the group consisting of:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group; and

a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group;

xd1 to xd3 may each independently be selected from 0, 1, 2, and 3; and

xd4 may be selected from 1, 2, 3, and 4.

The fluorescent dopant may include at least one of Compounds FD1 to FD8:

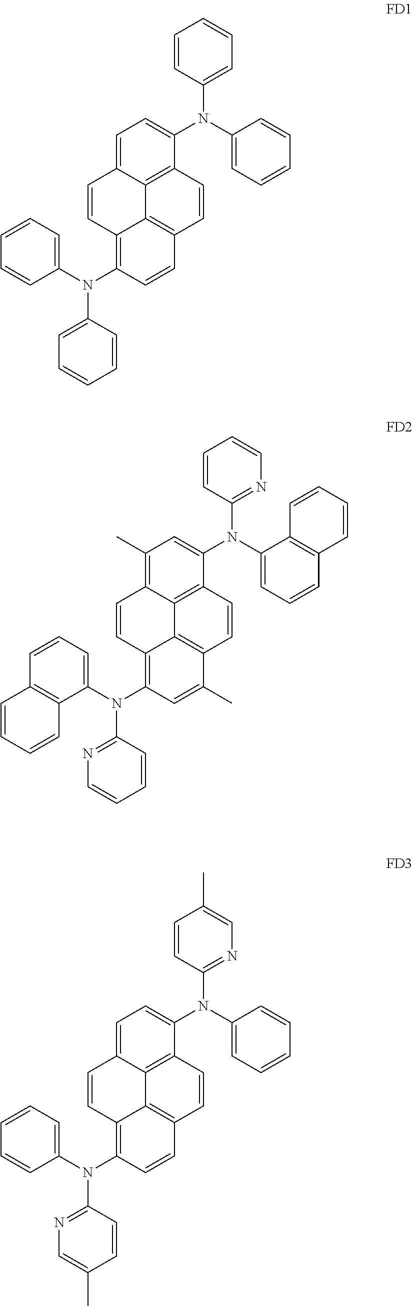 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

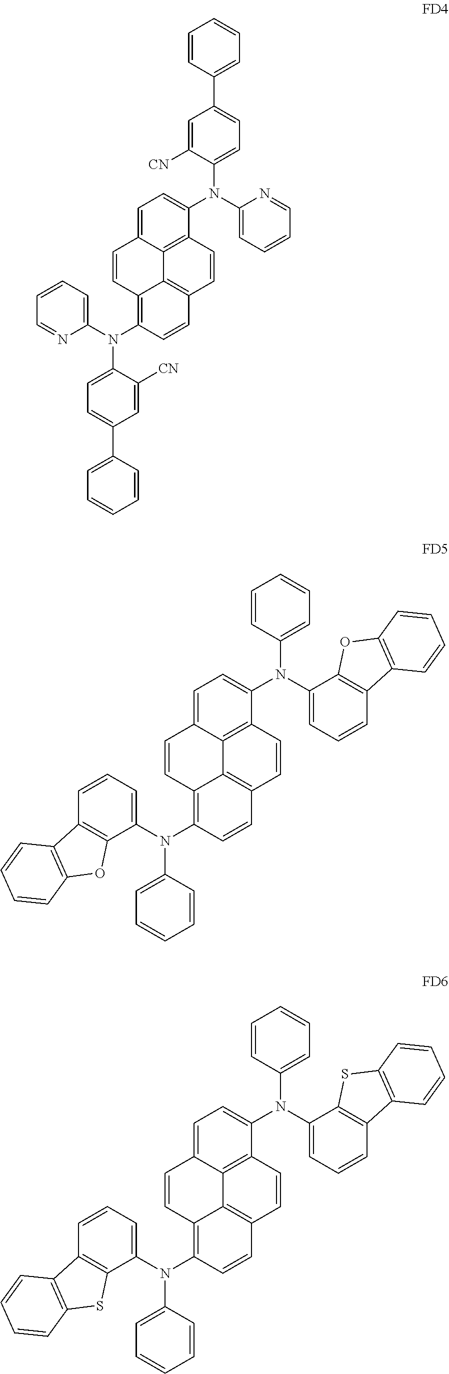

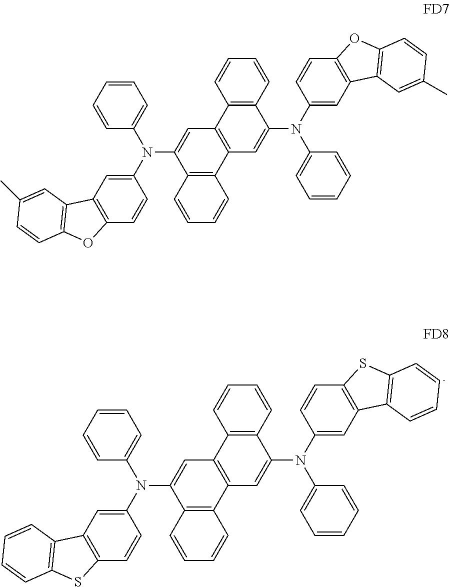

An amount of the dopant in the emission layer may be, for example, in a range of about 0.01 to about 15 parts by weight based on 100 parts by weight of the host, but is not limited thereto.

A thickness of the emission layer may be in a range of about 100 Å to about 1,000 Å, for example, about 200 Å to about 600 Å. When the thickness of the emission layer is within any of these ranges, excellent (or suitable) light-emission characteristics may be obtained without a substantial increase in driving voltage.

An electron transport region may be disposed (e.g., positioned) on the emission layer.

The electron transport region may include at least one selected from a hole blocking layer, an electron transport layer (ETL), and an electron injection layer, but is not limited thereto.

In some embodiments, the electron transport region may include the compound of Formula 1 according to an embodiment.

When the electron transport region includes a hole blocking layer, the hole blocking layer may be formed on the emission layer by using one or more suitable methods such as vacuum deposition, spin coating, casting, a Langmuir-Blodgett (LB) method, ink-jet printing, laser-printing, and/or laser-induced thermal imaging. When the hole blocking layer is formed by vacuum deposition and/or spin coating, deposition and coating conditions for the hole blocking layer may be determined by referring to the deposition and coating conditions for the hole injection layer.

The hole blocking layer may include, for example, at least one selected from BCP and Bphen, but is not limited thereto.

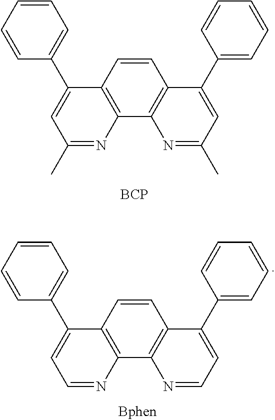

A thickness of the hole blocking layer may be in a range of about 20 Å to about 1,000 Å, for example, about 30 Å to about 300 Å. When the thickness of the hole blocking layer is within any of these ranges, the hole blocking layer may have excellent (or suitable) hole blocking characteristics without a substantial increase in driving voltage.

The electron transport region may be between the emission layer and the second electrode, and may include an electron transport layer and at least one layer selected from a hole blocking layer and an electron injection layer.

For example, the electron transport region may have a structure of electron transport layer/electron injection layer or a structure of hole blocking layer/electron transport layer/electron injection layer, wherein the layers of each structure are sequentially stacked from the emission layer in the stated order, but the structure of the electron transport region is not limited thereto.

According to an embodiment, the organic layer 150 of the organic light-emitting device includes an electron transport region between the emission layer and the second electrode 190, and the electron transport region may include an electron transport layer. The electron transport layer may include a plurality of layers. For example, the electron transport layer may include a first electron transport layer and a second electron transport layer.

The electron transport layer may include the compound of Formula 1 according to an embodiment.

A thickness of the electron transport layer may be in a range of about 100 Å to about 1,000 Å, for example, about 150 Å to about 500 Å. When the thickness of the electron transport layer is within any of the ranges described above, the electron transport layer may have satisfactory (or suitable) electron transport characteristics without a substantial increase in driving voltage.

The electron transport layer may further include, in addition to the materials described above, a metal-containing material.

The metal-containing material may include a Li complex. The Li complex may include, for example, Compound ET-D1 (lithium quinolate, LiQ) and/or Compound

ET-D2.

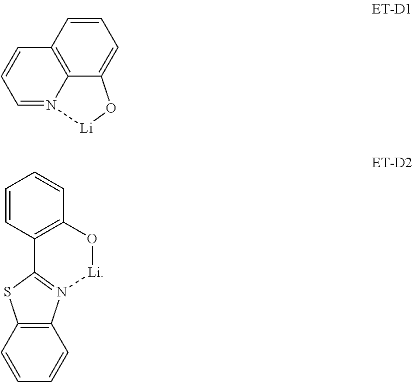

The electron transport region may include an electron injection layer that facilitates injection of electrons from the second electrode 190.

The electron injection layer may be formed on the electron transport layer by using one or more suitable methods such as vacuum deposition, spin coating, casting, a LB method, ink-jet printing, laser-printing, and/or laser-induced thermal imaging. When an electron injection layer is formed by vacuum deposition and/or spin coating, deposition and coating conditions for the electron injection layer may be the same as (or similar to) those for the hole injection layer.

The electron injection layer may include at least one selected from LiF, NaCl, CsF, Li₂O, BaO, and LiQ.

A thickness of the electron injection layer may be in a range of about 1 Å to about 100 Å, for example, about 3 Å to about 90 Å. When the thickness of the electron injection layer is within any of the ranges described above, the electron injection layer may have satisfactory (or suitable) electron injection characteristics without a substantial increase in driving voltage.

The second electrode 190 may be disposed (e.g., positioned) on the organic layer 150 having the structure according to embodiments of the present disclosure. The second electrode 190 may be a cathode (which is an electron injection electrode), and in this regard, a material for forming the second electrode 190 may be selected from a metal, an alloy, an electrically conductive compound, and a mixture thereof, which have a relatively low work function. Non-limiting examples of the material for forming the second electrode 190 include lithium (Li), magnesium (Mg), aluminum (Al), aluminum-lithium (Al—Li), calcium (Ca), magnesium-indium (Mg—In), and magnesium-silver (Mg—Ag). In some embodiments, the material for forming the second electrode 190 may be ITO and/or IZO. The second electrode 190 may be a reflective electrode, a semi-transmissive electrode, or a transmissive electrode.

An organic layer according to an embodiment may be formed by depositing the compound according to an embodiment, or may be formed by using a wet method in which the compound according to an embodiment is prepared in the form of a solution, and then the solution of the compound is used for coating.

An organic light-emitting device according to an embodiment may be used in various flat panel display apparatuses, such as a passive matrix organic light-emitting display apparatus and/or an active matrix organic light-emitting display apparatus. For example, when the organic light-emitting device is included in an active matrix organic light-emitting display apparatus, a first electrode disposed on a substrate may function as a pixel electrode and may be electrically connected (e.g., electrically coupled) to a source electrode or a drain electrode of a thin film transistor. In addition, the organic light-emitting device may be included in a flat panel display apparatus that can emit light in opposite directions (e.g., can emit light from both sides of the display panel).

Hereinbefore, the organic light-emitting device has been described with reference to the drawing, but is not limited thereto.

Hereinafter, definitions of substituents of compounds used herein will be presented. The number of carbon atoms used to restrict a substituent is not limited, and does not limit properties of the substituent, and unless defined otherwise, the definition of the substituent is consistent with a general definition thereof.

The term “C₁-C₆₀ alkyl group,” as used herein, may refer to a linear or branched aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an iso-amyl group, and a hexyl group. The term “C₁-C₆₀ alkylene group,” as used herein, may refer to a divalent group having the same structure as the C₁-C₆₀ alkyl group.

The term “C₁-C₆₀ alkoxy group,” as used herein, may refer to a monovalent group represented by —OA₁₀₁ (wherein A₁₀₁ is the C₁-C₆₀ alkyl group), and non-limiting examples thereof include a methoxy group, an ethoxy group, and an isopropoxy group.

The term “C₂-C₆₀ alkenyl group,” as used herein, may refer to a hydrocarbon group having at least one carbon double bond at one or more positions along the hydrocarbon chain of the C₂-C₆₀ alkyl group (e.g., in the middle and/or at either terminus of the C₂-C₆₀ alkyl group), and non-limiting examples thereof include an ethenyl group, a propenyl group, and a butenyl group. The term “C₂-C₆₀ alkenylene group,” as used herein, may refer to a divalent group having the same structure as the C₂-C₆₀ alkenyl group.

The term “C₂-C₆₀ alkynyl group,” as used herein, may refer to a hydrocarbon group having at least one carbon triple bond at one or more positions along the hydrocarbon chain of the C₂-C₆₀ alkyl group (e.g., in the middle and/or at either terminus of the C₂-C₆₀ alkyl group), and non-limiting examples thereof include an ethynyl group and a propynyl group. The term “C₂-C₆₀ alkynylene group,” as used herein, may refer to a divalent group having the same structure as the C₂-C₆₀ alkynyl group.

The term “C₃-C₁₀ cycloalkyl group,” as used herein, may refer to a monovalent hydrocarbon monocyclic group having 3 to 10 carbon atoms, and non-limiting examples thereof include a cyclopropyl group, a cyclobutyl group, a cyclopentyl group, a cyclohexyl group, and a cycloheptyl group. The term “C₃-C₁₀ cycloalkylene group,” as used herein, may refer to a divalent group having the same structure as the C₃-C₁₀ cycloalkyl group.

The term “C₂-C₁₀ heterocycloalkyl group,” as used herein, may refer to a monovalent monocyclic group having at least one heteroatom selected from N, O, P, and S as a ring-forming atom and 2 to 10 carbon atoms, and non-limiting examples thereof include a tetrahydrofuranyl group and a tetrahydrothiophenyl group. The term “C₂-C₁₀ heterocycloalkylene group,” as used herein, may refer to a divalent group having the same structure as the C₂-C₁₀ heterocycloalkyl group.

The term “C₃-C₁₀ cycloalkenyl group,” as used herein, may refer to a monovalent monocyclic group that has 3 to 10 carbon atoms and at least one double bond in the ring thereof, and does not have aromaticity, and non-limiting examples thereof include a cyclopentenyl group, a cyclohexenyl group, and a cycloheptenyl group. The term “C₃-C₁₀ cycloalkenylene group,” as used herein, may refer to a divalent group having the same structure as the C₃-C₁₀ cycloalkenyl group.

The term “C₂-C₁₀ heterocycloalkenyl group,” as used herein, may refer to a monovalent monocyclic group that has at least one heteroatom selected from N, O, P, and S as a ring-forming atom, 2 to 10 carbon atoms, and at least one double bond in its ring. Non-limiting examples of the C₂-C₁₀ heterocycloalkenyl group include a 2,3-hydrofuranyl group and a 2,3-hydrothiophenyl group. The term “C₂-C₁₀ heterocycloalkenylene group,” as used herein, may refer to a divalent group having the same structure as the C₂-C₁₀ heterocycloalkenyl group.

The term “C₆-C₆₀ aryl group,” used herein, may refer to a monovalent group having a carbocyclic aromatic system having 6 to 60 carbon atoms, and the term “C₆-C₆₀ arylene group,” as used herein, may refer to a divalent group having a carbocyclic aromatic system having 6 to 60 carbon atoms. Non-limiting examples of the C₆-C₆₀ aryl group include a phenyl group, a biphenyl group, a terphenyl group, a naphthyl group, an anthracenyl group, a phenanthrenyl group, a pyrenyl group, and a chrysenyl group. When the C₆-C₆₀ aryl group and the C₆-C₆₀ arylene group each independently include two or more rings, the respective rings may be fused to each other.

The term “C₁-C₆₀ heteroaryl group,” as used herein, may refer to a monovalent group having a carbocyclic aromatic system that has at least one heteroatom selected from N, O, P, and S as a ring-forming atom, and 1 to 60 carbon atoms. The term“C₁-C₆₀ heteroarylene group,” as used herein, may refer to a divalent group having a carbocyclic aromatic system that has at least one heteroatom selected from N, O, P, and S as a ring-forming atom and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each independently include two or more rings, the respective rings may be fused to each other.

A C₆-C₆₀ aryloxy group used herein may refer to a monovalent group represented by —OA₁₀₂ (wherein A₁₀₂ is the C₆-C₆₀ aryl group), and a C₆-C₆₀ arylthio group used herein may refer to a monovalent group represented by —SA₁₀₃ (wherein A₁₀₃ is the C₆-C₆₀ aryl group).

The term “monovalent non-aromatic condensed polycyclic group,” used herein, may refer to a monovalent group that has two or more rings condensed (e.g., fused) to each other, only carbon atoms as a ring-forming atoms (e.g., having 8 to 60 carbon atoms), and non-aromaticity in the entire molecular structure (e.g., does not have overall aromaticity). Non-limiting example of the monovalent non-aromatic condensed polycyclic group is a fluorenyl group. The term “divalent non-aromatic condensed polycyclic group,” used herein, may refer to a divalent group having the same structure as the monovalent non-aromatic condensed polycyclic group.

The term “monovalent non-aromatic condensed heteropolycyclic group,” used herein, may refer to a monovalent group that has two or more rings condensed (e.g., fused) to each other, has at least one heteroatom selected from N, O, P, and S, other than carbon atoms (e.g., 2 to 60 carbon atoms), as ring-forming atoms, and has non-aromaticity in the entire molecular structure (e.g., does not have overall aromaticity). The term “divalent non-aromatic condensed heteropolycyclic group,” used herein, may refer to a divalent group having the same structure as the monovalent non-aromatic condensed heteropolycyclic group.

At least one substituent of the substituted C₃-C₁₀ cycloalkylene group, substituted C₂-C₁₀ heterocycloalkylene group, substituted C₃-C₁₀ cycloalkenylene group, substituted C₂-C₁₀ heterocycloalkenylene group, substituted C₆-C₆₀ arylene group, substituted C₁-C₆₀ heteroarylene group, substituted divalent non-aromatic condensed polycyclic group, substituted divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₂-C₁₀ heterocycloalkyl group, substituted C₃-C₁₀ cycloalkenyl group, substituted C₂-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group may be selected from the group consisting of:

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂),—Si(Q₁₃)(Q₁₄)(Q₁₅), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅) and —B(Q₃₆)(Q₃₇),

wherein Q₁₁ to Q₁₇, Q₂₁ to Q₂₇, and Q₃₁ to Q₃₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

For example, at least one substituent of the substituted C₃-C₁₀ cycloalkylene group, substituted C₂-C₁₀ heterocycloalkylene group, substituted C₃-C₁₀ cycloalkenylene group, substituted C₂-C₁₀ heterocycloalkenylene group, substituted C₆-C₆₀ arylene group, substituted C₁-C₆₀ heteroarylene group, substituted divalent non-aromatic condensed polycyclic group, substituted divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₂-C₁₀ heterocycloalkyl group, substituted C₃-C₁₀ cycloalkenyl group, substituted C₂-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group may be selected from the group consisting of:

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —N(Q₁₁)(Q₁₂), —Si(Q₁₃)(Q₁₄)(Q₁₅) and —B(Q₁₆)(Q₁₇);

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group;

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇),

wherein Q₁₁ to Q₁₇, Q₂₁ to Q₂₇, and Q₃₁ to Q₃₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

The expression “Ph” used herein may refer to a phenyl group, the expression “Me” used herein may refer to a methyl group, the expression “Et” used herein may refer to an ethyl group, the expression “ter-Bu” or “Buₜ” used herein may refer to a tert-butyl group, and “D” may refer to deuterium.

Hereinafter, an organic light-emitting device according to an embodiment will be described in more detail with reference to Synthesis Examples and Examples. However, these examples are for illustrative purposes only and are not intended to limit the scope of the present disclosure.

### SYNTHESIS EXAMPLES

### Synthesis Example 1

### Synthesis of Compound 21

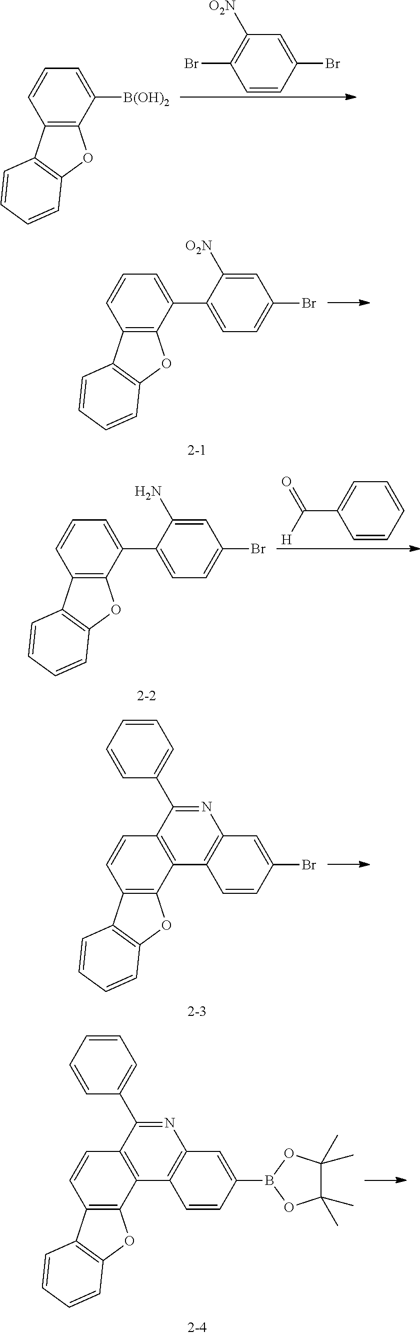

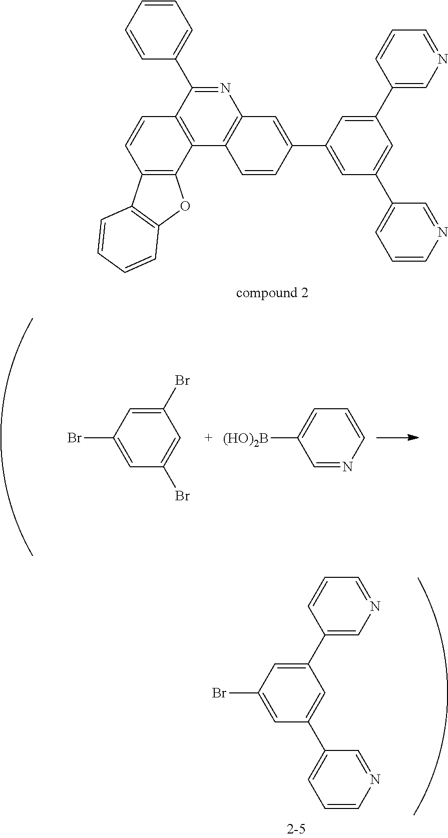 [SMILES: c1ccc(-c2nc3cc(-c4cc(-c5cccnc5)cc(-c5cccnc5)c4)ccc3c3c2ccc2c4ccccc4oc23)cc1 ; Brc1cc(Br)cc(Br)c1 ; OB(O)c1cccnc1 ; Brc1cc(-c2cccnc2)cc(-c2cccnc2)c1]

### Synthesis of Intermediate 2-1

2.12 g (10 mmol) of dibenzofuran-4-boronic acid, 3.37 g (12 mmol) of 1,4-dibromo-2-nitrobenzene, 0.58 g (0.50 mmol) of Pd(PPh₃)₄, 0.16 g (0.5 mmol) of TBAB (tetrabutylammonium bromide), and 3.18 g (30.0 mmol) of Na₂CO₃ were dissolved in 60 mL of a mixed solution of toluene/ethanol/H₂O (volume ratio: 3/3/1), and then, the resulting mixture was stirred at a temperature of 100° C. for 16 hours. The obtained reaction solution was cooled to room temperature, and then subjected to an extraction process three times by using 60 mL of water and 60 mL of diethylether. An organic layer obtained by the extraction process was dried by using magnesium sulfate, and then, the residual obtained by evaporating a solvent therefrom was separation-purified by silica gel column chromatography, thereby completing the preparation of 2.94 g (yield of 80%) of Intermediate 2-1. The obtained compound was identified by Liquid chromatography-mass spectrometry (LC-MS).

C₁₈H₁₀BrNO₃: M+1 366.98

### Synthesis of Intermediate 2-2

3.68 g (10.0 mmol) of Intermediate 2-1, 3.56 g (30 mmol) of tin, and 5 mL (50 mmol, conc. 36.5%) of HCl were dissolved in 60 mL of ethanol, and then, the resulting mixture was stirred at a temperature of 100° C. for 8 hours. The obtained reaction solution was cooled to room temperature, and then, filtered under reduced pressure to obtain a filtrate. Then, 3 g of sodium hydroxide dissolved in 10 mL of water was added thereto, and an extraction process was performed on the resultant three times by using 60 mL of water and 60 mL of dichloromethane. An organic layer obtained therefrom was dried by using magnesium sulfate, and then, the residual obtained by evaporating a solvent therefrom was separation-purified by silica gel column chromatography, thereby completing the preparation of 3.04 g (yield of 90%) of Intermediate 2-2. The obtained compound was identified by LC-MS.

C₁₈H₁₂BrNO: M+1 337.02

### Synthesis of Intermediate 2-3

3.38 g (10 mmol) of Intermediate 2-2 and 2.12 g (20 mmol) of benzaldehyde were dissolved in 10 mL of trifluoroacetic acid, and then, the resulting mixture was stirred in a sealed tube at a temperature of 130° C. for 3 days. The obtained reaction solution was cooled to room temperature, and then, the reaction was quenched by using NaHCO₃, and then, an extraction process was performed on the resultant by using 60 mL of water and 60 mL of dichloromethane. An organic layer obtained therefrom was dried by using magnesium sulfate, and then, the residual obtained by evaporating a solvent therefrom was separation-purified by silica gel column chromatography, thereby completing the preparation of 2.12 g (yield of 50%) of Intermediate 2-3. The obtained compound was identified by LC-MS.

C₂₅H₁₄BrNO : M+1 423.03

### Synthesis of Intermediate 2-4

4.24 g (10 mmol) of Intermediate 2-3 was dissolved in 200 mL of THF, and then, at a temperature of −78° C., 4 mL (2.5M in hexane) of normal butyllithium was added thereto. One hour after the addition, at the same temperature, 2.0 mL (10 mmol) of 2-isopropoxy-4,4,5,5-tetramethyl-1,3,2-dioxaborolane was added thereto. The resultant was stirred at room temperature for 5 hours, and then water was added thereto, and washed three times by using 100 mL of diethylether. The resultant diethylether was dried by using MgSO₄ and then dried under reduced pressure to obtain a product, which was then separation-purified by silica gel column chromatography to obtain 3.3 g (yield of 70%) of Intermediate 2-4. The obtained compound was confirmed by LC-MS.

C₃₁H₂₆BO₃: M+1 471.2

### Synthesis of Intermediate 2-5

3.15 g (10 mmol) of 1,3,5-tribromobenzene, 2.46 g (20 mmol) of pyridine-3-boronic acid, 1.16 g (1 mmol) of Pd(PPh₃)₄, and 8.3 g (60.0 mmol) of K₂CO₃ were dissolved in 100 mL of a mixed solution of THF/H₂O (volume ratio: 2/1), and then, the resulting mixture was stirred at a temperature of 80° C. for 16 hours. The obtained reaction solution was cooled to room temperature, and then subjected to an extraction process three times by using 60 mL of water and 60 mL of diethylether. An organic layer obtained by the extraction process was dried by using magnesium sulfate, and then, the residual obtained by evaporating a solvent therefrom was separation-purified by silica gel column chromatography, thereby completing the preparation of 2.18 g (yield of 70%) of Intermediate 2-5. The obtained compound was identified by LC-MS.

C₁₆H₁₁BrN₂: M+1 310.01

### Synthesis of Compound 2

4.71 g (10 mmol) of Intermediate 2-4, 3.11 g (10 mmol) of Intermediate 2-5, 0.58 g (0.5 mmol) of Pd(PPh₃)₄, and 4.15 g (30.0 mmol) of K₂CO₃ were dissolved in 100 mL of a mixed solution of THF/H₂O (volume ratio: 2/1), and then, the resulting mixture was stirred at a temperature of 80° C. for 16 hours. The obtained reaction solution was cooled to room temperature, and then subjected to an extraction process three times by using 60 mL of water and 60 mL of diethylether. An organic layer obtained by the extraction process was dried by using magnesium sulfate, and then, the residual obtained by evaporating a solvent therefrom was separation-purified by silica gel column chromatography, thereby completing the preparation of 4.03 g (yield of 70%) of Compound 2. The obtained compound was identified by LC-MS and ¹H NMR.

C₄₁H₂₅N₃O : M+1 575.21

“'Synthesis Example 2

### Synthesis of Compound 8

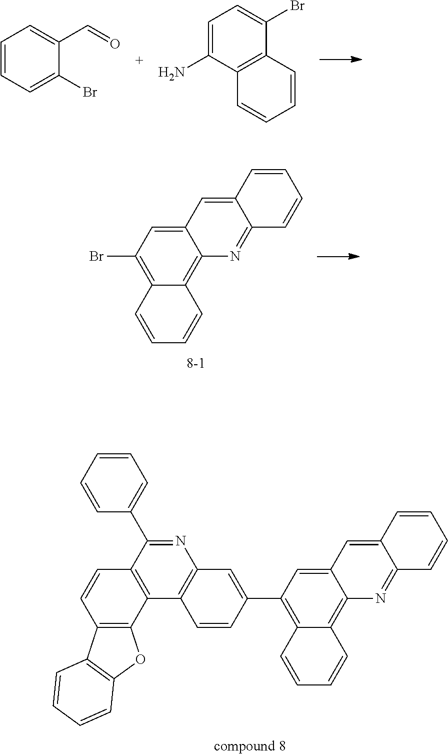 [SMILES: O=Cc1ccccc1Br ; Nc1ccc(Br)c2ccccc12 ; Brc1cc2cc3ccccc3nc2c2ccccc12 ; c1ccc(-c2nc3cc(-c4cc5cc6ccccc6nc5c5ccccc45)ccc3c3c2ccc2c4ccccc4oc23)cc1]

### Synthesis of Intermediate 8-1

2.67 g (12 mmol) of 4-bromo-1-amino naphthalene, 0.23 g (0.25 mmol) of Pd₂(dba)₃, 0.28 g (0.5 mmol) of dppf, and 2 g (20 mmol) of K₂CO₃ were dissolved in toluene, and then, 1.85 g (10.0 mmol) of 2-bromobenzaldehyde dissolved in toluene was added thereto in inert nitrogen gas atmosphere. The resulting mixture was stirred at a temperature of 80° C. for 12 hours. The resultant was cooled to room temperature, and then, filtered through celite, and subjected to an evaporation process under reduced pressure. 2.7 g (20 mmol) of AlCl₃ was added thereto, and then dissolved in toluene, followed by stirring the resultant at a temperature of 80° C. for 12 hours. The obtained reaction solution was cooled to room temperature, and then, an extraction process was performed thereon three times by using 60 mL of dichloromethane. An organic layer obtained by the extraction process was dried by using magnesium sulfate, and then, the residual obtained by evaporating a solvent therefrom was separation-purified by silicagel column chromatography, thereby completing the preparation of 1.54 g (yield of 50%) of Intermediate 8-1. The obtained compound was identified by LC-MS.

C₁₇H₁₀BrN : M+1 307

### Synthesis of Compound 8

4.01 g (yield of 70%) of Compound 8 was prepared in the same (or substantially the same) manner as the one used to synthesize Compound 2, except that Intermediate 8-1 was used instead of Intermediate 2-5. The obtained compound was identified by LC-MS and ¹H NMR.

C₄₂H₂₄N₂O : M+1 572.2

### Synthesis Example 3

### Synthesis of Compound 311

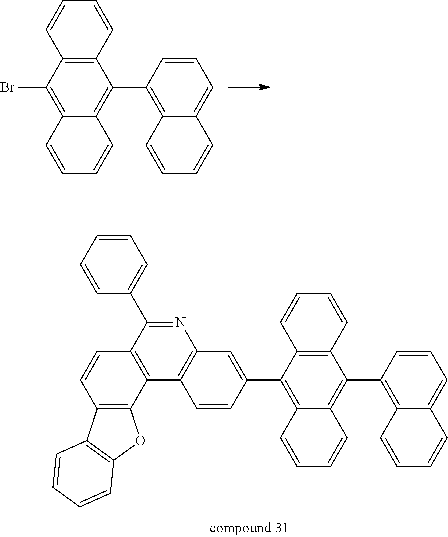 [SMILES: Brc1c2ccccc2c(-c2cccc3ccccc23)c2ccccc12 ; c1ccc(-c2nc3cc(-c4c5ccccc5c(-c5cccc6ccccc56)c5ccccc45)ccc3c3c2ccc2c4ccccc4oc23)cc1]

### Synthesis of Compound 31

4.53 g (yield of 70%) of Compound 31 was prepared in the same (or substantially the same) manner as the one used to synthesize Compound 2, except that 9-bromo-10-(naphthalene-1)anthracene was used instead of Intermediate 2-5. The obtained compound was identified by LC-MS and ¹H NMR.

C₄₉H₂₉NO : M+1 647.23

### Synthesis Example 4

### Synthesis of Compound 371

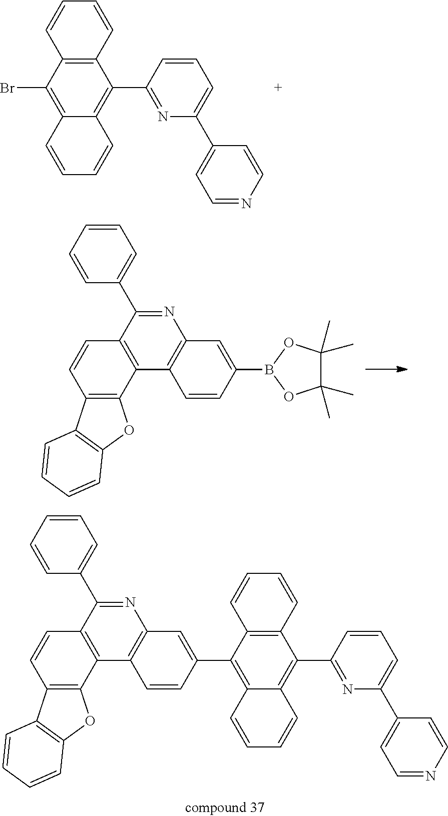

### Synthesis of Compound 37

4.05 g (yield of 60%) of Compound 37 was prepared in the same (or substantially the same) manner as the one used to synthesize Compound 2, except that 6-(10-bromoanthracene-9-yl)-2,4′-bipyridine was used instead of Intermediate 2-5. The obtained compound was identified by LC-MS and ¹H NMR.

C₄₉H₂₉N₃O : M+1 675.2

### Synthesis Example 5

### Synthesis of Compound 571

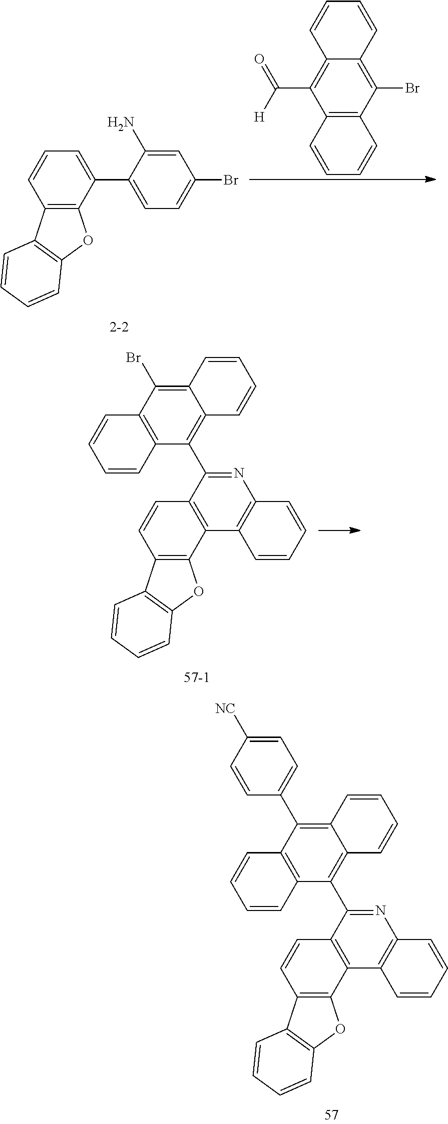 [SMILES: Nc1cc(Br)ccc1-c1cccc2c1oc1ccccc12 ; O=Cc1c2ccccc2c(Br)c2ccccc12 ; Brc1c2ccccc2c(-c2nc3ccccc3c3c2ccc2c4ccccc4oc23)c2ccccc12 ; N#Cc1ccc(-c2c3ccccc3c(-c3nc4ccccc4c4c3ccc3c5ccccc5oc34)c3ccccc23)cc1]

### Synthesis of Intermediate 57-1

2.62 g (yield of 50%) of Intermediate 57-1 was prepared in the same (or substantially the same) manner as the one used to synthesize Intermediate 2-3, except that 10-bromoanthracene-9-carboaldehyde was used instead of benzaldehyde. The obtained compound was identified by LC-MS and ¹H NMR.

C₃₃H₁₈BrNO : M+1 523.06

### Synthesis of Compound 57

3.83 g (yield of 70%) of Compound 57 was prepared in the same (or substantially the same) manner as the one used to synthesize Compound 2, except that (4-cyanophenyl)boronic acid and Intermediate 57-1 were used instead of Intermediate 2-4 and Intermediate 2-5. The obtained compound was identified by LC-MS and ¹H NMR.

C₄₀H₂₂N₂O : M+1 546.18

### Synthesis Example 6

### Synthesis of Compound 70

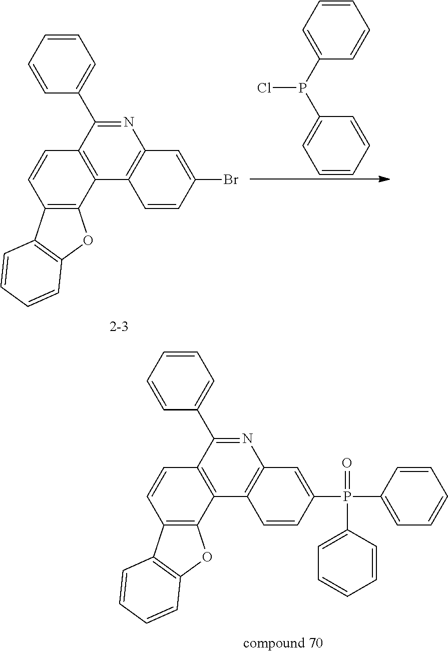 [SMILES: Brc1ccc2c(c1)nc(-c1ccccc1)c1ccc3c4ccccc4oc3c12 ; ClP(c1ccccc1)c1ccccc1 ; O=P(c1ccccc1)(c1ccccc1)c1ccc2c(c1)nc(-c1ccccc1)c1ccc3c4ccccc4oc3c12]

### Synthesis of Compound 70

4.24 g (10 mmol) of Intermediate 2-3 was dissolved in 100 mL of THF, and then, at a temperature of −78° C., 4 mL (2.5M in hexane) of normal butyllithium was added thereto. One hour after, 2.20 g (10 mmol) of chlorodiphenylphosphine was slowly added dropwise thereto. The resultant was stirred 3 hours, and then, the temperature thereof was raised to room temperature, and then, water was added thereto, and the resultant was washed three times by using 30 mL of ethylacetate. The resultant ethylacetate layer was dried by using MgSO₄, and then, dried under reduced pressure, and the resultant was dissolved in 40 mL of dichloromethane. 4 mL of hydrogen peroxide was added thereto, and then, stirred at room temperature for 20 hours. Then, 20 mL of water was added thereto, and an extraction process was performed thereon three times by using 20 mL of dichloromethane. An organic layer obtained therefrom was dried by using magnesium sulfate, and the residual obtained by evaporating a solvent therefrom was separation-purified by silica gel column chromatography, thereby completing the preparation of 3.54 g (yield of 64%) of Compound 70. The obtained compound was identified by LC-MS and ¹H NMR.

C₃₇H₂₄NO₂P : M+1 545.14

### Synthesis Example 7

### Synthesis of Compound 85

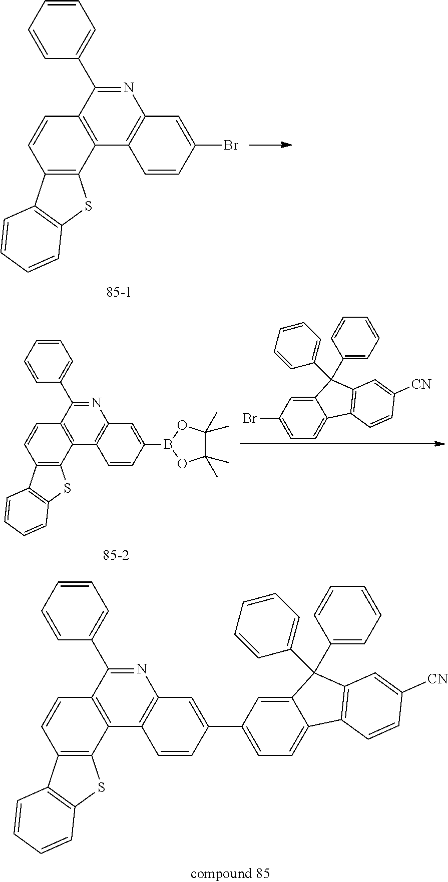

### Synthesis of Intermediate 85-1

Intermediate 85-1 was prepared in the same (or substantially the same) manner as the one used to synthesize Intermediates 2-1, 2-2, and 2-3, except that dibenzothiophene-4-boronic acid was used instead of dibenzofuran-4-boronic acid. The obtained compound was confirmed by LC-MS.

C₂₅H₁₄BrNS:M+1 439

### Synthesis of Intermediate 85-2

Intermediate 85-2 was prepared in the same (or substantially the same) manner as the one used to synthesize Intermediate 2-4, except that Intermediate 85-1 was used instead of Intermediate 2-3. The obtained compound was identified by LC-MS.

C₃₁F₂₆BNO₂S: M+1 487.18

### Synthesis of Compound 85

4.92 g (yield of 70%) of Compound 85 was prepared in the same (or substantially the same) manner as the one used to synthesize Compound 2, except that 7-bromo-9,9-diphenyl-9H-fluorene-2-carbonitrile and Intermediate 85-2 were used instead of Intermediate 2-4 and Intermediate 2-5. The obtained compound was identified by LC-MS and ¹H NMR.

C₅₁H₃₀N₂S:M+1 702.2

### Synthesis Example 8

### Synthesis of Compound 981

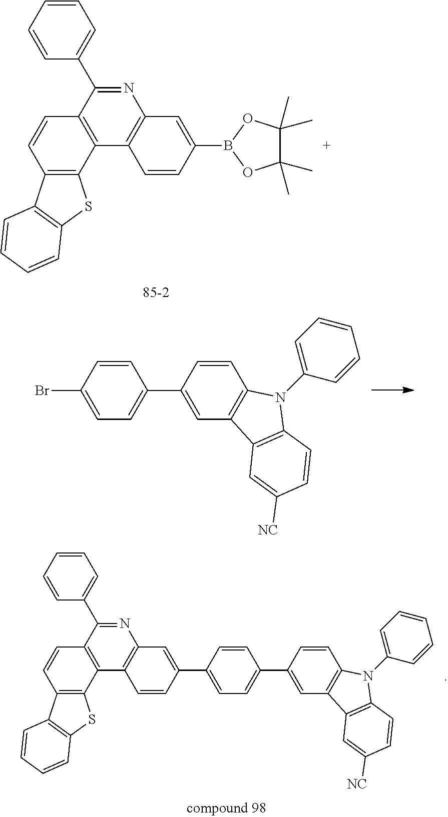 [SMILES: CC1(C)OB(c2ccc3c(c2)nc(-c2ccccc2)c2ccc4c5ccccc5sc4c23)OC1(C)C ; N#Cc1ccc2c(c1)c1cc(-c3ccc(Br)cc3)ccc1n2-c1ccccc1 ; N#Cc1ccc2c(c1)c1cc(-c3ccc(-c4ccc5c(c4)nc(-c4ccccc4)c4ccc6c7ccccc7sc6c45)cc3)ccc1n2-c1ccccc1]

### Synthesis of Compound 98

4.58 g (yield of 65%) of Compound 98 was prepared in the same (or substantially the same) manner as the one used to synthesize Compound 2, except that Intermediate 85-2 and 6-(4-bromophenyl)-9-phenyl-9H-carbazole-3-carbonitrile were used instead of Intermediate 2-4 and Intermediate 2-5. The obtained compound was identified by LC-MS and ¹H NMR.

C₅₀H₂₉N₃S:M+1 703.2 [Synthesis Example 9: Synthesis of Compound 1031

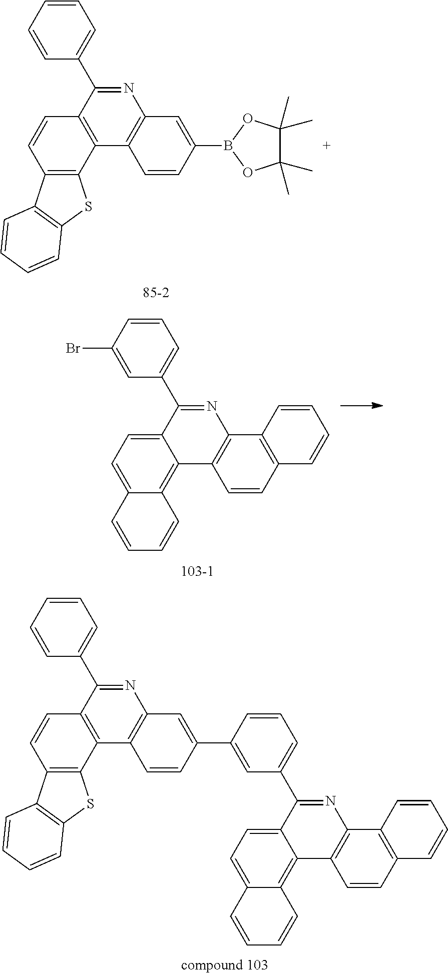 [SMILES: CC1(C)OB(c2ccc3c(c2)nc(-c2ccccc2)c2ccc4c5ccccc5sc4c23)OC1(C)C ; Brc1cccc(-c2nc3c4ccccc4ccc3c3c2ccc2ccccc23)c1 ; c1ccc(-c2nc3cc(-c4cccc(-c5nc6c7ccccc7ccc6c6c5ccc5ccccc56)c4)ccc3c3c2ccc2c4ccccc4sc23)cc1]

### Synthesis of Intermediate 103-1

The same (or substantially the same) synthesis method and synthesis processes as those used to prepare Intermediate 2-3 were used herein. In particular, Intermediate 103-1 was prepared in such a manner that, in synthesizing Intermediate 2-1, naphthalene-1-boronic acid was used instead of dibenzofuran-4-boronic acid, and 2-bromo-1-nitronaphthalene was used instead of 1,4-dibromo-2-nitrobenzene; and in synthesizing Intermediate 2-3, 3-bromobenzaldehyde was used instead of benzaldehyde. The obtained compound was confirmed by LC-MS.

C₂₇H₁₆BrN M+1 433.05

### Synthesis of Compound 103

Compound 103 was prepared in the same (or substantially the same) manner as the one used to synthesize Compound 2, except that Intermediate 85-2 and Intermediate 103-1 were used instead of Intermediate 2-4 and Intermediate 2-5. The obtained compound was identified by LC-MS and ¹H NMR.

C₅₂H₃₀N₂S:M+1 714.2

### Synthesis Example 10

### Synthesis of Compound 1161

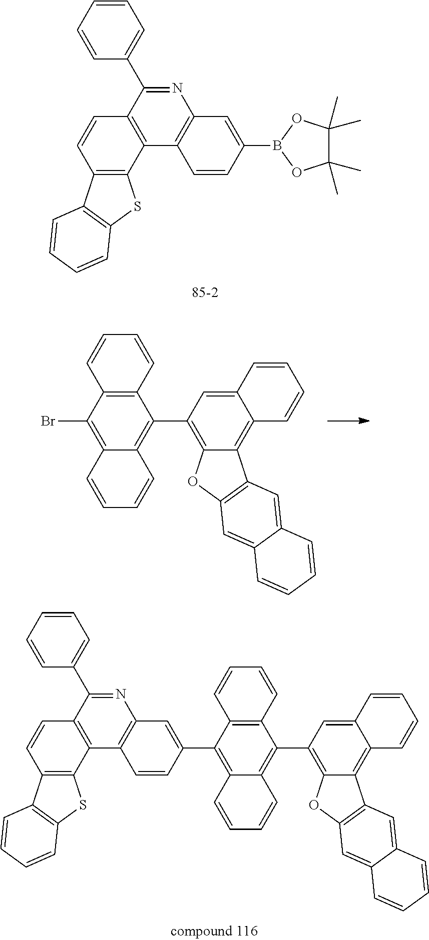 [SMILES: CC1(C)OB(c2ccc3c(c2)nc(-c2ccccc2)c2ccc4c5ccccc5sc4c23)OC1(C)C ; Brc1c2ccccc2c(-c2cc3ccccc3c3c2oc2cc4ccccc4cc23)c2ccccc12 ; c1ccc(-c2nc3cc(-c4c5ccccc5c(-c5cc6ccccc6c6c5oc5cc7ccccc7cc56)c5ccccc45)ccc3c3c2ccc2c4ccccc4sc23)cc1]

### Synthesis of Compound 116

5.6 g (yield of 69%) of Compound 116 was prepared in the same (or substantially the same) manner as used to synthesize Compound 2, except that Intermediate 85-2 and 6-(10-bromoanthracene-9-yl)dinaphtho[2,1-b:2′,3′-d]furan were used instead of Intermediate 2-4 and Intermediate 2-5. The obtained compound was identified by LC-MS and ¹H NMR.

C₅₉H₃₃NOS:M+1 803.25

### Synthesis Example 11

### Synthesis of Compound 1371

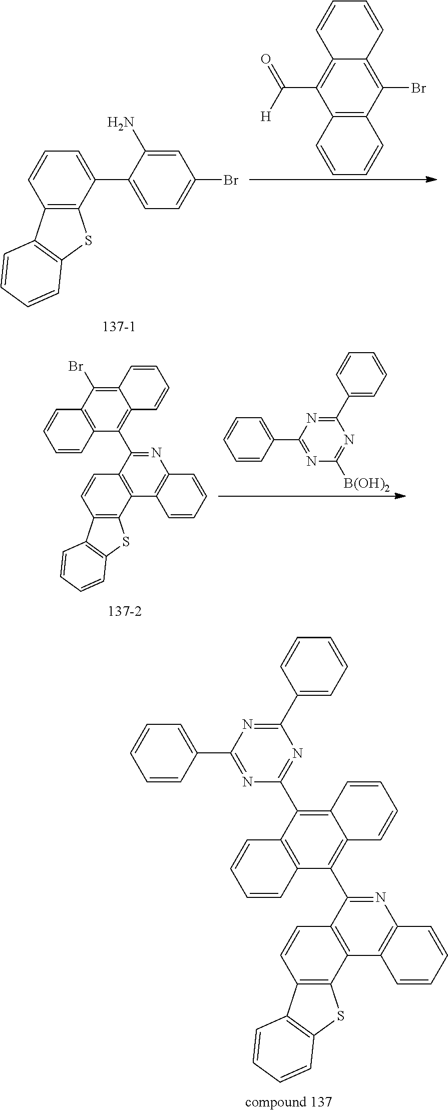

### Synthesis of Intermediate 137-1

2.5 g (yield of 70%) of Intermediate 137-1 was prepared in the same (or substantially the same) manner as the one used to synthesize Intermediate 2-2, except that in synthesizing Intermediate 2-1, dibenzothiophene-4-boronic acid was used instead of dibenzofuran-4-boronic acid. The obtained compound was confirmed by LC-MS.

C₁₈H₁₂BrNS:M+1 352.99

### Synthesis of Intermediate 137-2

2.7 g (yield of 50%) of Intermediate 137-2 was prepared in the same (or substantially the same) manner as the one used to synthesize Intermediate 57-1, except that Intermediate 137-1 was used instead of Intermediate 2-2. The obtained compound was confirmed by LC-MS.

C₃₃H₁₈BrNS:M+1 539.03

### Synthesis of Compound 137

4.85 g (yield of 70%) of Compound 137 was prepared in the same (or substantially the same) manner as the one used to synthesize Compound 2, except that Intermediate 137-2 and (4,6-diphenyl-1,3,5-triazine-2-yl)boronic acid were used instead of Intermediate 2-4 and Intermediate 2-5. The obtained compound was identified by LC-MS and ¹H NMR.

C₄₈H₂₈N₄S:M+1 692.18

### Synthesis Example 12

### Synthesis of Compound 1481

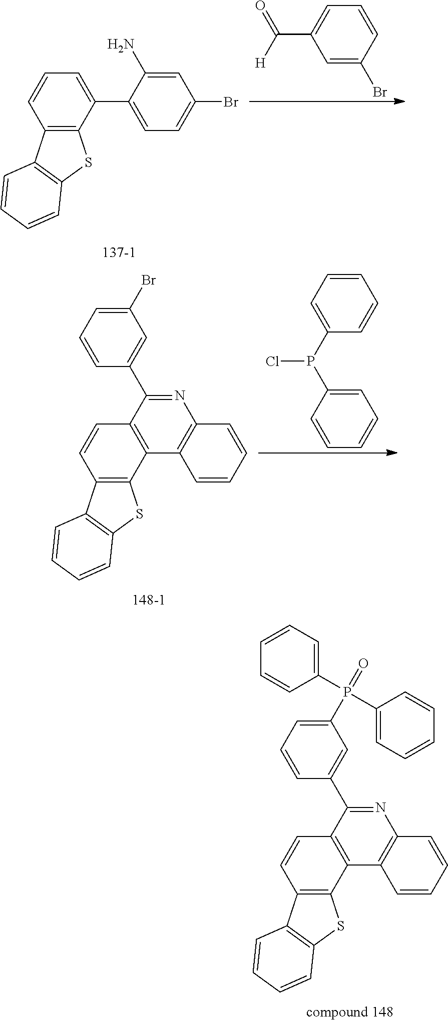 [SMILES: Nc1cc(Br)ccc1-c1cccc2c1sc1ccccc12 ; O=Cc1cccc(Br)c1 ; Brc1cccc(-c2nc3ccccc3c3c2ccc2c4ccccc4sc23)c1 ; ClP(c1ccccc1)c1ccccc1 ; O=P(c1ccccc1)(c1ccccc1)c1cccc(-c2nc3ccccc3c3c2ccc2c4ccccc4sc23)c1]

### Synthesis of Intermediate 148-1

2.2 g (yield of 50%) of Intermediate 148-1 was prepared in the same (or substantially the same) manner as the one used to synthesize Intermediate 2-3, except that Intermediate 137-1 and 3-bromobenzaldehyde were used instead of Intermediate 2-2 and benzaldehyde. The obtained compound was identified by LC-MS.

C₂₅H₁₄BrNS:M+1 439

### Synthesis of Compound 148

2.8 g (yield of 50%) of Compound 148 was prepared in the same (or substantially the same) manner as the one used to synthesize Compound 70, except that Intermediate 148-1 was used instead of Intermediate 2-3. The obtained compound was identified by LC-MS and ¹H NMR.

C₃₇H₂₄NOPS:M+1 561.14

Synthesis methods for compounds other than the compounds shown in Table 1 should be apparent to those of ordinary skill in the art by referring to the synthesis pathway and source materials described above.

TABLE 1

| | | MS/FAB | |
|---|---|---|---|
| Compound | ¹H NMR (CDCl₃, 400 MHz) | found | calc. |
| 2 | 8.96-8.92 (m, 2H), 8.69-8.65 (m, 3H), 8.33- | 575.21 | 575.2 |
| | 8.31 (m, 1H), 8.20-8.17 (m, 1H), 8.08-8.04 | | |
| | (m, 5H), 8.88-8.91 (m, 3H), 7.83-7.80 (m, | | |
| | 2H), 7.71-7.69 (m, 1H), 7.65-7.59 (m, 3H), | | |
| | 7.55-7.47 (m, 3H), 7.45-7.41 (m, 1H) | | |
| 8 | 9.08-9.04 (m, 1H), 8.64-8.61 (m, 1H), 8.47- | 572.2 | 572.19 |
| | 8.43 (m, 1H), 8.38-8.32 (m, 2H), 8.27-8.15 | | |
| | (m, 2H), 8.13-8.05 (m, 2H), 7.98-7.89 (m, | | |
| | 3H), 7.84-7.80 (m, 3H), 7.74-7.68 (m, 2H), | | |
| | 7.63-7.58 (m, 3H), 7.53-7.45 (m, 2H), 7.45- | | |
| | 7.40 (m, 1H), 7.18-7.12 (m, 1H) | | |
| 31 | 8.57-8.53 (m, 1H), 8.30-8.28 (m, 1H), 8.20- | 647.23 | 647.22 |
| | 8.17 (m, 1H), 8.11-8.06 (m, 2H), 7.95-7.90 | | |
| | (m, 3H), 7.85-7.78 (m, 5H), 7.72-7.67 (m, | | |
| | 3H), 7.65-7.59 (m, 3H), 7.55-7.50 (m, 2H), | | |
| | 7.47-7.35 (m, 4H), 7.35-7.29 (m, 3H), 7.00- | | |
| | 6.94 (m, 1H) | | |
| 37 | 8.66-8.62 (m, 2H), 8.58-8.54 (m, 1H), 8.30- | 675.2 | 675.23 |
| | 8.23 (m, 3H), 8.20-8.16 (m, 1H), 8.10-8.01 | | |
| | (m, 4H), 7.92-7.80 (m, 5H), 7.78-7.68 (m, | | |
| | 2H), 7.65-7.60 (m, 3H), 7.55-7.45 (m, 3H), | | |
| | 7.45-7.37(m, 3H), 7.27-7.23 (m, 2H) | | |
| 57 | 8.75-8.70 (m, 1H), 8.37-8.33 (m, 2H), 8.05- | 546.18 | 546.17 |
| | 8.01 (m, 3H), 7.99-7.92 (m, 3H), 7.91-7.84 | | |
| | (m, 4H), 7.70-7.65 (m, 3H), 7.53-7.50 (m, | | |
| | 1H), 7.45-7.36 (m, 3H), 7.27-7.23 (m, 2H) | | |
| 70 | 8.75-8.71 (m, 1H), 8.58-8.55 (m, 1H), 8.19- | 545.14 | 545.15 |
| | 8.05 (m, 2H), 7.91-7.88 (m, 2H), 7.85-7.80 | | |
| | (m, 2H), 7.72-7.68 (m, 5H), 7.64-7.60 (m, | | |
| | 3H), 7.53-7.47 (m, 3H), 7.45-7.40 (m, 5H) | | |
| 85 | 8.65-8.63 (m, 1H), 8.45-8.43 (m, 1H), 8.24- | 702.2 | 702.21 |
| | 8.19 (m, 2H), 7.99-7.90 (m, 4H), 7.87-7.81 | | |
| | (m, 3H), 7.72-7.68 (m, 1H), 7.65-7.54 (m, | | |
| | 5H), 7.45-7.36 (m, 2H), 7.35-7.29 (m, | | |
| | 5H), 7.13-7.08 (m, 6H) | | |
| 98 | 8.63-8.60 (m, 1H), 8.46-8.42 (m, 1H), 8.33- | 703.2 | 703.21 |
| | 8.29 (m, 1H), 8.25-8.20 (m, 3H), 8.01-7.95 | | |
| | (m, 4H), 7.92-7.86 (m, 3H), 7.85-7.82 (m, | | |
| | 3H), 7.65-7.58 (m, 6H), 7.53-7.46 (m, 4H), | | |
| | 7.46-7.42 (m, 1H), 7.31-7.25 (m, 2H) | | |
| 103 | 9.53-9.30 (m, 1H), 8.65-8.55 (m, 3H), 8.47- | 714.2 | 714.21 |
| | 8.43 (m, 1H), | | |
| | 8.35-8.26 (m, 2H), 8.23-8.17 (m, 2H), 8.04- | | |
| | 7.96 (m, 3H), 7.96-7.86 (m, 5H), 7.86-7.78 | | |
| | (m, 3H), 7.74-7.71 (m, 1H), 7.65-7.58 (m, | | |
| | 5H), 7.47-7.42 (m, 4H) | | |
| 116 | 8.99-8.95 (m, 1H), 8.68-8.64 (m, 1H), 8.54- | 803.25 | 803.23 |
| | 8.50 (m, 1H), 8.45-8.41 (m, 3H), 8.30-8.21 | | |
| | (m, 2H), 8.20-8.11 (m, 4H), 7.99-7.90 (m, | | |
| | 4H), 7.89-7.81 (m, 4H), 7.65-7.59 (m, 5H), | | |
| | 7.52-7.50 (m, 1H), 7.45-7.42 (m, 2H), 7.40- | | |
| | 7.30 (m, 5H) | | |
| 137 | 9.01-8.97 (m, 1H), 8.87-8.82 (m, 4H), 8.55- | 692.18 | 692.2 |
| | 8.52 (m, 1H), 8.44-8.40 (m, 2H), 8.27-8.20 | | |
| | (m, 2H), 7.93-7.86 (m, 2H), 7.86-7.82 (m, | | |
| | 1H), 7.75-7.68 (m, 3H), 7.68-7.58 (m, 7H), | | |
| | 7.45-7.30 (m, 5H) | | |
| 148 | 8.87-8.82 (m, 1H), 8.47-8.42 (m, 2H), 8.27- | 561.14 | 561.13 |
| | 8.21 (m, 2H), 8.06-7.90 (m, 3H), 7.86-7.80 | | |
| | (m, 3H), 7.67-7.58 (m, 5H), 7.50-7.40 (m, 8H) | | |

### Example 1

An anode was prepared by cutting a 15 Ωcm² (1,200 Å) ITO glass substrate (Corning Inc.) to a size of 50 mm×50 mm×0.7 mm, sonicating the glass substrate by using isopropyl alcohol and pure water, for 5 minutes each, and then irradiating UV light for 30 minutes thereto and exposing to ozone to clean. Then, the resulting anode was loaded onto a vacuum deposition apparatus.

Then, 2-TNATA, was vacuum deposited thereon to form a hole injection layer having a thickness of 600 Å, and then, 4,4′-bis[N-(1-naphthyl)-N-phenylamino]biphenyl (NPB) (as a hole transport compound) was deposited on the hole injection layer to form a hole transport layer having a thickness of 300 Å.

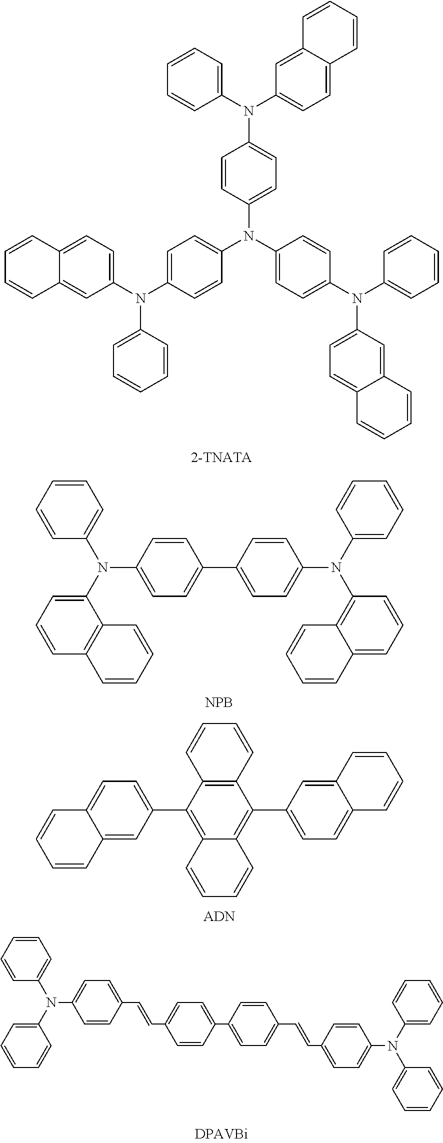

9,10-di-naphthalene-2-yl-anthracene (ADN) (as a blue fluorescent host), and 4,4′-bis[2-(4-(N,N-diphenylamino)phenyl)vinyl]biphenyl (DPAVBi) (as a blue fluorescent dopant), were co-deposited on the hole transport layer at a weight ratio of 98:2 to form an emission layer having a thickness of 300Å.

Then, Compound 2 according to the present embodiments was deposited on the emission layer to form an electron transport layer having a thickness of 300 Å, and then, LiF, which is a halogenated alkaline metal, was deposited on the electron transport layer to form an electron injection layer having a thickness of 10 Å, and Al was vacuum deposited thereon to a thickness of 3,000 Å (to form a cathode), thereby forming an LiF/AI electrode, thereby completing the manufacturing of an organic light-emitting device.

### Example 2

An organic light-emitting device was manufactured in the same (or substantially the same) manner as in Example 1, except that in forming the electron transport layer, Compound 8 was used instead of Compound 2.

### Example 3

An organic light-emitting device was manufactured in the same (or substantially the same) manner as in Example 1, except that in forming the electron transport layer, Compound 31 was used instead of Compound 2.

### Example 4

An organic light-emitting device was manufactured in the same (or substantially the same) manner as in Example 1, except that in forming the electron transport layer, Compound 37 was used instead of Compound 2.

### Example 5

An organic light-emitting device was manufactured in the same (or substantially the same) manner as in Example 1, except that in forming the electron transport layer, Compound 57 was used instead of Compound 2.

### Example 6

An organic light-emitting device was manufactured in the same (or substantially the same) manner as in Example 1, except that in forming the electron transport layer, Compound 70 was used instead of Compound 2.

### Example 7

An organic light-emitting device was manufactured in the same (or substantially the same) manner as in Example 1, except that in forming the electron transport layer, Compound 85 was used instead of Compound 2.

### Example 8

An organic light-emitting device was manufactured in the same (or substantially the same) manner as in Example 1, except that in forming the electron transport layer, Compound 98 was used instead of Compound 2.

### Example 9

An organic light-emitting device was manufactured in the same (or substantially the same) manner as in Example 1, except that in forming the electron transport layer, Compound 103 was used instead of Compound 2.

### Example 10

An organic light-emitting device was manufactured in the same (or substantially the same) manner as in Example 1, except that in forming the electron transport layer, Compound 116 was used instead of Compound 2.

### Example 11

An organic light-emitting device was manufactured in the same (or substantially the same) manner as in Example 1, except that in forming the electron transport layer, Compound 137 was used instead of Compound 2.

### Example 12

An organic light-emitting device was manufactured in the same (or substantially the same) manner as in Example 2, except that in forming the electron transport layer, Compound 148 was used instead of Compound 2.

### Comparative Example 1

An organic light-emitting device was manufactured in the same (or substantially the same) manner as in Example 1, except that in forming the electron transport layer, Alq3 was used instead of Compound 2.

The device prepared according to Comparative Example 1 had, at a current density of 50 mA/cm², a driving voltage of 7.35 V, a luminescence brightness of 2,065 cd/m², a luminescence efficiency of 4.13 cd/A, and a half-lifespan(hr @100 mA/cm²) of 145 hours.

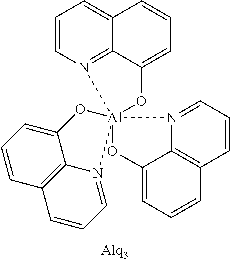

Evaluation results of the organic light-emitting devices prepared according to Examples and Comparative Example are shown in Table 2.

TABLE 2

| | Electron | Driving | Current | | | | |
|---|---|---|---|---|---|---|---|
| | transport | voltage | density | Luminance | Efficiency | Emission | Half lifespan |
| | material | (V) | (mA/cm²) | [cd/m²] | (cd/A) | color | @100 mA/cm²) |
| Example 1 | Compound | 4.88 | 50 | 3007 | 6.014 | blue | 295 hr |
| | 2 | | | | | | |
| Example 2 | Compound | 4.95 | 50 | 3200 | 6.4 | blue | 303 hr |
| | 8 | | | | | | |
| Example 3 | Compound | 5.08 | 50 | 2850 | 5.7 | blue | 365 hr |
| | 31 | | | | | | |
| Example 4 | Compound | 4.85 | 50 | 3087 | 6.174 | blue | 298 hr |
| | 37 | | | | | | |
| Example 5 | Compound | 4.97 | 50 | 3050 | 6.1 | blue | 321 hr |
| | 57 | | | | | | |
| Example 6 | Compound | 5.64 | 50 | 2850 | 5.7 | blue | 365 hr |
| | 70 | | | | | | |
| Example 7 | Compound | 5.13 | 50 | 3080 | 6.16 | blue | 306 hr |
| | 85 | | | | | | |
| Example 8 | Compound | 5.05 | 50 | 3100 | 6.2 | blue | 288 hr |
| | 98 | | | | | | |
| Example 9 | Compound | 5.22 | 50 | 2946 | 5.892 | blue | 318 hr |
| | 103 | | | | | | |
| Example 10 | Compound | 4.91 | 50 | 3105 | 6.21 | blue | 310 hr |
| | 116 | | | | | | |
| Example 11 | Compound | 5.31 | 50 | 2940 | 5.88 | blue | 292 hr |
| | 137 | | | | | | |
| Example 12 | Compound | 5.71 | 50 | 2794 | 5.588 | blue | 350 hr |
| | 148 | | | | | | |
| Comparative | Alq3 | 7.35 | 50 | 2065 | 4.13 | blue | 145 hr |
| Example 1 | | | | | | | |

Compounds represented by Formula 1 according to embodiments of the present disclosure were used as a material for forming a hole transport layer of organic light-emitting devices prepared according to Examples 1 to 12. As can be seen from the results shown in Table 2, organic light-emitting devices prepared according to Examples 1 to 12 including the compounds according to embodiments exhibited excellent current-voltage-luminance (I-V-L) characteristics, such as low driving voltage and high efficiency, and had substantially prolonged lifespan, as compared with the organic light-emitting device prepared according to Comparative Example 1. From the results shown in Table 2, it is believed that compounds according to embodiments are suitable for use as an electron transport material.

An organic light-emitting device according to embodiments of the present disclosure may have high efficiency, low voltage, high luminance, and a long lifespan.

As used herein, the terms “use,” “using,” and “used” may be considered synonymous with the terms “utilize,” “utilizing,” and “utilized,” respectively.

In addition, the terms “substantially,” “about,” and similar terms are used as terms of approximation and not as terms of degree, and are intended to account for the inherent deviations in measured or calculated values that would be recognized by those of ordinary skill in the art.

It will be understood that when an element such as a layer, film, region, or substrate is referred to as being “on” another element, it can be directly on the other element or intervening elements may also be present.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the drawing, it will be understood by those of ordinary skill in the art that various changes in form and details may be made therein without departing from the spirit and scope of the present disclosure as defined by the following claims and equivalents thereof.

What is claimed is:

1. A compound represented by Formula 1: 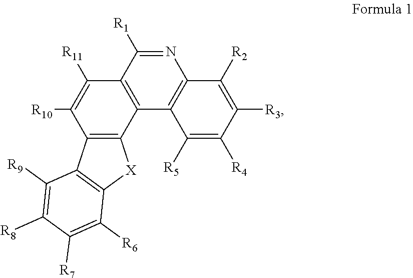
wherein, in Formula 1,
X is O or S;
R₁ to R₁₁ are independently selected from hydrogen, deuterium, a halogen, a nitro group, a cyano group, a substituted ed or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substitued or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;
wherein two adjacent substituents selected from R₁ to R₁₁ are optionally linked to each other to form a ring; and
at least one substituent of the substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₂-C₁₀ heterocycloalkyl group, substituted C₃-C₁₀ cycloalkenyl group, substituted C₂-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group is selected from the group consisting of:
deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic groupb and a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and
—P(═O)(Q₂₈)(Q₂₉),
wherein Q₁₁ to Q₁₇ and Q₂₁ to Q₂₉ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C_{6o heteroaryl group, a monovalent non-aromatic condensed polycyclic group, and a monovalent non-aromatic condensed heteropolycyclic group.}

2. The compound of claim 1, wherein
R₂ to R₆ in Formula 1 are each independently selected from hydrogen and deuterium.

3. The compound of claim 1, wherein in Formula 1,
R₁ is -(L₁)ₐ-(R₂₁)_{b},
R₃ is (L₃)_{c}-(R₂₃)_{d},
descriptions of R₂₁ and R₂₃ are each independently the same as the description provided in connection with R₁ to R₁₁;
L₁ and L₃ are each independently selected from an unsubstituted C₆-C₆₀ arylene group, an unsubstituted C₁-C₆₀ heteroarylene group, an unsubstituted divalent non-aromatic condensed polycyclic group, and an unsubstituted divalent non-aromatic condensed heteropolycyclic group; and
a, b, c, and d are each independently an integer selected from 0 to 3.

4. The compound of claim 3, wherein
L₁ and L₃ are each independently represented by one of Formulae 2a to 2e:

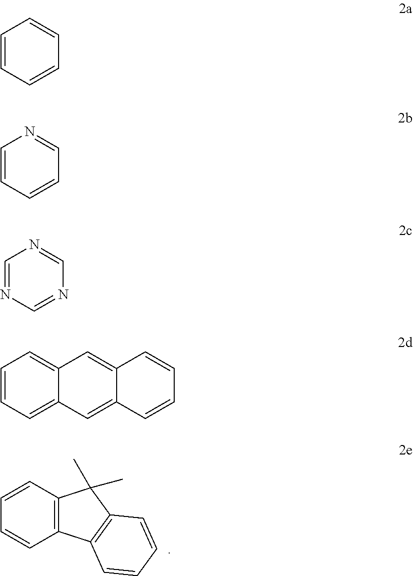

5. The compound of claim 3, wherein
R₂₁ and R₂₃ are each independently selected from hydrogen, deuterium, and a group represented by one of Formulae 3a to 3k: 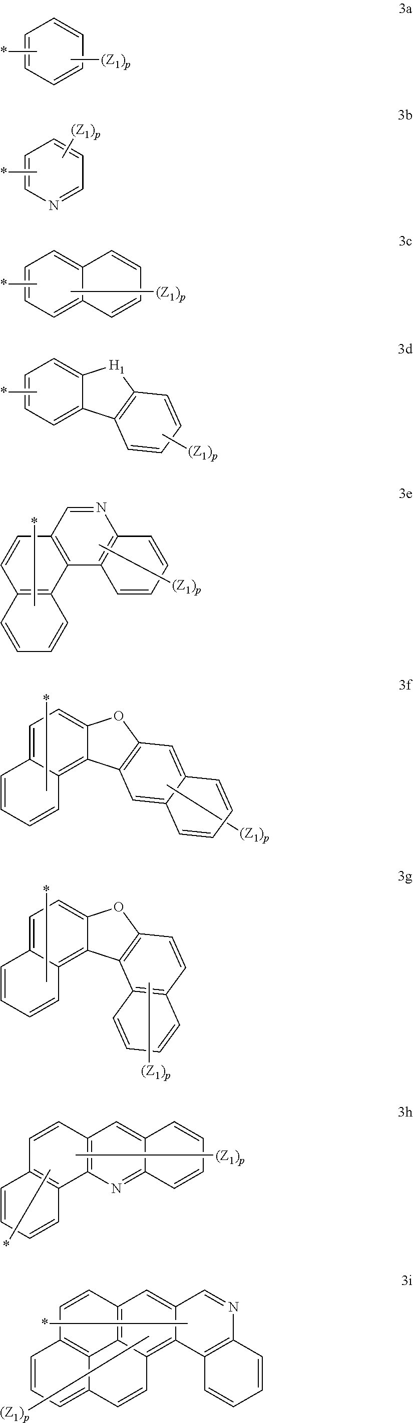 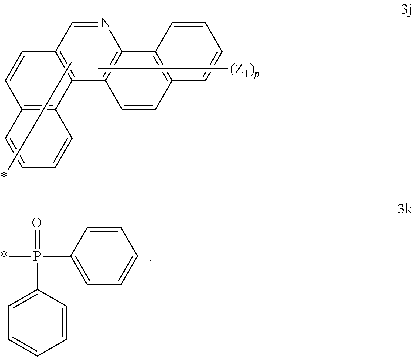
wherein, in Formulae 3a to 3k,
H₁ is CR₃₁R₃₂, NR₃₃, O, or S,
R₃₁ to R₃₃, and Z₁ are each independently selected from hydrogen, deuterium, a halogen, a cyano group, a nitro group, a hydroxyl group, a carboxy group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, and a substituted or unsubstituted monovalent non-aromatic condensed heteropolycyclic group;
p in Formulae 3a and 3e is an integer selected from 1 to 5, p in Formulae 3b and 3d is an integer selected from 1 to 4, p in Formulae 3c, 3h, and 3j is an integer selected from 1 to 7, p in Formulae 3f, 3g, and 3i is an integer selected from 1 to 6, and when p is two or more, a plurality of Zᵢ(s) is identical to or different from each other; and
*indicates a bonding site.

6. The compound of claim 5, wherein
when H₁ in Formula 3d is CR₃₁R₃₂, R₃₁ and R₃₂ are linked to each other to form a ring.

7. The compound of claim 1, wherein
the compound represented by Formula 1 is represented by Formula 2: 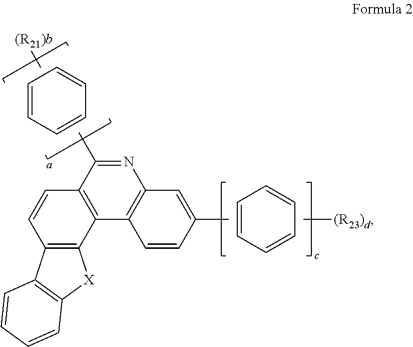
wherein, in Formula 2,
descriptions of R₂₁ and R₂₃ are each independently the same as the description provided in connection with R₁ to R₁₁; and
a, b, c, and d are each independently an integer selected from 0 to 3.

8. The compound of claim 1, wherein
the compound represented by Formula 1 is represented by Formula 3: 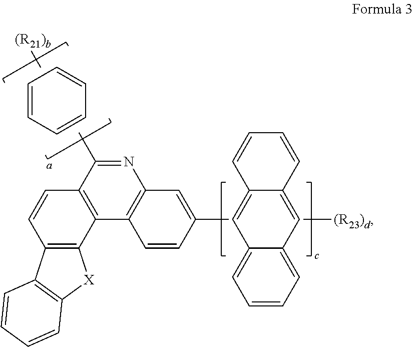
wherein, in Formula 3,
descriptions of R₂₁ and R₂₃ are each independently the same as the description provided in connection with R₁ to R₁₁; and
a, b, c, and d are each independently an integer selected from 0 to 3.

9. The compound of claim 1, wherein
the compound represented by Formula 1 is represented by Formula 4: 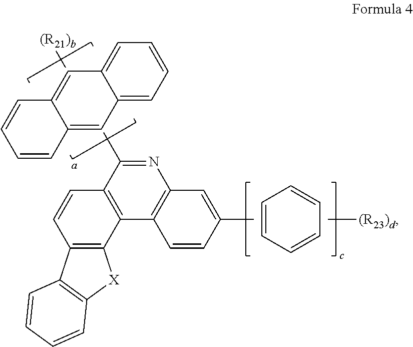
wherein, in Formula 4,
descriptions of R₂₁ and R₂₃ are each independently the same as the description provided in connection with R₁ to R₁₁; and
a, b, c, and d are each independently an integer selected from 0 to 3.

10. The compound of claim 1, wherein
the compound represented by Formula 1 is represented by Formula 5: 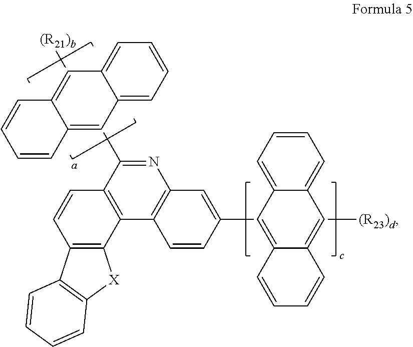
wherein, in Formula 5,
descriptions of R₂₁ and R₂₃ are each independently the same as the description provided in connection with R₁ to R₁₁; and
a, b, c, and d are each independently an integer selected from 0 to 3.

11. The compound of claim 1, wherein
the compound represented by Formula 1 is represented by Formula 6: 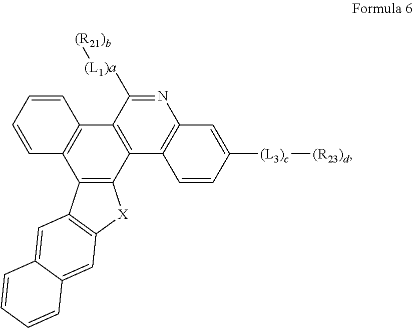
wherein, in Formula 6,
descriptions of R₂₁ and R₂₃ are each independently the same as the description provided in connection with R₁ to R₁₁;
L₁ and L₃ are each independently selected from an unsubstituted C₆-C₆₀ arylene group, an unsubstituted C₁-C₆₀ heteroarylene group, an unsubstituted divalent non-aromatic condensed polycyclic group, and an unsubstituted divalent non-aromatic condensed heteropolycyclic group; and
a, b, c, and d are each independently an integer selected from 0 to 3.

12. The compound of claim 1, wherein
the compound represented by Formula 1 is represented by Formula 7: 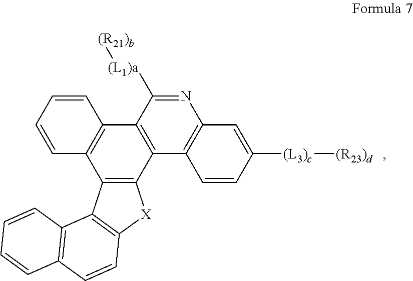
wherein, in Formula 7,
descriptions of R₂₁ and R₂₃ are each independently the same as the description provided in connection with R₁ to R₁₁;
L₁ and L₃ are each independently selected from an unsubstituted C₆-C₆₀ arylene group, an unsubstituted C₁-C₆₀ heteroarylene group, an unsubstituted divalent non-aromatic condensed polycyclic group, and an unsubstituted divalent non-aromatic condensed heteropolycyclic group; and
a, b, c, and d are each independently an integer selected from 0 to 3.

13. The compound of claim 1, wherein
the compound represented by Formula 1 is one selected from the following Compounds 1 to 172:

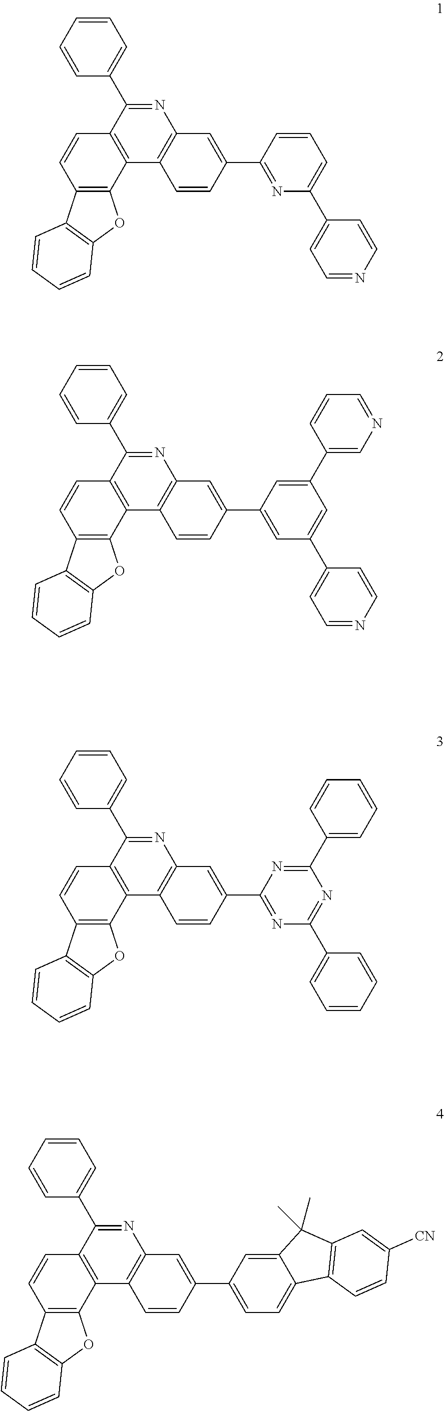 [SMILES: c1ccc(-c2nc3cc(-c4cccc(-c5ccncc5)n4)ccc3c3c2ccc2c4ccccc4oc23)cc1 ; c1ccc(-c2nc3cc(-c4cc(-c5ccncc5)cc(-c5cccnc5)c4)ccc3c3c2ccc2c4ccccc4oc23)cc1 ; c1ccc(-c2nc(-c3ccccc3)nc(-c3ccc4c(c3)nc(-c3ccccc3)c3ccc5c6ccccc6oc5c34)n2)cc1 ; CC1(C)c2cc(C#N)ccc2-c2ccc(-c3ccc4c(c3)nc(-c3ccccc3)c3ccc5c6ccccc6oc5c34)cc21]

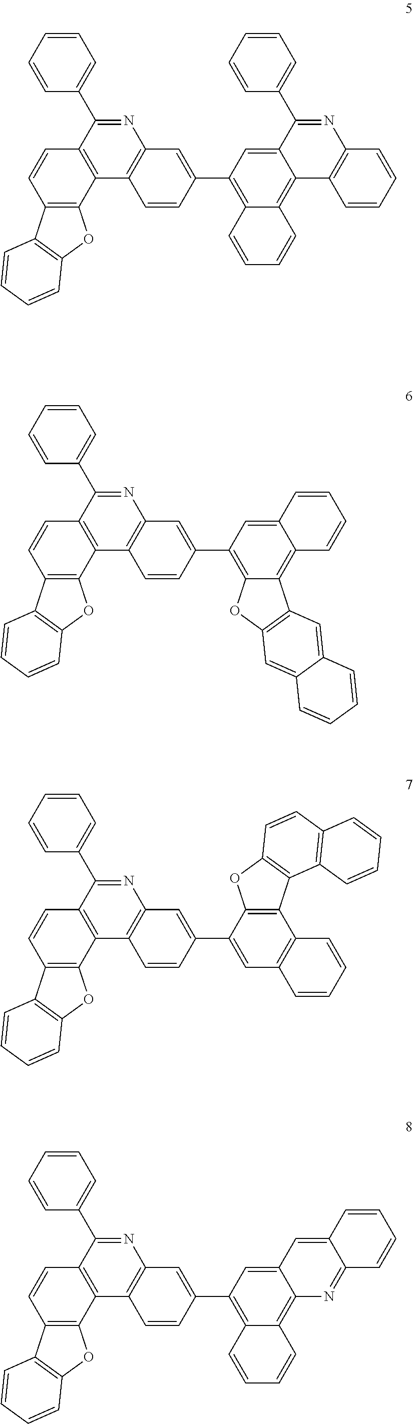 [SMILES: c1ccc(-c2nc3ccccc3c3c2cc(-c2ccc4c(c2)nc(-c2ccccc2)c2ccc5c6ccccc6oc5c24)c2ccccc23)cc1 ; c1ccc(-c2nc3cc(-c4cc5ccccc5c5c4oc4cc6ccccc6cc45)ccc3c3c2ccc2c4ccccc4oc23)cc1 ; c1ccc(-c2nc3cc(-c4cc5ccccc5c5c4oc4ccc6ccccc6c45)ccc3c3c2ccc2c4ccccc4oc23)cc1 ; c1ccc(-c2nc3cc(-c4cc5cc6ccccc6nc5c5ccccc45)ccc3c3c2ccc2c4ccccc4oc23)cc1]

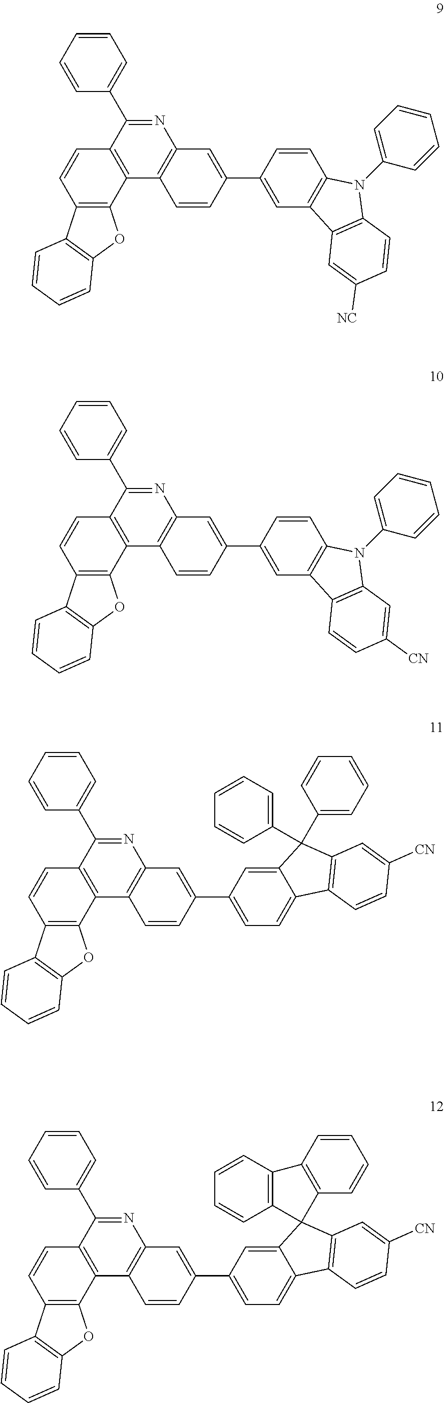 [SMILES: N#Cc1ccc2c(c1)c1cc(-c3ccc4c(c3)nc(-c3ccccc3)c3ccc5c6ccccc6oc5c34)ccc1n2-c1ccccc1 ; N#Cc1ccc2c3cc(-c4ccc5c(c4)nc(-c4ccccc4)c4ccc6c7ccccc7oc6c45)ccc3n(-c3ccccc3)c2c1 ; N#Cc1ccc2c(c1)C(c1ccccc1)(c1ccccc1)c1cc(-c3ccc4c(c3)nc(-c3ccccc3)c3ccc5c6ccccc6oc5c34)ccc1-2 ; N#Cc1ccc2c(c1)C1(c3ccccc3-c3ccccc31)c1cc(-c3ccc4c(c3)nc(-c3ccccc3)c3ccc5c6ccccc6oc5c34)ccc1-2]

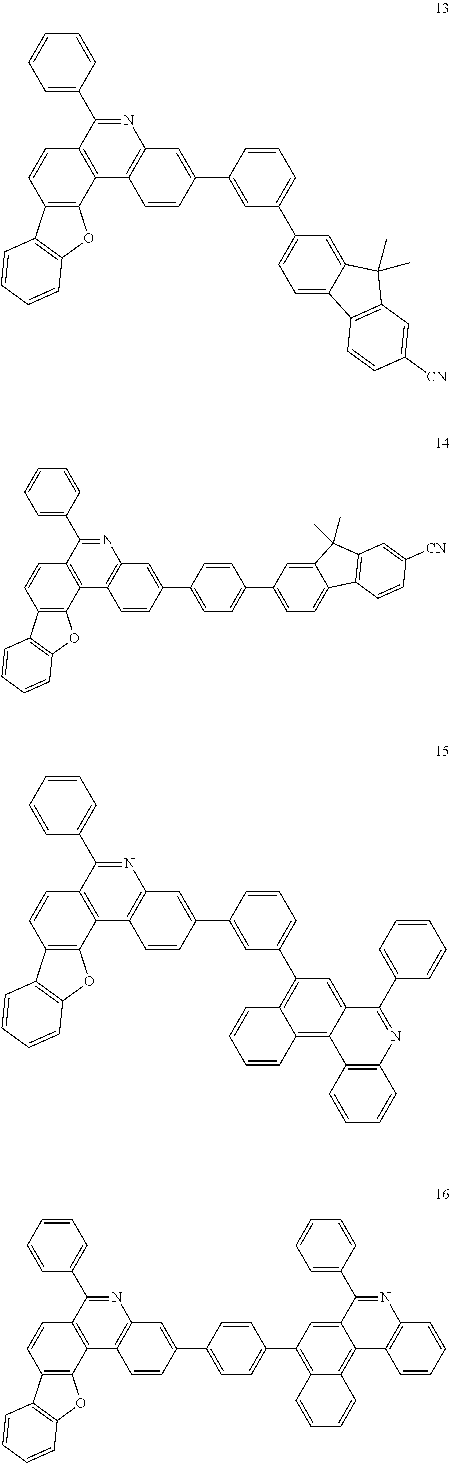 [SMILES: CC1(C)c2cc(C#N)ccc2-c2ccc(-c3cccc(-c4ccc5c(c4)nc(-c4ccccc4)c4ccc6c7ccccc7oc6c45)c3)cc21 ; CC1(C)c2cc(C#N)ccc2-c2ccc(-c3ccc(-c4ccc5c(c4)nc(-c4ccccc4)c4ccc6c7ccccc7oc6c45)cc3)cc21 ; c1ccc(-c2nc3ccccc3c3c2cc(-c2cccc(-c4ccc5c(c4)nc(-c4ccccc4)c4ccc6c7ccccc7oc6c45)c2)c2ccccc23)cc1 ; c1ccc(-c2nc3ccccc3c3c2cc(-c2ccc(-c4ccc5c(c4)nc(-c4ccccc4)c4ccc6c7ccccc7oc6c45)cc2)c2ccccc23)cc1]

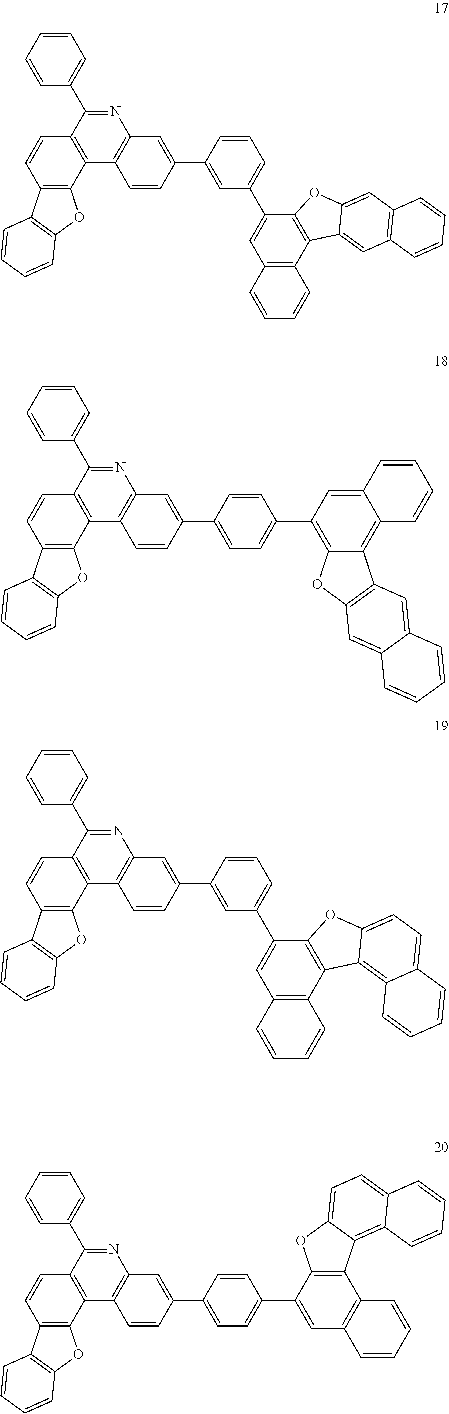 [SMILES: c1ccc(-c2nc3cc(-c4cccc(-c5cc6ccccc6c6c5oc5cc7ccccc7cc56)c4)ccc3c3c2ccc2c4ccccc4oc23)cc1 ; c1ccc(-c2nc3cc(-c4ccc(-c5cc6ccccc6c6c5oc5cc7ccccc7cc56)cc4)ccc3c3c2ccc2c4ccccc4oc23)cc1 ; c1ccc(-c2nc3cc(-c4cccc(-c5cc6ccccc6c6c5oc5ccc7ccccc7c56)c4)ccc3c3c2ccc2c4ccccc4oc23)cc1 ; c1ccc(-c2nc3cc(-c4ccc(-c5cc6ccccc6c6c5oc5ccc7ccccc7c56)cc4)ccc3c3c2ccc2c4ccccc4oc23)cc1]

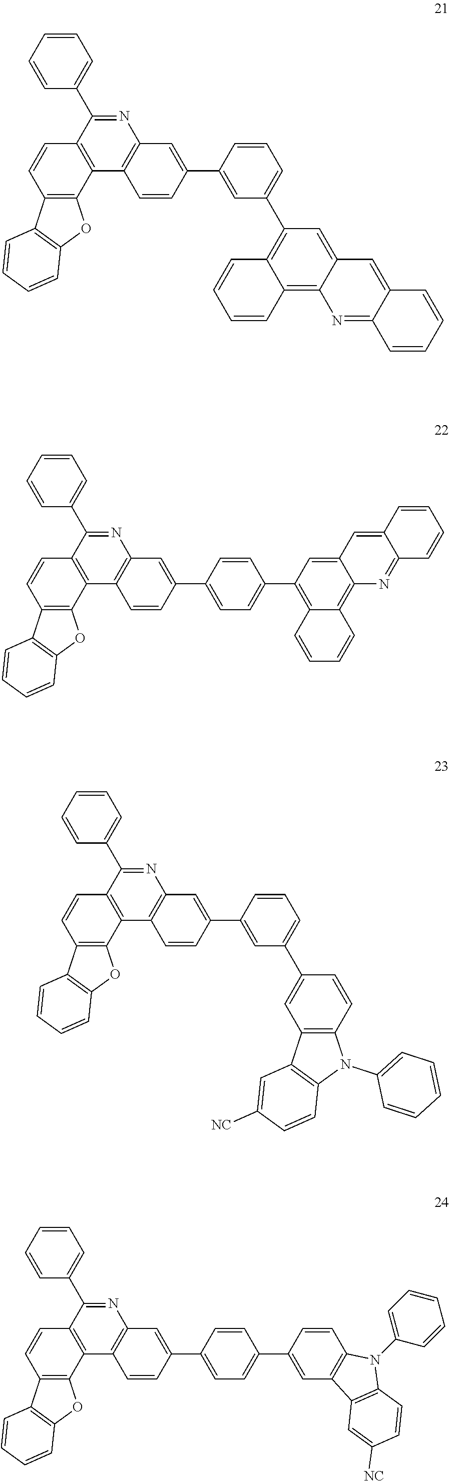

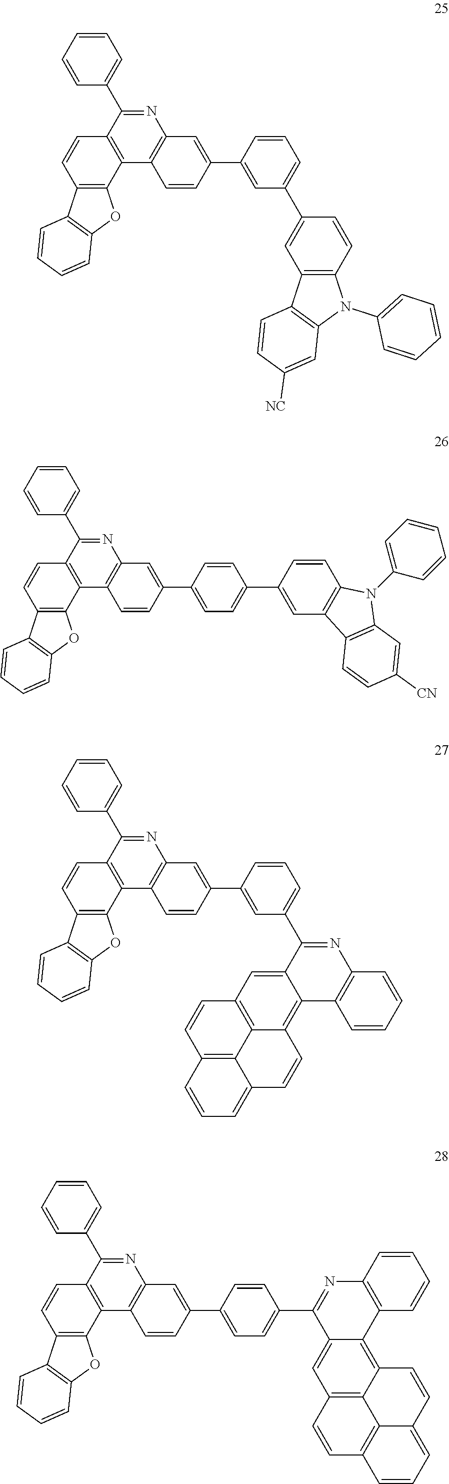 [SMILES: N#Cc1ccc2c3cc(-c4cccc(-c5ccc6c(c5)nc(-c5ccccc5)c5ccc7c8ccccc8oc7c56)c4)ccc3n(-c3ccccc3)c2c1 ; N#Cc1ccc2c3cc(-c4ccc(-c5ccc6c(c5)nc(-c5ccccc5)c5ccc7c8ccccc8oc7c56)cc4)ccc3n(-c3ccccc3)c2c1 ; c1ccc(-c2nc3cc(-c4cccc(-c5nc6ccccc6c6c5cc5ccc7cccc8ccc6c5c78)c4)ccc3c3c2ccc2c4ccccc4oc23)cc1 ; c1ccc(-c2nc3cc(-c4ccc(-c5nc6ccccc6c6c5cc5ccc7cccc8ccc6c5c78)cc4)ccc3c3c2ccc2c4ccccc4oc23)cc1]

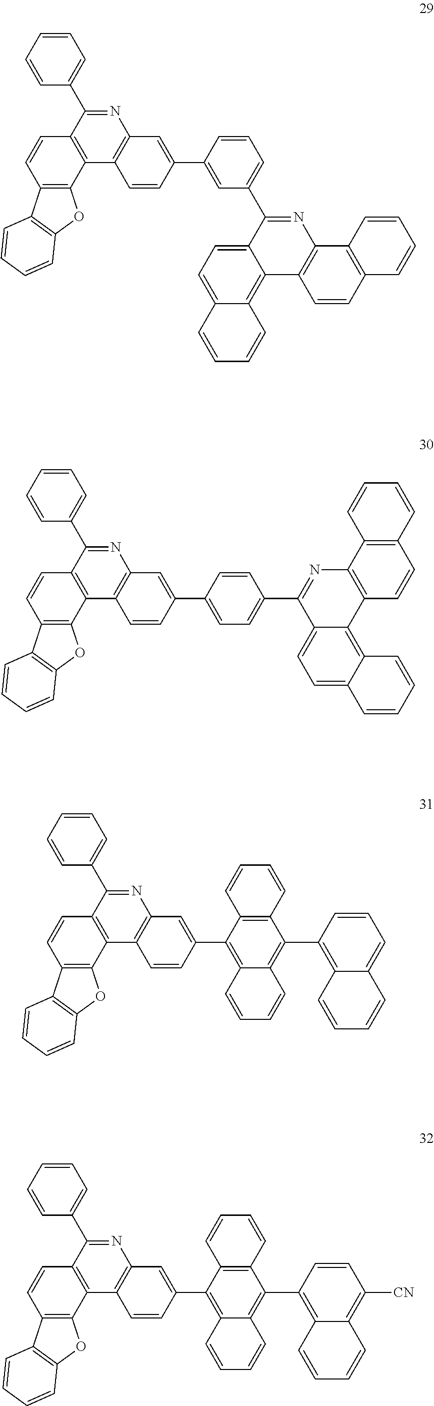 [SMILES: c1ccc(-c2nc3cc(-c4cccc(-c5nc6c7ccccc7ccc6c6c5ccc5ccccc56)c4)ccc3c3c2ccc2c4ccccc4oc23)cc1 ; c1ccc(-c2nc3cc(-c4ccc(-c5nc6c7ccccc7ccc6c6c5ccc5ccccc56)cc4)ccc3c3c2ccc2c4ccccc4oc23)cc1 ; c1ccc(-c2nc3cc(-c4c5ccccc5c(-c5cccc6ccccc56)c5ccccc45)ccc3c3c2ccc2c4ccccc4oc23)cc1 ; N#Cc1ccc(-c2c3ccccc3c(-c3ccc4c(c3)nc(-c3ccccc3)c3ccc5c6ccccc6oc5c34)c3ccccc23)c2ccccc12]

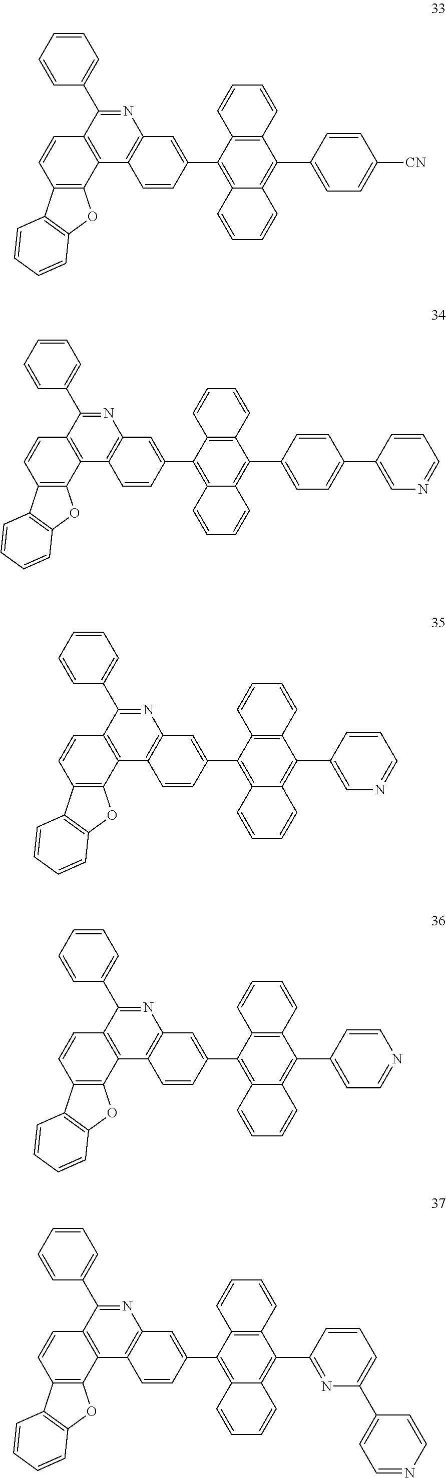 [SMILES: N#Cc1ccc(-c2c3ccccc3c(-c3ccc4c(c3)nc(-c3ccccc3)c3ccc5c6ccccc6oc5c34)c3ccccc23)cc1 ; c1ccc(-c2nc3cc(-c4c5ccccc5c(-c5ccc(-c6cccnc6)cc5)c5ccccc45)ccc3c3c2ccc2c4ccccc4oc23)cc1 ; c1ccc(-c2nc3cc(-c4c5ccccc5c(-c5cccnc5)c5ccccc45)ccc3c3c2ccc2c4ccccc4oc23)cc1 ; c1ccc(-c2nc3cc(-c4c5ccccc5c(-c5ccncc5)c5ccccc45)ccc3c3c2ccc2c4ccccc4oc23)cc1 ; c1ccc(-c2nc3cc(-c4c5ccccc5c(-c5cccc(-c6ccncc6)n5)c5ccccc45)ccc3c3c2ccc2c4ccccc4oc23)cc1]

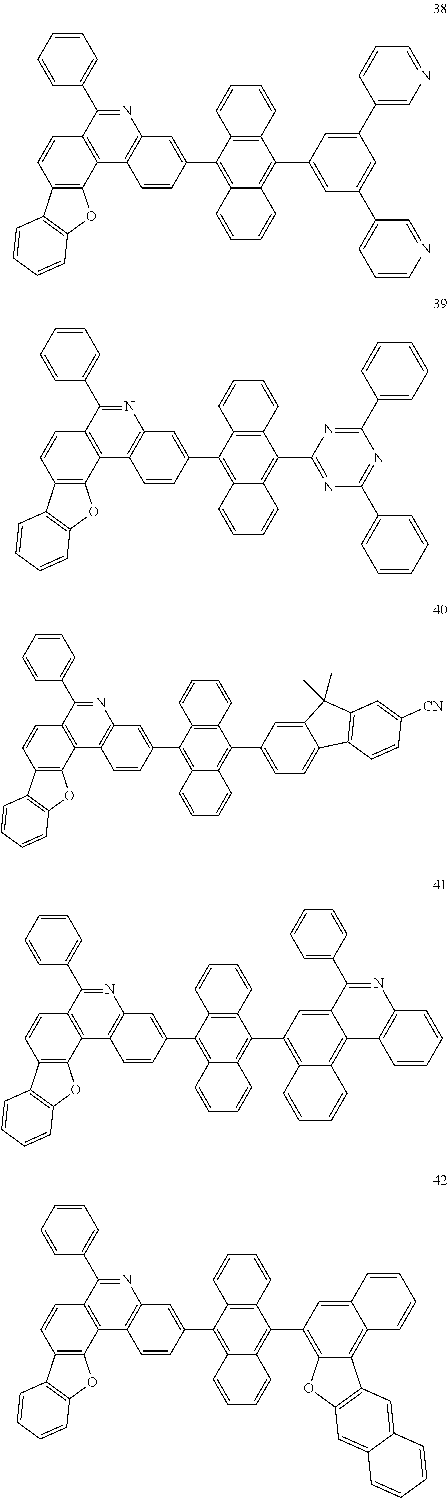

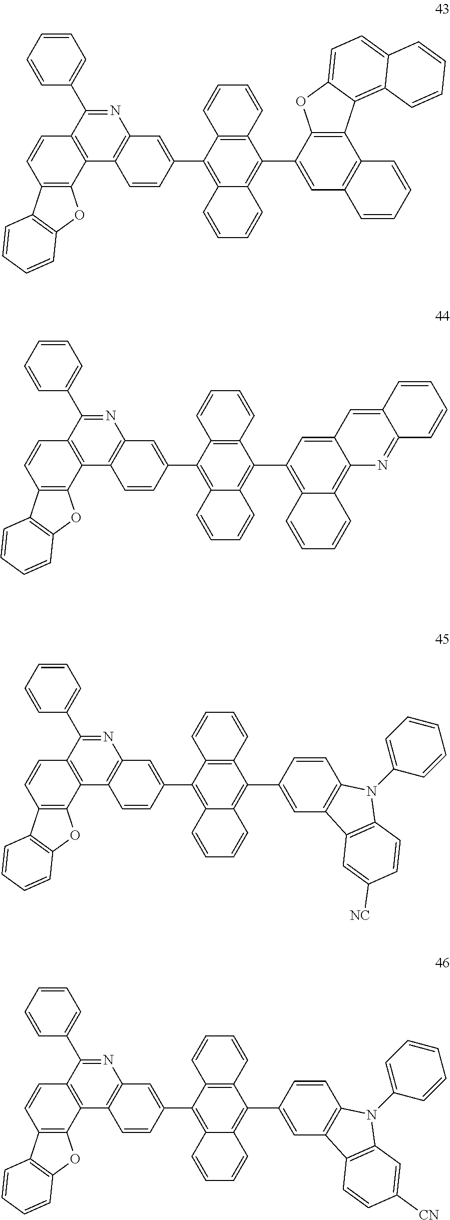 [SMILES: c1ccc(-c2nc3cc(-c4c5ccccc5c(-c5cc6ccccc6c6c5oc5ccc7ccccc7c56)c5ccccc45)ccc3c3c2ccc2c4ccccc4oc23)cc1 ; c1ccc(-c2nc3cc(-c4c5ccccc5c(-c5cc6cc7ccccc7nc6c6ccccc56)c5ccccc45)ccc3c3c2ccc2c4ccccc4oc23)cc1 ; N#Cc1ccc2c(c1)c1cc(-c3c4ccccc4c(-c4ccc5c(c4)nc(-c4ccccc4)c4ccc6c7ccccc7oc6c45)c4ccccc34)ccc1n2-c1ccccc1 ; N#Cc1ccc2c3cc(-c4c5ccccc5c(-c5ccc6c(c5)nc(-c5ccccc5)c5ccc7c8ccccc8oc7c56)c5ccccc45)ccc3n(-c3ccccc3)c2c1]

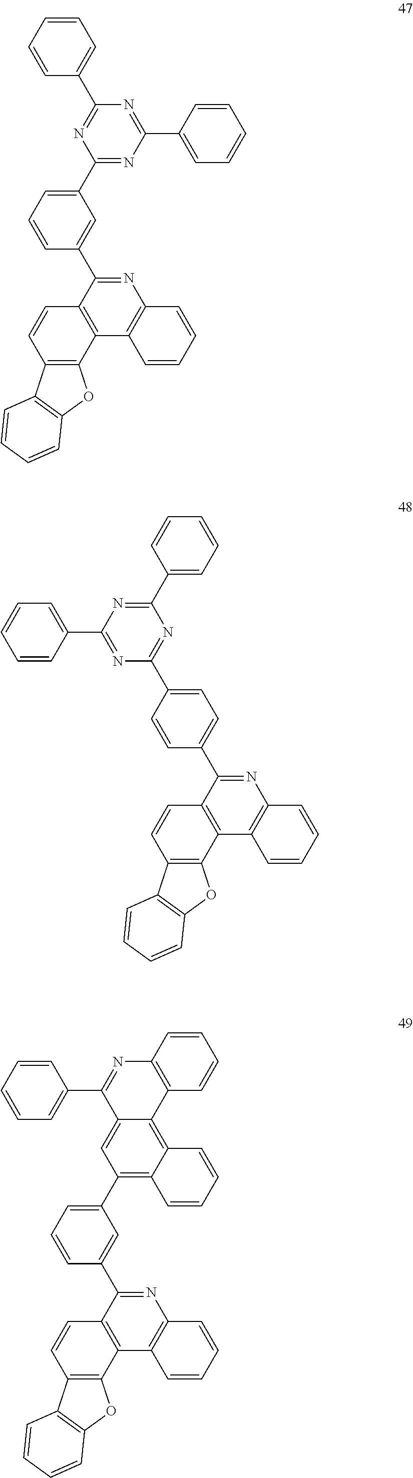 [SMILES: c1ccc(-c2nc(-c3ccccc3)nc(-c3cccc(-c4nc5ccccc5c5c4ccc4c6ccccc6oc45)c3)n2)cc1 ; c1ccc(-c2nc(-c3ccccc3)nc(-c3ccc(-c4nc5ccccc5c5c4ccc4c6ccccc6oc45)cc3)n2)cc1 ; c1ccc(-c2nc3ccccc3c3c2cc(-c2cccc(-c4nc5ccccc5c5c4ccc4c6ccccc6oc45)c2)c2ccccc23)cc1]

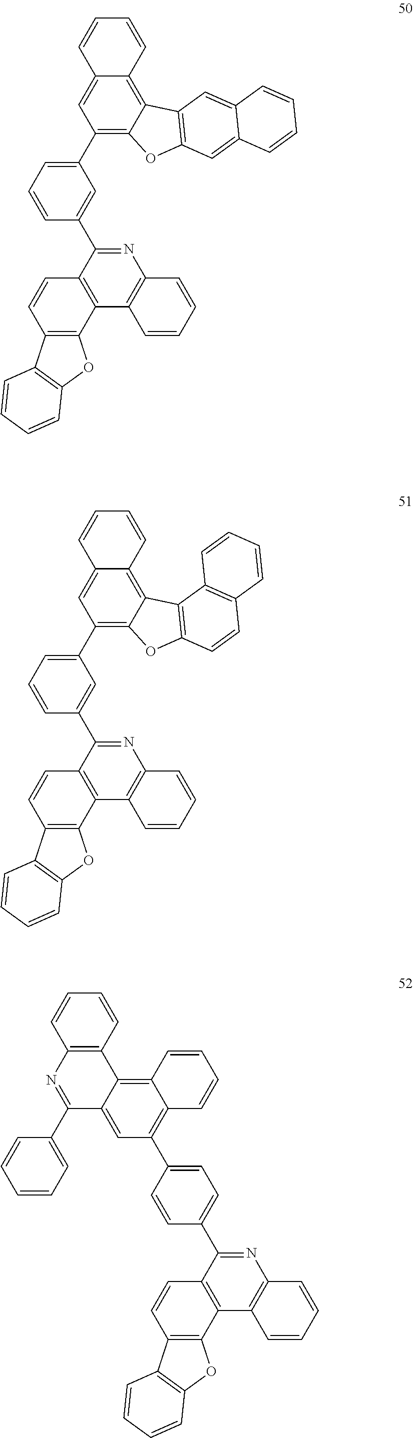 [SMILES: c1cc(-c2nc3ccccc3c3c2ccc2c4ccccc4oc23)cc(-c2cc3ccccc3c3c2oc2cc4ccccc4cc23)c1 ; c1cc(-c2nc3ccccc3c3c2ccc2c4ccccc4oc23)cc(-c2cc3ccccc3c3c2oc2ccc4ccccc4c23)c1 ; c1ccc(-c2nc3ccccc3c3c2cc(-c2ccc(-c4nc5ccccc5c5c4ccc4c6ccccc6oc45)cc2)c2ccccc23)cc1]

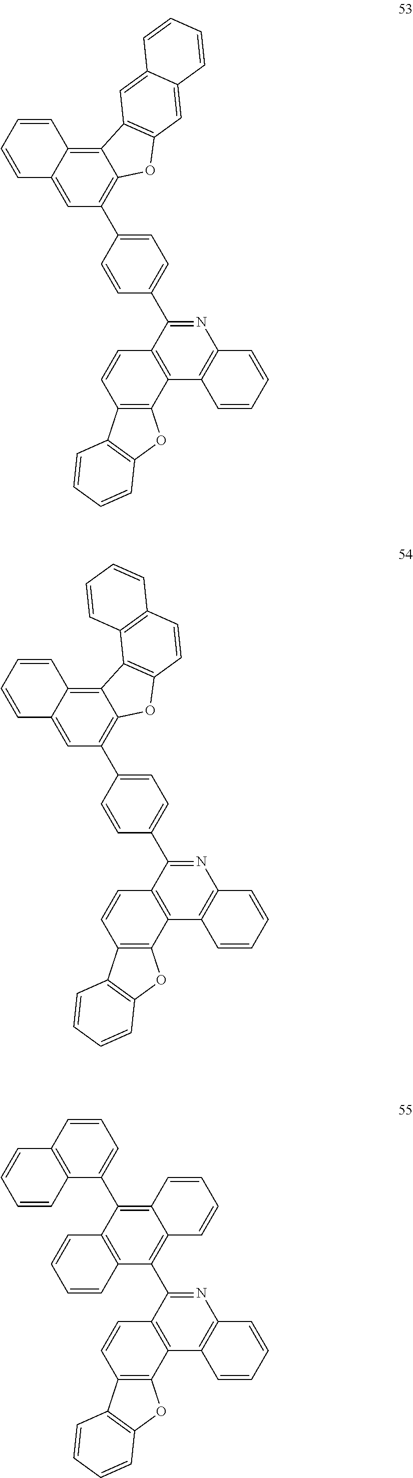 [SMILES: c1ccc2cc3c(cc2c1)oc1c(-c2ccc(-c4nc5ccccc5c5c4ccc4c6ccccc6oc45)cc2)cc2ccccc2c13 ; c1ccc2c(c1)ccc1oc3c(-c4ccc(-c5nc6ccccc6c6c5ccc5c7ccccc7oc56)cc4)cc4ccccc4c3c12 ; c1ccc2c(-c3c4ccccc4c(-c4nc5ccccc5c5c4ccc4c6ccccc6oc45)c4ccccc34)cccc2c1]

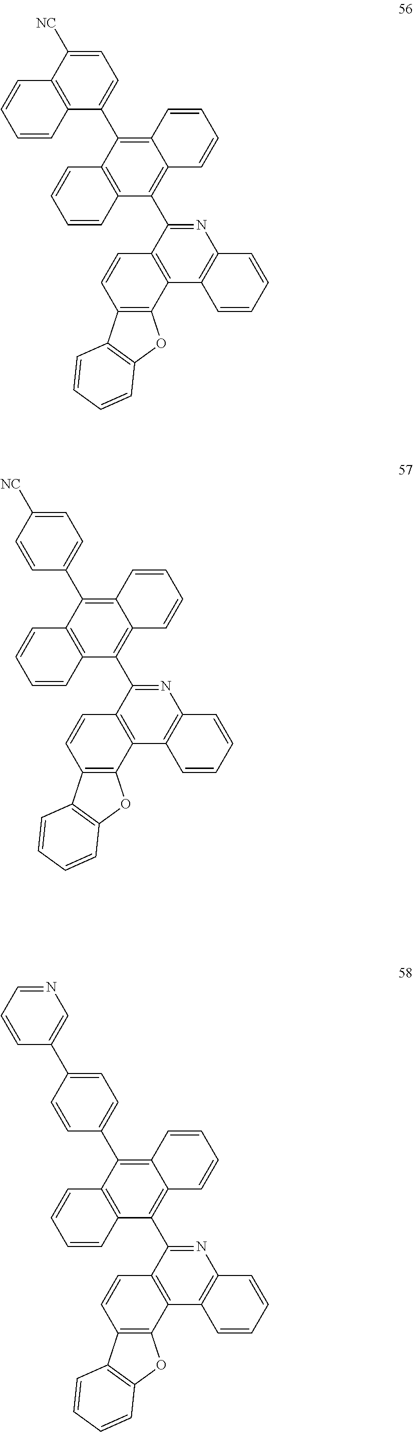 [SMILES: N#Cc1ccc(-c2c3ccccc3c(-c3nc4ccccc4c4c3ccc3c5ccccc5oc34)c3ccccc23)c2ccccc12 ; N#Cc1ccc(-c2c3ccccc3c(-c3nc4ccccc4c4c3ccc3c5ccccc5oc34)c3ccccc23)cc1 ; c1cncc(-c2ccc(-c3c4ccccc4c(-c4nc5ccccc5c5c4ccc4c6ccccc6oc45)c4ccccc34)cc2)c1]

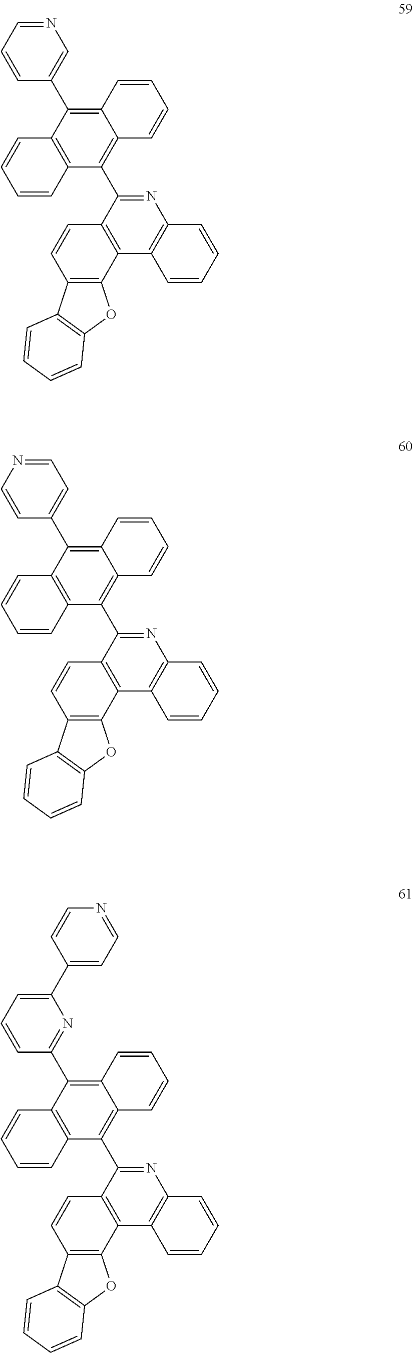 [SMILES: c1cncc(-c2c3ccccc3c(-c3nc4ccccc4c4c3ccc3c5ccccc5oc34)c3ccccc23)c1 ; c1ccc2c(c1)nc(-c1c3ccccc3c(-c3ccncc3)c3ccccc13)c1ccc3c4ccccc4oc3c12 ; c1cc(-c2ccncc2)nc(-c2c3ccccc3c(-c3nc4ccccc4c4c3ccc3c5ccccc5oc34)c3ccccc23)c1]

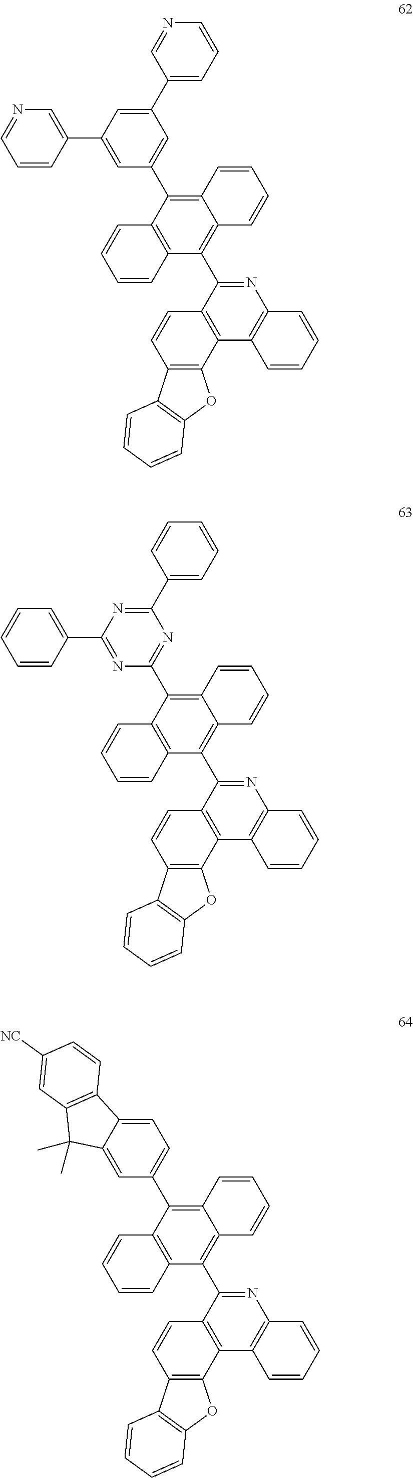 [SMILES: c1cncc(-c2cc(-c3cccnc3)cc(-c3c4ccccc4c(-c4nc5ccccc5c5c4ccc4c6ccccc6oc45)c4ccccc34)c2)c1 ; c1ccc(-c2nc(-c3ccccc3)nc(-c3c4ccccc4c(-c4nc5ccccc5c5c4ccc4c6ccccc6oc45)c4ccccc34)n2)cc1 ; CC1(C)c2cc(C#N)ccc2-c2ccc(-c3c4ccccc4c(-c4nc5ccccc5c5c4ccc4c6ccccc6oc45)c4ccccc34)cc21]

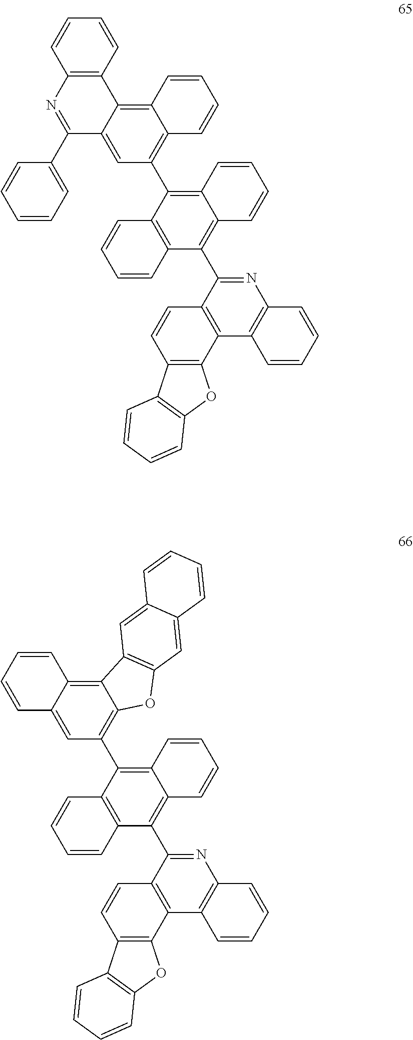 [SMILES: c1ccc(-c2nc3ccccc3c3c2cc(-c2c4ccccc4c(-c4nc5ccccc5c5c4ccc4c6ccccc6oc45)c4ccccc24)c2ccccc23)cc1 ; c1ccc2cc3c(cc2c1)oc1c(-c2c4ccccc4c(-c4nc5ccccc5c5c4ccc4c6ccccc6oc45)c4ccccc24)cc2ccccc2c13]

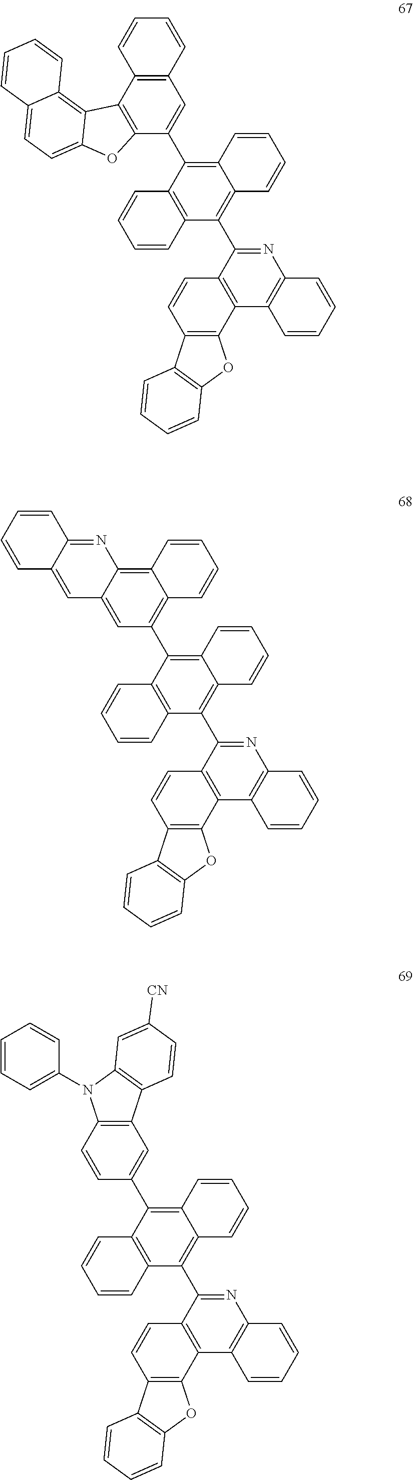 [SMILES: c1ccc2c(c1)ccc1oc3c(-c4c5ccccc5c(-c5nc6ccccc6c6c5ccc5c7ccccc7oc56)c5ccccc45)cc4ccccc4c3c12 ; c1ccc2nc3c(cc(-c4c5ccccc5c(-c5nc6ccccc6c6c5ccc5c7ccccc7oc56)c5ccccc45)c4ccccc43)cc2c1 ; N#Cc1ccc2c3cc(-c4c5ccccc5c(-c5nc6ccccc6c6c5ccc5c7ccccc7oc56)c5ccccc45)ccc3n(-c3ccccc3)c2c1]

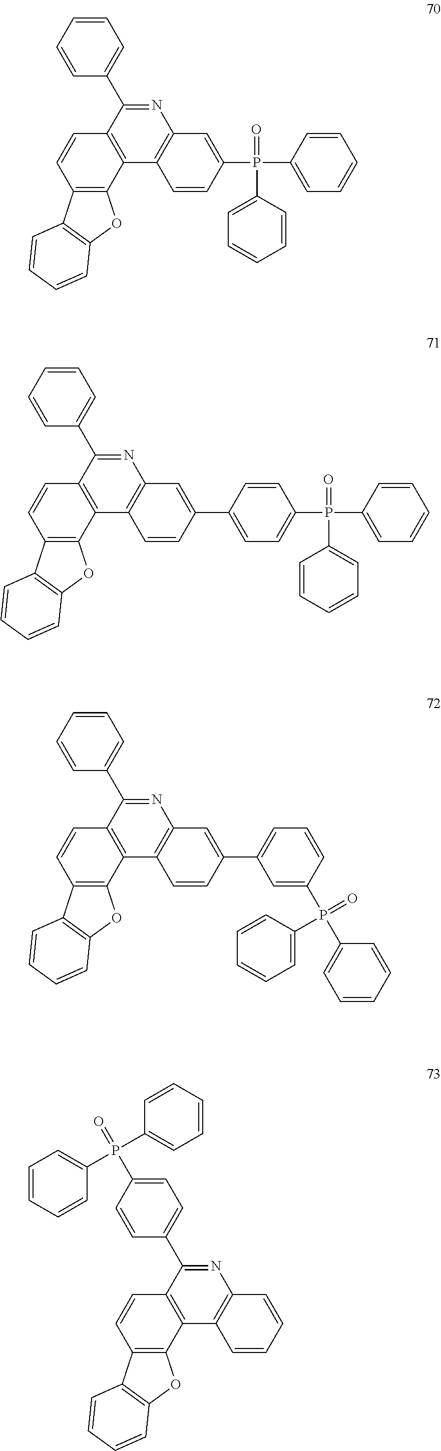 [SMILES: O=P(c1ccccc1)(c1ccccc1)c1ccc2c(c1)nc(-c1ccccc1)c1ccc3c4ccccc4oc3c12 ; O=P(c1ccccc1)(c1ccccc1)c1ccc(-c2ccc3c(c2)nc(-c2ccccc2)c2ccc4c5ccccc5oc4c23)cc1 ; O=P(c1ccccc1)(c1ccccc1)c1cccc(-c2ccc3c(c2)nc(-c2ccccc2)c2ccc4c5ccccc5oc4c23)c1 ; O=P(c1ccccc1)(c1ccccc1)c1ccc(-c2nc3ccccc3c3c2ccc2c4ccccc4oc23)cc1]

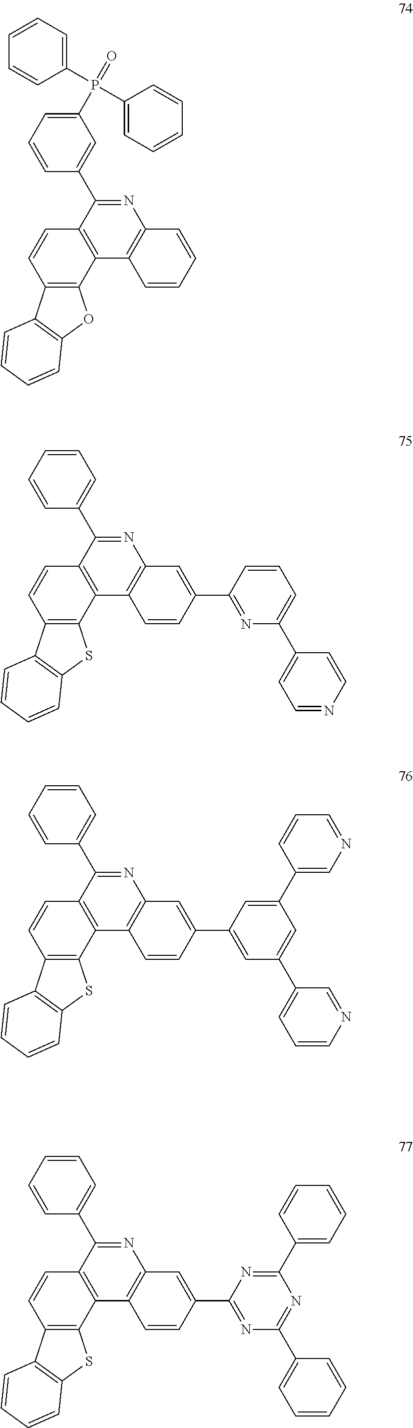 [SMILES: O=P(c1ccccc1)(c1ccccc1)c1cccc(-c2nc3ccccc3c3c2ccc2c4ccccc4oc23)c1 ; c1ccc(-c2nc3cc(-c4cccc(-c5ccncc5)n4)ccc3c3c2ccc2c4ccccc4sc23)cc1 ; c1ccc(-c2nc3cc(-c4cc(-c5cccnc5)cc(-c5cccnc5)c4)ccc3c3c2ccc2c4ccccc4sc23)cc1 ; c1ccc(-c2nc(-c3ccccc3)nc(-c3ccc4c(c3)nc(-c3ccccc3)c3ccc5c6ccccc6sc5c34)n2)cc1]

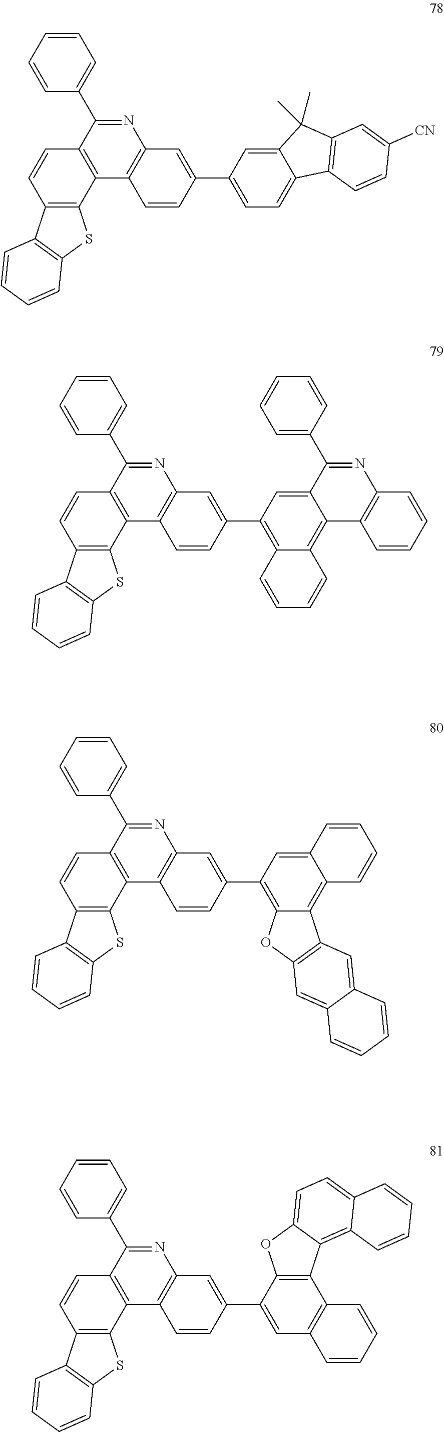 [SMILES: CC1(C)c2cc(C#N)ccc2-c2ccc(-c3ccc4c(c3)nc(-c3ccccc3)c3ccc5c6ccccc6sc5c34)cc21 ; c1ccc(-c2nc3ccccc3c3c2cc(-c2ccc4c(c2)nc(-c2ccccc2)c2ccc5c6ccccc6sc5c24)c2ccccc23)cc1 ; c1ccc(-c2nc3cc(-c4cc5ccccc5c5c4oc4cc6ccccc6cc45)ccc3c3c2ccc2c4ccccc4sc23)cc1 ; c1ccc(-c2nc3cc(-c4cc5ccccc5c5c4oc4ccc6ccccc6c45)ccc3c3c2ccc2c4ccccc4sc23)cc1]

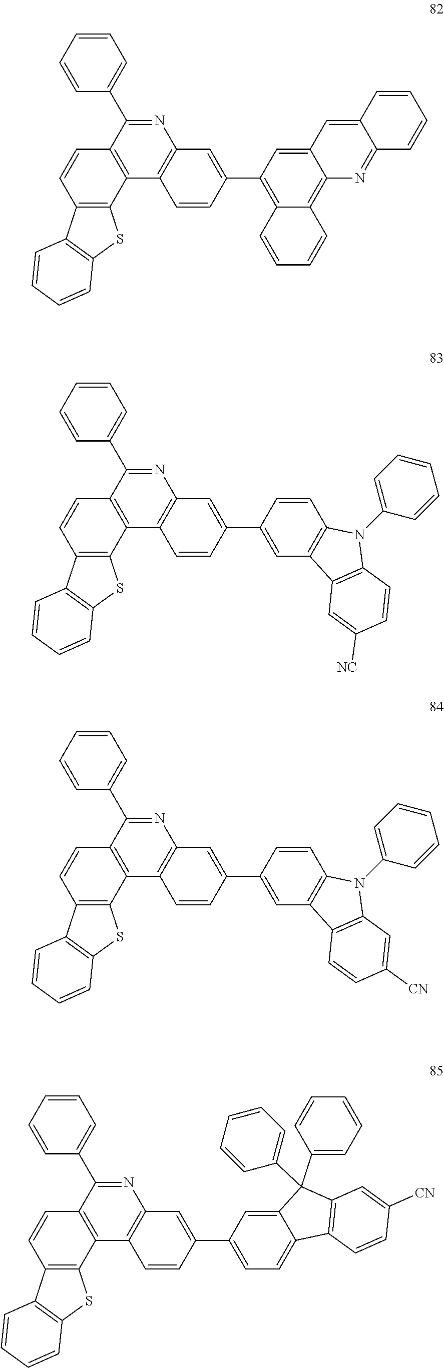 [SMILES: c1ccc(-c2nc3cc(-c4cc5cc6ccccc6nc5c5ccccc45)ccc3c3c2ccc2c4ccccc4sc23)cc1 ; N#Cc1ccc2c(c1)c1cc(-c3ccc4c(c3)nc(-c3ccccc3)c3ccc5c6ccccc6sc5c34)ccc1n2-c1ccccc1 ; N#Cc1ccc2c3cc(-c4ccc5c(c4)nc(-c4ccccc4)c4ccc6c7ccccc7sc6c45)ccc3n(-c3ccccc3)c2c1 ; N#Cc1ccc2c(c1)C(c1ccccc1)(c1ccccc1)c1cc(-c3ccc4c(c3)nc(-c3ccccc3)c3ccc5c6ccccc6sc5c34)ccc1-2]

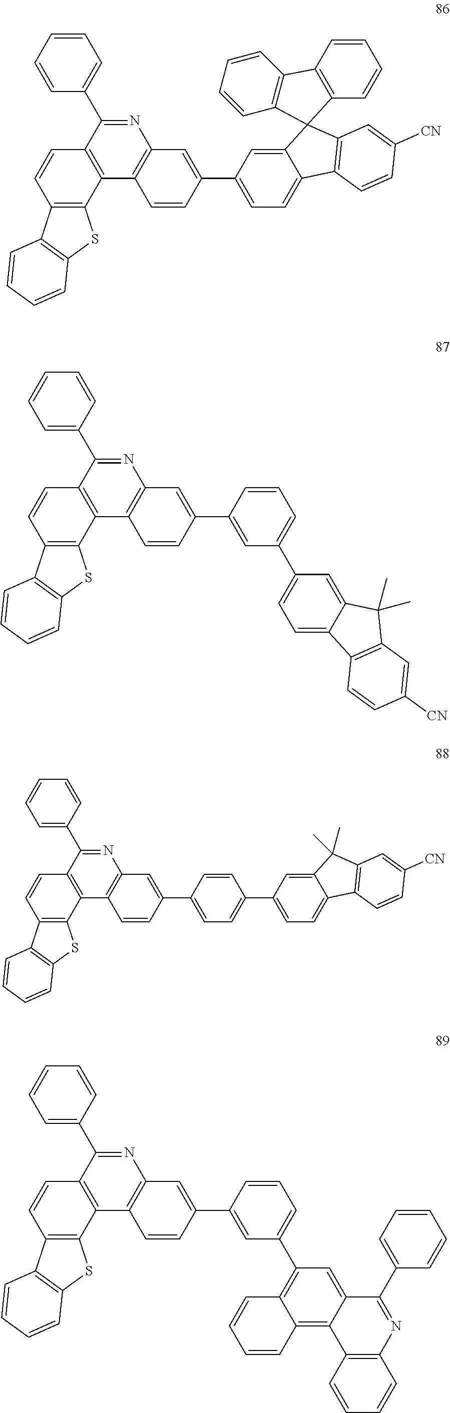 [SMILES: N#Cc1ccc2c(c1)C1(c3ccccc3-c3ccccc31)c1cc(-c3ccc4c(c3)nc(-c3ccccc3)c3ccc5c6ccccc6sc5c34)ccc1-2 ; CC1(C)c2cc(C#N)ccc2-c2ccc(-c3cccc(-c4ccc5c(c4)nc(-c4ccccc4)c4ccc6c7ccccc7sc6c45)c3)cc21 ; CC1(C)c2cc(C#N)ccc2-c2ccc(-c3ccc(-c4ccc5c(c4)nc(-c4ccccc4)c4ccc6c7ccccc7sc6c45)cc3)cc21 ; c1ccc(-c2nc3ccccc3c3c2cc(-c2cccc(-c4ccc5c(c4)nc(-c4ccccc4)c4ccc6c7ccccc7sc6c45)c2)c2ccccc23)cc1]

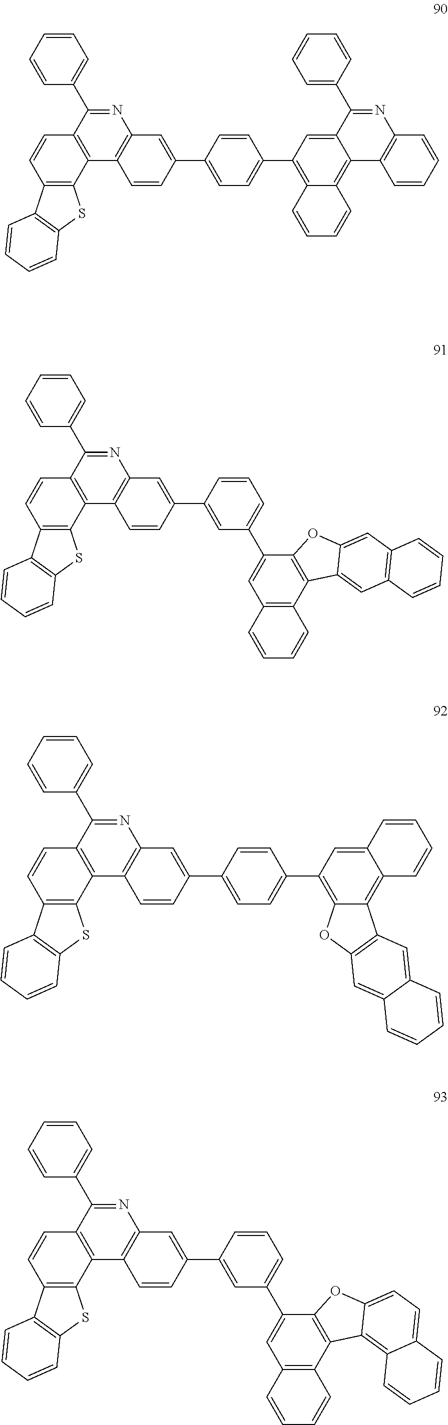 [SMILES: c1ccc(-c2nc3ccccc3c3c2cc(-c2ccc(-c4ccc5c(c4)nc(-c4ccccc4)c4ccc6c7ccccc7sc6c45)cc2)c2ccccc23)cc1 ; c1ccc(-c2nc3cc(-c4cccc(-c5cc6ccccc6c6c5oc5cc7ccccc7cc56)c4)ccc3c3c2ccc2c4ccccc4sc23)cc1 ; c1ccc(-c2nc3cc(-c4ccc(-c5cc6ccccc6c6c5oc5cc7ccccc7cc56)cc4)ccc3c3c2ccc2c4ccccc4sc23)cc1 ; c1ccc(-c2nc3cc(-c4cccc(-c5cc6ccccc6c6c5oc5ccc7ccccc7c56)c4)ccc3c3c2ccc2c4ccccc4sc23)cc1]

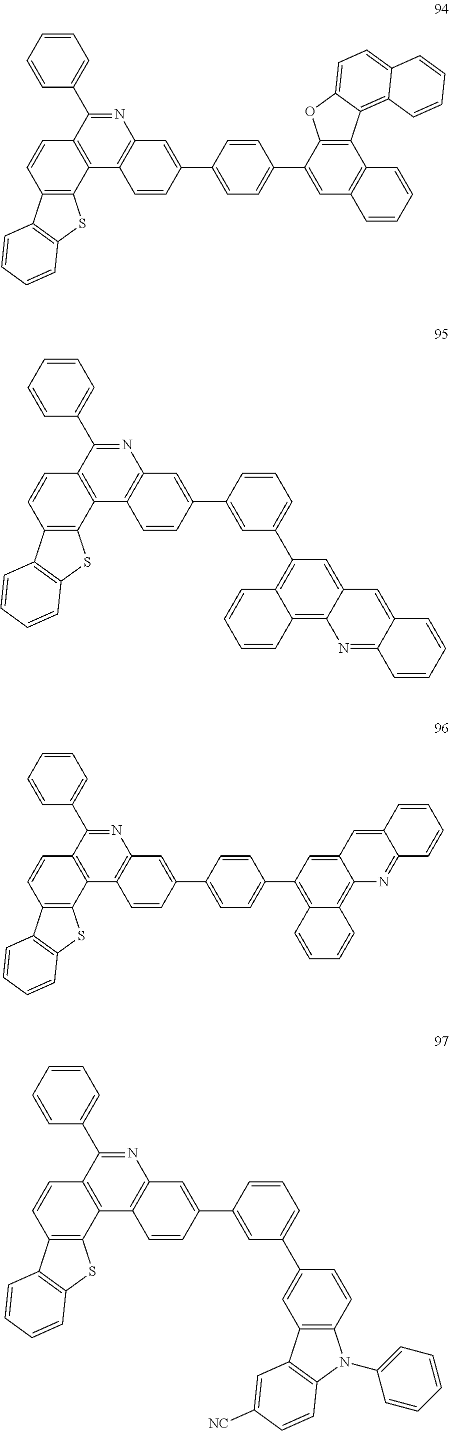 [SMILES: c1ccc(-c2nc3cc(-c4ccc(-c5cc6ccccc6c6c5oc5ccc7ccccc7c56)cc4)ccc3c3c2ccc2c4ccccc4sc23)cc1 ; c1ccc(-c2nc3cc(-c4cccc(-c5cc6cc7ccccc7nc6c6ccccc56)c4)ccc3c3c2ccc2c4ccccc4sc23)cc1 ; c1ccc(-c2nc3cc(-c4ccc(-c5cc6cc7ccccc7nc6c6ccccc56)cc4)ccc3c3c2ccc2c4ccccc4sc23)cc1 ; N#Cc1ccc2c(c1)c1cc(-c3cccc(-c4ccc5c(c4)nc(-c4ccccc4)c4ccc6c7ccccc7sc6c45)c3)ccc1n2-c1ccccc1]

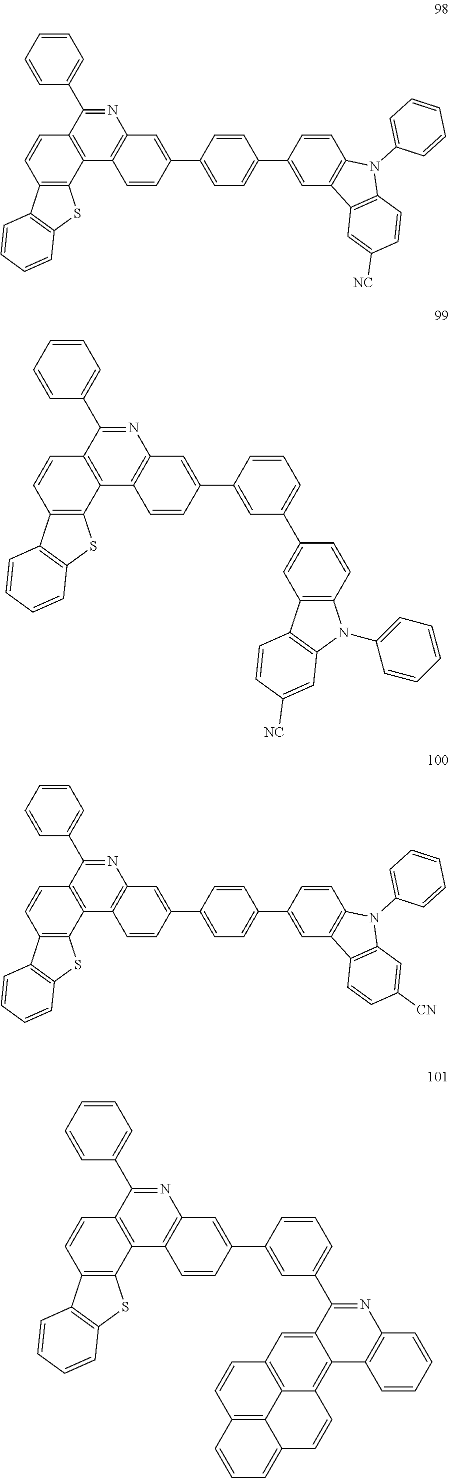 [SMILES: N#Cc1ccc2c(c1)c1cc(-c3ccc(-c4ccc5c(c4)nc(-c4ccccc4)c4ccc6c7ccccc7sc6c45)cc3)ccc1n2-c1ccccc1 ; N#Cc1ccc2c3cc(-c4cccc(-c5ccc6c(c5)nc(-c5ccccc5)c5ccc7c8ccccc8sc7c56)c4)ccc3n(-c3ccccc3)c2c1 ; N#Cc1ccc2c3cc(-c4ccc(-c5ccc6c(c5)nc(-c5ccccc5)c5ccc7c8ccccc8sc7c56)cc4)ccc3n(-c3ccccc3)c2c1 ; c1ccc(-c2nc3cc(-c4cccc(-c5nc6ccccc6c6c5cc5ccc7cccc8ccc6c5c78)c4)ccc3c3c2ccc2c4ccccc4sc23)cc1]

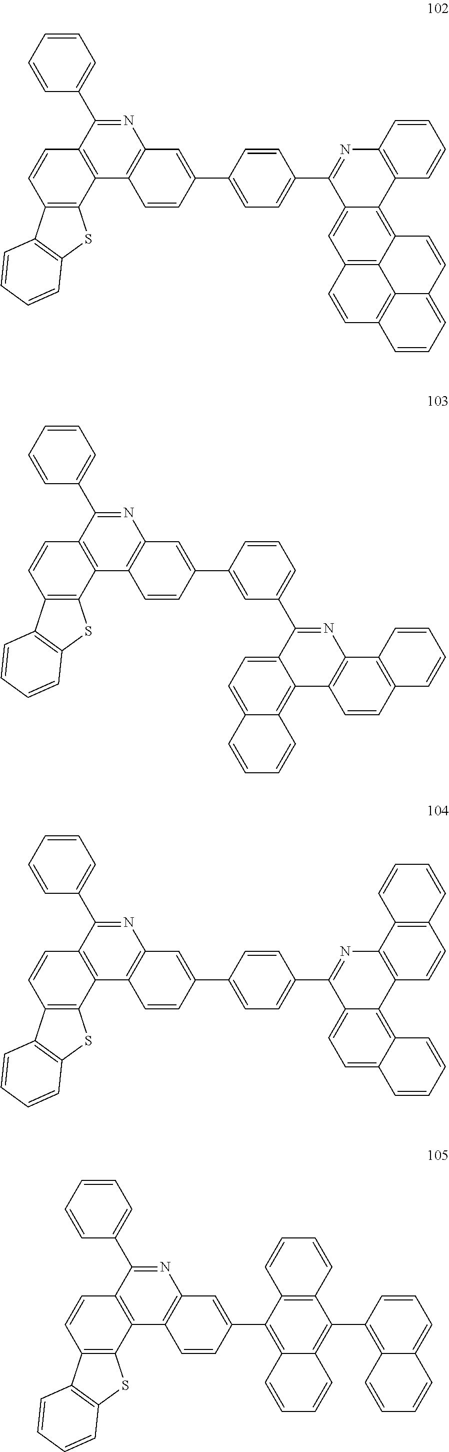 [SMILES: c1ccc(-c2nc3cc(-c4ccc(-c5nc6ccccc6c6c5cc5ccc7cccc8ccc6c5c78)cc4)ccc3c3c2ccc2c4ccccc4sc23)cc1 ; c1ccc(-c2nc3cc(-c4cccc(-c5nc6c7ccccc7ccc6c6c5ccc5ccccc56)c4)ccc3c3c2ccc2c4ccccc4sc23)cc1 ; c1ccc(-c2nc3cc(-c4ccc(-c5nc6c7ccccc7ccc6c6c5ccc5ccccc56)cc4)ccc3c3c2ccc2c4ccccc4sc23)cc1 ; c1ccc(-c2nc3cc(-c4c5ccccc5c(-c5cccc6ccccc56)c5ccccc45)ccc3c3c2ccc2c4ccccc4sc23)cc1]

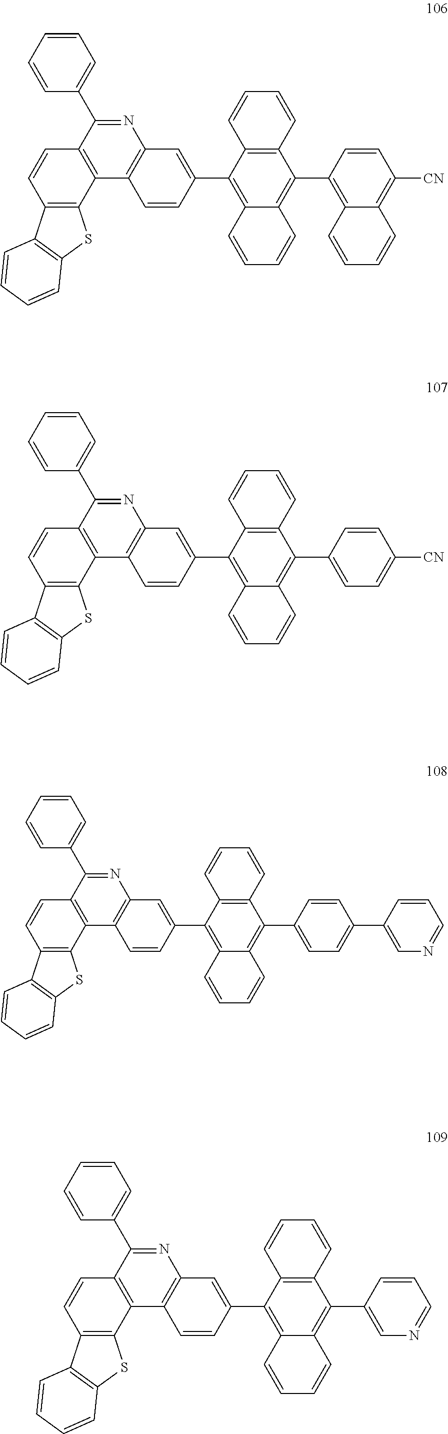 [SMILES: N#Cc1ccc(-c2c3ccccc3c(-c3ccc4c(c3)nc(-c3ccccc3)c3ccc5c6ccccc6sc5c34)c3ccccc23)c2ccccc12 ; N#Cc1ccc(-c2c3ccccc3c(-c3ccc4c(c3)nc(-c3ccccc3)c3ccc5c6ccccc6sc5c34)c3ccccc23)cc1 ; c1ccc(-c2nc3cc(-c4c5ccccc5c(-c5ccc(-c6cccnc6)cc5)c5ccccc45)ccc3c3c2ccc2c4ccccc4sc23)cc1 ; c1ccc(-c2nc3cc(-c4c5ccccc5c(-c5cccnc5)c5ccccc45)ccc3c3c2ccc2c4ccccc4sc23)cc1]

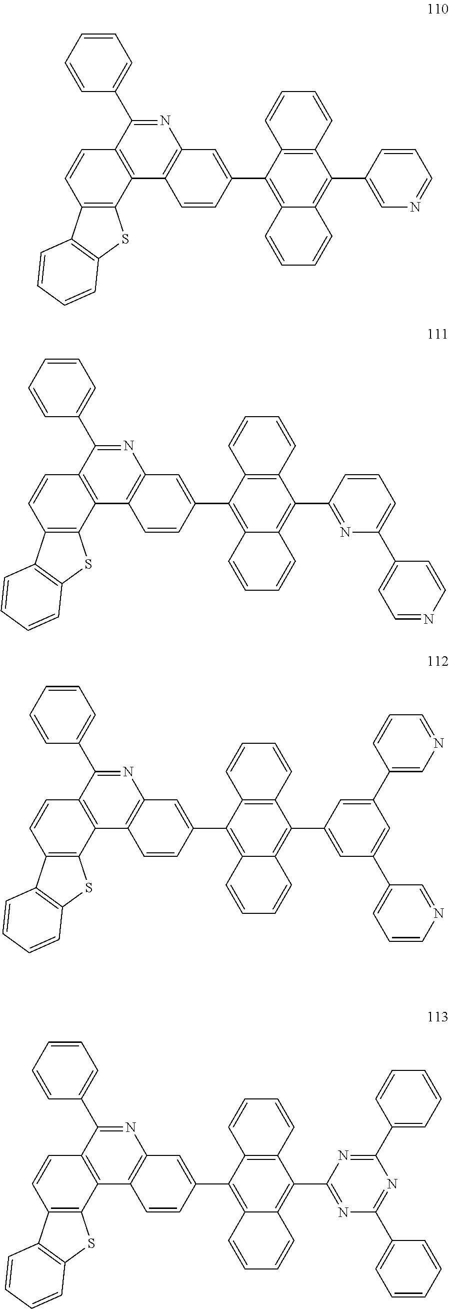 [SMILES: c1ccc(-c2nc3cc(-c4c5ccccc5c(-c5cccnc5)c5ccccc45)ccc3c3c2ccc2c4ccccc4sc23)cc1 ; c1ccc(-c2nc3cc(-c4c5ccccc5c(-c5cccc(-c6ccncc6)n5)c5ccccc45)ccc3c3c2ccc2c4ccccc4sc23)cc1 ; c1ccc(-c2nc3cc(-c4c5ccccc5c(-c5cc(-c6cccnc6)cc(-c6cccnc6)c5)c5ccccc45)ccc3c3c2ccc2c4ccccc4sc23)cc1 ; c1ccc(-c2nc(-c3ccccc3)nc(-c3c4ccccc4c(-c4ccc5c(c4)nc(-c4ccccc4)c4ccc6c7ccccc7sc6c45)c4ccccc34)n2)cc1]

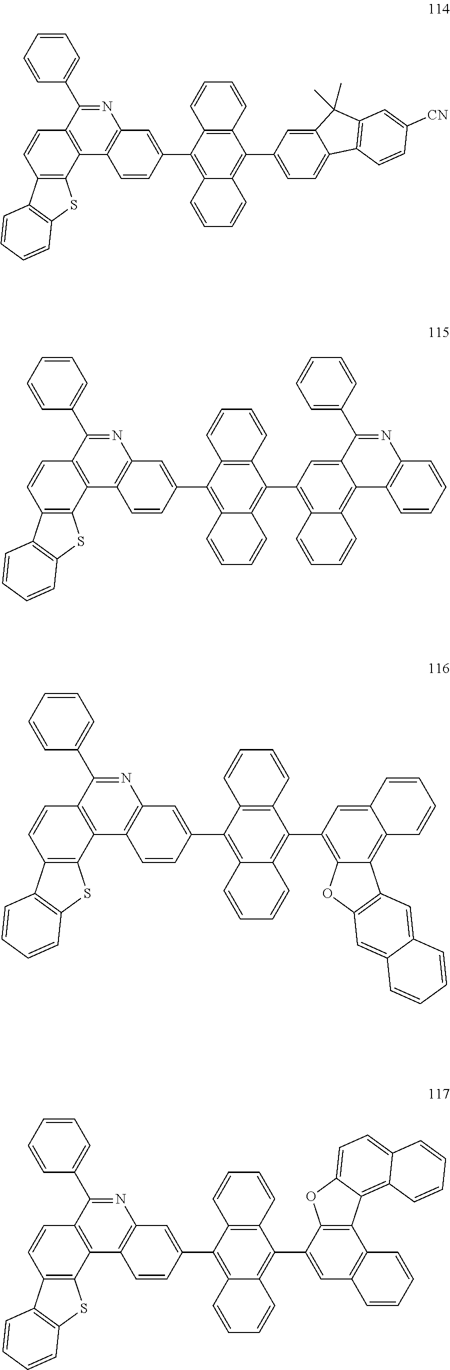 [SMILES: CC1(C)c2cc(C#N)ccc2-c2ccc(-c3c4ccccc4c(-c4ccc5c(c4)nc(-c4ccccc4)c4ccc6c7ccccc7sc6c45)c4ccccc34)cc21 ; c1ccc(-c2nc3ccccc3c3c2cc(-c2c4ccccc4c(-c4ccc5c(c4)nc(-c4ccccc4)c4ccc6c7ccccc7sc6c45)c4ccccc24)c2ccccc23)cc1 ; c1ccc(-c2nc3cc(-c4c5ccccc5c(-c5cc6ccccc6c6c5oc5cc7ccccc7cc56)c5ccccc45)ccc3c3c2ccc2c4ccccc4sc23)cc1 ; c1ccc(-c2nc3cc(-c4c5ccccc5c(-c5cc6ccccc6c6c5oc5ccc7ccccc7c56)c5ccccc45)ccc3c3c2ccc2c4ccccc4sc23)cc1]

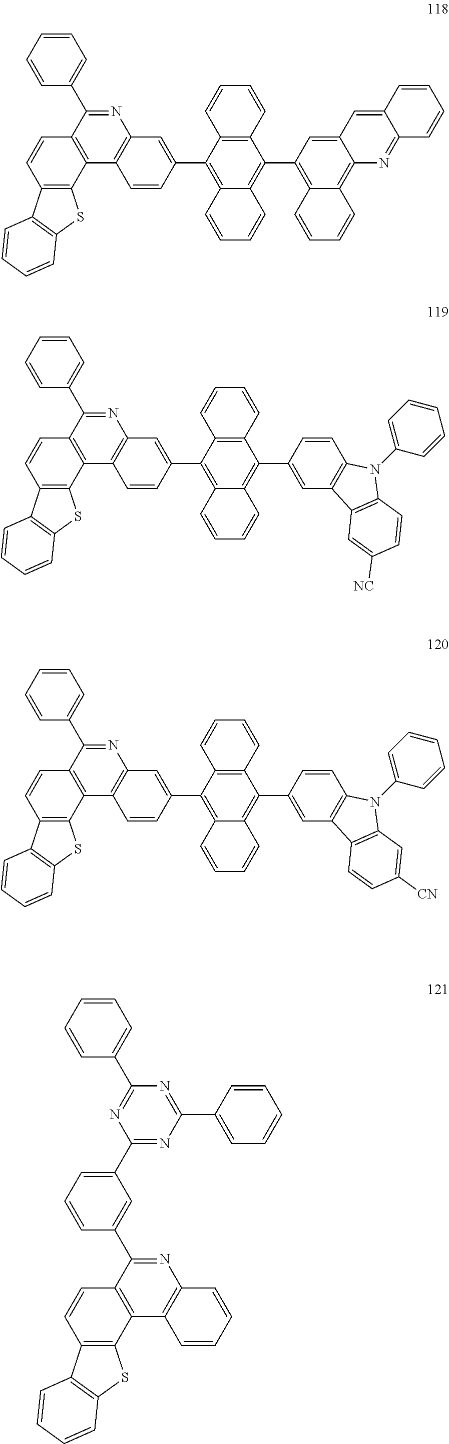 [SMILES: c1ccc(-c2nc3cc(-c4c5ccccc5c(-c5cc6cc7ccccc7nc6c6ccccc56)c5ccccc45)ccc3c3c2ccc2c4ccccc4sc23)cc1 ; N#Cc1ccc2c(c1)c1cc(-c3c4ccccc4c(-c4ccc5c(c4)nc(-c4ccccc4)c4ccc6c7ccccc7sc6c45)c4ccccc34)ccc1n2-c1ccccc1 ; N#Cc1ccc2c3cc(-c4c5ccccc5c(-c5ccc6c(c5)nc(-c5ccccc5)c5ccc7c8ccccc8sc7c56)c5ccccc45)ccc3n(-c3ccccc3)c2c1 ; c1ccc(-c2nc(-c3ccccc3)nc(-c3cccc(-c4nc5ccccc5c5c4ccc4c6ccccc6sc45)c3)n2)cc1]

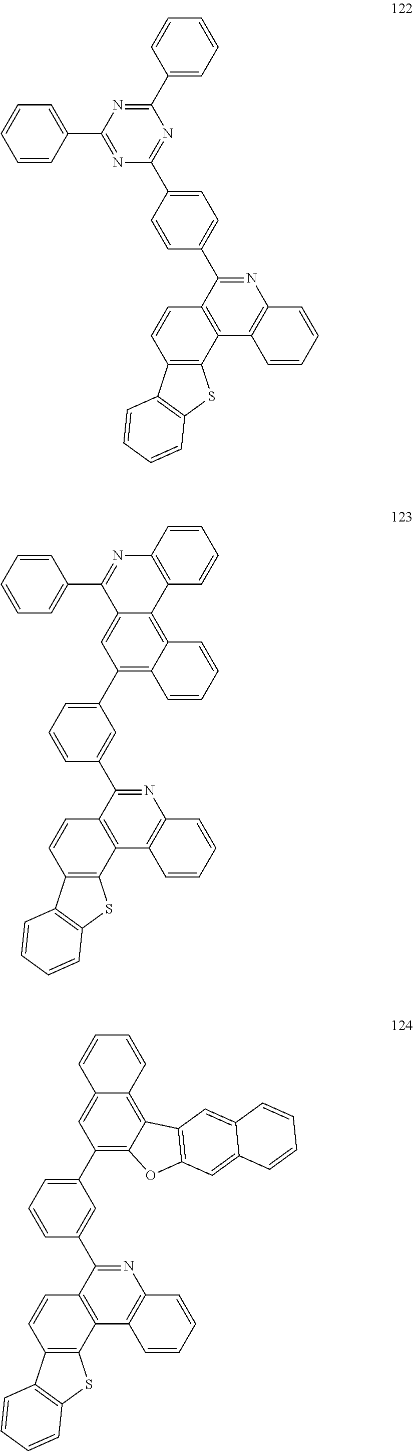 [SMILES: c1ccc(-c2nc(-c3ccccc3)nc(-c3ccc(-c4nc5ccccc5c5c4ccc4c6ccccc6sc45)cc3)n2)cc1 ; c1ccc(-c2nc3ccccc3c3c2cc(-c2cccc(-c4nc5ccccc5c5c4ccc4c6ccccc6sc45)c2)c2ccccc23)cc1 ; c1cc(-c2nc3ccccc3c3c2ccc2c4ccccc4sc23)cc(-c2cc3ccccc3c3c2oc2cc4ccccc4cc23)c1]

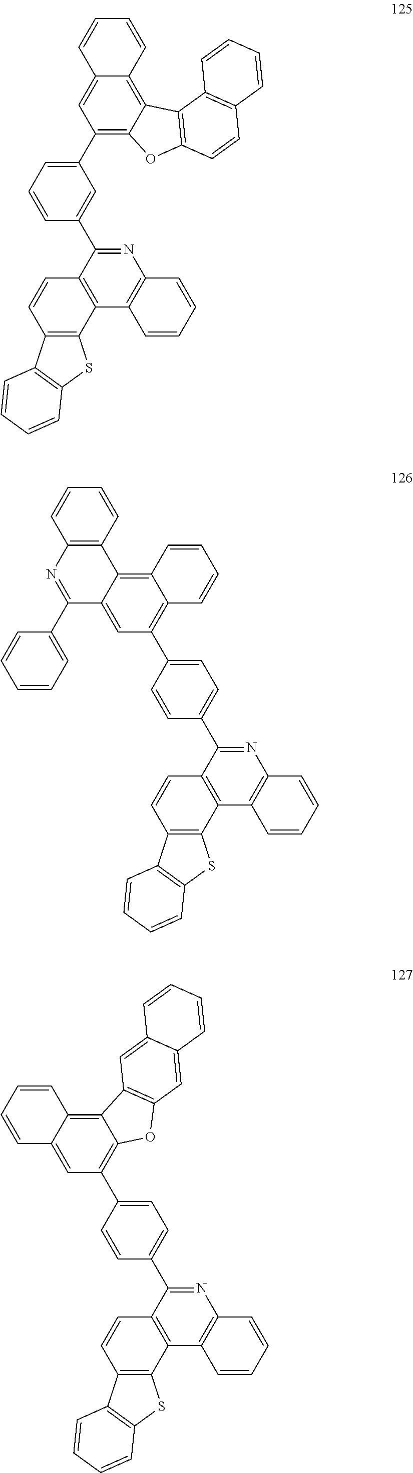 [SMILES: c1cc(-c2nc3ccccc3c3c2ccc2c4ccccc4sc23)cc(-c2cc3ccccc3c3c2oc2ccc4ccccc4c23)c1 ; c1ccc(-c2nc3ccccc3c3c2cc(-c2ccc(-c4nc5ccccc5c5c4ccc4c6ccccc6sc45)cc2)c2ccccc23)cc1 ; c1ccc2cc3c(cc2c1)oc1c(-c2ccc(-c4nc5ccccc5c5c4ccc4c6ccccc6sc45)cc2)cc2ccccc2c13]

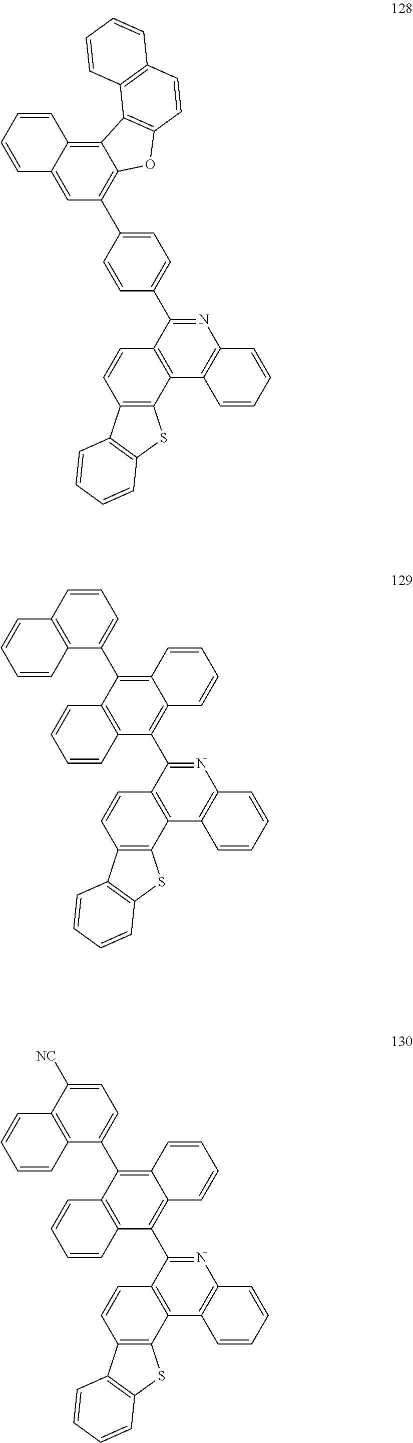 [SMILES: c1ccc2c(c1)ccc1oc3c(-c4ccc(-c5nc6ccccc6c6c5ccc5c7ccccc7sc56)cc4)cc4ccccc4c3c12 ; c1ccc2c(-c3c4ccccc4c(-c4nc5ccccc5c5c4ccc4c6ccccc6sc45)c4ccccc34)cccc2c1 ; N#Cc1ccc(-c2c3ccccc3c(-c3nc4ccccc4c4c3ccc3c5ccccc5sc34)c3ccccc23)c2ccccc12]

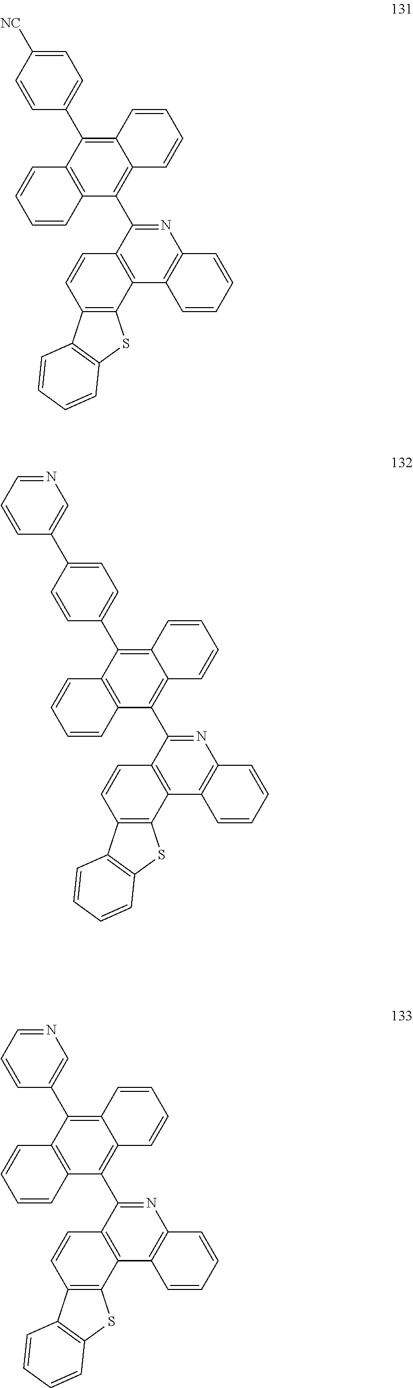 [SMILES: N#Cc1ccc(-c2c3ccccc3c(-c3nc4ccccc4c4c3ccc3c5ccccc5sc34)c3ccccc23)cc1 ; c1cncc(-c2ccc(-c3c4ccccc4c(-c4nc5ccccc5c5c4ccc4c6ccccc6sc45)c4ccccc34)cc2)c1 ; c1cncc(-c2c3ccccc3c(-c3nc4ccccc4c4c3ccc3c5ccccc5sc34)c3ccccc23)c1]

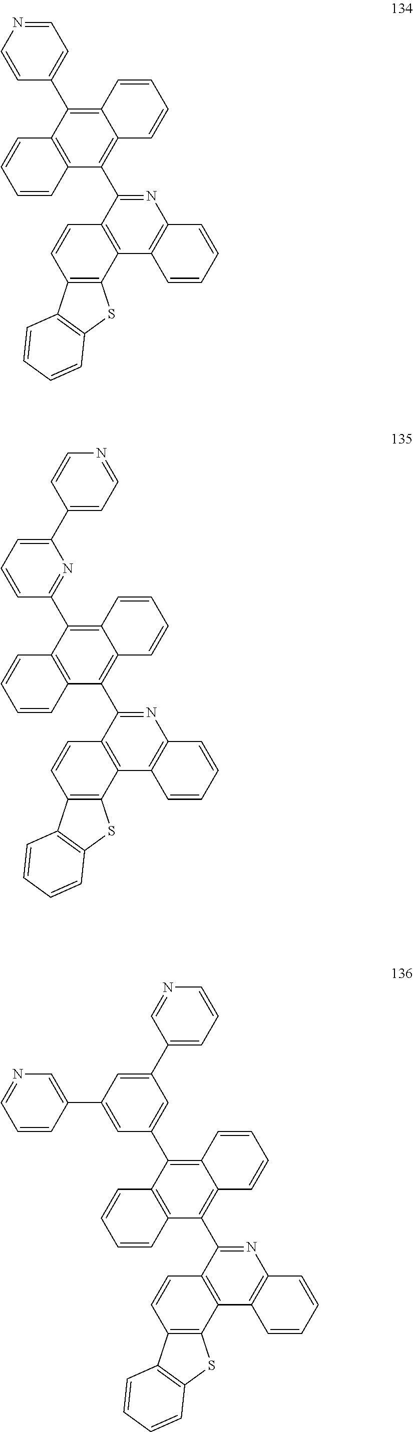 [SMILES: c1ccc2c(c1)nc(-c1c3ccccc3c(-c3ccncc3)c3ccccc13)c1ccc3c4ccccc4sc3c12 ; c1cc(-c2ccncc2)nc(-c2c3ccccc3c(-c3nc4ccccc4c4c3ccc3c5ccccc5sc34)c3ccccc23)c1 ; c1cncc(-c2cc(-c3cccnc3)cc(-c3c4ccccc4c(-c4nc5ccccc5c5c4ccc4c6ccccc6sc45)c4ccccc34)c2)c1]

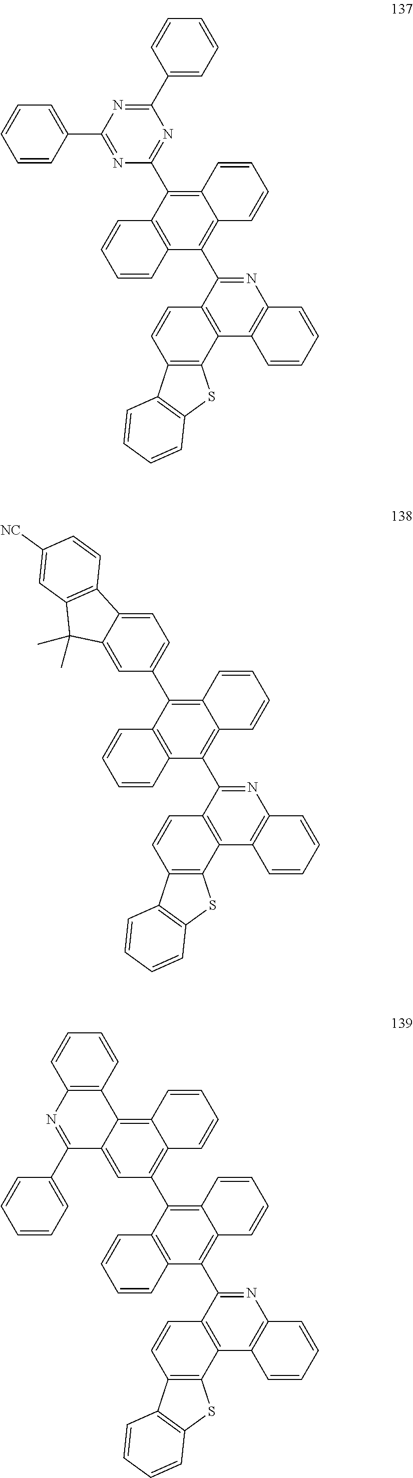 [SMILES: c1ccc(-c2nc(-c3ccccc3)nc(-c3c4ccccc4c(-c4nc5ccccc5c5c4ccc4c6ccccc6sc45)c4ccccc34)n2)cc1 ; CC1(C)c2cc(C#N)ccc2-c2ccc(-c3c4ccccc4c(-c4nc5ccccc5c5c4ccc4c6ccccc6sc45)c4ccccc34)cc21 ; c1ccc(-c2nc3ccccc3c3c2cc(-c2c4ccccc4c(-c4nc5ccccc5c5c4ccc4c6ccccc6sc45)c4ccccc24)c2ccccc23)cc1]

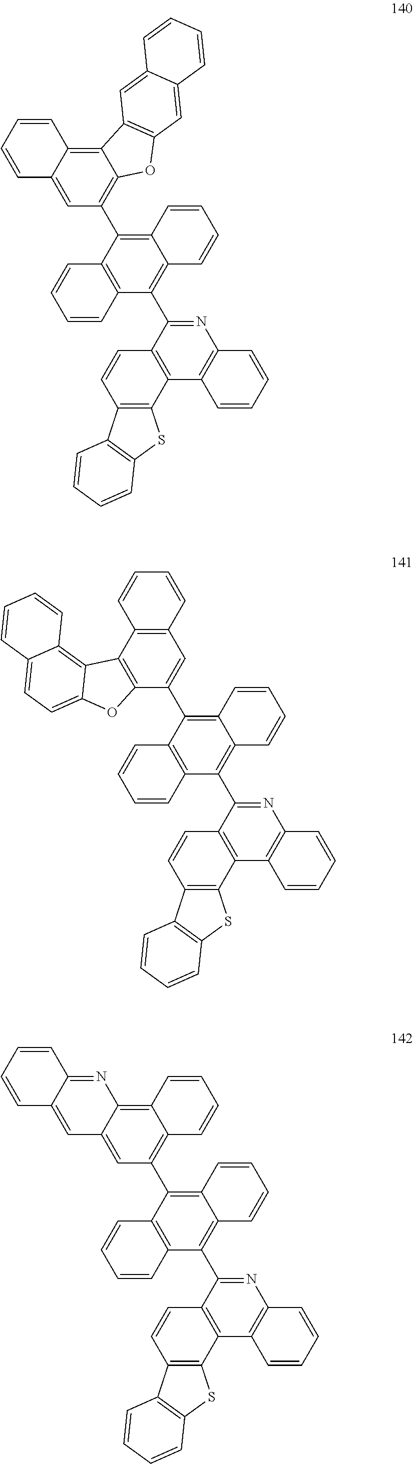 [SMILES: c1ccc2cc3c(cc2c1)oc1c(-c2c4ccccc4c(-c4nc5ccccc5c5c4ccc4c6ccccc6sc45)c4ccccc24)cc2ccccc2c13 ; c1ccc2c(c1)ccc1oc3c(-c4c5ccccc5c(-c5nc6ccccc6c6c5ccc5c7ccccc7sc56)c5ccccc45)cc4ccccc4c3c12 ; c1ccc2nc3c(cc(-c4c5ccccc5c(-c5nc6ccccc6c6c5ccc5c7ccccc7sc56)c5ccccc45)c4ccccc43)cc2c1]

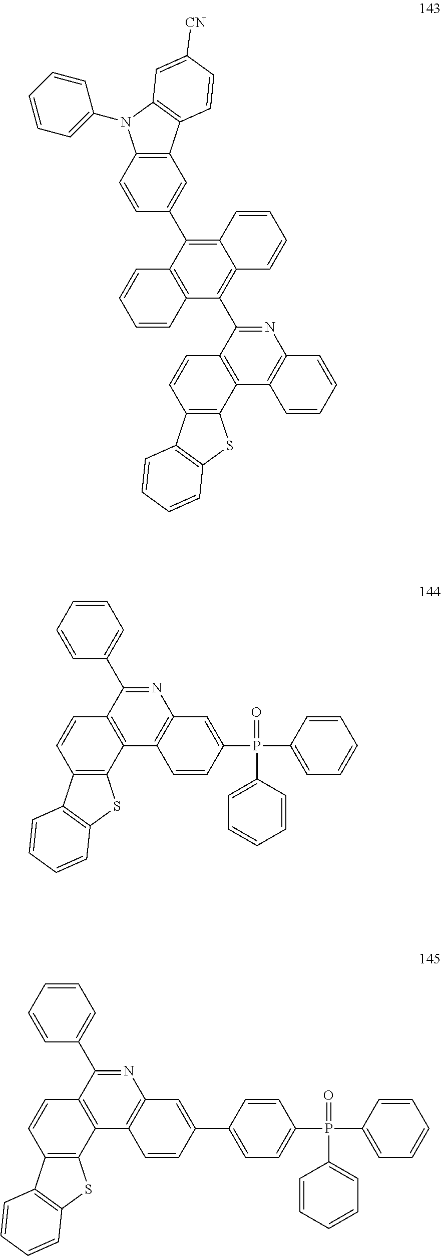 [SMILES: N#Cc1ccc2c3cc(-c4c5ccccc5c(-c5nc6ccccc6c6c5ccc5c7ccccc7sc56)c5ccccc45)ccc3n(-c3ccccc3)c2c1 ; O=P(c1ccccc1)(c1ccccc1)c1ccc2c(c1)nc(-c1ccccc1)c1ccc3c4ccccc4sc3c12 ; O=P(c1ccccc1)(c1ccccc1)c1ccc(-c2ccc3c(c2)nc(-c2ccccc2)c2ccc4c5ccccc5sc4c23)cc1]

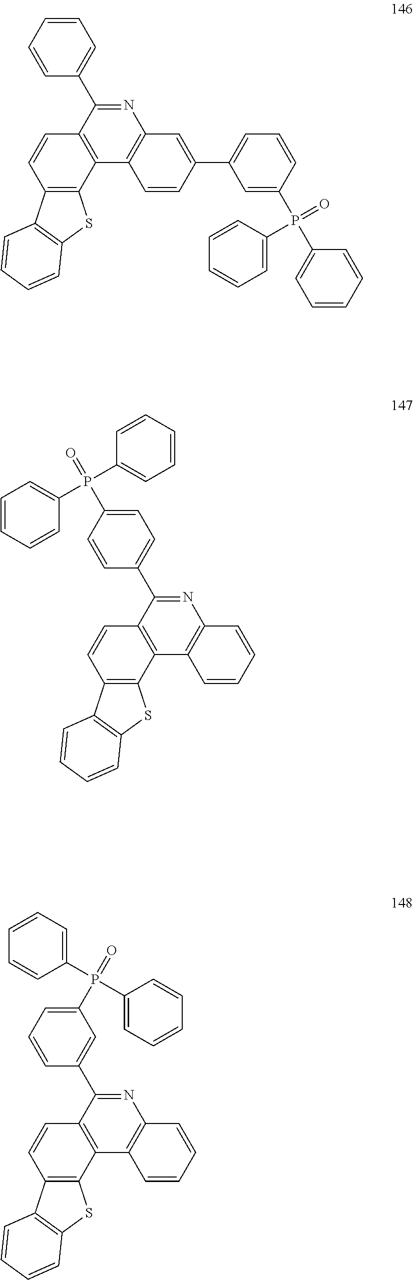 [SMILES: O=P(c1ccccc1)(c1ccccc1)c1cccc(-c2ccc3c(c2)nc(-c2ccccc2)c2ccc4c5ccccc5sc4c23)c1 ; O=P(c1ccccc1)(c1ccccc1)c1ccc(-c2nc3ccccc3c3c2ccc2c4ccccc4sc23)cc1 ; O=P(c1ccccc1)(c1ccccc1)c1cccc(-c2nc3ccccc3c3c2ccc2c4ccccc4sc23)c1]

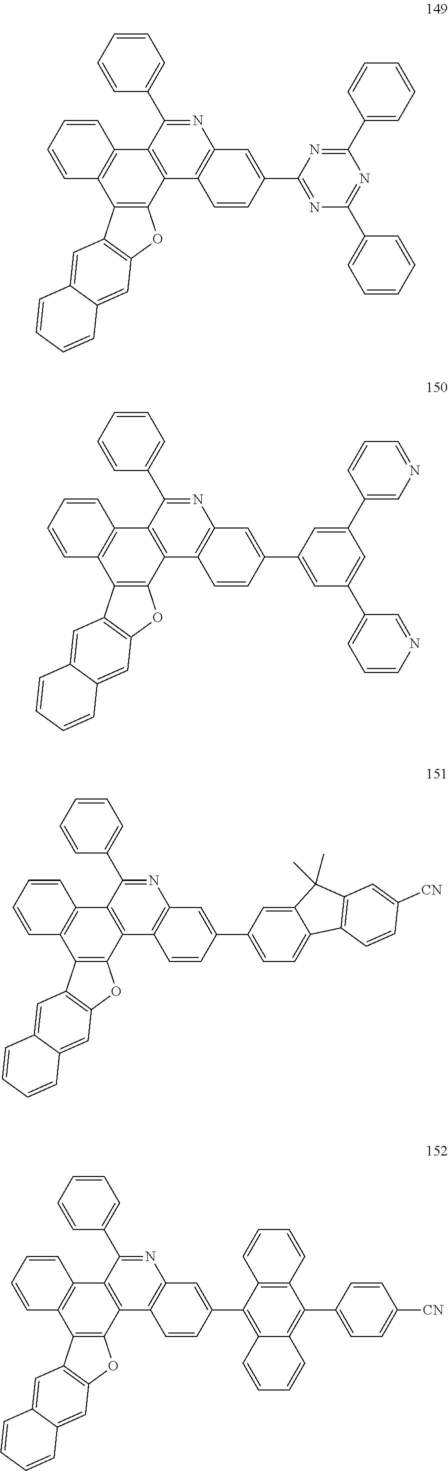 [SMILES: c1ccc(-c2nc(-c3ccccc3)nc(-c3ccc4c(c3)nc(-c3ccccc3)c3c5ccccc5c5c6cc7ccccc7cc6oc5c43)n2)cc1 ; c1ccc(-c2nc3cc(-c4cc(-c5cccnc5)cc(-c5cccnc5)c4)ccc3c3c4oc5cc6ccccc6cc5c4c4ccccc4c23)cc1 ; CC1(C)c2cc(C#N)ccc2-c2ccc(-c3ccc4c(c3)nc(-c3ccccc3)c3c5ccccc5c5c6cc7ccccc7cc6oc5c43)cc21 ; N#Cc1ccc(-c2c3ccccc3c(-c3ccc4c(c3)nc(-c3ccccc3)c3c5ccccc5c5c6cc7ccccc7cc6oc5c43)c3ccccc23)cc1]

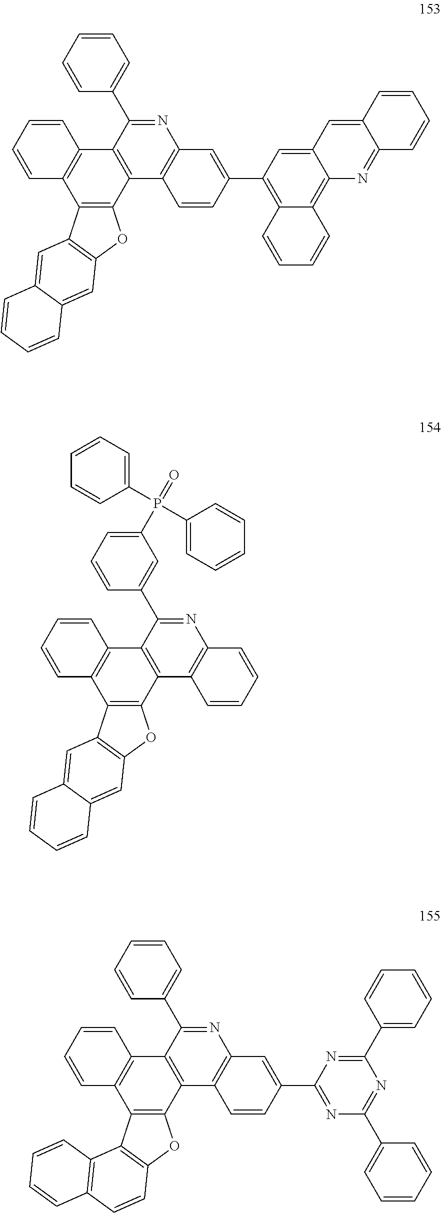 [SMILES: c1ccc(-c2nc3cc(-c4cc5cc6ccccc6nc5c5ccccc45)ccc3c3c4oc5cc6ccccc6cc5c4c4ccccc4c23)cc1 ; O=P(c1ccccc1)(c1ccccc1)c1cccc(-c2nc3ccccc3c3c4oc5cc6ccccc6cc5c4c4ccccc4c23)c1 ; c1ccc(-c2nc(-c3ccccc3)nc(-c3ccc4c(c3)nc(-c3ccccc3)c3c5ccccc5c5c(oc6ccc7ccccc7c65)c43)n2)cc1]

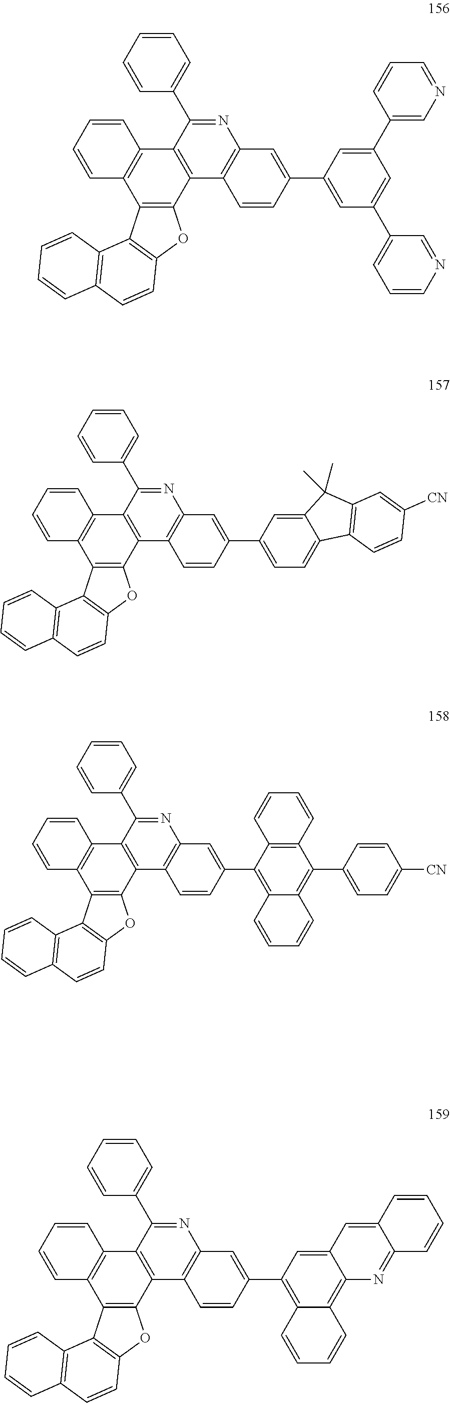 [SMILES: c1ccc(-c2nc3cc(-c4cc(-c5cccnc5)cc(-c5cccnc5)c4)ccc3c3c4oc5ccc6ccccc6c5c4c4ccccc4c23)cc1 ; CC1(C)c2cc(C#N)ccc2-c2ccc(-c3ccc4c(c3)nc(-c3ccccc3)c3c5ccccc5c5c(oc6ccc7ccccc7c65)c43)cc21 ; N#Cc1ccc(-c2c3ccccc3c(-c3ccc4c(c3)nc(-c3ccccc3)c3c5ccccc5c5c(oc6ccc7ccccc7c65)c43)c3ccccc23)cc1 ; c1ccc(-c2nc3cc(-c4cc5cc6ccccc6nc5c5ccccc45)ccc3c3c4oc5ccc6ccccc6c5c4c4ccccc4c23)cc1]

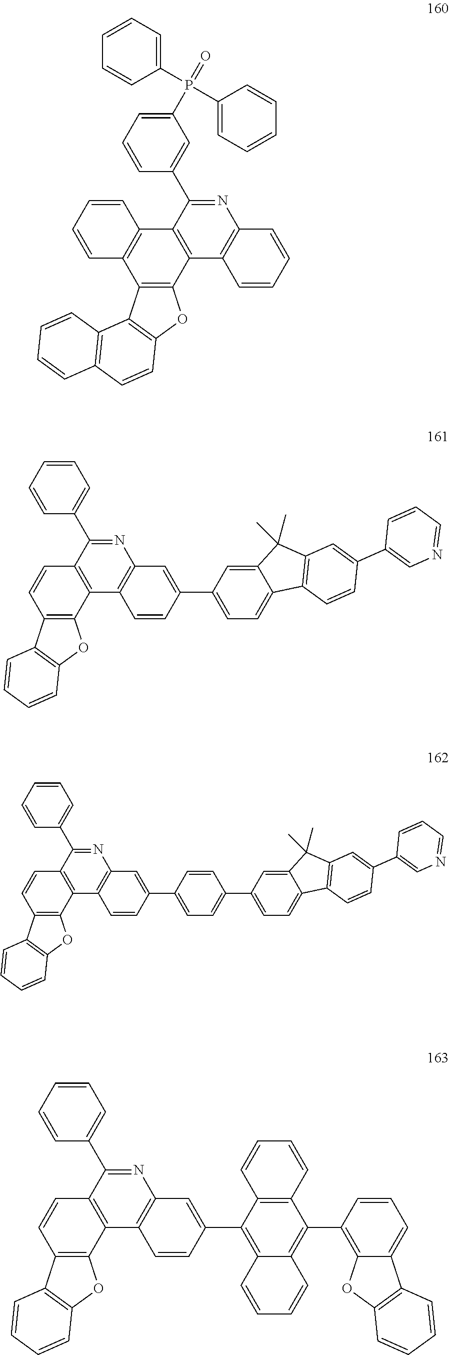 [SMILES: O=P(c1ccccc1)(c1ccccc1)c1cccc(-c2nc3ccccc3c3c4oc5ccc6ccccc6c5c4c4ccccc4c23)c1 ; CC1(C)c2cc(-c3cccnc3)ccc2-c2ccc(-c3ccc4c(c3)nc(-c3ccccc3)c3ccc5c6ccccc6oc5c34)cc21 ; CC1(C)c2cc(-c3ccc(-c4ccc5c(c4)nc(-c4ccccc4)c4ccc6c7ccccc7oc6c45)cc3)ccc2-c2ccc(-c3cccnc3)cc21 ; c1ccc(-c2nc3cc(-c4c5ccccc5c(-c5cccc6c5oc5ccccc56)c5ccccc45)ccc3c3c2ccc2c4ccccc4oc23)cc1]

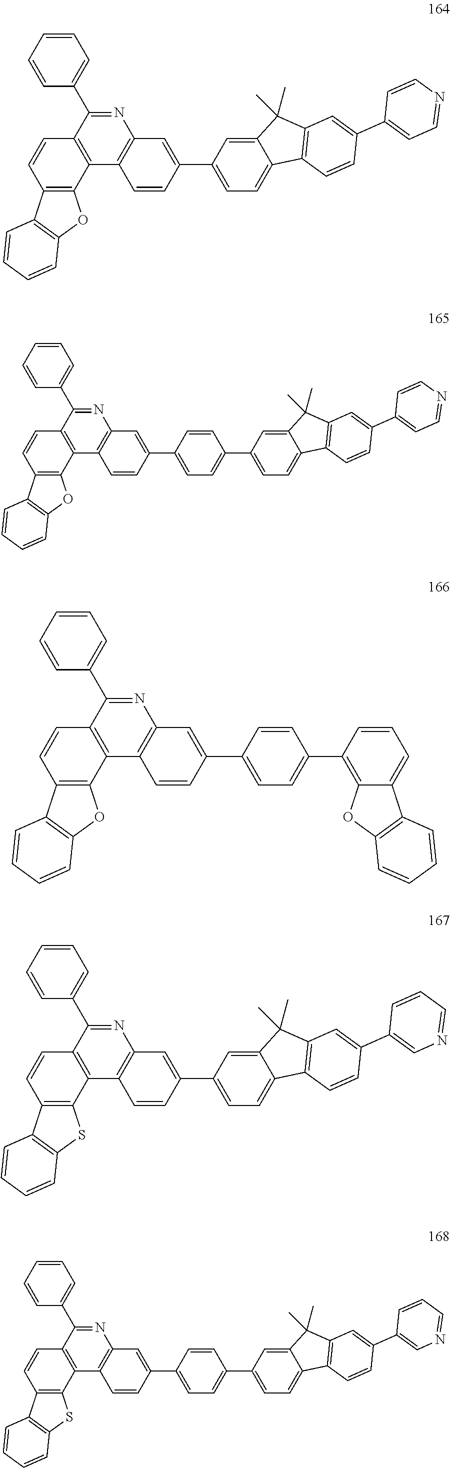 [SMILES: CC1(C)c2cc(-c3ccncc3)ccc2-c2ccc(-c3ccc4c(c3)nc(-c3ccccc3)c3ccc5c6ccccc6oc5c34)cc21 ; CC1(C)c2cc(-c3ccncc3)ccc2-c2ccc(-c3ccc(-c4ccc5c(c4)nc(-c4ccccc4)c4ccc6c7ccccc7oc6c45)cc3)cc21 ; c1ccc(-c2nc3cc(-c4ccc(-c5cccc6c5oc5ccccc56)cc4)ccc3c3c2ccc2c4ccccc4oc23)cc1 ; CC1(C)c2cc(-c3cccnc3)ccc2-c2ccc(-c3ccc4c(c3)nc(-c3ccccc3)c3ccc5c6ccccc6sc5c34)cc21 ; CC1(C)c2cc(-c3ccc(-c4ccc5c(c4)nc(-c4ccccc4)c4ccc6c7ccccc7sc6c45)cc3)ccc2-c2ccc(-c3cccnc3)cc21]

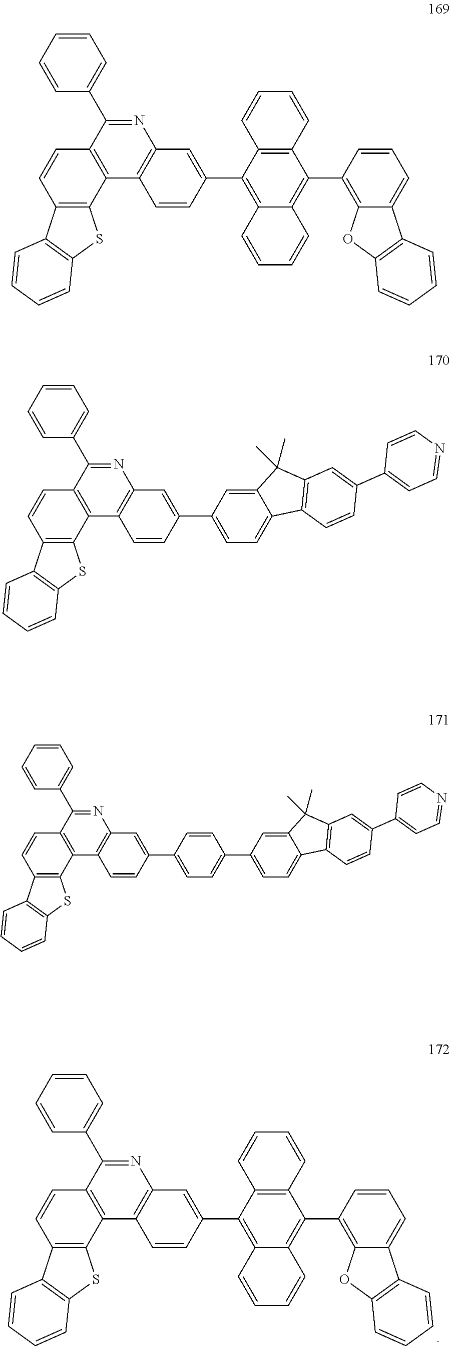 [SMILES: c1ccc(-c2nc3cc(-c4c5ccccc5c(-c5cccc6c5oc5ccccc56)c5ccccc45)ccc3c3c2ccc2c4ccccc4sc23)cc1 ; CC1(C)c2cc(-c3ccncc3)ccc2-c2ccc(-c3ccc4c(c3)nc(-c3ccccc3)c3ccc5c6ccccc6sc5c34)cc21 ; CC1(C)c2cc(-c3ccncc3)ccc2-c2ccc(-c3ccc(-c4ccc5c(c4)nc(-c4ccccc4)c4ccc6c7ccccc7sc6c45)cc3)cc21 ; c1ccc(-c2nc3cc(-c4c5ccccc5c(-c5cccc6c5oc5ccccc56)c5ccccc45)ccc3c3c2ccc2c4ccccc4sc23)cc1]

14. An organic light-emitting device comprising:
a first electrode;
a second electrode facing the first electrode; and
an organic layer between the first electrode and the second electrode, the organic layer comprising an emission layer,
wherein the organic layer comprises the compound of claim 1.

15. The organic light-emitting device of claim 14, wherein
the first electrode is an anode,
the second electrode is a cathode, and
the organic layer comprises:
i) a hole transport region between the first electrode and the emission layer, the hole transport region comprising at least one selected from a hole transport layer, a hole injection layer, and an electron blocking layer, and
ii) an electron transport region between the emission layer and the second electrode, the electron transport region comprising an electron transport layer and at least one selected from a hole blocking layer and an electron injection layer.

16. The organic light-emitting device of claim 15, wherein
the electron transport region comprises the compound.

17. The organic light-emitting device of claim 15, wherein
the electron transport layer comprises the compound.

18. The organic light-emitting device of claim 15, wherein
the hole transport region comprises a charge-generating material.

19. The organic light-emitting device of claim 15, wherein
the electron transport region comprises a metal-containing material.

20. A display apparatus comprising the organic light-emitting device of claim 14, wherein the first electrode of the organic light-emitting device is electrically coupled to a source electrode or a drain electrode of a thin film transistor.
